(19) United States
(12) Patent Application Publication
(10) Pub. No.: US 2024/0335536 A1
(43) Pub. Date: Oct. 10, 2024

(54) METHODS OF ENGINEERING IMMUNE CELLS FOR ENHANCED POTENCY AND PERSISTENCE AND USES OF ENGINEERED CELLS IN IMMUNOTHERAPY

(71) Applicant: Nkarta, Inc., South San Francisco, CA (US)

(72) Inventors: James Barnaby Trager, Albany, CA (US); Ivan Chan, Millbrae, CA (US); Don-Hong Wang, Palo Alto, CA (US); Guangnan Li, Foster City, CA (US); Alexandra Leida Liana Lazetic, San Jose, CA (US); Chao Guo, San Francisco, CA (US)

(21) Appl. No.: 18/255,222

(22) PCT Filed: Dec. 2, 2021
(86) PCT No.: PCT/US2021/072715

§ 371 Date: May 31, 2023

Related U.S. Application Data
(60) Provisional application No. 63/264,234, filed on Nov. 17, 2021.
(60) Provisional application No. 63/264,223, filed on Nov. 17, 2021.
(60) Provisional application No. 63/236,167, filed on Aug. 23, 2021.
(60) Provisional application No. 63/201,159, filed on Apr. 15, 2021.
(60) Provisional application No. 63/121,206, filed on Dec. 3, 2020.

(51) Int. Cl. A61K 39/00; C07K 14/54; C07K 14/725; C07K 14/74; C07K 16/28; C12N 5/0783

(52) U.S. Cl. CPC A61K 39/4634; A61K 39/001112; A61K 39/4611; A61K 39/4613; A61K 39/4631; A61K 39/4635; C07K 14/5443; C07K 14/7051; C07K 14/70539; C07K 16/2803; C12N 5/0636; C12N 5/0646; A61K 2239/26; C12N 2510/00

(57) ABSTRACT

Several embodiments of the methods and compositions disclosed herein relate to immune cells that are engineered to Mexpress chimeric antigen receptors as well as genetically edited or otherwise engineered to alter the effectiveness with which another immune cell can target and induce cytotoxic effects on the engineered cells. In several embodiments, the methods and compositions disclosed herein reduce fratricide within a therapeutic cell population, which in some instances comprises mixture of immune cell types. Additionally, the methods and compositions disclosed herein enhance one or more aspects of the efficacy of the immune cells in cellular immunotherapy including cytotoxicity and/or persistence, as well as reduced graft versus host or host versus graft effects.

### RELATED APPLICATIONS

This application claims priority to U.S. Provisional Patent Application No. 63/121,206, filed Dec. 3, 2020, U.S. Provisional Patent Application No. 63/201,159, filed Apr. 15, 2021, U.S. Provisional Patent Application No. 63/236,167, filed Aug. 23, 2021, U.S. Provisional Patent Application No. 63/264,234, filed Nov. 17, 2021, and U.S. Provisional Patent Application No. 63/264,223, filed Nov. 17, 2021, the entire contents of each of which is incorporated by reference herein.

### FIELD

Several embodiments disclosed herein relate to methods and compositions comprising genetically engineered cells for cancer immunotherapy, in particular combinations of engineered immune cell types. In several embodiments, the present disclosure relates to cells engineered to express chimeric antigen receptors. In several embodiments, further engineering is performed to enhance the efficacy and/or reduce potential side effects when the cells are used in cancer immunotherapy.

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered immune cells are one approach to treating cancers.

### INCORPORATION BY REFERENCE OF MATERIAL IN SEQUENCE LISTING FILE

This application incorporates by reference the Sequence Listing contained in the following ASCII text file being submitted concurrently herewith: File name: NKT068NP_ST25_update.txt; created Mar. 6, 2024, which is 2,468,748 bytes in size.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric receptors in immune cells and further engineering or genetically editing the cells to avoid adverse immune responses against the therapeutic cells in order to achieve the efficient and persistent targeted recognition and destruction of aberrant cells of interest.

In several embodiments, there is provided a population of genetically engineered and gene edited immune cells, comprising genetically engineered immune cells that express a cytotoxic receptor comprising that targets a tumor marker expressed by a target tumor cell, wherein the immune cells are genetically edited at one or more target locations in a CISH gene that encodes a CIS protein, wherein the edits yield reduced expression and/or function of CIS as compared to an immune cell not edited at the location or locations in the CISH gene, wherein the immune cells are genetically edited at one or more target locations in an additional gene encoding an additional protein, wherein the edits to the additional gene yield reduced expression and/or function of the additional protein as compared to an immune cell not edited at the location or locations in the additional gene; and wherein the genetically engineered and edited immune cells exhibit one or more of enhanced expansion capability, enhanced cytotoxicity against target cells, enhanced persistence, or other beneficial characteristic, as compared to immune cells that do not comprise said genetic edits at the CISH and additional genes.

In several embodiments, the cells are edited at an additional target site in a Cbl proto-oncogene B protein (CBLB) gene. In several embodiments, the cells are edited at an additional target site in a transforming growth factor receptor beta 2 (TGFBR2) gene. In several embodiments, cells are edited at an additional target site in a TIGIT gene. Combinations of edits are provided for as well, such as cells edited at CISH/CBLB/TGFBR2, CISH/CBLB/TIGIT, CISH/TGFBR2/TIGIT, and the like. In several embodiments, a guide sequence of any of SEQ ID NO: 153-157 or 562-565 is used to target the CISH gene. In several embodiments, a guide sequence of any of SEQ ID NO: 164 to 166 or 552-555 is used to target the CBLB gene. In several embodiments, a guide sequence of any of SEQ ID NO: 147 to 152 or 544-547 is used to target the TGFBR2 gene. In several embodiments, a guide sequence of any of SEQ ID NO: 507-510 is used to target the TIGIT gene. In several embodiments, the cells are further edited at one or more additional target sites in a gene encoding ADARA2, SMAD3, MAPKAPK3, CEACAMI, DDIT4, NKG2A, SOCS2, B2M, PD-1, TIM-3, CD38, or TCR alpha.

For example, in several embodiments, the cells are further edited at one or more of: (i) a gene encoding NKG2A and wherein a guide sequence of any of SEQ ID NO: 548-551 is used to target the NKG2A gene, (ii) a gene encoding SOCS2 and wherein a guide sequence of any of SEQ ID NO: 556-561 is used to target the SOCS2 gene, (iii) a gene encoding B2M and wherein a guide sequence of any of SEQ ID NO: 199-208 is used to target the B2M gene, (iv) a gene encoding PD-1 and wherein a guide sequence of any of SEQ ID NO: 511-514 is used to target the PD-1 gene, (v) a gene encoding TIM-3 and wherein a guide sequence of any of SEQ ID NO: 515-518 is used to target the TIM-3 gene, and (vi) a gene encoding TCR alpha and wherein a guide sequence of any of SEQ ID NO: 566-568 is used to target the TCR alpha gene. In several embodiments, the cells are further edited at one or more of: (i) a gene encoding an adenosine A2 receptor and a guide sequence of any of SEQ ID NO: 503-506 is used to target the adenosine A2 receptor gene, (ii) a gene encoding SMAD3 and wherein a guide sequence of any of SEQ ID NO: 491-493 is used to target the SMAD3 gene, (iii) a gene encoding MAPKAPK3 and wherein a guide sequence of any of SEQ ID NO: 494-496 is used to target the MAPKAPK3 gene, (iv) a gene encoding CEACAMI and wherein a guide sequence of any of SEQ ID NO: 497-499 is used to target the CEACAMI gene, (v) a gene encoding DDIT4 and wherein a guide sequence of any of SEQ ID NO: 500-502 is used to target the DDIT4 gene, and (vi) a gene encoding CD38 and the wherein a guide sequence of any of SEQ ID NO: 519-522 is used to target the CD38 gene. Other guide sequences targeting those genes are also used, in several embodiments. In several embodiments, the cells are not edited at a CD70 gene target site.

In several embodiments, the cytotoxic receptor targets one or more of ligands of NKG2D, CD19, CD70, BCMA, CD38, GPRC5D, CD138, DLL3, EGFR, PSMA, FLT3, KREMEN2, or any combination thereof expressed by target tumor cells. However, in several embodiments, the cells are not engineered to express a cytotoxic receptor complex that targets CD70. In several embodiments, the cells are not engineered to express a cytotoxic receptor complex that targets CD19. In additional embodiments, the cells are not engineered to express a cytotoxic receptor complex that targets ligands of an NKG2D receptor.

In several embodiments, several embodiments, at least a portion of the genetically engineered immune cells are engineered to express membrane bound IL-15. In several embodiments, a fusion of mblL15 with a native receptor (e.g., an IL-15 receptor unit or subunit) are provided for. In several embodiments, the genetically engineered and edited immune cells are suitable for use in allogeneic cancer cell therapy with reduced risk of graft versus host disease and/or with greater cytotoxicity directed against the tumor cells (thereby resulting in a more efficacious therapy).

Methods for treatment of cancer in a subject are provided for herein, and in several embodiments, comprise administering to the subject at least a portion of a population of genetically engineered and gene edited immune cells as provided for herein. Also provided for is the use of genetically engineered and edited immune cells as provided for herein for the treatment of cancer and/or for the preparation of a medicament for the treatment of cancer.

Also provided for herein are methods of manufacturing a population of genetically edited immune cells for cancer immunotherapy, comprising contacting the population of immune cells with a first gene editing complex, wherein the first gene editing complex edits at one or more target sites in a CISH gene of the immune cell to yield reduced levels of expression of CIS protein encoded by the CISH gene as compared to an immune cell not edited at the CISH gene, and contacting the population of immune cells with a second gene editing complex, wherein the second gene editing complex edits at one or more target sites in an additional gene of the immune cell to yield reduced levels of expression of a protein encoded by the additional gene as compared to an immune cell not edited at the additional gene, and wherein the genetically edited immune cells exhibit one or more of enhanced expansion capability, enhanced cytotoxicity against target cells, and enhanced persistence, as compared to immune cells that do not comprise said genetically edited target site or sites.

In several embodiments, the wherein the second gene editing complex edits at one or more target sites in CBLB gene of the immune cell to yield reduced levels of expression of CBLB protein encoded by the CBLB gene as compared to an immune cell not edited at the CBLB gene. In several embodiments, the second gene editing complex edits at one or more target sites in a TGFBR2 gene of the immune cell to yield reduced levels of expression of TGFBR2 protein encoded by the TGFBR2 gene as compared to an immune cell not edited at the TGFBR2 gene. In several embodiments, the second gene editing complex edits at one or more target sites in a TIGIT gene of the immune cell to yield reduced levels of expression of TIGIT protein encoded by the TIGIT gene as compared to an immune cell not edited at the TIGIT gene. As discussed herein, combinations of edits are provided for as well. In several embodiments, an additional edit in one or more genes encoding one of ADARA2, SMAD3, MAPKAPK3, CEACAMI, DDIT4, NKG2A, SOCS2, B2M, PD-1, TIM-3, CD38, or TCR alpha.

In several embodiments, the method further comprise contacting at least a portion of the population of immune cells with a vector comprising a polynucleotide encoding a cytotoxic receptor comprising an extracellular ligand binding domain, a transmembrane domain, and a cytotoxic signaling complex. In several embodiments, the cytotoxic receptor targets one or more of ligands of NKG2D, CD19, CD70, BCMA, CD38, GPRC5D, CD138, DLL3, EGFR, PSMA, FLT3, KREMEN2, or any combination thereof expressed by target tumor cells. However, in several embodiments, the cytotoxic receptor does not target CD70, does not target CD19, and/or does not target NKG2D ligands. Further, in some embodiments, the method does not comprise editing a gene encoding CD70.

In several embodiments, there is a provided a method for treating cancer in a subject comprising, administering to the subject a population of genetically engineered immune cells, wherein the immune cells express a cytotoxic receptor comprising an extracellular ligand binding domain, a transmembrane domain, and a cytotoxic signaling complex, wherein the immune cells are genetically edited at one or more target locations in a CISH gene that encodes a CIS protein, wherein the edits yield reduced expression and/or function of CIS as compared to an immune cell not edited at the location or locations in the CISH gene, wherein the immune cells are edited at one or more a CBLB gene, a TGFBR2 gene, and/or a TIGIT gene, to yield reduced levels of expression or activity of a CBLB protein, a TGFBR2 protein and/or a TIGIT protein as compared to an immune cell not edited at a CBLB gene, a TGFBR2 gene or a TIGIT gene, and wherein the genetically engineered and edited immune cells exhibit one or more of enhanced expansion capability, enhanced cytotoxicity against target cells, and enhanced persistence, as compared to immune cells that do not comprise said genetically edited target site or sites.

In several embodiments, the method further comprises editing the immune cells at least one additional target gene. In several embodiments, the at least one additional target gene is not a gene encoding CD70. In several embodiments, the additional target gene encodes ADAR2A, SMAD3, MAPKAPK3, CEACAM1, DDIT4, NKG2A, SOCS2, B2M, PD-1, TIM-3, CD38, or TCR alpha. In several embodiments, the cytotoxic receptor expressed by the immune cells binds to one or more epitopes of CD19, CD70, BCMA, CD38, GPRC5D, CD138, DLL3, EGFR, PSMA, FLT3, KREMEN2, or combinations thereof. However, in some embodiments, the cytotoxic receptor expressed by the immune cells does not target any of CD19, CD70 or ligands of NKG2D.

In several embodiments, the immune cells comprise Natural Killer (NK) cells, T cells, induced pluripotent stem cells (iPSCs), iPSC-derived NK cells, NK-92 cells, or combinations thereof. In several embodiments, the immune cells comprise a mixture of NK cells and T cells or a mixture of iPSC-derived NK cells and T cells. In several embodiments, the immune cells comprise a purified or substantially purified population of NK cells. In several embodiments, the administered immune cells are allogeneic with respect the subject. In several embodiments, the method further comprises administering IL2.

In several embodiments, there is provided a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein at least a portion of the genetically engineered immune cells are engineered to express membrane bound IL-15, wherein the immune cells are genetically edited at one or more target sites in the genome of the immune cell to yield reduced levels of expression of the protein which is encoded by a gene which comprises an edited target site as compared to a non-edited immune cell, and wherein the genetically engineered and edited immune cells exhibit one or more of enhanced expansion capability, enhanced cytotoxicity against target cells, and enhanced persistence, as compared to immune cells that do not comprise said genetically edited target site or sites. In several embodiments, the immune cells are genetically edited at a target site in a gene encoding an adenosine A2 receptor to yield reduced levels of adenosine receptor A2 expression as compared to a non-edited immune cell, and wherein a guide sequence of any of SEQ ID NO: 503-506 is used to target the adenosine receptor A2 gene. In several embodiments, the cells are further edited at a gene encoding one or more of CISH, SMAD3, MAPKAPK3, CEACAM1, DDIT4, TGFBR2R, NKG2A, SOCS2, CBLB, B2M, TIGIT, PD-1, TIM-3, CD38, and TCR alpha. For example, in several embodiments, the cells are further edited at: (i) a gene encoding CIS and wherein a guide sequence of any of SEQ ID NO: 562-565 is used to target the gene encoding CIS, (ii) a gene encoding SMAD3 and wherein a guide sequence of any of SEQ ID NO: 491-493 is used to target the SMAD3 gene, (iii) a gene encoding MAPKAPK3 and wherein a guide sequence of any of SEQ ID NO: 494-496 is used to target the MAPKAPK3 gene, (iv) a gene encoding CEACAM1 and wherein a guide sequence of any of SEQ ID NO: 497-499, (v) a gene encoding DDIT4 and wherein a guide sequence of any of SEQ ID NO: 500-502 is used to target the DDIT4 gene, (vi) a gene encoding TGFBR2 and wherein a guide sequence of any of SEQ ID NO: 544-547 is used to target the TGFBR2 gene, (vii) a gene encoding NKG2A and wherein a guide sequence of any of SEQ ID NO: 548-551 is used to target the NKG2A gene, (viii) a gene encoding SOCS2 and wherein a guide sequence of any of SEQ ID NO: 556-561 is used to target the SOCS2 gene, (ix) a gene encoding CBLB and wherein a guide sequence of any of SEQ ID NO: 552-555 is used to target the CBLB gene, (x) a gene encoding B2M and wherein a guide sequence of any of SEQ ID NO: 290-299 is used to target the B2M gene, (xi) a gene encoding TIGIT and wherein a guide sequence of any of SEQ ID NO: 507-510 is used to target the TIGIT gene, (xii) a gene encoding PD-1 and wherein a guide sequence of any of SEQ ID NO: 511-514 is used to target the PD-1 gene, (xiii) a gene encoding TIM-3 and wherein a guide sequence of any of SEQ ID NO: 515-518 is used to target the TIM-3 gene, (xiv) a gene encoding CD38 and wherein a guide sequence of any of SEQ ID NO: 519-522 is used to target the CD38 gene, and/or (xv) a gene encoding TCR alpha and wherein a guide sequence of any of SEQ ID NO: 566-569 is used to target the TCR alpha gene.

In several embodiments, the extracellular ligand binding domain comprises a receptor that is directed against a tumor marker selected from the group consisting of MICA, MICB, ULBP1, ULBP2, ULBP3, ULBP4, ULBP5, and ULBP6. For example, in some embodiments, the cytotoxic receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain.

In some embodiments, the cytotoxic receptor targets one or more of NKG2D receptor ligands, CD19, CD70, BCMA, CD38, GPRC5D, CD138 DLL3, EGFR, PSMA, FLT3, or KREMEN2 expressed by target tumor cells. In several embodiments, the cytotoxic signaling complex comprises an OX40 subdomain or a 4-1 BB domain, and a CD3zeta subdomain.

In several embodiments, the population of immune cells comprise Natural Killer (NK) cells, T cells, induced pluripotent stem cells (iPSCs), iPSC-derived NK cells, NK-92 cells, or combinations thereof. In several embodiments, the immune cells are suitable for use in allogeneic cancer cell therapy with reduced risk of graft versus host disease and/or are suitable for use in allogeneic cancer cell therapy with reduced risk of cytotoxic activity between the genetically engineered immune cells. Methods for treatment of cancer by administering such cells are provided for herein, as is the use of such cells for the treatment of cancer or for the preparation of a medicament for the treatment of cancer.

Further provided for herein is a method of manufacturing a population of genetically engineered immune cells for cancer immunotherapy, comprising contacting a population of immune cells with a vector comprising a polynucleotide encoding a cytotoxic receptor comprising an extracellular ligand binding domain, a transmembrane domain, and a cytotoxic signaling complex; contacting the population of immune cells with a gene editing complex; wherein the gene editing complex edits at one or more target sites in the genome of the immune cell to yield reduced levels of expression of the protein which is encoded by a gene which comprises an edited target site as compared to a non-edited immune cell, and wherein the genetically engineered and edited immune cells exhibit one or more of enhanced expansion capability, enhanced cytotoxicity against target cells, and enhanced persistence, as compared to immune cells that do not comprise said genetically edited target site or sites. In several embodiments, an edit (or edits) is made in a gene encoding one or more of CISH, ADARA2, SMAD3, MAPKAPK3, CEACAM1, DDIT4, TGFBR2R, NKG2A, SOCS2, CBLB, B2M, TIGIT, PD-1, TIM-3, CD38, and TCR alpha.

Also provided for is a method for treating cancer in a subject comprising, administering to the subject a population of genetically engineered immune cells, wherein the immune cells express a cytotoxic receptor comprising an extracellular ligand binding domain, a transmembrane domain, and a cytotoxic signaling complex, wherein the cytotoxic signaling complex comprises an OX-40 subdomain and a CD3zeta subdomain, wherein the cells are engineered to express membrane bound IL-15, wherein the immune cells are genetically edited at one or more target sites in the genome of the immune cell to yield reduced levels of expression of the protein which is encoded by a gene which comprises an edited target site as compared to a non-edited immune cell, and wherein the genetically engineered and edited immune cells exhibit one or more of enhanced expansion capability, enhanced cytotoxicity against target cells, and enhanced persistence, as compared to immune cells that do not comprise said genetically edited target site or sites. In several embodiments, the cells are edited at least two genes selected from those genes encoding CISH, ADAR2A, SMAD3, MAPKAPK3, CEACAM1, DDIT4, TGFBR2R, NKG2A, SOCS2, CBLB, B2M, TIGIT, PD-1, TIM-3, CD38, or TCR alpha. In several embodiments, the cytotoxic receptor comprises (i) an NKG2D ligand-binding domain or a binding domain that binds to one or more epitopes of CD19, CD70, BCMA, CD38, GPRC5D, CD138 DLL3, EGFR, PSMA, FLT3, or KREMEN2 expressed by target tumor cells, (ii) a CD8 transmembrane domain, and (iii) a signaling complex that comprises an OX40 co-stimulatory subdomain and a CD3z co-stimulatory subdomain. In several embodiments, the immune cells comprise Natural Killer (NK) cells, T cells, induced pluripotent stem cells (iPSCs), iPSC-derived NK cells, NK-92 cells, or combinations thereof. Additionally, in several embodiments, the administered immune cells are allogeneic with respect the subject.

Provided for in several embodiments is a population of genetically engineered immune cells for cancer immunotherapy, comprising genetically engineered immune cells that express a cytotoxic receptor comprising an extracellular ligand binding domain, a transmembrane domain, and a cytotoxic signaling complex, wherein at least a portion of the genetically engineered immune cells are engineered to express membrane bound IL-15, wherein the genetically engineered immune cells are engineered to express at least one immunosuppressive effector, wherein the at least one immunosuppressive effector exerts suppressive effects on undesired cytotoxic activity of suppressive cells, and wherein the genetically engineered immune cells exhibit one or more of enhanced expansion capability, enhanced cytotoxicity against target cells, and enhanced persistence, as compared to cells that do not comprise said immunosuppressive effector. In several embodiments, the population comprises one or more of genetically engineered NK cells and genetically engineered T cells. In several embodiments, the suppressive cells comprise host cells or one or more of non-engineered natural killer cells, non-engineered T cells, or suppressive engineered cells. In several embodiments, the cells that do not comprise said immunosuppressive effector are either non-engineered or engineered cells.

In several embodiments, the genetically engineered immune cells are also genetically edited to reduce expression of beta-2 microglobulin (B2M), and wherein the reduced expression of B2M enables the immune cells to be used in allogeneic cancer immunotherapy with reduced host versus graft rejection as compared to immune cells expressing endogenous levels of B2M.

In several embodiments, the at least one immunosuppressive effector comprises a virally-derived peptide, optionally derived from a retrovirus, optionally derived from an envelope protein of a retrovirus. In some embodiments, the at least one immunosuppressive effector is bound to an extracellular membrane of the immune cells, wherein the at least one immunosuppressive effector comprises a transmembrane protein, and wherein the transmembrane protein is selected from CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor.

In several embodiments, the immunosuppressive effector comprises one or more peptides having at least 90% sequence identity to one or more of SEQ ID NOs: 199-215, 219, 220, 223, 225, 228, 230, 235, 238, 240, 243, 250, or 253. In several embodiments, the at least one immunosuppressive effector comprises at least a portion of a human protein and/or at least a portion of a human protein complex. In several embodiments, the immunosuppressive effector comprises a peptide having at least 90% sequence identity to one or more of SEQ ID NO: 248, 245, 273, 276, 278, 279, 286, 287, 288, and 289. In several embodiments, the at least one immunosuppressive effector comprises a chimeric construct comprises at least one virally-derived peptide and at least a portion of a human protein and/or at least a portion of a human protein complex. In several embodiments, the chimeric immunosuppressive effector construct comprises: (i) two or more of a truncated human CD47, a p15E peptide, an HIV peptide, and an HTLV peptide, (ii) a truncated human CD47 domain and at least one p15E peptide, an HIV peptide, and an HTLV peptide, (iii) a truncated human CD47 domain and at least one HIV peptide, (iv) a truncated human CD47 domain and at least HTLV peptide, (v) a sequence having at least 95% sequence identity to one or more of SEQ ID Nos: 256, 259, 262, 265, 268, 271, (vi) a viral UL18 protein. In several embodiments, the chimeric immunosuppressive effector construct comprises a viral UL18 protein and has a sequence having at least 95% sequence identity to SEQ ID NO: 280, or wherein the chimeric immunosuppressive effector construct comprises a viral UL18 protein and a human B2M domain and comprises a sequence having at least 95% sequence identity to SEQ ID NO: 283 or 285. In several embodiments, the immunosuppressive effector construct is integrated into the cytotoxic receptor between the transmembrane domain and the extracellular ligand-binding domain, within the extracellular ligand-binding domain, into a linker region of an scFv when the extracellular ligand-binding domain comprises an scFv and the chimeric immunosuppressive effector construct is integrated into a linker region of the scFv, within an N-terminal region of the cytotoxic receptor distally positioned from the extracellular ligand-binding domain, or at a plurality of locations within an extracellular region of the cytotoxic receptor. In several embodiments, the at least one chimeric immunosuppressive effector construct is bound to an extracellular membrane of the immune cells. In several embodiments, the cytotoxic receptor comprises at least one immunosuppressive effector and the genetically engineered immune cells express at least one membrane-bound immunosuppressive effector.

In several embodiments, the genetically engineered immune cells are suitable for use in allogeneic cancer cell therapy with reduced risk of graft versus host disease. In additional embodiments, the genetically engineered immune cells are suitable for use in allogeneic cancer cell therapy with reduced risk of cytotoxic activity between the genetically engineered immune cells.

Provided for herein also is a method of manufacturing a population of genetically engineered immune cells for cancer immunotherapy, comprising contacting a population of immune cells with a polynucleotide encoding a cytotoxic receptor comprising an extracellular ligand binding domain, a transmembrane domain, and a cytotoxic signaling complex, contacting the population of immune cells with an additional polynucleotide encoding at least one immunosuppressive effector, wherein the at least one immunosuppressive effector exerts suppressive effects on the cytotoxic activity of suppressive cells, and wherein the genetically engineered immune cells exhibit one or more of enhanced expansion capability, enhanced cytotoxicity against target cells, and enhanced persistence, as compared to cells that do not comprise said at least one immunosuppressive effector.

Also provided is a method of engineering a population of genetically engineered immune cells for cancer immunotherapy, comprising contacting a population of immune cells with a polynucleotide encoding a cytotoxic receptor comprising an extracellular ligand binding domain, a transmembrane domain, and a cytotoxic signaling complex and encoding at least one immunosuppressive effector, wherein the at least one immunosuppressive effector exerts suppressive effects on the cytotoxic activity of suppressive cells, and wherein the genetically engineered immune cells exhibit one or more of enhanced expansion capability, enhanced cytotoxicity against target cells, and enhanced persistence, as compared to cells that do not comprise said at least one immunosuppressive effector. In several embodiments, the polynucleotide further encodes membrane-bound IL15. In several embodiments, the method further comprises contacting the population of immune cells with a polynucleotide encoding a membrane-bound immunosuppressive effector.

In several embodiments, there is provided a method of reducing fratricide among a mixed population of genetically engineered immune cells for cancer immunotherapy, comprising contacting a first subpopulation of immune cells from a population of mixed immune cells with a polynucleotide encoding at least one immunosuppressive effector, wherein expression of the at least one immunosuppressive effector by the first subpopulation of immune cells reduces suppressive activity of a second subpopulation of immune cells that are directed against the first subpopulation of immune cells and contacting the second subpopulation from a population of mixed immune cells with a polynucleotide encoding at least an additional immunosuppressive effector, wherein expression of the at least an additional immunosuppressive effector by the second subpopulation of immune cells reduces suppressive activity of the first subpopulation of immune cells that are directed against the second subpopulation of immune cells, and wherein the expression of the immunosuppressive effector by the first subpopulation and the second subpopulation reduces fratricide among the cells within the population of genetically engineered immune cells.

A method of engineering a population of genetically engineered immune cells for enhanced allogeneic cancer immunotherapy, is also provided and comprises, in several embodiments, genetically editing a mixed population of immune cells comprising NK cells and T cells to reduce expression Human Leukocyte Antigen (HLA) on the surface of the immune cells, wherein reduced expression of HLA on the surface of the immune cells reduces T cell-mediated cytotoxicity against the edited population of immune cells, wherein reduced expression of HLA on the surface of the cells renders the edited population of immune cells susceptible to NK-mediated cytotoxicity against the edited immune cells; and genetically engineering the edited cells to express one or more immunosuppressive effectors that reduce NK-mediated cytotoxicity against the mixed population of immune cells, wherein the one or more immunosuppressive effectors comprises one or more of a viral immunosuppressive peptide, a viral protein that is an HLA homolog, HLA-E, HLA-G, a human protein or fragment thereof that reduces phagocytosis of cells, a chimeric construct comprising a viral immunosuppressive peptide and a human protein or fragment thereof that reduces phagocytosis of cells, or combinations thereof, wherein the reduced T cell-mediated cytotoxicity reduces engineered T cell-mediated fratricidal cytotoxicity against engineered NK cells and, upon administration, host T-cell mediated cytotoxicity against engineered NK cells, and wherein the wherein the reduced NK cell-mediated cytotoxicity reduces engineered NK cell-mediated fratricidal cytotoxicity against engineered T cells and, upon administration, host NK cell-mediated cytotoxicity against engineered NK and engineered T cells, thereby allowing for enhanced persistence of the engineering mixed population of immune cells upon administration to an allogeneic subject and allowing for enhanced allogeneic cancer immunotherapy. In several embodiments, the method further comprises genetically editing the DNA of the genetically engineered immune cells to alter the expression of one or more of a CISH gene, a B2M gene, a CD70 gene, an adenosine receptor gene, an NKG2A gene, a CIITA gene, a TGFBR gene, or any combination thereof.

There is provided for herein, in several embodiments, a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell, wherein the immune cells are genetically edited at one or more target locations in a CISH gene that encodes a CIS protein, wherein the edits yield reduced expression and/or function of CIS as compared to an immune cell not edited at the location or locations in the CISH gene, wherein the immune cells are edited at one or more additional target sites in the genome of the immune cell to yield reduced levels of expression of the protein which is encoded by a gene which comprises an edited target site as compared to a non-edited immune cell, wherein the edits to the CISH gene and the one or more additional target sites are made using a Crispr/Cas9 system, a Crispr/CasX system, a Crispr/CasY system, or another an RNA-guided endonuclease, and wherein the genetically engineered and edited immune cells exhibit one or more of enhanced expansion capability, enhanced cytotoxicity against target cells, and enhanced persistence, as compared to immune cells that do not comprise said genetically edited target site or sites. In several embodiments, a guide sequence of any of SEQ ID NO: 153-157 or 562-565 is used to target the CISH gene.

In several embodiments, the cells are edited at an additional target site in a CBLB gene to yield cells comprising edits within at least CISH and CBLB. In several embodiments, a guide sequence of any of SEQ ID NO: 164 to 166 or 552-555 is used to target the CBLB gene.

In several embodiments, the cells are edited at an additional target site in a TGFBR2 gene to yield cells comprising edits within at least CISH and TGFBR2. In several embodiments, a guide sequence of any of SEQ ID NO: 147 to 152 or 544-547 is used to target the TGFBR2 gene.

In several embodiments, the cells are edited at an additional target site in a TIGIT gene to yield cells comprising edits within at least CISH and TIGIT. In several embodiments, a guide sequence of any of SEQ ID NO: 507-510 is used to target the TIGIT gene.

In several embodiments, the population of genetically engineered and gene edited immune are not edited at a CD70 gene target site. In several embodiments, however, they are edited at CD70 and also at one or more additional genes, for example CD70/CISH/CBLB, CD70/CISH/TGFBR2, CD70/CISH/TIGIT, and the like. Any combination of target genes may be edited according to embodiments disclosed herein

In several embodiments, at least a portion of the genetically engineered immune cells are engineered to express membrane bound IL-15.

In several embodiments, the population of genetically engineered and gene edited immune cells are not engineered to express a cytotoxic receptor complex that targets CD70. In several embodiments, the cells are not engineered to express a cytotoxic receptor complex that targets CD19. In several embodiments, the cells are not engineered to express a cytotoxic receptor complex that targets ligands of an NKG2D receptor. However, in several embodiments, the cells are engineered to express an anti-CD70 CAR encoded by a polynucleotide at least 95% identical to one or more of SEQ ID NOs: 300-382. In several embodiments, the polynucleotide encodes an amino acid sequence at least 95% identical to one or more of SEQ ID NO: 383-465. In several embodiments, the cells are engineered to express an anti-CD70 CAR at least 95% identical to one or more of SEQ ID NO: 912-985. In several embodiments, the cells are engineered to express an anti-CD19 CAR encoded by a polynucleotide at least 95% identical to one or more of SEQ ID NOs: 175, 177, 179, 181, 183, 185, 187, 189, 191, 193, 195, or 197. In several embodiments, the cells are engineered to express an anti-CD19 CAR at least 95% identical to one or more of SEQ ID NO: 900-911. In several embodiments, the cells are engineered to express a chimeric receptor that targets NKG2D ligands encoded by a polynucleotide at least 95% identical to SEQ ID NO: 475. In several embodiments, the cells are engineered to express a chimeric receptor that targets NKG2D ligands at least 95% identical to SEQ ID NO. 899.

In several embodiments, the cells are further edited at a one or more genes encoding an adenosine A2 receptor, SMAD3, MAPKAPK3, CEACAM1, DDIT4, TGFBR2R, NKG2A, SOCS2, B2M, TIGIT, PD-1, TIM-3, CD38, or TCR alpha.

In several embodiments, the cells are further edited at a gene encoding an adenosine A2 receptor and the nuclease is guided to the adenosine A2 receptor target sequence of any of SEQ ID NO: 503-506. In several embodiments, the cells are further edited at a gene encoding SMAD3 and the nuclease is guided to the target sequence of any of SEQ ID NO: 491-493. In several embodiments, the cells are further edited at a gene encoding MAPKAPK3 and the nuclease is guided to the target sequence of any of SEQ ID NO: 494-496. In several embodiments, the cells are further edited at a gene encoding CEACAMI and the nuclease is guided to the target sequence of any of SEQ ID NO: 497-499. In several embodiments, the cells are further edited at a gene encoding DDIT4 and the nuclease is guided to the target sequence of any of SEQ ID NO: 500-502. In several embodiments, the cells are further edited at a gene encoding NKG2A and the nuclease is guided to the target sequence of any of SEQ ID NO: 548-551. In several embodiments, the cells are further edited at a gene encoding SOCS2 and the nuclease is guided to the target sequence of any of SEQ ID NO: 556-561. In several embodiments, the cells are further edited at a gene encoding B2M and the nuclease is guided to the target sequence of any of SEQ ID NO: 290-299. In several embodiments, the cells are further edited at a gene encoding PD-1 and the nuclease is guided to the target sequence of any of SEQ ID NO: 511-514. In several embodiments, the cells are further edited at a gene encoding TIM-3 and the nuclease is guided to the target sequence of any of SEQ ID NO: 515-518. In several embodiments, the cells are further edited at a gene encoding CD38 and the nuclease is guided to the target sequence of any of SEQ ID NO: 519-522. In several embodiments, the cells are further edited at a gene encoding TCR alpha and the nuclease is guided to the target sequence of any of SEQ ID NO: 566-569.

Depending on the embodiment, the population of genetically engineered and gene edited immune cells may comprise Natural Killer (NK) cells, T cells, induced pluripotent stem cells (iPSCs), iPSC-derived NK cells, NK-92 cells, or combinations thereof. In several embodiments, the genetically engineered and edited immune cells are suitable for use in allogeneic cancer cell therapy with reduced risk of graft versus host disease.

Also provided for herein is a method for the treatment of cancer in a subject comprising administering to the subject at least a portion of population of immune cells according to embodiments discussed herein. Likewise, provided for is the use of use of genetically engineered and edited immune cells provided for herein for the treatment of cancer and/or for the preparation of a medicament for the treatment of cancer.

Also provided for herein is a method of manufacturing a population of genetically edited immune cells for cancer immunotherapy, comprising contacting the population of immune cells with a first Cas-gRNA ribonucleoprotein complex (RNP), wherein the RNP edits at one or more target sites in a CISH gene of the immune cell to yield reduced levels of expression of CIS protein encoded by the CISH gene as compared to an immune cell not edited at the CISH gene, wherein the Cas of the RNP comprises Cas9, CasX, CasY, or combinations thereof, and contacting the population of immune cells with a second RNP complex, wherein the second RNP edits at one or more target sites in CBLB gene of the immune cell to yield reduced levels of expression of CBLB protein encoded by the CBLB gene as compared to an immune cell not edited at the CBLB gene, wherein the Cas of the RNP comprises Cas9, CasX, CasY, or combinations thereof and wherein the genetically edited immune cells exhibit one or more of enhanced expansion capability, enhanced cytotoxicity against target cells, and enhanced persistence, as compared to immune cells that do not comprise said genetically edited target site or sites.

Also provided for herein is a method of manufacturing a population of genetically edited immune cells for cancer immunotherapy, comprising contacting the population of immune cells with a first Cas-gRNA ribonucleoprotein complex (RNP), wherein the RNP edits at one or more target sites in a CISH gene of the immune cell to yield reduced levels of expression of CIS protein encoded by the CISH gene as compared to an immune cell not edited at the CISH gene, wherein the Cas of the RNP comprises Cas9, CasX, CasY, or combinations thereof, and contacting the population of immune cells with a second RNP complex, wherein the second RNP edits at one or more target sites in a TGFBR2 gene of the immune cell to yield reduced levels of expression of TGFBR2 protein encoded by the TGFBR2 gene as compared to an immune cell not edited at the TGFBR2 gene, wherein the Cas of the RNP comprises Cas9, CasX, CasY, or combinations thereof and wherein the genetically edited immune cells exhibit one or more of enhanced expansion capability, enhanced cytotoxicity against target cells, and enhanced persistence, as compared to immune cells that do not comprise said genetically edited target site or sites.

Also provided for herein is a method of manufacturing a population of genetically edited immune cells for cancer immunotherapy, comprising contacting the population of immune cells with a first Cas-gRNA ribonucleoprotein complex (RNP), wherein the RNP edits at one or more target sites in a CISH gene of the immune cell to yield reduced levels of expression of CIS protein encoded by the CISH gene as compared to an immune cell not edited at the CISH gene, wherein the Cas of the RNP comprises Cas9, CasX, CasY, or combinations thereof, and contacting the population of immune cells with a second RNP complex, wherein the second RNP edits at one or more target sites in a TIGIT gene of the immune cell to yield reduced levels of expression of TIGIT protein encoded by the TIGIT gene as compared to an immune cell not edited at the TIGIT gene, wherein the Cas of the RNP comprises Cas9, CasX, CasY, or combinations thereof and wherein the genetically edited immune cells exhibit one or more of enhanced expansion capability, enhanced cytotoxicity against target cells, and enhanced persistence, as compared to immune cells that do not comprise said genetically edited target site or sites.

Also provided for herein is a method of manufacturing a population of genetically edited immune cells for cancer immunotherapy, comprising contacting the population of immune cells with a first Cas-gRNA ribonucleoprotein complex (RNP), wherein the RNP edits at one or more target sites in a CISH gene of the immune cell to yield reduced levels of expression of CIS protein encoded by the CISH gene as compared to an immune cell not edited at the CISH gene, wherein the Cas of the RNP comprises Cas9, CasX, CasY, or combinations thereof, and contacting the population of immune cells with a second RNP complex, wherein the second RNP edits at one or more target sites in an additional gene of the immune cell to yield reduced levels of expression of a protein encoded by the additional gene as compared to an immune cell not edited at the additional gene, wherein the Cas of the RNP comprises Cas9, CasX, CasY, or combinations thereof, and wherein the genetically edited immune cells exhibit one or more of enhanced expansion capability, enhanced cytotoxicity against target cells, and enhanced persistence, as compared to immune cells that do not comprise said genetically edited target site or sites.

In several embodiments, the methods further comprise contacting the population of immune cells with a vector comprising a polynucleotide encoding a cytotoxic receptor comprising an extracellular ligand binding domain, a transmembrane domain, and a cytotoxic signaling complex. In several embodiments, the cytotoxic receptor does not target CD70. In other embodiments, the cytotoxic receptor does target CD70. In several embodiments, the methods do not comprise editing a gene encoding CD70. In some embodiments, at least CD70 is edited. In several embodiments, the cytotoxic receptor does not target CD19. In several embodiments, the cytotoxic receptor does target CD19. In several embodiments, the cytotoxic receptor does not target NKG2D ligands. In several embodiments, the cytotoxic receptor does target NKG2D ligands.

In several embodiments, the method further comprises making an additional edit in one or more genes encoding one of ADARA2, SMAD3, MAPKAPK3, CEACAM1, DDIT4, TGFBR2R, NKG2A, SOCS2, B2M, PD-1, TIM-3, CD38, or TCR alpha.

In several embodiments, the cytotoxic receptor targets one or more of ligands of NKG2D, CD19, CD70, BCMA, CD38, GPRC5D, CD138 DLL3, EGFR, PSMA, FLT3, KREMEN2, or any combination thereof expressed by target tumor cells. Depending on the embodiments, the immune cells can comprise Natural Killer (NK) cells, T cells, induced pluripotent stem cells (iPSCs), iPSC-derived NK cells, NK-92 cells, or combinations thereof.

In several embodiments, there is provided a method for treating cancer in a subject comprising administering to the subject a population of genetically engineered immune cells, wherein the immune cells express a cytotoxic receptor comprising an extracellular ligand binding domain, a transmembrane domain, and a cytotoxic signaling complex, wherein the immune cells are genetically edited at one or more target locations in a CISH gene that encodes a CIS protein, wherein the edits yield reduced expression and/or function of CIS as compared to an immune cell not edited at the location or locations in the CISH gene, wherein the immune cells are edited at one or more a CBLB gene, a TGFBR2 gene, and/or a TIGIT gene, to yield reduced levels of expression or activity of a CBLB protein, a TGFBR2 protein and/or a TIGIT protein as compared to an immune cell not edited at a CBLB gene, a TGFBR2 gene or a TIGIT gene, wherein the edits are made using an RNA-guided endonuclease, and wherein the genetically engineered and edited immune cells exhibit one or more of enhanced expansion capability, enhanced cytotoxicity against target cells, and enhanced persistence, as compared to immune cells that do not comprise said genetically edited target site or sites.

In several embodiments, the RNA-guided endonuclease is a Crispr/CasX, Crispr/CasY, or Crispr/Cas9 system. In several embodiments, the method further comprises editing the immune cells at least one additional target gene. In some embodiments, the at least one additional target gene is not a gene encoding CD70. In several embodiments, the cells are edited at an additional target gene encoding ADAR2A, SMAD3, MAPKAPK3, CEACAM1, DDIT4, NKG2A, SOCS2, B2M, PD-1, TIM-3, CD38, or TCR alpha. In several embodiments, the cytotoxic receptor expressed by the immune cells does not target CD70, does not target CD19, or does not target NKG2D ligands. However, in several embodiments, the cytotoxic receptor expressed by the immune cells binds to one or more epitopes of CD19, CD70, BCMA, CD38, GPRC5D, CD138 DLL3, EGFR, PSMA, FLT3, KREMEN2, or combinations thereof.

In several embodiments, the immune cells comprise Natural Killer (NK) cells, T cells, induced pluripotent stem cells (iPSCs), iPSC-derived NK cells, NK-92 cells, or combinations thereof. In several embodiments, the immune cells comprise a mixture of NK cells and T cells or a mixture of iPSC-derived NK cells and T cells. In several embodiments, the administered immune cells are allogeneic with respect the subject. In several embodiments, the method further comprises administering IL2.

In several embodiments, there is therefore provided a population of genetically engineered immune cells for cancer immunotherapy, comprising genetically engineered immune cells that express a cytotoxic receptor, wherein the immune cells are genetically edited at one or more target sites in the genome of the immune cell to yield reduced levels of expression of a protein encoded by the edited gene, wherein the genetically engineered immune cells are also engineered to express at least one immunosuppressive effector that exerts suppressive effects on the cytotoxic activity of natural killer cells and/or T cells, and wherein the resulting genetically engineered and edited immune cells exhibit one or more of enhanced expansion capability, enhanced cytotoxicity against target cells, and enhanced persistence, as compared to cells that do not comprise said immunosuppressive effector and said edited gene.

Also provided for herein is a population of genetically engineered immune cells for cancer immunotherapy, comprising one or more of genetically engineered NK cells and genetically engineered T cells, wherein the genetically engineered immune cells are engineered to express a cytotoxic receptor, herein the immune cells are genetically edited at one or more target sites in the genome of the immune cell to yield reduced levels of expression of a protein which is encoded by an edited gene (or genes), wherein the genetically engineered immune cells are further genetically engineered to express at least one immunosuppressive effector that exerts suppressive effects on the cytotoxic activity of undesired cells, such as non-engineered natural killer cells, non-engineered T cells, or undesired engineered cells, and wherein the genetically engineered and edited immune cells exhibit one or more of enhanced expansion capability, enhanced cytotoxicity against target cells, and enhanced persistence, as compared to cells that do not comprise said immunosuppressive effector and said edited gene.

Further provided for herein is a population of genetically engineered immune cells for cancer immunotherapy, comprising one or more of genetically engineered NK cells and genetically engineered T cells, wherein the plurality of genetically engineered immune cells are engineered to express a cytotoxic receptor, wherein the immune cells are also genetically edited at one or more target sites in the genome to yield reduced levels of expression of a protein which is encoded by an edited gene which comprises an edited target site as compared to a non-edited immune cell, wherein immune cells are further genetically engineered to express at least one immunosuppressive effector configured to produce suppressive effects on undesired cytotoxic activity of suppressive cells, and wherein the genetically engineered immune cells exhibit one or more of enhanced expansion capability, enhanced cytotoxicity against target cells, and enhanced persistence, as compared to cells that do not comprise said immunosuppressive effector.

In several embodiments, the suppressive cells comprise host cells. In several embodiments, the suppressive cells comprise one or more of non-engineered natural killer cells, non-engineered T cells, or suppressive engineered cells. In several embodiments, the suppressive engineered cells comprise the genetically engineered immune cells (e.g., other cells within the population of cells to be used for therapy). In several embodiments, the cells that do not comprise said immunosuppressive effector are either non-engineered (e.g., host cells) or engineered cells (other engineered cells that were not edited or engineered with the immunosuppressive effector).

In several embodiments, the cytotoxic receptor expressed by the cells is a chimeric antigen receptor or other chimeric receptor comprising an extracellular ligand binding domain, a transmembrane domain, and a cytotoxic signaling complex. In several embodiments, the cytotoxic signaling complex comprises an OX40 subdomain or a 4-1 BB domain, and a CD3zeta subdomain. In several embodiments, the cytotoxic receptor targets one or more of NKG2D, CD19, BCMA, CD70, and CD38 expressed by target tumor cells.

In several embodiments, the edits are made using a targeted endonuclease. In several embodiments, the endonuclease is an RNA-guided endonuclease. In several embodiments, the edits are made using a Crispr/Cas system.

According to some embodiments, the genetically engineered immune cells are also genetically edited to reduce expression of beta-2 microglobulin (B2M). In several embodiments, the reduced expression of B2M enables the immune cells to be used in allogeneic cancer immunotherapy with reduced host versus graft rejection as compared to immune cells expressing endogenous levels of B2M.

In several embodiments, the at least one immunosuppressive effector comprises a virally-derived peptide. In several embodiments, the immunosuppressive effector comprises a peptide derived from a retrovirus, such as a peptide derived from an envelope protein of a retrovirus. In additional embodiments, the at least one immunosuppressive effector comprises at least a portion of a human protein and/or at least a portion of a human protein complex. In several embodiments, the at least one immunosuppressive effector comprises at least a portion of human protein. In still further embodiments, the at least one immunosuppressive effector comprises a chimeric construct comprises at least one virally-derived peptide and at least a portion of a human protein and/or at least a portion of a human protein complex.

In several embodiments, the at least one immunosuppressive effector is integrated into the cytotoxic receptor at one or more positions within the receptor. For example, in several embodiments, the at least one immunosuppressive effector is integrated into the cytotoxic receptor between the transmembrane domain and the extracellular ligand-binding domain. In other embodiments, the at least one immunosuppressive effector is integrated into the cytotoxic receptor within the extracellular ligand-binding domain. In still further embodiments, when the wherein the extracellular ligand-binding domain comprises an scFv, the at least one immunosuppressive effector is integrated into a linker region of the scFv. In several embodiments, the at least one immunosuppressive effector is integrated into the cytotoxic receptor within an N-terminal region of the cytotoxic receptor distally positioned from the extracellular ligand-binding domain. In some embodiments, the at least one immunosuppressive effector is integrated into the cytotoxic receptor at a plurality of locations within an extracellular region of the cytotoxic receptor, for example, multiple copies of the effector are positioned at various locations within the receptor, inducing enhanced immunosuppressive effects.

In addition to, or in place of the effectors integrated into the receptor complex, in several embodiments, the at least one immunosuppressive effector is bound to an extracellular membrane of the immune cells. In several such embodiments, the at least one immunosuppressive effector comprises a transmembrane protein. Non-limiting examples of transmembrane proteins that can be used according to embodiments disclosed herein are CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. Combinations of transmembrane domains are used, according to several embodiments. In several embodiments, the transmembrane protein comprises a CD8α transmembrane protein. In additional embodiments, the at least one immunosuppressive effector is expressed on the immune cells by a disulfide trap single chain trimer (dtSCT).

In several embodiments, the immunosuppressive effector is encoded by a nucleic acid or comprises a peptide having at least 85% sequence identity to one or more of the nucleotide or amino acid sequences of SEQ ID NOs: 683-894. In several embodiments, the immunosuppressive effector comprises a peptide having at least 95% sequence identity to SEQ ID NO: 689. In several embodiments, the immunosuppressive effector is encoded by a nucleic acid having at least 95% sequence identity to SEQ ID NO: 690.

In several embodiments, the genetically engineered immune cells comprise genetically engineered Natural Killer (NK) cells, genetically engineered T cells, or combinations thereof. In several embodiments, the genetically engineered immune cells are suitable for use in allogeneic cancer cell therapy with reduced risk of graft versus host disease. Furthermore, in several embodiments, the genetically engineered immune cells are suitable for use in allogeneic cancer cell therapy with reduced risk of cytotoxic activity between the genetically engineered immune cells. In several embodiments, at least a portion (e.g., at least 50%, at least 60%, at least 75%, or more) of the genetically engineered immune cells are engineered to express membrane bound IL-15. In several embodiments, at least about 60% of the NK cells are positive for NKG2A. In several embodiments, at least about 80% of the NK cells are positive for NKG2A. In several embodiments, the elevated expression levels of NKG2A enhance the efficacy of the engineered and edited cells in cancer immunotherapy by virtue of reduced cytotoxicity within the cells of the population administered to a subject.

In several embodiments, there are also provided methods for the treatment of cancer in a subject comprising administering to the subject the genetically engineered and edited immune cells according to embodiments disclosed herein. In several embodiments, there is also provided for the use of the genetically engineered and edited immune cells according embodiments discussed herein for the treatment of cancer or for the preparation of a medicament for the treatment of cancer.

In several embodiments, there is provided a method of manufacturing a population of genetically engineered immune cells for cancer immunotherapy, comprising contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell; contacting the population of immune cells with a polynucleotide encoding a cytotoxic receptor comprising an extracellular ligand binding domain, a transmembrane domain, and a cytotoxic signaling complex; contacting the population of immune cells with an additional polynucleotide encoding at least one immunosuppressive effector, wherein the immunosuppressive effector is encoded by a nucleic acid or comprises a peptide having at least 85% sequence identity to one or more of the nucleotide or amino acid sequences of SEQ ID NOs: 683-894; wherein the at least one immunosuppressive effector exerts suppressive effects on the cytotoxic activity of suppressive cells, and wherein the genetically engineered immune cells exhibit one or more of enhanced expansion capability, enhanced cytotoxicity against target cells, and enhanced persistence, as compared to cells that do not comprise said at least one immunosuppressive effector. In several embodiments, the target of the genetic editing is one or more of a CISH gene, a B2M gene, a CD70 gene, an adenosine receptor gene, an NKG2A gene, a CIITA gene, a TGFBR gene, or any combination thereof.

Also provided for herein are methods of engineering a population of genetically engineered immune cells for enhanced allogeneic cancer immunotherapy, comprising genetically editing a mixed population of immune cells comprising NK cells and T cells to disrupt Beta-2 microglobulin (B2M) expression and coordinately reduce expression Human Leukocyte Antigen (HLA) on the surface of the immune cells, wherein reduced expression of HLA on the surface of the immune cells reduces T cell-mediated cytotoxicity against the edited population of immune cells, wherein reduced expression of HLA on the surface of the cells renders the edited population of immune cells susceptible to NK-mediated cytotoxicity against the edited immune cells, and genetically engineering the edited cells to express one or more immunosuppressive effectors that reduce NK-mediated cytotoxicity against the mixed population of immune cells, wherein the one or more immunosuppressive effectors comprises one or more of a viral immunosuppressive peptide, a viral protein that is an HLA homolog, HLA-E, HLA-G, a human protein or fragment thereof that reduces phagocytosis of cells, a chimeric construct comprising a viral immunosuppressive peptide and a human protein or fragment thereof that reduces phagocytosis of cells, or combinations thereof, wherein the reduced T cell-mediated cytotoxicity reduces engineered T cell-mediated fratricidal cytotoxicity against engineered NK cells and, upon administration, host T-cell mediated cytotoxicity against engineered NK cells, and wherein the wherein the reduced NK cell-mediated cytotoxicity reduces engineered NK cell-mediated fratricidal cytotoxicity against engineered T cells and, upon administration, host NK cell-mediated cytotoxicity against engineered NK and engineered T cells, thereby allowing for enhanced persistence of the engineering mixed population of immune cells upon administration to an allogeneic subject and allowing for enhanced allogeneic cancer immunotherapy. In several embodiments, at least about 60% of the NK cells in the mixed population are positive for NKG2A expression.

In several embodiments, the method further comprises contacting the mixed population of immune cells with a polynucleotide encoding a cytotoxic receptor comprising an extracellular ligand binding domain, a transmembrane domain, and a cytotoxic signaling complex. Depending on the embodiment, the polynucleotide encodes a cytotoxic receptor targeting one or more of NKG2D, CD19, CD70, BCMA, and CD38 expressed by target tumor cells.

In several embodiments, the method further comprises genetically editing the DNA of the genetically engineered immune cells to alter the expression of one or more of a CISH gene, a CD70 gene, an adenosine receptor gene, an NKG2A gene, a CIITA gene, a TGFBR gene, or any combination thereof. In several embodiments, the gene editing to reduce expression or the gene editing to induce expression is made using a CRISPR-Cas system. In several embodiments, the Cas is Cas9, Cas12, Cas13, CasX or CasY.

Based on the production methods disclosed herein, there is also provided for a population of genetically engineered immune cells, comprising one or more of genetically engineered NK cells and genetically engineered T cells, wherein at least about 70% of the genetically engineered NK cells express NKG2A, wherein the plurality of genetically engineered immune cells are engineered to express a cytotoxic receptor comprising an extracellular ligand binding domain, a transmembrane domain, and a cytotoxic signaling complex, wherein the genetically engineered immune cells are genetically engineered to express at least one immunosuppressive effector derived from a Human Leukocyte Antigen (HLA), wherein the at least one immunosuppressive effector exerts suppressive effects on undesired cytotoxic activity of suppressive cells, wherein, optionally, the genetically engineered immune cells are further engineered to express one or more of the following: IL-15, mblL-15, at least one viral immunosuppressive peptide integrated within an extracellular region, or extracellular regions, of the cytotoxic receptor, at least one viral immunosuppressive peptide expressed on a cell membrane of the genetically engineered immune cells, at least one portion of a human immunosuppressive protein or protein complex integrated within an extracellular region, or extracellular regions, of the cytotoxic receptor, at least one portion of a human immunosuppressive protein or protein complex expressed on a cell membrane of the genetically engineered immune cells, and/or at least one chimeric viral-human immunosuppressive construct expressed on a cell membrane of the genetically engineered immune cells and/or integrated at one or more extracellular regions of the cytotoxic receptor. In several embodiments, the immunosuppressive effector derived from an HLA comprises at least one HLA-E peptide.

In several embodiments there is provided herein a population of genetically engineered and gene edited immune cells, wherein the cells are genetically engineered in one or more respects and wherein the cells are also gene edited in one or respects. In several embodiments, the cells are genetically engineered to express one or more cytotoxic receptor that targets a tumor marker expressed by a target tumor cell. In several embodiments, the engineered immune cells are genetically edited at one or more target sites in the immune cell genome of the immune cell to yield reduced levels of expression of the protein which is encoded by the target gene which comprises an edited target site as compared to a non-edited immune cell, thereby altering one or more of the genotype, phenotype or function of the cell. In several embodiments, the genetic edits are made using a guided nuclease.

In several embodiments there is provided herein a population of genetically engineered and gene edited immune cells, comprising genetically engineered immune cells that express a cytotoxic receptor comprising an extracellular ligand binding domain, a transmembrane domain, and a cytotoxic signaling complex, wherein the extracellular ligand binding domain targets a tumor marker expressed by a target tumor cell. In several embodiments, the immune cells are genetically edited at one or more target sites in the genome of the immune cell to yield reduced levels of expression of the protein which is encoded by a gene which comprises an edited target site as compared to a non-edited immune cell. In several embodiments, the genetic edits are made using a CRISPR/CasX system. In several embodiments, the genetic edits are made using a CRISPR/CasY system. In several embodiments, the genetic edits are made using a CRISPR/Cas9 system. In several embodiments, the CRISPR/Cas systems used herein are configured to reduce potentially deleterious off-target effects. In several embodiments, the genetic edits are made using a non-Cas guided nuclease.

In several embodiments, at least a portion of the genetically engineered immune cells are engineered to express membrane bound IL-15.

In several embodiments, the genetically engineered and gene edited immune cells exhibit one or more of enhanced expansion capability, enhanced cytotoxicity against target cells, and enhanced persistence, as compared to immune cells that do not comprise genetically edited target site or sites.

In several embodiments, within the population of immune cells, the cells are edited at a gene encoding one or more of an adenosine A2 receptor CISH, SMAD3, MAPKAPK3, CEACAM1, DDIT4, TGFBR2, NKG2A, SOCS2, CBLB, B2M, TIGIT, PD-1, TIM-3, CD38, and TCR alpha to yield reduced levels of expression as compared to a non-edited immune cell. In several embodiments, within the population of immune cells, the nuclease is guided to the adenosine A2 receptor target sequence of any of SEQ ID NOs: 503-506. In several embodiments, within the population of immune cells, the cells are edited at a gene encoding CIS and the nuclease is guided to the target sequence of any of the SEQ ID NOs: 562-565. In several embodiments, within the population of immune cells, the cells are edited at a gene encoding SMAD3 and the nuclease is guided to the target sequence of any of the SEQ ID NOs: 491-493. In several embodiments, within the population of immune cells, the cells are edited at a gene encoding MAPKAPK3 and the nuclease is guided to the target sequence of any of the SEQ ID NOs: 494-496. In several embodiments, within the population of immune cells, the cells are edited at a gene encoding CEACAM1 and the nuclease is guided to the target sequence of any of the SEQ ID NOs: 497-499. In several embodiments, within the population of immune cells, the cells are edited at a gene encoding DDIT4 and the nuclease is guided to the target sequence of any of the SEQ ID NOs: 500-502. In several embodiments, within the population of immune cells, the cells are edited at a gene encoding TGFBR2 and the nuclease is guided to the target sequence of any of the SEQ ID NOs: 544-547. In several embodiments, within the population of immune cells, the cells are edited at a gene encoding NKG2A and the nuclease is guided to the target sequence of any of the SEQ ID NOs: 548-551. In several embodiments, within the population of immune cells, the cells are edited at a gene encoding SOCS2 and the nuclease is guided to the target sequence of any of the SEQ ID NOs: 556-561. In several embodiments, within the population of immune cells, the cells are edited at a gene encoding CBLB and the nuclease is guided to the target sequence of any of the SEQ ID NOs: 552-555. In several embodiments, within the population of immune cells, the cells are edited at a gene encoding B2M and the nuclease is guided to the target sequence of any of the SEQ ID NOs: 290-299. In several embodiments, within the population of immune cells, the cells are edited at a gene encoding TIGIT and the nuclease is guided to the target sequence of any of the SEQ ID NOs: 507-510. In several embodiments, within the population of immune cells, the cells are edited at a gene encoding PD-1 and the nuclease is guided to the target sequence of any of the SEQ ID NOs: 511-514. In several embodiments, within the population of immune cells, the cells are edited at a gene encoding TIM-3 and the nuclease is guided to the target sequence of any of the SEQ ID NOs: 515-518. In several embodiments, within the population of immune cells, the cells are edited at a gene encoding CD38 and the nuclease is guided to the target sequence of any of the SEQ ID NO: 519-522. In several embodiments, within the population of immune cells, the cells are edited at a gene encoding TCR alpha and the nuclease is guided to the target sequence of any of the SEQ ID NO: 566-569.

In several embodiments, the extracellular ligand binding domain comprises a receptor that is directed against at least one tumor marker selected from MICA, MICB, ULBP1, ULBP2, ULBP3, ULBP4, ULBP5, and ULBP6. In several embodiments the cytotoxic receptor expressed by NK cells comprises (i) an NKG2D ligand-binding domain, (ii) a CD8 transmembrane domain, and (iii) a signaling complex. In some embodiments, the cytotoxic signaling complex comprises an OX40 subdomain and/or a 4-1 BB domain, and a CD3zeta subdomain.

In several embodiments, the cytotoxic receptor targets one or more of NKG2D, CD19, CD70, BCMA, CD38, GPRCSD, CD138 DLL3, EGFR, PSMA, FLT3, ALPPL2, CLDN4, CLDN6, or KREMEN2 expressed by target tumor cells.

In several embodiments, the population of immune cells can comprise Natural Killer (NK) cells, T cells, induced pluripotent stem cells (iPSCs), iPSC-derived NK cells, NK-92 cells, or combinations thereof. In several embodiments, the population of immune cells comprise engineered and edited immune cells suitable for use in allogeneic cancer cell therapy with reduced risk of graft versus host disease. In several embodiments, the population of immune cells comprise engineered and edited immune cells suitable for use in allogeneic cancer cell therapy with reduced risk of cytotoxic activity between the genetically engineered immune cells. In several embodiments, the population of immune cells comprise engineered and edited immune cells for the treatment of cancer, or the preparation of a medicament for the treatment of cancer.

In several embodiments, there are provided methods for treating cancer in a subject comprising, administering to the subject a population of genetically engineered and gene edited immune cells.

In several embodiments there is provided herein a method of manufacturing a population of genetically engineered and gene edited immune cells, wherein the cells are genetically engineered in one or more respects and wherein the cells are also gene edited in one or respects. In several embodiments, the vector comprises a polynucleotide, wherein the polynucleotide encodes a cytotoxic receptor. In several embodiments, the cytotoxic receptor comprises an extracellular ligand binding domain, a transmembrane domain, and a cytotoxic signaling complex. In several embodiments, the method further includes contacting the population of immune cells with a Cas-gRNA ribonucleoprotein complex (RNP). In several embodiments, the RNP edits at one or more target sites in the genome of the immune cell to yield reduced levels of expression of the protein which is encoded by a gene which comprises an edited target site as compared to a non-edited immune cell.

Also provided for herein is a method of manufacturing a population of genetically engineered immune cells for cancer immunotherapy, comprising contacting a population of immune cells with a vector comprising a polynucleotide encoding a cytotoxic receptor comprising an extracellular ligand binding domain, a transmembrane domain, and a cytotoxic signaling complex, and contacting the population of immune cells with a Cas-gRNA ribonucleoprotein complex (RNP), wherein the RNP edits at one or more target sites in the genome of the immune cell to yield reduced levels of expression of the protein which is encoded by a gene which comprises an edited target site as compared to a non-edited immune cell, wherein the Cas of the RNP comprises Cas9, CasX, CasY, or combinations thereof, and wherein the genetically engineered and edited immune cells exhibit one or more of enhanced expansion capability, enhanced cytotoxicity against target cells, and enhanced persistence, as compared to immune cells not comprising genetically edited target sites.

In several embodiments, the method further includes gene edits that are made in a gene encoding one or more of CISH, ADARA2, SMAD3, MAPKAPK3, CEACAM1, DDIT4, TGFBR2, NKG2A, SOCS2, CBLB, B2M, TIGIT, PD-1, TIM-3, CD38, and TCR alpha. In several embodiments, the population of immune cells is contacted with a vector comprising a polynucleotide encoding a cytotoxic receptor comprising an extracellular ligand binding domain, a transmembrane domain, and a cytotoxic signaling complex, wherein the extracellular ligand binding domain comprises a receptor directed against a tumor marker selected from a group consisting of MICA, MICB, ULBP1, ULBP2, ULBP3, ULBP4, ULBP5, and ULBP6. In several embodiments, the population of immune cells is contacted with a vector comprising a polynucleotide encoding a cytotoxic receptor, wherein the cytotoxic receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the population of immune cells is contacted with a vector comprising a polynucleotide encoding a cytotoxic receptor, wherein the cytotoxic receptor targets one or more of NKG2D, CD19, CD70, BCMA, CD38, GPRC5D, CD138 DLL3, EGFR, PSMA, FLT3, ALPPL2, CLDN4, CLDN6, or KREMEN2 expressed by target tumor cells.

In several embodiments, the methods further include contacting a population of immune cells with a vector comprising a polynucleotide encoding a cytotoxic receptor comprising an extracellular ligand binding domain, a transmembrane domain, and a cytotoxic signaling complex, wherein the cytotoxic signaling complex comprises an OX40 subdomain and/or a 4-1 BB domain, and a CD3zeta subdomain.

In several embodiments, the methods further include contacting a population of immune cells with a vector comprising a polynucleotide, wherein the polynucleotide bicistronically encodes membrane bound IL15. In several embodiments, the vector is a retroviral vector.

In several embodiments, the methods further include contacting a population of immune cells, wherein the immune cells comprise Natural Killer (NK) cells, T cells, induced pluripotent stem cells (iPSCs), iPSC-derived NK cells, NK-92 cells, or combinations thereof.

Also provided for herein is a method of treating cancer in a subject comprising administering to the subject a population of genetically engineered and/or genetically edited immune cells.

In several embodiments, the immune cells express a cytotoxic receptor comprising an extracellular ligand binding domain, a transmembrane domain, and a cytotoxic signaling complex. In some embodiments, the cytotoxic signaling complex comprises an OX-40 subdomain, and a CD3zeta subdomain. In some embodiments, the method further includes where cells are engineered to express membrane bound IL-15.

In some embodiments, the immune cells are genetically edited. In some embodiments, the immune cells are genetically edited at one or more target sites in the genome of the immune cell to yield reduced levels of expression of the protein which is encoded by a gene which comprises an edited target site as compared to a non-edited immune cell. In some embodiments, the edits are made using a Crispr/CasX, Crispr/CasY, or Crispr/Cas9 system, or a combination thereof. In some embodiments, the genetically engineered and edited immune cells exhibit one or more of enhanced expansion capability, enhanced cytotoxicity against target cells, and enhanced persistence, as compared to immune cells without or not comprising genetically edited target sites.

Some embodiments relate to a method wherein immune cells are edited at a gene encoding one or more of CISH, ADAR2A, SMAD3, MAPKAPK3, CEACAM1, DDIT4, TGFBR2, NKG2A, SOCS2, CBLB, B2M, TIGIT, PD-1, TIM-3, CD38, and TCR alpha.

In several embodiments, the cytotoxic receptor expressed by the immune cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the cytotoxic receptor expressed by immune cells comprises (i) a binding domain that binds to one or more epitopes of CD19, CD70, BCMA, CD38, GPRC5D, CD138 DLL3, EGFR, PSMA, FLT3, ALPPL2, CLDN4, CLDN6, or KREMEN2 expressed by target tumor cells, (ii) a CD8 transmembrane domain, and (iii) a signaling complex that comprises an OX40 co-stimulatory subdomain and a CD3z co-stimulatory subdomain.

In several embodiments, provided is a method of treating cancer in a subject comprising administering to the subject a population of genetically engineered immune cells, wherein the immune cells comprise Natural Killer (NK) cells, T cells, induced pluripotent stem cells (iPSCs), iPSC-derived NK cells, NK-92 cells, or combinations thereof. In several embodiments, the population of genetically engineered immune cells comprise a mixture of NK cells and T cells, or a mixture of iPSC-derived NK cells and T cells.

In several embodiments, the method further provides that the NK cells and/or the iPSC-derived NK cells are edited at least to reduce CISH expression, and/or the T cells target CD19 or NKG2D. In several embodiments, method includes administering immune cells wherein the immune cells are allogeneic with respect to the subject. In several embodiments, the method includes further administering IL2.

As disclosed herein, host immune responses against administered cells (e.g., allogeneic engineered cells) can limit the efficacy and persistence of the administered cells. In some embodiments, as disclosed herein, a mixed population of immune cells are used for immunotherapy. In such cases, not only does the host immune response present a challenge, but so does the potential for the administered cells to act on one another in a manner that suppresses the efficacy of the immunotherapy, as well as against the host tissues (e.g., off target effects). In several embodiments, a mixed cell immunotherapy approach therefore requires additional engineering and/or gene editing in order to reduce graft versus host effects, host versus graft rejection, and maintenance of compatibility between the mixed immune cell types in the therapeutic. In an allogeneic immunotherapy context, an engineered T cell expressing a CAR can cause graft versus host rejection because the T cell recognizes host cells as non-self cells. This recognition occurs through the graft T cell receptor interacting with host cell HLA. Thus, in several embodiments, gene editing to disrupt the T cell receptor can eliminate T cell-based graft versus host rejection. In the inverse condition, host versus graft rejection, the host T cells recognize the graft immune cells (e.g., NK cell and/or T cells) as non-self and destroy the administered cells, thus reducing the persistence and efficacy of the immunotherapy. As editing the T cell receptor out of host cells is not practical, gene editing of the immune cells to be administered can be performed, as described herein. In several embodiments, gene editing to reduce/eliminate HLA expression on the engineered immune cells allows them to avoid being identified as non-self by host cells. Beta-2 microglobulin (B2M) gene editing/knockout is used to accomplish this, according to several embodiments disclosed herein. However, if B2M knockout is complete, this renders the edited cells susceptible to NK cell cytotoxicity, not only from host NK cells, but from NK cells within the administered immune cells (host versus graft and fratricide). This is because editing to remove B2M expression causes loss of all HLA molecules, including the normally expressed signals that would function to inhibit NK cell activity (KIR molecules). Thus, as disclosed in more detail below, in several embodiments, additional modifications are made to mask an allogeneic mixed immune cell product from both the host immune system and from fratricide from other immune cells in the mixed immune cell product. In several embodiments, various proteins can be expressed on the surface of the allogeneic immune cells that inhibits their being target by NK cells (both host and allogeneic cells). For example “don't eat me” signals that ordinarily allow cells to avoid being phagocytosed by macrophages can be expressed on allogeneic CAR-expressing NK and/or T cells to decrease targeting by NK cells. Other approaches that provide multiple benefits are also disclosed herein, for example reducing NK cell-mediated suppression while also enhancing the persistence of engineered NK cells. As discussed in more detail below, in several embodiments, immune cells (e.g., CAR expressing T cells) are engineered to express HLA-E, which gives these cells the ability to interact with inhibitory NKG2A receptor present on NK cells (host or engineered allogeneic). This interaction induces a temporary inhibition of NK cell activity, whether host or engineered allogeneic NK cells. Regarding the host NK cells, this suppression reduces the host NK cell activity against the T cells and allows the T cells to act against target tumor cells. The temporary suppression of the engineered allogeneic NK cells helps prevent NK cell exhaustion, as the engineered allogeneic NK cells has a period of reduced cytotoxic activity (e.g., a rest period). When the temporary suppression eases the engineered allogeneic NK cells return to exerting cytotoxic effects on target tumor cells. Thus, in several embodiments, the suppressive effects actually serve to enhance the persistence of the allogeneic NK cells in a mixed immune cell population for immunotherapy. In additional embodiments, peptides or proteins derived from viruses can be expressed by the engineered allogeneic cells to temporarily exert suppressive activity on NK cells (both host and those engineered NK cells within the mixed population) and thus increase the overall persistence of the allogeneic mixed immune cell population. In some embodiments, the combined acute activity of the engineered NK cells (even with the periods of suppression) and longer duration activity of the engineered T cells results in cytotoxic lysis of tumor cells. Resulting tumor debris is processed by host immune cells (e.g., dendritic cells) and tumor antigens are thus presented to T cells, thereby recruiting host immune cells to fight the tumor. In several embodiments, engineered cells are designed to undergo a temporary suppression (e.g., a period of reduced cytotoxic activity or a rest period). This suppression can be a reduction of cytotoxic or other activity by at least 10-90% (e.g., 10-30%, 30-50%, 50-70%, 70-90% and overlapping ranges therein). In one embodiment, over 90% suppression is achieved. Temporary can include, for example, minutes, hours, or days. Cells may be permanently suppressed in one embodiment.

There is provided for herein, in several embodiments, a population of genetically engineered immune cells for cancer immunotherapy, comprising genetically engineered immune cells that express a cytotoxic receptor comprising an extracellular ligand binding domain, a transmembrane domain, and a cytotoxic signaling complex, wherein the genetically engineered immune cells are engineered to express at least one immunosuppressive effector, wherein the at least one immunosuppressive effector exerts suppressive effects on the cytotoxic activity of natural killer cells and/or T cells, wherein the genetically engineered immune cells exhibit one or more of enhanced expansion capability, enhanced cytotoxicity against target cells, and enhanced persistence, as compared to cells that do not comprise said immunosuppressive effector.

Also provided for herein is a population of genetically engineered immune cells for cancer immunotherapy, comprising one or more of genetically engineered NK cells and genetically engineered T cells, wherein the genetically engineered immune cells are engineered to express a cytotoxic receptor, wherein the genetically engineered immune cells are genetically engineered to express at least one immunosuppressive effector, wherein the at least one immunosuppressive effector exerts suppressive effects on the cytotoxic activity of undesired cells, wherein the undesired cells comprise one or more of non-engineered natural killer cells, non-engineered T cells, or undesired engineered cells, and wherein the genetically engineered immune cells exhibit one or more of enhanced expansion capability, enhanced cytotoxicity against target cells, and enhanced persistence, as compared to cells that do not comprise said immunosuppressive effector.

Additionally, provided for herein, in several embodiments, is a population of genetically engineered immune cells for cancer immunotherapy, comprising one or more of genetically engineered NK cells and genetically engineered T cells, wherein the plurality of genetically engineered immune cells are engineered to express a cytotoxic receptor, wherein the genetically engineered immune cells are genetically engineered to express at least one immunosuppressive effector, wherein the at least one immunosuppressive effector exerts suppressive effects on undesired cytotoxic activity of suppressive cells, wherein the genetically engineered immune cells exhibit one or more of enhanced expansion capability, enhanced cytotoxicity against target cells, and enhanced persistence, as compared to cells that do not comprise said immunosuppressive effector.

There is also provided for herein, in several embodiments, a population of genetically engineered immune cells, comprising one or more of genetically engineered NK cells and genetically engineered T cells, wherein the plurality of genetically engineered immune cells are engineered to express a cytotoxic receptor comprising an extracellular ligand binding domain, a transmembrane domain, and a cytotoxic signaling complex, wherein the genetically engineered immune cells are genetically engineered to express at least one immunosuppressive effector, wherein the at least one immunosuppressive effector exerts suppressive effects on undesired cytotoxic activity of suppressive cells, and wherein, optionally, the genetically engineered immune cells are further engineered to express one or more of the following: IL-15, mblL-15, at least one viral immunosuppressive peptide integrated within an extracellular region, or extracellular regions, of the cytotoxic receptor, at least one viral immunosuppressive peptide expressed on a cell membrane of the genetically engineered immune cells, at least one portion of a human immunosuppressive protein or protein complex integrated within an extracellular region, or extracellular regions, of the cytotoxic receptor, at least one portion of a human immunosuppressive protein or protein complex expressed on a cell membrane of the genetically engineered immune cells, and/or at least one chimeric viral-human immunosuppressive construct expressed on a cell membrane of the genetically engineered immune cells and/or integrated at one or more extracellular regions of the cytotoxic receptor.

In several embodiments, the suppressive cells comprise host cells (e.g., NK cells and/or T cells). In several embodiments, the suppressive cells comprise one or more of non-engineered natural killer cells, non-engineered T cells, or suppressive engineered cells. In several embodiments, the suppressive engineered cells comprise the genetically engineered immune cells (e.g. those to be administered to a patient for cancer immunotherapy. In several embodiments, the cells that do not comprise said immunosuppressive effector are either non-engineered or engineered cells. In several embodiments, the immunosuppressive effector exerts transient immunosuppressive effects. In several embodiments, the transient immunosuppressive effects are beneficial for reducing the potential exhaustion of one or more of the genetically engineered immune cells. In several embodiments, the immunosuppressive effector (or effectors) are not engineered to be expressed in a matched manner across the various cell types within the genetically engineered immune cells. For example, in several embodiments, a T cell may be engineered to express a certain immunosuppressive effector (or effectors) while an NK cells is not so engineered and expresses a different profile of immunosuppressive effector (or effectors).

In several embodiments, the cytotoxic receptor comprises an extracellular ligand binding domain, a transmembrane domain, and a cytotoxic signaling complex. In several embodiments, the cytotoxic receptor targets one or more of NKG2D, CD19, and CD70 expressed by target tumor cells. In several embodiments, the cytotoxic signaling complex comprises an OX40 subdomain or a 4-1 BB domain, and a CD3zeta subdomain, or any combination thereof (including replicates of one or more subdomains).

In addition, in several embodiments, at least a portion of the genetically engineered immune cells are engineered to express membrane bound IL 5. In several embodiments, a portion of the cells are not engineered to express membrane-bound IL15. In several embodiments, NK cells are engineered to express mbIL15, while T cells are not. In several embodiments, both NK and T cells are engineered to express mbIL15. In several embodiments, T cells, not NK cells are engineered to express mbIL15, but NK cells are not. In several embodiments, NK cells and/or T cells are engineered to express soluble IL15, in addition to, or in place of mbIL15. In several embodiments, the mbIL15 is encoded by the nucleic acid sequence of SEQ ID NO: 489, or a sequence having at least 80%, 85%, 90%, 95%, 96%, 97%, 98%, or 99% sequence identity to SEQ ID NO: 489. In several embodiments, the mbIL15 has the amino acid sequence of SEQ ID NO: 490, or a sequence having at least 80%, 85%, 90%, 95%, 96%, 97%, 98%, or 99% sequence identity to SEQ ID NO: 490.

In several embodiments, the genetically engineered immune cells are also genetically edited to reduce expression of beta-2 microglobulin (B2M). In several embodiments, the reduced expression of B2M enables the immune cells to be used in allogeneic cancer immunotherapy with reduced host versus graft rejection as compared to immune cells expressing endogenous levels of B2M.

In several embodiments, the at least one immunosuppressive effector comprises a virally-derived peptide. In some embodiments, the at least one immunosuppressive effector comprises a peptide derived from a retrovirus. In several embodiments, the at least one immunosuppressive effector comprises a peptide derived from an envelope protein of a retrovirus. In several embodiments, the at least one immunosuppressive effector comprises at least a portion of a human protein and/or at least a portion of a human protein complex. In some embodiments, the at least one immunosuppressive effector comprises at least a portion of human protein. In several embodiments, the at least one immunosuppressive effector comprises a chimeric construct comprises at least one virally-derived peptide and at least a portion of a human protein and/or at least a portion of a human protein complex. In several embodiments, the chimeric immunosuppressive effector construct comprises two or more of a truncated human CD47, a p15E peptide, an HIV peptide, and an HTLV peptide. In several embodiments, the chimeric immunosuppressive effector construct comprises a truncated human CD47 domain and at least one of p15E peptide, an HIV peptide, and an HTLV peptide. In several embodiments, the chimeric immunosuppressive effector construct comprises a truncated human CD47 domain and at least one HIV peptide. In several embodiments, the chimeric immunosuppressive effector construct comprises a truncated human CD47 domain and at least HTLV peptide. In several embodiments, the chimeric immunosuppressive effector construct comprises a viral UL18 protein. In several embodiments, the chimeric immunosuppressive construct comprises a human B2M domain.

In several embodiments, the at least one immunosuppressive effector is integrated into the cytotoxic receptor. In several embodiments, the at least one immunosuppressive effector is integrated into the cytotoxic receptor between the transmembrane domain and the extracellular ligand-binding domain. In several embodiments, the at least one immunosuppressive effector is integrated into the cytotoxic receptor within the extracellular ligand-binding domain. In several embodiments, the extracellular ligand-binding domain comprises an scFv and the at least one immunosuppressive effector is integrated into a linker region of the scFv. In several embodiments, the at least one immunosuppressive effector is integrated into the cytotoxic receptor within an N-terminal region of the cytotoxic receptor distally positioned from the extracellular ligand-binding domain. Depending on the embodiment, an immunosuppressive effector is integrated into the cytotoxic receptor at a plurality of locations within an extracellular region of the cytotoxic receptor. In several embodiments, the immunosuppressive effector in a first location is different from the immunosuppressive effector at a different location(s). In some embodiments, the same immunosuppressive effector is integrated at multiple locations. In several embodiments, different immunosuppressive effectors are integrated at various locations.

In several embodiments, the at least one immunosuppressive effector is bound to an extracellular membrane of the immune cells. In several embodiments, the at least one immunosuppressive effector comprises a transmembrane protein. In several embodiments, the transmembrane protein is selected from CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. In several embodiments, the transmembrane protein comprises a CD8α transmembrane protein.

In several embodiments, the genetically engineered immune cells include a cytotoxic receptor comprising at least one immunosuppressive effector and at least one membrane-bound immunosuppressive effector.

In several embodiments, whether membrane bound or integrated into the CAR, a spacer sequence (also referred to as a hinge) is used to separate the immunosuppressive effector, or the CAR, from the transmembrane domain. In several embodiments, the spacer is selected from a CD8α, IgG1, IgG2, IgG3, IgG4, or CD28 spacer or is derived from CD8α, IgG1, IgG2, IgG3, IgG4, CD28, or can be a fully synthetic sequence. In several embodiments, the hinge region comprises one or more of SEQ ID NOs: 479-487. In several embodiments, the hinge region comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence with any of SEQ ID NOs: 479-486.

In several embodiments, the immunosuppressive effector comprises one or more peptides having at least about 80%, at least about 85%, at least about 90%, at least about 94%, or at least about 95% sequence identity to one or more of SEQ ID NOs: 199-215. In several embodiments, the immunosuppressive effector comprises a peptide having at least about 80%, at least about 85%, at least about 90%, at least about 94%, or at least about 95% sequence identity to SEQ ID NO: 199. In several embodiments, the immunosuppressive effector comprises an amino acid having at least about 80%, at least about 85%, at least about 90%, at least about 94%, or at least about 95% sequence identity to SEQ ID NO: 219. In several embodiments, the immunosuppressive effector comprises a peptide having at least about 80%, at least about 85%, at least about 90%, at least about 94%, or at least about 95% sequence identity to one or more of SEQ ID NO: 220, 225, 230, 235, and 250. In several embodiments, the immunosuppressive effector comprises an amino acid having at least about 80%, at least about 85%, at least about 90%, at least about 94%, or at least about 95% sequence identity to one or more of SEQ ID NO: 223, 228, 233, 238, and 253. In several embodiments, the immunosuppressive effector comprises a peptide having at least about 80%, at least about 85%, at least about 90%, at least about 94%, or at least about 95% sequence identity to SEQ ID NO: 240. In several embodiments, the immunosuppressive effector comprises an amino acid having at least about 80%, at least about 85%, at least about 90%, at least about 94%, or at least about 95% sequence identity to SEQ ID NO: 243.

In several embodiments, the immunosuppressive effector comprises a peptide having at least about 80%, at least about 85%, at least about 90%, at least about 94%, or at least about 95% sequence identity to one or more of SEQ ID NO: 245, 273, 276, 278, 279, 286, 287, 288, and 289. In several embodiments, the immunosuppressive effector comprises an amino acid having at least about 80%, at least about 85%, at least about 90%, at least about 94%, or at least about 95% sequence identity to SEQ ID NO: 248.

In several embodiments, the immunosuppressive effector comprises a chimeric immunosuppressive effector construct that is membrane bound and comprises a sequence having at least about 80%, at least about 85%, at least about 90%, at least about 94%, or at least about 95% sequence identity to one or more of SEQ ID Nos: 256, 259, 262, 265, 268, and 271.

In several embodiments, the chimeric immunosuppressive effector construct comprises a viral UL18 protein and comprises a sequence having at least about 80%, at least about 85%, at least about 90%, at least about 94%, or at least about 95% sequence identity to SEQ ID NO: 280. In several embodiments, the chimeric immunosuppressive effector construct comprises a viral UL18 protein and a human B2M domain and comprises a sequence having at least about 80%, at least about 85%, at least about 90%, at least about 94%, or at least about 95% sequence identity to SEQ ID NO: 283 or 285.

In several embodiments, the genetically engineered immune cells comprise genetically engineered Natural Killer (NK) cells, genetically engineered T cells, or combinations thereof. Other immune cells may be included as well, in some embodiments. In several embodiments, the genetically engineered immune cells are suitable for use in allogeneic cancer cell therapy with reduced risk of graft versus host disease. Advantageously, in several embodiments, the genetically engineered immune cells are suitable for use in allogeneic cancer cell therapy with reduced risk of cytotoxic activity between the genetically engineered immune cells.

Also provided for herein is a method for the treatment of cancer in a subject comprising administering to the subject genetically engineered immune cells according to embodiments of the present disclosure. Also provided is the use of genetically engineered immune cells according to embodiments of the present disclosure for the treatment of cancer as well their use in the preparation of a medicament for the treatment of cancer.

Additionally, in several embodiments, there is provided a method of manufacturing a population of genetically engineered immune cells for cancer immunotherapy, comprising contacting a population of immune cells with a polynucleotide encoding a cytotoxic receptor, and contacting the population of immune cells with an additional polynucleotide encoding at least one immunosuppressive effector. In several embodiments, there is provided a method of engineering a population of genetically engineered immune cells for cancer immunotherapy, comprising contacting a population of immune cells with a polynucleotide encoding a cytotoxic receptor and encoding at least one immunosuppressive effector. In several embodiments, the at least one immunosuppressive effector exerts suppressive effects on the cytotoxic activity of suppressive cells and in several embodiments, the genetically engineered immune cells exhibit one or more of enhanced expansion capability, enhanced cytotoxicity against target cells, and enhanced persistence, as compared to cells that do not comprise said at least one immunosuppressive effector. In several embodiments, the method further comprises genetically editing the DNA of the genetically engineered immune cells to alter the expression of one or more of a CISH gene, a B2M gene, a CD70 gene, an adenosine receptor gene, an NKG2A gene, a CIITA gene, a TGFBR gene, or any combination thereof. In several embodiments, the polynucleotide optionally further encodes membrane-bound IL15. In several embodiments, the method further comprises contacting the population of immune cells with a polynucleotide encoding a membrane-bound immunosuppressive effector.

Also provided for herein are methods of reducing fratricide among a mixed population of genetically engineered immune cells for cancer immunotherapy, comprising contacting a first subpopulation of immune cells from a population of mixed immune cells with a polynucleotide encoding at least one immunosuppressive effector, and contacting the second subpopulation from a population of mixed immune cells with a polynucleotide encoding at least an additional immunosuppressive effector, wherein the expression of the immunosuppressive effector by the first subpopulation and the second subpopulation reduces fratricide among the population of genetically engineered immune cells.

In several embodiments, the expression of the at least one immunosuppressive effector by the first subpopulation of immune cells reduces, at least temporarily, suppressive activity of a second subpopulation of immune cells that are directed against the first subpopulation of immune cells and expression of the at least an additional immunosuppressive effector by the second subpopulation of immune cells reduces, at least temporarily, suppressive activity of the first subpopulation of immune cells that are directed against the second subpopulation of immune cells. In several embodiments, the method further comprises contacting the population of immune cells with a polynucleotide encoding a cytotoxic receptor comprising an extracellular ligand binding domain, a transmembrane domain, and a cytotoxic signaling complex. In several embodiments, the method optionally further comprises genetically editing the DNA of the genetically engineered immune cells to alter the expression of one or more of a CISH gene, a B2M gene, a CD70 gene, an adenosine receptor gene, an NKG2A gene, a CIITA gene, a TGFBR gene, or any combination thereof.

In several embodiments, there is provided a method of engineering a population of genetically engineered immune cells for enhanced allogeneic cancer immunotherapy, comprising genetically editing a mixed population of immune cells comprising NK cells and T cells to reduce expression Human Leukocyte Antigen (HLA) on the surface of the immune cells and genetically engineering the edited cells to express one or more immunosuppressive effectors that reduce NK-mediated cytotoxicity against the mixed population of immune cells. In several embodiments, the reduced expression of HLA on the surface of the immune cells reduces T cell-mediated cytotoxicity against the edited population of immune cells. In several embodiments, the reduced expression of HLA on the surface of the cells renders the edited population of immune cells susceptible to NK-mediated cytotoxicity against the edited immune cells. In several embodiments, the one or more immunosuppressive effectors comprises one or more of a viral immunosuppressive peptide, a viral protein that is an HLA homolog, HLA-E, HLA-G, a human protein or fragment thereof that reduces phagocytosis of cells, a chimeric construct comprising a viral immunosuppressive peptide and a human protein or fragment thereof that reduces phagocytosis of cells, or combinations thereof. In several embodiments, the reduced T cell-mediated cytotoxicity reduces, at least temporarily, engineered T cell-mediated fratricidal cytotoxicity against engineered NK cells and, upon administration, host T-cell mediated cytotoxicity against engineered NK cells. In several embodiments, the reduced NK cell-mediated cytotoxicity reduces, at least temporarily, engineered NK cell-mediated fratricidal cytotoxicity against engineered T cells and, upon administration, host NK cell-mediated cytotoxicity against engineered NK and engineered T cells, thereby allowing for enhanced persistence of the engineering mixed population of immune cells upon administration to an allogeneic subject and allowing for enhanced allogeneic cancer immunotherapy.

In several embodiments, the method further comprises contacting the mixed population of immune cells with a polynucleotide encoding a cytotoxic receptor comprising an extracellular ligand binding domain, a transmembrane domain, and a cytotoxic signaling complex. In several embodiments, the polynucleotide encodes a cytotoxic receptor targeting one or more of NKG2D, CD19, and CD70 expressed by target tumor cells. In several embodiments, the cytotoxic signaling complex comprises an OX40 subdomain or a 4-1BB domain, and a CD3zeta subdomain, or any combination thereof. In several embodiments, the method further comprising genetically editing the DNA of the genetically engineered immune cells to alter the expression of one or more of a CISH gene, a B2M gene, a CD70 gene, an adenosine receptor gene, an NKG2A gene, a CIITA gene, a TGFBR gene, or any combination thereof. In several embodiments, the gene editing to reduce expression or the gene editing to induce expression is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, CasX, CasY and combinations thereof. In several embodiments, the Cas is Cas9.

In several embodiments, cells for immunotherapy are genetically modified to enhance one or more characteristics of the cells that results in a more effective therapeutic. In several embodiments, one or more of the expansion potential, cytotoxicity and/or persistence of the genetically modified immune cells is enhanced. In several embodiments, the immune cells are also engineered to express a cytotoxic receptor that targets a tumor. There is provided for herein, in several embodiments, a population of genetically engineered natural killer (NK) cell for cancer immunotherapy, comprising a plurality of NK cells, wherein the plurality of NK cells are engineered to express a cytotoxic receptor comprising an extracellular ligand binding domain, a transmembrane domain, and a cytotoxic signaling complex, wherein the NK cells are genetically edited to express reduced levels of a cytokine-inducible SH2-containing (CIS) protein encoded by a CISH gene as compared to a non-engineered NK cell, wherein the reduced CIS expression was engineered through editing of a CISH gene, and wherein the genetically engineered NK cells exhibit one or more of enhanced expansion capability, enhanced cytotoxicity against target cells, and enhanced persistence, as compared to NK cells expressing native levels of CIS. In several embodiments, the cytotoxic signaling complex comprises an OX-40 subdomain and a CD3zeta subdomain. In several embodiments, the NK cells are engineered to express membrane bound IL-15. In several embodiments, T cells are engineered and used in place of, or in addition to NK cells. In several embodiments, NKT cells are not included in the engineered immune cell population. In several embodiments, the population of immune cells comprises, consists of, or consists essentially of engineered NK cells.

In several embodiments, the extracellular ligand binding domain comprises a receptor that is directed against a tumor marker selected from the group consisting of MICA, MICB, ULBP1, ULBP2, ULBP3, ULBP4, ULBP5, and ULBP6. In several embodiments, the cytotoxic receptor expressed by the NK cells comprises, consists of, or consists essentially of (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the cytotoxic receptor is encoded by a polynucleotide having at least 80%, 85%, 90%, 95%, 96%, 97%, 98%, or 99% sequence identity to SEQ ID NO: 145. In several embodiments, the cytotoxic receptor has at least 80%, 85%, 90%, 95%, 96%, 97%, 98%, or 99% sequence identity to SEQ ID NO: 174 or 899.

In several embodiments, the cytotoxic receptor expressed by the NK cells comprises a chimeric antigen receptor (CAR) that comprises, consists of, or consists essentially of (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the anti-CD19 antibody comprises a variable heavy (VH) domain of a single chain Fragment variable (scFv) and a variable light (VL) domain of a scFv, wherein the VH domain comprises the amino acid sequence of SEQ ID NO: 120, and wherein the encoded VL domain comprises the amino acid sequence of SEQ ID NO: 118. In several embodiments, the CAR expressed by the T cells has at least 95% sequence identity to the amino acid sequence set forth in SEQ ID NO: 178 or 901. In several embodiments, the anti-CD19 antibody fragment is designed (e.g., engineered) to reduce potential antigenicity of the encoded protein and/or enhance one or more characteristics of the encoded protein (e.g., target recognition and/or binding characteristics) Thus, according to several embodiments, the anti-CD19 antibody fragment does not comprise certain sequences. For example, according to several embodiments the anti-CD19 antibody fragment is not encoded by SEQ ID NO: 116, nor does it comprise the VL regions of SEQ ID NO: 105 or 107, or the VH regions of SEQ ID NO: 104 or 106. In several embodiments, the anti-CD19 antibody fragment does not comprise one or more CDRs selected from SEQ ID NO: 108 to 115.

In several embodiments, the expression of CIS is substantially reduced as compared to a non-engineered NK cell. According to certain embodiments provided for herein, gene editing can reduce expression of a target protein, like CIS (or others disclosed herein) by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. Thus, in several embodiments, immune cells (e.g., NK cells) do not express a detectable level of CIS protein.

In several embodiments, the NK cells are further genetically engineered to express a reduced level of a transforming growth factor beta receptor (TGFBR) as compared to a non-engineered NK cell. In several embodiments, at least 50% of the population of NK cells do not express a detectable level of the TGFBR. In several embodiments, the NK cells are further genetically edited to express a reduced level of beta-2 microglobulin (B2M) as compared to a non-engineered NK cell. In several embodiments, at least 50% of the population of NK cells do not express a detectable level of B2M surface protein. In several embodiments, the NK cells are further genetically edited to express a reduced level of CIITA (class II major histocompatibility complex transactivator) as compared to a non-engineered NK cell. In several embodiments, at least 50% of the population of NK cells do not express a detectable level of CIITA. In several embodiments, the NK cells are further genetically edited to express a reduced level of a Natural Killer Group 2, member A (NKG2A) receptor as compared to a non-engineered NK cell. In several embodiments, at least 50% of the population of NK cells do not express a detectable level of NKG2A. In several embodiments, the NK cells are further genetically edited to express a reduced level of a Cbl proto-oncogene B protein encoded by a CBLB gene as compared to a non-engineered NK cell. In several embodiments, at least 50% of the population of NK cells do not express a detectable level of Cbl proto-oncogene B protein. In several embodiments, the NK cells are further genetically edited to express a reduced level of a tripartite motif-containing protein 29 protein encoded by a TRIM29 gene as compared to a non-engineered NK cell. In several embodiments, at least 50% of the population of NK cells do not express a detectable level of TRIM29 protein. In several embodiments, the NK cells are further genetically edited to express a reduced level of a suppressor of cytokine signaling 2 protein encoded by a SOCS2 gene as compared to a non-engineered NK cell. In several embodiments, at least 50% of the population of NK cells do not express a detectable level of SOCS2 protein. Depending on the embodiment, any combination of the above-referenced target proteins/genes can be edited to a desired level, including in combination with CIS, including such that the proteins are not expressed at a detectable level. In several embodiments, there may remain some amount of protein that is detectable, but the function of the protein is disrupted, substantially disrupted, eliminated or substantially eliminated. In several embodiments, even if some functionality remains, the positive effects imparted to the engineered immune cell (e.g., NK cell or T cell) remain and serve to enhance one or more anti-cancer aspects of the cells.

In several embodiments, the NK cells are further genetically edited to disrupt expression of at least one immune checkpoint protein by the NK cells. In several embodiments, the at least one immune checkpoint protein is selected from CTLA4, PD-1, lymphocyte activation gene (LAG-3), NKG2A receptor, KIR2DL-1, KIR2DL-2, KIR2DL-3, KIR2DS-1 and/or KIR2DA-2, and combinations thereof.

In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). For example in several embodiments, the NK cells are further genetically edited to express CD47. In several embodiments, the NK cells are further genetically engineered to express HLA-E. Any genes that are knocked in can be knocked in in combination with any of the genes that are knocked out or otherwise disrupted.

In several embodiments, the population of genetically engineered NK cells further comprises a population of genetically engineered T cells. In several embodiments, the population of T cells is at least partially, if not substantially, non-alloreactive. In several embodiments, the non-alloreactive T cells comprise at least one genetically edited subunit of a T Cell Receptor (TCR) such that the non-alloreactive T cells do not exhibit alloreactive effects against cells of a recipient subject. In several embodiments, the population of T cells is engineered to express a chimeric antigen receptor (CAR) directed against a tumor marker, wherein the tumor marker is one or more of CD19, CD123, CD70, Her2, mesothelin, Claudin 6, BCMA, PD-L1, EGFR. Combinations of two or more of these tumor markers can be targeted, in some embodiments. In several embodiments, the CAR expressed by the T cells is directed against CD19. In several embodiments, the CAR expressed by the T cells has at least 80%, 85%, 90%, 95%, 96%, 97%, 98%, or 99% sequence identity to the amino acid sequence set forth in SEQ ID NO: 178 or 901. In several embodiments, the CAR targets CD19. In several embodiments, the CAR is designed (e.g., engineered) to reduce potential antigenicity of the encoded protein and/or enhance one or more characteristics of the encoded protein (e.g., target recognition and/or binding characteristics) Thus, according to several embodiments, anti-CD19 CAR does not comprise certain sequences. For example, according to several embodiments the anti-CD19 CAR does not comprise by SEQ ID NO: 116, SEQ ID NO: 105, 107, 104 or 106. In several embodiments, the anti-CD19 antibody fragment does not comprise one or more CDRs selected from SEQ ID NO: 108 to 115.

In several embodiments, the TCR subunit of the T cells modified is TCRα. In several embodiments, the modification to the TCR of the T cells results in at least 80%, 85%, or 90% of the population of T cells not expressing a detectable level of the TCR. As with the edited NK cells disclosed herein, in several embodiments, the T cells are further genetically edited to reduce expression of one or more of CIS, TGFBR, B2M, CIITA, TRIM29 and SOCS2 as compared to non-engineered T cells, or to express CD47 or HLA-E. In several embodiments, the T cells are further genetically edited to disrupt expression of at least one immune checkpoint protein by the T cells, wherein the at least one immune checkpoint protein is selected from CTLA4, PD-1, and lymphocyte activation gene (LAG-3).

Depending on the embodiment, the gene editing of the NK cells and/or the T cells in order to reduce expression and/or the gene editing to induce expression is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, the gene editing of the NK cells and/or the T cells in order to reduce expression and/or the gene editing to induce expression is made using a zinc finger nuclease (ZFN). In several embodiments, the gene editing of the NK cells and/or the T cells in order to reduce expression and/or the gene editing to induce expression is made using a Transcription activator-like effector nuclease (TALEN).

In several embodiments, the genetically engineered NK cells and/or engineered T cells have an OX40 subdomain encoded by a sequence having at least 80%, 85%, 90%, or 95% sequence identity to SEQ ID NO. 5. In several embodiments, the genetically engineered NK cells and/or genetically engineered T cells have a CD3 zeta subdomain encoded by a sequence having at least 80%, 85%, 90%, or 95% sequence identity to SEQ ID NO. 7. In several embodiments, the genetically engineered NK cells and/or genetically engineered T cells have an mbIL15 encoded by a sequence having at least 80%, 85%, 90%, or 95% sequence identity to SEQ ID NO. 11 or 489.

Also provided for herein are methods of treating cancer in a subject, comprising administering to the subject a population of genetically engineered NK cells (and/or a population of genetically engineered T cells) as disclosed herein. Provided for herein is also a use of the population of genetically engineered NK cells (and/or a population of genetically engineered T cells) as disclosed herein in the treatment of cancer. Provided for herein is also a use of the population of genetically engineered NK cells (and/or a population of genetically engineered T cells) as disclosed herein in the manufacture of a medicament for the treatment of cancer.

Methods of treating cancer are also provided for herein. In several embodiments, there is provided a method for treating cancer in a subject comprising administering to the subject a population of genetically engineered immune cells, comprising (i) a plurality of NK cells, wherein the plurality of NK cells are engineered to express a cytotoxic receptor comprising an extracellular ligand binding domain, a transmembrane domain, and a cytotoxic signaling complex, wherein the NK cells are genetically edited to express reduced levels of cytokine-inducible SH2-containing (CIS) protein encoded by a CISH gene by the cells as compared to a non-engineered NK cell, wherein the reduced CIS expression was engineered through genetic editing of a CISH gene, and wherein the genetically engineered NK cells exhibit one or more of enhanced expansion capability, enhanced cytotoxicity against target cells, and enhanced persistence, as compared to NK cells expressing native levels of CIS; and optionally (ii) a plurality of T cells.

In several embodiments, the cytotoxic signaling complex comprises an OX-40 subdomain and a CD3zeta subdomain. In several embodiments, the NK cells are also engineered to express membrane bound IL-15.

In several embodiments, when included, the plurality of T cells are substantially non-alloreactive. Advantageously, in several embodiments, the non-alloreactive T cells comprise at least one modification to a subunit of a T Cell Receptor (TCR) such that the non-alloreactive T cells do not exhibit alloreactive effects against cells of a recipient subject. In several embodiments, the T cells are also engineered to express a chimeric antigen receptor (CAR) directed against a tumor marker, which can be selected from CD19, CD123, CD70, Her2, mesothelin, Claudin 6, BCMA, PD-L1, EGFR, and combinations thereof.

In several embodiments, the cytotoxic receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the cytotoxic receptor is encoded by a polynucleotide having at least 80%, 85%, 90%, or 95% sequence identity to SEQ ID NO: 145. In several embodiments, the cytotoxic receptor has at least 80%, 85%, 90%, or 95% sequence identity to SEQ ID NO: 174 or 899. In several embodiments, the cytotoxic receptor expressed by the NK cells is directed against CD19. In several embodiments, the cytotoxic receptor expressed by the NK cells has at least 80%, 85%, 90%, or 95% sequence identity to the amino acid sequence set forth in SEQ ID NO: 178 or 901. In several embodiments, the CAR expressed by the T cells is directed against CD19. In several embodiments, the CAR expressed by the T cells (and or the NK cells) comprises (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the polynucleotide encoding the CAR also encodes for membrane bound IL15. In several embodiments, the anti-CD19 antibody fragment comprises a variable heavy (VH) domain of a single chain Fragment variable (scFv) and a variable light (VL) domain of a scFv. In several embodiments, the VH domain comprises the amino acid sequence of SEQ ID NO: 120 and wherein the VL domain comprises the amino acid sequence of SEQ ID NO: 118.

In several embodiments, the NK cells and/or the T cells are further genetically edited to reduce expression of one or more of CIS, TGFBR, B2M, CIITA, TRIM29 and SOCS2 as compared to a non-engineered T cells, or to express CD47 or HLA-E.

In several embodiments, the NK cells and/or the T cells are further genetically edited to disrupt expression of at least one immune checkpoint protein by the cells, wherein the at least one immune checkpoint protein is selected from CTLA4, PD-1, and lymphocyte activation gene (LAG-3), NKG2A receptor, KIR2DL-1, KIR2DL-2, KIR2DL-3, KIR2DS-1 and/or KIR2DA-2.

In several embodiments, the OX40 subdomain is encoded by a sequence having at least 80%, 85%, 90%, or 95% sequence identity to SEQ ID NO. 5. In several embodiments, the CD3 zeta subdomain is encoded by a sequence having at least 80%, 85%, 90%, or 95% sequence identity to SEQ ID NO. 7. In several embodiments, mbIL15 is encoded by a sequence having at least 80%, 85%, 90%, or 95% sequence identity to SEQ ID NO. 11.

Depending on the embodiment of the methods disclosed herein that are applied, the gene editing of the NK cells and/or the T cells in order to reduce expression and/or the gene editing to induce expression is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, the gene editing of the NK cells and/or the T cells in order to reduce expression and/or the gene editing to induce expression is made using a zinc finger nuclease (ZFN). In several embodiments, the gene editing of the NK cells and/or the T cells in order to reduce expression and/or the gene editing to induce expression is made using a Transcription activator-like effector nuclease (TALEN).

Additionally provided for herein is a mixed population of engineered immune cells for cancer immunotherapy, comprising a plurality of NK cells, wherein the plurality of NK cells are engineered to express a cytotoxic receptor comprising an extracellular ligand binding domain, a transmembrane domain, and a cytotoxic signaling complex, wherein the NK cells are genetically edited to express reduced levels of cytokine-inducible SH2-containing (CIS) protein encoded by a CISH gene by the cells as compared to a non-engineered NK cell, wherein the reduced CIS expression was engineered through genetic editing of a CISH gene, and wherein the genetically engineered NK cells exhibit one or more of enhanced expansion capability, enhanced cytotoxicity against target cells, and enhanced persistence, as compared to NK cells expressing native levels of CIS, and a plurality of T cells that are substantially non-alloreactive through at least one modification to a subunit of a T Cell Receptor (TCR), wherein the population of T cells is engineered to express a chimeric antigen receptor (CAR) directed against a tumor marker selected from one or more of CD19, CD123, CD70, Her2, mesothelin, Claudin 6, BCMA, PD-L1, and EGFR. In several embodiments, the cytotoxic signaling complex of the cytotoxic receptor and/or CAR comprises an OX-40 subdomain and a CD3zeta subdomain. In several embodiments, the NK cells and/or the T cells are engineered to express membrane bound IL-15. In several embodiments, the cytotoxic receptor expressed by the NK cells has at least 80%, 85%, 90%, or 95% sequence identity to SEQ ID NO: 174 or 899. In several embodiments, the cytotoxic receptor expressed by the NK cells has at least 80%, 85%, 90%, or 95% sequence identity to the amino acid sequence set forth in SEQ ID NO: 178 or 901. In several embodiments, the CAR expressed by the T cells has at least 80%, 85%, 90%, or 95% sequence identity to the amino acid sequence set forth in SEQ ID NO: 178 or 901.

Provided for herein, in several embodiments, is a population of genetically altered immune cells for cancer immunotherapy, comprising a population of immune cells that are genetically modified to reduce the expression of a cytokine-inducible SH2-containing protein encoded by a CISH gene by the immune cell, genetically modified to reduce the expression of a transforming growth factor beta receptor by the immune cell, genetically modified to reduce the expression of a Natural Killer Group 2, member A (NKG2A) receptor by the immune cell, genetically modified to reduce the expression of a Cbl proto-oncogene B protein encoded by a CBLB gene by the immune cell, genetically modified to reduce the expression of a tripartite motif-containing protein 29 protein encoded by a TRIM29 gene by the immune cell, and/or genetically modified to reduce the expression of a suppressor of cytokine signaling 2 protein encoded by a SOCS2 gene by the immune cell, and genetically engineered to express a chimeric antigen receptor (CAR) directed against a tumor marker present on a target tumor cell. In several embodiments, the population comprises, consists of, or consists essentially of Natural Killer cells. In several embodiments, the population further comprises T cells. In several embodiments, the CAR is directed against CD19. In several embodiments, the CAR comprises one or more humanized CDR sequences. In several embodiments, the CAR is directed against an NKG2D ligand. In several embodiments, the genetic modification to the cells is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the modification is to CISH and the CRISPR-Cas system is guided by one or more guide RNAs selected from those comprising a sequence of SEQ ID NO. 153, 154, 155, 156, or 157; the modification is to the TGFBR2 and the CRISPR-Cas system is guided by one or more guide RNAs selected from those comprising a sequence of SEQ ID NO. 147, 148, 149, 150, 151, or 152; the modification is to NKG2A and the CRISPR-Cas system is guided by one or more guide RNAs selected from those comprising a sequence of SEQ ID NO. 158, 159, or 160; the modification is to CBLB and the CRISPR-Cas system is guided by one or more guide RNAs selected from those comprising a sequence of SEQ ID NO. 164, 165, or 166; the modification is to TRIM29 and the CRISPR-Cas system is guided by one or more guide RNAs selected from those comprising a sequence of SEQ ID NO. 167, 168, or 169, and/or the modification is to SOCS2 and the CRISPR-Cas system is guided by one or more guide RNAs selected from those comprising a sequence of SEQ ID NO. 171, 172, or 173.

In several embodiments, the genetic modification(s) is made using a zinc finger nuclease (ZFN). In several embodiments, the genetic modification(s) is made using a Transcription activator-like effector nuclease (TALEN).

In several embodiments, the genetically altered immune cells exhibit increased cytotoxicity, increased viability and/or increased anti-tumor cytokine release profiles as compared to unmodified immune cells. In several embodiments, the genetically altered immune cells have been further genetically modified to reduce alloreactivity against the cells when administered to a subject that was not the donor of the cells.

Also provided for herein is a mixed population of immune cells for cancer immunotherapy, comprising a population of T cells that are substantially non-alloreactive through at least one modification to a subunit of a T Cell Receptor (TCR) selected from TCRα, TCRβ, TCRγ, and TCRδ such that the TCR does not recognize major histocompatibility complex differences between the T cells of a recipient subject to which the mixed population of immune cells was administered, wherein the population of T cells is engineered to express a chimeric antigen receptor (CAR) directed against a tumor marker, wherein the tumor marker is selected from the group consisting of CD19, CD123, CD70, Her2, mesothelin, Claudin 6, BCMA, PD-L1, EGFR, and combinations thereof; and a population of natural killer (NK) cells, wherein the population of NK cells is engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6. In several embodiments, the TCR subunit modified is TCRα.

In several embodiments, the T cells and/or the NK cells are modified such that they express reduced levels of MHC I and/or MHC II molecules and thereby induce reduced immune response from a recipient subject's immune system to which the NK cells and T cells are allogeneic. In several embodiments, the MHC I and/or MHC II molecule is beta-microglobulin and/or CIITA (class II major histocompatibility complex transactivator). In several embodiments, the T cells and/or the NK cells further comprise a modification that disrupts expression of at least one immune checkpoint protein by the T cells and/or the NK cells. Depending on the embodiment the at least one immune checkpoint protein is selected from CTLA4, PD-1, lymphocyte activation gene (LAG-3), NKG2A receptor, KIR2DL-1, KIR2DL-2, KIR2DL-3, KIR2DS-1 and/or KIR2DA-2, and combinations thereof.

In several embodiments, the NK cells and/or T cells are further modified to reduce or substantially eliminate expression and/or function of CIS. In several embodiments, the NK cells are further engineered to express membrane bound IL-15.

In several embodiments, the CAR expressed by the T cells comprises (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the T cells also express membrane bound IL15. In several embodiments, mbIL15 is encoded by the same polynucleotide encoding the CAR. In several embodiments, the anti-CD19 antibody comprises a variable heavy (VH) domain of a single chain Fragment variable (scFv) and a variable light (VL) domain of a scFv. In some such embodiments, the VH domain comprises, consists of, or consists essentially of the amino acid sequence of SEQ ID NO: 120. In several embodiments, the encoded VL domain comprises, consists of, or consists essentially of the amino acid sequence of SEQ ID NO: 118. In several embodiments, the OX40 subdomain is encoded by a sequence having at least 80%, 85%, 90%, or 95% sequence identity to SEQ ID NO. 5. In several embodiments, the CD3 zeta subdomain is encoded by a sequence having at least 80%, 85%, 90%, or 95% sequence identity to SEQ ID NO. 7. In several embodiments, mbIL15 is encoded by a sequence having at least 80%, 85%, 90%, or 95% sequence identity to SEQ ID NO. 11. In several embodiments, the CAR expressed by the T cells has at least 80%, 85%, 90%, or 95% sequence identity to the amino acid sequence set forth in SEQ ID NO: 178 or 901. In several embodiments, chimeric receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the NK cells are further engineered to express membrane bound IL15 (which is optionally encoded by the same polynucleotide encoding the chimeric receptor). In several embodiments, the chimeric receptor is encoded by a polynucleotide having at least 80%, 85%, 90%, or 95% sequence identity to SEQ ID NO: 145. In several embodiments, the chimeric receptor has at least 80%, 85%, 90%, or 95% sequence identity to SEQ ID NO: 174 or 899.

In several embodiments, the modification to the TCR results in at least 80% of the population of T cells not expressing a detectable level of the TCR, but at least 70% of the population of T cells express a detectable level of the CAR. In several embodiments, the T cells and/or NK cells are further modified to reduce expression of one or more of a B2M surface protein, a cytokine-inducible SH2-containing protein (CIS) encoded by a CISH gene, a transforming growth factor beta receptor, a Natural Killer Group 2, member A (NKG2A) receptor, a Cbl proto-oncogene B protein encoded by a CBLB gene, a tripartite motif-containing protein 29 protein encoded by a TRIM29 gene, a suppressor of cytokine signaling 2 protein encoded by a SOCS2 gene by the T cells and/or NK cells. In several embodiments, gene editing can reduce expression of any of these target proteins by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, target protein expression can be enhanced by about 30%, about 40%, about 50%, about 60%, about 70%, about 75%, about 80%, about 85%, about 90%, about 95%, about 97%, about 98%, about 99%, or more (including any amount between those listed). For example in several embodiments, the T cells and/or NK cells are further genetically edited to express CD47. In several embodiments, the NK cells are further genetically engineered to express HLA-E. Any genes that are knocked in can be knocked in in combination with any of the genes that are knocked out or otherwise disrupted.

In several embodiments, the modification(s) to the TCR, or the further modification of the NK cells or T cells is made using a CRISPR-Cas system. In several embodiments, the CRISPR-Cas system comprises a Cas selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof.

In several embodiments, the modification(s) to the TCR, or the further modification of the NK cells or T cells is made using a zinc finger nuclease (ZFN). In several embodiments, the modification(s) to the TCR, or the further modification of the NK cells or T cells is made using a Transcription activator-like effector nuclease (TALEN).

Also provided for herein is a mixed population of immune cells for cancer immunotherapy, comprising a population of T cells that are substantially non-alloreactive due to at least one modification to a subunit of a T Cell Receptor (TCR) such that the non-alloreactive T cells do not exhibit alloreactive effects against cells of a recipient subject, wherein the population of T cells is engineered to express a chimeric antigen receptor (CAR) directed against a tumor marker selected from CD19, CD123, CD70, Her2, mesothelin, Claudin 6, BCMA, PD-L1, EGFR, and combinations thereof, and a population of natural killer (NK) cells, wherein the population of NK cells is engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6.

Also provided herein are methods of treating cancer in a subject without inducing graft versus host disease, comprising administering to the subject the mixed population of immune cells according to the present disclosure. Provided for herein are uses of the mixed population of immune cells according to the present disclosure in the treatment of cancer. Provided for herein are uses of the mixed population of immune cells according to the present disclosure in the manufacture of a medicament for the treatment of cancer.

In several embodiments, there is provided a method for treating cancer in a subject comprising administering to the subject at least a first dose of a mixed population of immune cells, wherein the mixed population of cells comprises a population of substantially non-alloreactive T cells engineered to express a chimeric antigen receptor (CAR) directed against a tumor marker selected from CD19, CD123, CD70, Her2, mesothelin, Claudin 6, BCMA, PD-L1, EGFR, and combinations thereof and a population of natural killer (NK) cells engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6.

In several embodiments, the non-alloreactive T cells comprise at least one modification to a subunit of a T Cell Receptor (TCR) such that the non-alloreactive T cells do not exhibit alloreactive effects against cells of a recipient subject. In several embodiments, the CAR expressed by the T cells is directed against CD19. In several embodiments, the CAR expressed by the T cells comprises (i) an tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain. In several embodiments, the polynucleotide encoding the CAR also encodes membrane bound IL115. In several embodiments, the anti-CD19 antibody comprises a variable heavy (VH) domain of a single chain Fragment variable (scFv) and a variable light (VL) domain of a scFv. In several embodiments, the VH domain comprises, consists of, or consists essentially of the amino acid sequence of SEQ ID NO: 120 and wherein the VL domain comprises, consists of, or consists essentially of the amino acid sequence of SEQ ID NO: 118. In several embodiments, the CAR expressed by the T cells has at least 80%, 85%, 90%, or 95% sequence identity to the amino acid sequence set forth in SEQ ID NO: 178 or 901. In several embodiments, the chimeric receptor expressed by the NK cells comprises (i) an NKG2D ligand-binding domain, (ii) a CD8 transmembrane domain, and (iii) a signaling complex that comprises an OX40 co-stimulatory subdomain and a CD3z co-stimulatory subdomain. In several embodiments, the polynucleotide encoding the chimeric receptor also encodes membrane bound IL15. In several embodiments, the chimeric receptor is encoded by a polynucleotide having at least 80%, 85%, 90%, or 95% sequence identity to SEQ ID NO: 145. In several embodiments, the chimeric receptor has at least 95%80%, 85%, 90%, or 95% sequence identity to SEQ ID NO: 174 or 899. In several embodiments, the OX40 subdomain of the CAR and/or chimeric receptor is encoded by a sequence having at least 80%, 85%, 90%, or 95% sequence identity to SEQ ID NO. 5. In several embodiments, the CD3 zeta subdomain of the CAR and/or chimeric receptor is encoded by a sequence having at least 80%, 85%, 90%, or 95% sequence identity to SEQ ID NO. 7. In several embodiments, the mbIL15 expressed by the T cells and/or the NK cells is encoded by a sequence having at least 80%, 85%, 90%, or 95% sequence identity to SEQ ID NO. 11.

In several embodiments, the cytotoxic receptor expressed by immune cells targets CD19 and has at least 80%, 85%, 90%, or 95% sequence identity to the nucleic acid sequence set forth in SEQ ID NO: 179, 181, 183, 185, 187, 189, 191, 193, 195, and/or 197. In several embodiments, the CAR expressed by the immune cells targets CD19 and has at least 80%, 85%, 90%, or 95% sequence identity to the amino acid sequence set forth in SEQ ID NO: 180, 182, 184, 186, 188, 190, 192, 194, 196, 198, and/or any of 900-911.

In several embodiments, there is provided a mixed population of immune cells for cancer immunotherapy, wherein the mixed population comprises a population of T cells that express a CAR directed against a tumor antigen, the T cells having been genetically modified to be substantially non-alloreactive and a population of NK cells expressing a CAR directed against the same tumor antigen. In several embodiments, there is provided a mixed population of immune cells for cancer immunotherapy, wherein the mixed population comprises a population of T cells that express a CAR directed against a tumor antigen, the T cells having been genetically modified to be substantially non-alloreactive and a population of NK cells expressing a CAR directed against an additional tumor antigen. In several embodiments, there is provided a mixed population of immune cells for cancer immunotherapy, wherein the mixed population comprises a population of T cells that are substantially non-alloreactive and a population of NK cells expressing a chimeric receptor targeting a tumor ligand.

In several embodiments, the non-alloreactive T cells comprise at least one modification to a subunit of a T Cell Receptor (TCR) such that the TCR recognizes an antigen without recognition of major histocompatibility complex differences between the T cells of a subject to which the mixed population of immune cells was administered. In several embodiments, the population of non-alloreactive T cells is engineered to express a chimeric antigen receptor (CAR) directed against a tumor marker (e.g., a tumor associated antigen or a tumor antigen). Depending on the embodiment, the CAR can be engineered to target one or more of CD19, CD123, CD70, Her2, mesothelin, Claudin 6 (but not other Claudins), BCMA, PD-L1, EGFR.

In several embodiments, the population of NK cells is engineered to express a chimeric receptor comprising an extracellular ligand binding domain, a transmembrane domain, a cytotoxic signaling complex and wherein the extracellular ligand binding domain a that is directed against a tumor marker selected from the group consisting of MICA, MICB, ULBP1, ULBP2, ULBP3, ULBP4, ULBP5, and ULBP6. In several embodiments, the NK cells can also be engineered to express a CAR, the CAR can be engineered to target one or more of CD19, CD123, CD70, Her2, mesothelin, Claudin 6 (but not other Claudins), BCMA, PD-L1, EGFR (or any other antigen such that both T cells and NK cells are targeting the same antigen of interest).

In several embodiments, the T cells further comprise a mutation that disrupts expression of at least one immune checkpoint protein by the T cells. For example, the T cells may be mutated with respect to an immune checkpoint protein selected from CTLA4, PD-1 and combinations thereof. In several embodiments, blocking of B7-1/B7-2 to CTLA4 is also used to reduce T cells being maintained in an inactive state. Thus, in several embodiments, T cells are modified such that they express a mismatched or mutated CTLA4, while in some embodiments, an exogenous agent can be used to, for example, bind to and/or otherwise inhibit the ability of B7-1/B7-2 on antigen presenting cells to interact with CTLA4. Likewise, in several embodiments, NK cells can be modified to disrupt expression of at least one checkpoint inhibitor. In several embodiments, for example CDTLA4 or PD-1 are modified, e.g., mutated, in order to decrease the ability of such checkpoint inhibitors to reduce NK cell cytotoxic responses. In several embodiments, Lymphocyte activation gene 3 (LAG-3, CD223), is disrupted in NK cells (and/or T cells). In several embodiments, the inhibitory NKG2A receptor is mutated, knocked-out or inhibited, for example by an antibody. Monalizumab, by way of non-limiting example, is used in several embodiments to disrupt inhibitory signaling by the NKG2A receptor. In several embodiments, one or more of the killer inhibitory receptors (KIRs) on a NK cells is disrupted (e.g., through genetic modification) and/or blocked. For example, in several embodiments, one or more of KIR2DL-1, KIR2DL-2, KIR2DL-3, KIR2DS-1 and/or KIR2DA-2, are disrupted or blocked, thereby preventing their binding to HLA-C MHC I molecules. In addition, in several embodiments, TIM3 is modified, mutated (e.g., through gene editing) or otherwise functionally disrupted (e.g., blocked by an antibody) such that its normal function of suppressing the responses of immune cells upon ligand binding is disrupted. In several such embodiments, disruption of TIM3 expression or function (e.g., through CRISPr or other methods disclosed herein), optionally in combination with disruption of one or more immune checkpoint modulator, administered T cells and/or NK cells have enhanced anti-tumor activity. Tim-3 participates in galectin-9 secretion, the latter functioning to impair the anti-cancer activity of cytotoxic lymphoid cells including natural killer (NK) cells. TIM3 is also expressed in a soluble form, which prevents secretion of interleukin-2 (IL-2). Thus, in several embodiments, the disruption of TIM3, expression, secretion, or pathway functionality provides enhanced T cell and/or NK cell activity.

In several embodiments, TIGIT (also called VSTM3) is modified, mutated (e.g., through gene editing) or otherwise functionally disrupted (e.g., blocked by an antibody) such that its normal function of suppressing the responses of immune cells upon ligand binding is disrupted. CD155 is a ligand for TIGIT. In several embodiments, TIGIT expression is reduced or knocked out. In several embodiments, TIGIT is blocked by a non-activating ligand or its activity is reduced through a competitive inhibitor of CD155 (that inhibitor not activating TIGIT). TIGIT contains an inhibit ITIM motif, which in some embodiments is excised, for example, through gene editing with CRISPr, or other methods disclosed herein. In such embodiments, the function of TIGIT is reduced, which allows for enhanced T cell and/or NK cell activity.

In several embodiments, the adenosine receptor A1 is modified, mutated (e.g., through gene editing) or otherwise functionally disrupted (e.g., blocked by an antibody) such that its normal function of suppressing the responses of immune cells upon ligand binding is disrupted. Adenosine signaling is involved in tumor immunity, as a result of its function as an immunosuppressive metabolite. Thus, in several embodiments, the Adenosine Receptor A1 expression is reduced or knocked out. In several embodiments, the adenosine receptor A1 is blocked by a non-activating ligand or its activity is reduced through a competitive inhibitor of adenosine (that inhibitor not activating adenosine signaling pathways). In several embodiments, the adenosine receptor is modified, for example, through gene editing with CRISPr, or other methods disclosed herein to reduce its function or expression, which allows for enhanced T cell and/or NK cell activity.

In several embodiments, the TCR subunit modified is selected from TCRα, TCRβ, TCRγ, and TCRδ. In several embodiments, the TCR subunit modified is TCRα.

In several embodiments, the modification to the TCR is made using a CRISPR-Cas system. In several embodiments, the disruption of expression of at least one immune checkpoint protein by the T cells or NK cells is made using a CRISPR-Cas system. For example, a Cas can be selected from Cas9, Csn2, Cas4, Cpf1, C2c1, C2c3, Cas13a, Cas13b, Cas13c, and combinations thereof. In several embodiments, the Cas is Cas9. In several embodiments, the CRISPR-Cas system comprises a Cas selected from Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof.

In several embodiments, the modification to the TCR is made using a zinc finger nuclease (ZFN). In several embodiments, the disruption of expression of the at least one immune checkpoint protein by the T cells or NK cells is made using a zinc finger nuclease (ZFN). In several embodiments, the modification to the TCR is made using a Transcription activator-like effector nuclease (TALEN). In several embodiments, the disruption of expression of the at least one immune checkpoint protein by the T cells or NK cells is made using a Transcription activator-like effector nuclease (TALEN). Combinations of ZFNs and TALENs (and optionally CRISPR-Cas) are used in several embodiments to modify either or both NK cells and T cells.

According to several embodiments, either the NK cells, the non-alloreactive T cells, or both, are further engineered to express membrane bound IL-15.

Advantageously, the mixed cell populations are useful in the methods provided for herein, wherein cancer in a subject can be treated without inducing graft versus host disease. In several embodiments, the methods comprise administering to the subject mixed population of non-alloreactive T cells expressing a CAR and engineered NK cells expressing a chimeric receptor. Also provided for are uses of a mixed population of non-alloreactive T cells expressing a CAR and engineered NK cells expressing a chimeric receptor in the treatment of cancer and/or in the manufacture of a medicament for the treatment of cancer. In still additional embodiments, the NK cells and T cells are allogeneic with respect to the subject receiving them. In several embodiments, such combinations involved NK cells and T cells directed against the same target antigen. For example, in several embodiments both the NK cells and T cells (e.g., non-alloreactive T cells) are allogeneic with respect to the subject receiving them and are engineered to express a CAR that targets the same antigen—for example CD19. In some embodiments, the NK cells and T cells are configured to both target cells expressing another marker, such as CD123, CD70, Her2, mesothelin, Claudin 6 (but not other Claudins), BCMA, PD-L1, EGFR (or any other antigen such that both T cells and NK cells are targeting the same antigen of interest).

In several embodiments, the modification to the TCR results in at least 75%, at least 80%, at least 85%, at least 90%, or at least 95% of the population of T cells that do not express a detectable level of the TCR, while at the same time at least 55%, at least 60%, at least 65%, at least 70%, or at least 75% of the population of T cells express a detectable level of the CAR. These cells are thus primarily non-alloreactive and armed with an anti-tumor-directed CAR. Further aiding in limiting immune reactions from the allogeneic T cells, in several embodiments, wherein at least 50% of the engineered T cells express a detectable level of the CAR and do not express a detectable level of TCR surface protein or B2M surface protein.

In several embodiments, NK cells are genetically modified to reduce the immune response that an allogeneic host might develop against non-self NK cells. In several embodiments, the NK cells are engineered such that they exhibit reduced expression of one or more MCH Class I and/or one or more MHC Class II molecule. In several embodiments, the expression of beta-microglobulin is substantially, significantly or completely reduced in at least a portion of NK cells that express (or will be modified to express) a CAR directed against a tumor antigen, such as CD19 (or any other antigen disclosed herein). In several embodiments, the expression of CIITA (class II major histocompatibility complex transactivator) is substantially, significantly or completely reduced in at least a portion of NK cells that express (or will be modified to express) a CAR directed against a tumor antigen, such as CD19 (or any other antigen disclosed herein). In several embodiments, such genetically modified NK cells are generated using CRISPr-Cas systems, TALENs, zinc fingers, RNAi, or other gene editing techniques. As discussed herein, in several embodiments, the NK cells with reduced allogenicity are used in combination with non-alloreactive T cells. In several embodiments, NK cells are modified to express CD47, which aids in the modified NK cell avoiding detection by endogenous innate immune cells of a recipient. In several embodiments, T cells are modified in a like fashion. In several embodiments, both NK cells and T cells are modified to express CD47, which aids in NK and/or T cell persistence in a recipient, thus enhancing anti-tumor effects. In several embodiments, NK cells are modified to express HLA-G, which aids in the modified NK cell avoiding detection by endogenous innate immune cells of a recipient. In several embodiments, T cells are modified in a like fashion. In several embodiments, both NK cells and T cells are modified to express HLA-G, which aids in NK and/or T cell persistence in a recipient, thus enhancing anti-tumor effects. In several embodiments, T cells and NK cells with reduced alloreactivity and engineered to express CARs against the same antigen are used to treat a cancer in an allogeneic patient.

In several embodiments, there is provided a population of genetically altered immune cells for cancer immunotherapy, comprising a population of immune cells that are genetically modified to reduce the expression of a transforming growth factor beta receptor by the immune cell, and genetically engineered to express a chimeric antigen receptor (CAR) directed against a tumor marker present on a target tumor cell. In additional embodiments, there is provided a population of genetically altered immune cells for cancer immunotherapy, comprising a population of immune cells that are genetically modified to reduce the expression of a Natural Killer Group 2, member A (NKG2A) receptor by the immune cell, and genetically engineered to express a chimeric antigen receptor (CAR) directed against a tumor marker present on a target tumor cell. In additional embodiments, there is provided a population of genetically altered immune cells for cancer immunotherapy, comprising a population of immune cells that are genetically modified to reduce the expression of a cytokine-inducible SH2-containing protein encoded by a CISH gene by the immune cell, and genetically engineered to express a chimeric antigen receptor (CAR) directed against a tumor marker present on a target tumor cell. CISH is an inhibitory checkpoint in NK cell-mediated cytotoxicity. In additional embodiments, there is provided a population of genetically altered immune cells for cancer immunotherapy, comprising a population of immune cells that are genetically modified to reduce the expression of a Cbl proto-oncogene B protein encoded by a CBLB gene by the immune cell, and genetically engineered to express a chimeric antigen receptor (CAR) directed against a tumor marker present on a target tumor cell. CBLB is an E3 ubiquitin ligase and a negative regulator of NK cell activation. In additional embodiments, there is provided a population of genetically altered immune cells for cancer immunotherapy, comprising a population of immune cells that are genetically modified to reduce the expression of a tripartite motif-containing protein 29 protein encoded by a TRIM29 gene by the immune cell, and genetically engineered to express a chimeric antigen receptor (CAR) directed against a tumor marker present on a target tumor cell. TRIM29 is an E3 ubiquitin ligase and a negative regulator of NK cell function after activation. In additional embodiments, there is provided a population of genetically altered immune cells for cancer immunotherapy, comprising a population of immune cells that are genetically modified to reduce the expression of a suppressor of cytokine signaling 2 protein encoded by a SOCS2 gene by the immune cell, and genetically engineered to express a chimeric antigen receptor (CAR) directed against a tumor marker present on a target tumor cell. SOCS2 is a negative regulator of NK cell function. In several embodiments the population of genetically altered immune cells comprises NK cells, T cells, or combinations thereof. In several embodiments, additional immune cell are also included, such as gamma delta T cells, NK T cells, and the like. In several embodiments, the CAR is directed against CD19. In some such embodiments, the CAR comprises one or more humanized CDR sequences. In additional embodiments, the CAR is directed against CD123. In several embodiments, the genetically modified cells are engineered to express more than one CAR that is directed to more than one target. Optionally, a mixed population of T cells and NK cells is used, in which the T cell and NK cells can each express at least one CAR, which may or may not be directed against the same cancer marker, depending on the embodiment. In several embodiments the cells express a CAR directed against an NKG2D ligand.

As discussed above, in several embodiments, the cells are edited using a CRISPr-based approach. In several embodiments, the modification is to TGFBR2 and the CRISPR-Cas system is guided by one or more guide RNAs selected from those comprising a sequence of SEQ ID NO. 147, 148, 149, 150 ,151, or 152 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 147, 148, 149, 150, 151, or 152. In several embodiments, the modification is to NKG2A and the CRISPR-Cas system is guided by one or more guide RNAs selected from those comprising a sequence of SEQ ID NO. 158, 159, or 160 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 158, 159, or 160. In several embodiments, the modification is to CISH and the CRISPR-Cas system is guided by one or more guide RNAs selected from those comprising a sequence of SEQ ID NO. 153, 154, 155, 156, or 157 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 153, 154, 155, 156, or 157. In several embodiments, the modification is to CBLB and the CRISPR-Cas system is guided by one or more guide RNAs selected from those comprising a sequence of SEQ ID NO. 164, 165 or 166 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 164, 165, or 166. In several embodiments, the modification is to TRIM29 and the CRISPR-Cas system is guided by one or more guide RNAs selected from those comprising a sequence of SEQ ID NO. 167, 168, or 169 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 167, 168, or 169. In several embodiments, the modification is to SOCS2 and the CRISPR-Cas system is guided by one or more guide RNAs selected from those comprising a sequence of SEQ ID NO. 171, 172, or 173 or a sequence that has at least 80%, 85%, 86%, 87%, 88%, 89%, 90%, 91%, 92%, 93%, 94%, 95%, 96%, 97%, 98%, or 99% homology to a sequence comprising a sequence of SEQ ID NO. 171, 172, or 173. In some embodiments, the guide RNA is 1, 2, 3, 4, 5, 6, 7, 8, 9, 10, 11, 12, 13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides long.

In several embodiments, there is provided a method for producing an engineered T cell suitable for allogenic transplantation, the method comprising delivering to a T cell an RNA-guided nuclease, a gRNA targeting a T Cell Receptor gene, and a vector comprising a donor template that comprises a nucleic acid encoding a CAR, wherein the CAR comprises (i) a tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain, and (iv) membrane bound IL15, wherein the nucleic acid encoding the CAR is flanked by left and right homology arms to the T Cell Receptor gene locus; and (b) expanding the engineered T cells in culture.

Also provided is an additional method for an engineered T cell suitable for allogenic transplantation, the method comprising delivering to a T cell an RNA-guided nuclease, and a gRNA targeting a T Cell Receptor gene, in order to disrupt the expression of at least one subunit of the TCR, and delivering to the T cell a vector comprising a nucleic acid encoding a CAR, wherein the CAR comprises (i) a tumor binding domain that comprises an anti-CD19 antibody fragment, (ii) a CD8 transmembrane domain, and (iii) a signaling complex that comprises an OX40 co-stimulatory subdomain and a CD3z co-stimulatory subdomain, and (iv) membrane bound IL15 and expanding the engineered T cells in culture.

Further methods are also provided, for example a method for producing an engineered T cell suitable for allogenic transplantation, the method comprising delivering to a T cell a nuclease capable of inducing targeted double stranded DNA breaks at a target region of a T Cell Receptor gene, in order to disrupt the expression of at least one subunit of the TCR, delivering to the T cell a vector comprising a nucleic acid encoding a CAR, wherein the CAR comprises (i) a tumor binding domain that comprises an antibody fragment that recognizes one or more of CD19, CD123, CD70, Her2, mesothelin, Claudin 6, BCMA, PD-L1, and EGFR, (ii) a CD8 transmembrane domain, and (iii) a signaling complex that comprises an OX40 co-stimulatory subdomain and a CD3z co-stimulatory subdomain, and (iv) membrane bound IL15; and expanding the engineered T cells in culture. In several embodiments, the method further comprises modifying T-cells by inactivating at least a first gene encoding an immune checkpoint protein. In several embodiments, the immune checkpoint gene is selected from the group consisting of: PD1, CTLA-4, LAG3, Tim3, BTLA, BY55, TIGIT, B7H5, LAIR1, SIGLEC10, and 2B4.

Methods for treating cancers are provided, the methods comprising generating T cells suitable for allogeneic transplant according embodiments disclosed herein, wherein the T cells are from a donor, transducing a population of NK cells expanded from the same donor to express an activating chimeric receptor that comprises an extracellular ligand binding domain a that is directed against a tumor marker selected from the group consisting of MICA, MICB, ULBP1, ULBP2, ULBP3, ULBP4, ULBP5, and ULBP6 to generate an engineered NK cell population, optionally further expanding the T cells and/or the engineered NK cell population, combining the T cells suitable for allogeneic transplant with the engineered NK cell population, and administering the combined NK and T cell population to a subject allogeneic with respect to the donor.

Methods for treating cancers are provided, the methods comprising generating T cells suitable for allogeneic transplant according embodiments disclosed herein, wherein the T cells are from a donor and are modified to express a CAR directed against CD19, CD123, CD70, Her2, mesothelin, Claudin 6 (but not other Claudins), BCMA, PD-L1, or EGFR; transducing a population of NK cells expanded from the same donor to express a CAR directed against CD19, CD123, CD70, Her2, mesothelin, Claudin 6 (but not other Claudins), BCMA, PD-L1, or EGFR to generate an engineered NK cell population, optionally further expanding the T cells and/or the engineered NK cell population, combining the T cells suitable for allogeneic transplant with the engineered NK cell population, and administering the combined NK and T cell population to a subject allogeneic with respect to the donor.

There is also provided an additional method for treating a subject for cancer, the method comprising generating T cells suitable for allogeneic transplant according to embodiments disclosed herein, wherein the T cells are from a first donor, transducing a population of NK cells expanded from a second donor to express an activating chimeric receptor that comprises an extracellular ligand binding domain a that is directed against a tumor marker selected from the group consisting of MICA, MICB, ULBP1, ULBP2, ULBP3, ULBP4, ULBP5, and ULBP6 to generate an engineered NK cell population, optionally further expanding the T cells and/or the engineered NK cell population, combining the T cells suitable for allogeneic transplant with the engineered NK cell population, administering the combined NK and T cell population to a subject allogeneic with respect to the first and the second donor.

In several embodiments, there is provided herein an immune cell, and also populations of immune cells, that expresses a CD19-directed chimeric receptor, the chimeric receptor comprising an extracellular anti-CD19 binding moiety, a hinge and/or transmembrane domain, and an intracellular signaling domain. Also provided for herein are polynucleotides (as well as vectors for transfecting cells with the same) encoding a CD19-directed chimeric antigen receptor, the chimeric antigen receptor comprising an extracellular anti-CD19 binding moiety, a hinge and/or transmembrane domain, and an intracellular signaling domain.

Also provided for herein, in several embodiments, is a polynucleotide encoding a CD19-directed chimeric antigen receptor, the chimeric antigen receptor comprising an extracellular anti-CD19 binding moiety, wherein the anti-CD19 binding moiety comprises a scFv, a hinge, wherein the hinge is a CD8 alpha hinge, a transmembrane domain, and an intracellular signaling domain, wherein the intracellular signaling domain comprises a CD3 zeta ITAM.

Also provided for herein, in several embodiments, is a polynucleotide encoding a CD19-directed chimeric antigen receptor, the chimeric antigen receptor comprising an extracellular anti-CD19 binding moiety, wherein the anti-CD19 binding moiety comprises a variable heavy chain of a scFv or a variable light chain of a scFv, a hinge, wherein the hinge is a CD8 alpha hinge, a transmembrane domain, wherein the transmembrane domain comprises a CD8 alpha transmembrane domain, and an intracellular signaling domain, wherein the intracellular signaling domain comprises a CD3 zeta ITAM.

In several embodiments, the transmembrane domain comprises a CD8 alpha transmembrane domain. In several embodiments, the transmembrane domain comprises an NKG2D transmembrane domain. In several embodiments, the transmembrane domain comprises a CD28 transmembrane domain.

In several embodiments the intracellular signaling domain comprises or further comprises a CD28 signaling domain. In several embodiments, the intracellular signaling domain comprises or further comprises a 4-1 BB signaling domain. In several embodiments, the intracellular signaling domain comprises and/or further comprises OX40 domain. In several embodiments, the intracellular signaling domain comprises or further comprises a 4-1BB signaling domain. In several embodiments, the intracellular signaling domain comprises or further comprises a domain selected from ICOS, CD70, CD161, CD40L, CD44, and combinations thereof.

In several embodiments, the polynucleotide also encodes a truncated epidermal growth factor receptor (EGFRt). In several embodiments, the EGFRt is expressed in a cell as a soluble factor. In several embodiments, the EGFRt is expressed in a membrane bound form. In several embodiments, the polynucleotide also encodes membrane-bound interleukin-15 (mbIL15). Also provided for herein are engineered immune cells (e.g., NK or T cells, or mixtures thereof) that express a CD19-directed chimeric antigen receptor encoded by a polynucleotide disclosed herein. Further provided are methods for treating cancer in a subject comprising administering to a subject having cancer engineered immune cells expressing the chimeric antigen receptors disclosed herein. In several embodiments, there is provided the use of the polynucleotides disclosed herein in the treatment of cancer and/or in the manufacture of a medicament for the treatment of cancer.

In several embodiments, the anti-CD19 binding moiety comprises a heavy chain variable (VH) domain and a light chain variable (VL) domain. In several embodiments, the VH domain has at least 95% identity to the VH domain amino acid sequence set forth in SEQ ID NO: 33. In several embodiments, the VL domain has at least 95% identity to the VL domain amino acid sequence set forth in SEQ ID NO: 32. In several embodiments, the anti-CD19 binding moiety is derived from the VH and/or VL sequences of SEQ ID NO: 33 or 32. For example, in several embodiments, the VH and VL sequences for SEQ ID NO: 33 and/or 32 are subject to a humanization campaign and therefore are expressed more readily and/or less immunogenic when administered to human subjects. In several embodiments, the anti-CD19 binding moiety comprises a scFv that targets CD19 wherein the scFv comprises a heavy chain variable region comprising the sequence of SEQ ID NO. 35 or a sequence at least 95% identical to SEQ ID NO: 35. In several embodiments, the anti-CD19 binding moiety comprises an scFv that targets CD19 comprises a light chain variable region comprising the sequence of SEQ ID NO. 36 or a sequence at least 95% identical to SEQ ID NO: 36. In several embodiments, the anti-CD19 binding moiety comprises a light chain CDR comprising a first, second and third complementarity determining region (LC CDR1, LC CDR2, and LC CDR3, respectively) and/or a heavy chain CDR comprising a first, second and third complementarity determining region (HC CDR1, HC CDR2, and HC CDR3, respectively). Depending on the embodiment, various combinations of the LC CDRs and HC CDRs are used. For example, in one embodiment the anti-CD19 binding moiety comprises LC CDR1, LC CDR3, HC CD2, and HC, CDR3. Other combinations are used in some embodiments. In several embodiments, the LC CDR1 comprises the sequence of SEQ ID NO. 37 or a sequence at least about 95% homologous to the sequence of SEQ NO. 37. In several embodiments, the LC CDR2 comprises the sequence of SEQ ID NO. 38 or a or a sequence at least about 95% homologous to the sequence of SEQ NO. 38. In several embodiments, the LC CDR3 comprises the sequence of SEQ ID NO. 39 or a sequence at least about 95% homologous to the sequence of SEQ NO. 39. In several embodiments, the HC CDR1 comprises the sequence of SEQ ID NO. 40 or a sequence at least about 95% homologous to the sequence of SEQ NO. 40. In several embodiments, the HC CDR2 comprises the sequence of SEQ ID NO. 41, 42, or 43 or a sequence at least about 95% homologous to the sequence of SEQ NO. 41, 42, or 43. In several embodiments, the HC CDR3 comprises the sequence of SEQ ID NO. 44 or a sequence at least about 95% homologous to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48 or a sequence at least about 95% homologous to the sequence of SEQ NO. 45, 46, 47, or 48. In several embodiments, the VH region comprises the sequence of SEQ ID NO. 49, 50, 51 or 52 or a sequence at least about 95% homologous to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53 or a sequence at least about 95% homologous to the sequence of SEQ NO. 53. In several embodiments, the LC CDR2 comprises the sequence of SEQ ID NO. 54 or a sequence at least about 95% homologous to the sequence of SEQ NO. 54. In several embodiments, the LC CDR3 comprises the sequence of SEQ ID NO. 55 or a sequence at least about 95% homologous to the sequence of SEQ NO. 55. In several embodiments, the HC CDR1 comprises the sequence of SEQ ID NO. 56 or a sequence at least about 95% homologous to the sequence of SEQ NO. 56. In several embodiments, the HC CDR2 comprises the sequence of SEQ ID NO. 57 or a sequence at least about 95% homologous to the sequence of SEQ NO. 57. In several embodiments, the HC CDR3 comprises the sequence of SEQ ID NO. 58 or a sequence at least about 95% homologous to the sequence of SEQ NO. 58.

In several embodiments, the intracellular signaling domain of the chimeric receptor comprises an OX40 subdomain. In several embodiments, the intracellular signaling domain further comprises a CD3zeta subdomain. In several embodiments, the OX40 subdomain comprises the amino acid sequence of SEQ ID NO: 6 (or a sequence at least about 95% homologous to the sequence of SEQ ID NO. 6) and the CD3zeta subdomain comprises the amino acid sequence of SEQ ID NO: 8 (or a sequence at least about 95% homologous to the sequence of SEQ ID NO: 8).

In several embodiments, the hinge domain comprises a CD8α hinge domain. In several embodiments, the CD8α hinge domain, comprises the amino acid sequence of SEQ ID NO: 2 or a sequence at least about 95% homologous to the sequence of SEQ ID NO: 2).

In several embodiments, the immune cell also expresses membrane-bound interleukin-15 (mbIL15). In several embodiments, the mbIL15 comprises the amino acid sequence of SEQ ID NO: 12 or a sequence at least about 95% homologous to the sequence of SEQ ID NO: 12.

In several embodiments, wherein the chimeric receptor further comprises an extracellular domain of an NKG2D receptor. In several embodiments, the immune cell expresses a second chimeric receptor comprising an extracellular domain of an NKG2D receptor, a transmembrane domain, a cytotoxic signaling complex and optionally, mbIL15. In several embodiments, the extracellular domain of the NKG2D receptor comprises a functional fragment of NKG2D comprising the amino acid sequence of SEQ ID NO: 26 or a sequence at least about 95% homologous to the sequence of SEQ ID NO: 26. In various embodiments, the immune cell engineered to express the chimeric antigen receptor and/or chimeric receptors disclosed herein is an NK cell. In some embodiments, T cells are used. In several embodiments, combinations of NK and T cells (and/or other immune cells) are used.

In several embodiments, there are provided herein methods of treating cancer in a subject comprising administering to the subject having an engineered immune cell targeting CD19 as disclosed herein. Also provided for herein is the use of an immune cell targeting CD19 as disclosed herein for the treatment of cancer. Likewise, there is provided for herein the use of an immune cell targeting CD19 as disclosed herein in the preparation of a medicament for the treatment of cancer. In several embodiments, the cancer treated is acute lymphocytic leukemia.

Some embodiments of the methods and compositions described herein relate to an immune cell. In some embodiments, the immune cell expresses a CD19-directed chimeric receptor comprising an extracellular anti-CD19 moiety, a hinge and/or transmembrane domain, and/or an intracellular signaling domain. In some embodiments, the immune cell is a natural killer (NK) cell. In some embodiments, the immune cell is a T cell.

In some embodiments, the hinge domain comprises a CD8α hinge domain. In some embodiments, the hinge domain comprises an Ig4 SH domain.

In some embodiments, the transmembrane domain comprises a CD8α transmembrane domain. In some embodiments, the transmembrane domain comprises a CD28 transmembrane domain. In some embodiments, the transmembrane domain comprises a CD3 transmembrane domain.

In some embodiments, the signaling domain comprises an OX40 signaling domain. In some embodiments, the signaling domain comprises a 4-1 BB signaling domain. In some embodiments, the signaling domain comprises a CD28 signaling domain. In some embodiments, the signaling domain comprises an NKp80 signaling domain. In some embodiments, the signaling domain comprises a CD16 IC signaling domain. In some embodiments, the signaling domain comprises a CD3zeta or CD3(ITAM signaling domain. In some embodiments, the signaling domain comprises an mblL-15 signaling domain. In some embodiments, the signaling domain comprises a 2A cleavage domain. In some embodiments, the mIL-15 signaling domain is separated from the rest or another portion of the CD19-directed chimeric receptor by a 2A cleavage domain.

Some embodiments relate to a method comprising administering an immune cell as described herein to a subject in need. In some embodiments, the subject has cancer. In some embodiments, the administration treats, inhibits, or prevents progression of the cancer.

### BRIEF DESCRIPTION OF THE DRAWINGS

FIG. 1 depicts non-limiting examples of tumor-directed chimeric antigen receptors.

FIG. 2 depicts additional non-limiting examples of tumor-directed chimeric antigen receptors.

FIG. 3 depicts additional non-limiting examples of tumor-directed chimeric antigen receptors.

FIG. 4 depicts additional non-limiting examples of tumor-directed chimeric antigen receptors.

FIG. 5 depicts additional non-limiting examples of tumor-directed chimeric antigen receptors.

FIG. 6 depicts non-limiting examples of tumor-directed chimeric antigen receptors directed against non-limiting examples of tumor markers.

FIG. 7 depicts additional non-limiting examples of tumor-directed chimeric antigen receptors directed against non-limiting examples of tumor markers.

FIGS. 8A-8I schematically depict various pathways that are altered through the gene editing techniques disclosed herein. FIG. 8A shows a schematic of the inhibitory effects of TGF-beta release by tumor cells in the tumor microenvironment. FIG. 8B shows a schematic of the CIS/CISH negative regulatory pathways on IL-15 function. FIG. 8C depicts a non-limiting schematic process flow for generation of a engineered non-alloreactive T cells and engineered NK cells for use in a combination therapy according to several embodiments disclosed herein. FIG. 8D shows a schematic of the signaling pathways that can lead to graft vs. host disease. FIG. 8E shows a schematic of how several embodiments disclosed herein can reduce and/or eliminate graft vs. host disease. FIG. 8F shows a schematic of the signaling pathways that can lead to host vs. graft rejection. FIG. 8G shows a schematic of several embodiments disclosed herein that can reduce and/or eliminate host vs. graft rejection. FIG. 8H shows a schematic of how edited immune cells can act against other edited immune cells in mixed cell product. FIG. 8I shows a schematic of how several embodiments disclosed herein can reduce and/or eliminate host immune effects against edited immune cells.

FIGS. 9A-9G show flow cytometry data related to the use of various guide RNAs to reduce expression of TGFBR2 by NK cells. FIG. 9A shows control data. FIG. 9B shows data resulting from use of guide RNA 1; FIG. 9C shows data resulting from use of guide RNA 2; FIG. 9D shows data resulting from use of guide RNA 3; FIG. 9E shows data resulting from use of guide RNA 1 and guide RNA 2; FIG. 9F shows data resulting from use of guide RNA 1 and guide RNA 3; and FIG. 9G shows data resulting from use of guide RNA 2 and guide RNA 3. Expression was evaluated 7 days after electroporation with the indicated guide RNAs.

FIGS. 10A-10G show next generation sequence data related to the reduction of expression of TGFBR2 by NK cells in response to electroporation with various guide RNAs. FIG. 10A shows control data. FIG. 10B shows data resulting from use of guide RNA 1; FIG. 10C shows data resulting from use of guide RNA 2; FIG. 10D shows data resulting from use of guide RNA 3; FIG. 10E shows data resulting from use of guide RNA 1 and guide RNA 2; FIG. 10F shows data resulting from use of guide RNA 1 and guide RNA 3; and FIG. 10G shows data resulting from use of guide RNA 2 and guide RNA 3.

FIGS. 11A-11D show data comparing the cytotoxicity of NK cells against tumor cells in the presence or absence of TGFbeta after knockdown of TGFBR2 expression by CRISPr/Cas9. FIG. 11A shows the change in cytotoxicity after TGFBR2 knockdown using guide RNAs 1 and 2. FIG. 11B shows the change in cytotoxicity after TGFBR2 knockdown using guide RNAs 1 and 3 FIG. 11C shows the change in cytotoxicity after TGFBR2 knockdown using guide RNAs 2 and 3. FIG. 11D shows data for mock TGFBR2 knockdown.

FIGS. 12A-12F show flow cytometry data related to the reduced expression of TGFBR2 by additional guide RNAs. FIG. 12A shows an unstained control of the same cells expressing TGFBR2. FIG. 12B shows positive control data for NK cells expressing TGFBR2 in the absence of electroporation with the CRISPr/Cas9 gene editing elements. FIG. 12C shows knockdown of TGFBR2 expression when guide RNA 4 was used. FIG. 12D shows knockdown of TGFBR2 expression when guide RNA 5 was used. FIG. 12E shows knockdown of TGFBR2 expression when guide RNA 6 was used. FIG. 12F shows knockdown of TGFBR2 expression when a 1:1 ratio of guide RNA 2 and 3 was used. Data were collected at 4 days post electroporation with the CRISPr/Cas9 gene editing elements.

FIGS. 13A-13F show flow cytometry data related to the expression of a non-limiting example of a chimeric antigen receptor (here an anti-CD19 CAR, NK19-1) by NK cells when subject to CRISPr/Cas9-mediated knockdown of TGFBR2. FIG. 13A shows a negative control for NK cells not engineered to express NK19-1. FIG. 13B shows positive control data for NK cells engineered to express NK19-1, but not electroporated with the CRISPr/Cas9 gene editing elements. FIG. 13C shows data related to NK19-1 expression on NK cells subjected to electroporation with guide RNA 4 to knock down TGFBR2 expression. FIG. 13D shows data related to NK19-1 expression on NK cells subjected to electroporation with guide RNA 5 to knock down TGFBR2 expression. FIG. 13E shows data related to NK19-1 expression on NK cells subjected to electroporation with guide RNA 6 to knock down TGFBR2 expression. FIG. 13F shows data related to NK19-1 expression on NK cells subjected to electroporation with guide RNAs 2 and 3 to knock down TGFBR2 expression. Data were collected at 4 days post-transduction with the vector encoding NK19-1.

FIGS. 14A-14D show data related to the resistance of NK cells expressing a non-limiting example of a CAR (here an anti-CD19 CAR, NK19-1) to TGFbeta inhibition as a result of single guide RNA knockdown of TGFBR2 expression. FIG. 14A shows cytotoxicity of the NK cells against Nalm6 tumor cells where the NK cells were cultured with the Nalm6 cells in TGFbeta in order to recapitulate the tumor microenvironment. FIGS. 14B and 14C show control data (14C) where the TGFB2 receptor was not knocked out and FIG. 14C shows selected data curves extracted from 14A in order to show the selected curves more clearly. FIG. 14D shows a schematic of the treatment of the NK cells. NK cells were subject to electroporation with CRISPr/Cas9 and a single guide RNA at Day 0 and were cultured in high IL-2 media for 1 day, followed by low-IL-2 culture with feeder cells (e.g., modified K562 cells expressing, for example, 4-1BBL and/or mbIL15). At Day 7, NK cells were transduced with a virus encoding the NK19-1 CAR construct. At Day 14, the cytotoxicity of the resultant NK cells was evaluated.

FIGS. 15A-15D show data related to the enhanced cytokine secretion by primary and NK19-1-expressing NK cells. FIG. 15A shows data related to secretion of IFNgamma. FIG. 15B shows data related to secretion of GM-CSF. FIG. 15C shows data related to secretion of Granzyme B. FIG. 15D shows data related to secretion of TNF-alpha.

FIGS. 16A-16D show data related to knockout of NKG2A expression by NK cells through use of CRISPr/Cas9. FIG. 16A shows expression of NKG2A by NK cells subjected to a mock gene editing protocol. FIG. 16B shows NKG2A expression by NK cells after editing with CRISPr/Cas9 and guide RNA 1. FIG. 16C shows NKG2A expression by NK cells after editing with CRISPr/Cas9 and guide RNA 2. FIG. 16D shows NKG2A expression by NK cells after editing with CRISPr/Cas9 and guide RNA 3.

FIGS. 17A-17B show data related to the cytotoxicity of NK cells with knocked-out NKG2A expression (as compared to mock cells). FIG. 17A shows cytotoxicity of the NKG2A-edited NK cells against REH cells at 7 days post-electroporation with the CRISPr/Cas9 gene editing elements. FIG. 17B shows flow cytometry data related to the degree of HLA-E expression on REH cells.

FIG. 18 shows data related to the cytotoxicity of mock NK cells or NK cells where Cytokine-inducible SH2-containing protein (CIS) expression was knocked out by gene editing of the CISH gene, which encodes CIS in humans. CIS is an inhibitory checkpoint in NK cell-mediated cytotoxicity. NK-cell cytotoxicity against REH tumor cells was measured at 7 days post-electroporation with the CRISPr/Cas9 gene editing elements.

FIGS. 19A-19E show data related to the impact of CISH-knockout on expression of a non-limiting example of a chimeric antigen receptor construct (here an anti-CD19 CAR, NK19-1) by NK cells. FIG. 19A shows CD19 CAR expression (as measured by FLAG expression, which is included in this construct for detection purposes, while additional embodiments of the CAR do not comprise a tag) in control (untransduced) NK cells. FIG. 19B shows anti-CD19 CAR expression in NK cells subjected to CISH knockdown using CRISPr/Cas9 and guide RNA 1. FIG. 19C shows anti-CD19 CAR expression in NK cells subjected to CISH knockdown using CRISPr/Cas9 and guide RNA 2. FIG. 19D shows anti-CD19 CAR expression in NK cells subjected to mock gene-editing conditions (electroporation only). FIG. 19E shows a Western Blot depicting the loss of the CIS protein band at 35 kDa, indicating knockout of the CISH gene.

FIGS. 20A-20B show data from a cytotoxicity assay using donor NK cells modified through gene editing and/or engineered to express a CAR against Nalm6 tumor cells. FIG. 20A shows data from a single challenge assay at a 1:2 effector:target ratio with data collected 7 days post-transduction of the indicated CAR constructs. FIG. 20B shows data from a double challenge model, where the control, edited, and/or edited/engineered NK cells were challenged with Nalm6 tumor cells at two time points.

FIGS. 21A-21B show data related CISH knockout NK cell survival and cytotoxicity over extended time in culture. FIG. 21A shows NK cell survival data over time when NK cells were treated as indicated. FIG. 21B shows NK cell cytotoxicity data against tumor cells after being cultured for 100 days.

FIGS. 22A-22E show cytokine release data by NK cells treated with the indicated control, gene editing, or gene editing+ engineered to express a CAR conditions. FIG. 22A shows data related to interferon gamma release. FIG. 22B shows data related to tumor necrosis factor alpha release. FIG. 22C shows data related to GM-CSF release. FIG. 22D shows data related to Granzyme B release. FIG. 22E shows data related to perforin release.

FIGS. 23A-23C show data from a cytotoxicity assay of mock NK cells or NK cells where either Cbl proto-oncogene B (CBLB) or tripartite motif-containing protein 29 (TRIM29) expression was knocked out by CRISPR/Cas9 gene editing. FIG. 23A shows cytotoxicity data for NK cells knocked out with three different CBLB gRNAs, CISH gRNA 5, or mock NK cells. FIG. 23B shows cytotoxicity data for NK cells knocked out with three different TRIM19 gRNAs, CISH gRNA 5, or mock NK cells. FIG. 23C shows the timeline for electroporation and cytotoxicity assay.

FIGS. 24A-24C show data from a time course cytotoxicity assay of mock NK cells or NK cells where either suppressor of cytokine signaling 2 (SOCS2) or CISH expression was knocked out by CRISPR/Cas9 gene editing. FIG. 24A shows time course cytotoxicity data for NK cells knocked out with three different SOCS2 gRNAs, CISH gRNA 2, or CD45 gRNA using the MaxCyte electroporation system. FIG. 24B shows time course cytotoxicity data for NK cells knocked out with three different SOCs2 gRNAs, CISH gRNA 2 or CD45 gRNA using the Lonza electroporation system. FIG. 24C shows the timeline for electroporation and cytotoxicity assay.

FIGS. 25A-25D show schematic depictions of non-limiting modifications made to CARs according to embodiments disclosed herein. FIG. 25A shows a non-limiting embodiment in which a single immunosuppressive effector is integrated into the hinge domain of the CAR. FIG. 25B shows a non-limiting embodiment in which multiple domains are integrated into the CAR, with a first immunosuppressive effector being of a different type than a second immunosuppressive effector. In some embodiments, a plurality of immunosuppressive effectors are included (e.g., 2, 3, 4, 5 or more), with the immunosuppressive effectors optionally being the same, or different, from any other immunosuppressive effector in the CAR. FIG. 25C shows a non-limiting embodiment wherein the immunosuppressive effector is positioned the target binding region (e.g., on the linker between the heavy and light chains of an scFv). FIG. 25D shows a non-limiting embodiment wherein the immunosuppressive effector is positioned at the N-terminus of a chimeric antigen receptor.

FIGS. 26A-26J show schematic depictions of non-limiting modifications made to immune cells, which are optionally allogeneic immune cells. FIG. 26A shows an immune cell engineered to express an immunosuppressive effector in a membrane-bound format, based on being tethered to a transmembrane protein (or fragment thereof). FIG. 26B shows an immune cell engineered to express multiple immunosuppressive effectors in a membrane-bound format. FIG. 26C shows an immune cell engineered to express multiple immunosuppressive effectors in a membrane-bound format, with each of the two effectors differing from one another. FIG. 26D shows an immune cell engineered to express multiple immunosuppressive effectors tethered to a single transmembrane protein. FIG. 26E shows an immune cell engineered to express multiple immunosuppressive effectors tethered to a single transmembrane protein, wherein the domains differ from one another. FIG. 26F shows an immune cell engineered to express multiple immunosuppressive effectors in a membrane-bound format, with each of the two effectors tethered to one of the transmembrane proteins differing from one another. FIG. 26G shows an immune cell engineered to express a membrane-bound immunosuppressive effector in conjunction with a CAR comprising one or more immunosuppressive effectors (though in some embodiments the CAR does not include an immunosuppressive effector domain). FIG. 26H shows an immune cell engineered to express two immunosuppressive effectors in a membrane-bound format in conjunction with a CAR comprising one or more immunosuppressive effectors. FIG. 26I shows an immune cell engineered to express a membrane-bound immunosuppressive effector in conjunction with a CAR comprising one or more immunosuppressive effectors and also to express one or more of HLA-E and/or HLA-G. In some embodiments like that schematically depicted, the engineered cells are NK cells that have been gene edited to disrupt B2M expression. FIG. 26J shows an immune cell engineered to express multiple immunosuppressive effectors, a CAR comprising at least one immunosuppressive effector, as well HLA-E and/or HLA-G, CD47, PD-L1, and/or the Poliovirus Receptor (PVR). While all are depicted on a single cell, any combination of the immunosuppressive effectors, or any one alone, shown in the Figures or disclosed herein may be used.

FIGS. 27A-27JJ depict schematics of non-limiting embodiments of immunosuppressive effector constructs as provided for herein.

FIG. 28 depicts a non-limiting schematic of an immune cell engineered to express a chimeric UL18-B2M construct.

FIGS. 29A-29C depict schematic diagrams related to constructs and experimental design disclosed herein. FIG. 29A shows a schematic diagram of various scenarios in which immunosuppression edits or constructs are made and the resultant self versus non-self outcome. FIG. 29B shows a schematic of a disulfide trap single chain trimer (dtSCT) used to express various immune evasion peptides (modified from Hansen et al. Current Protocols in Immunology, 2009). FIG. 29C shows a schematic of an experimental design to assess the efficacy of the hypoimmune constructs provided for herein

FIGS. 30A-30C show data related to the survival of B2M knockout T cells (as a percentage of live T cells) after co-culturing with NK cells engineered to express a non-limiting anti-CD19 CAR. FIG. 30A shows survival levels at an NK:T cell ration of 1:4. FIG. 30B shows survival at an NK:T ratio of 1:2. FIG. 30C shows survival at an NK:T ratio of 1:1.

FIGS. 31A-31C shows additional data related to HLA-E expression and effectiveness of reducing B2M deficient T cell elimination using T cell from an additional donor. FIG. 31A shows T cell survival at an NK:T cell ratio of 1:4. FIG. 31B shows T cell survival at an NK:T cell ratio of 1:2. FIG. 31C shows T cell survival at an NK:T cell ratio of 1:1.

FIG. 32 shows a schematic of the pathways by which HLA-E acts on NK cells.

FIGS. 33A-33D show data related to expression of NKG2A on NK cells and B2M on T cells for two donors after co-culturing the NK and T cells at various ratios.

FIG. 34 provides phenotypic characteristics around the characteristics of NK cell cells to be tested for their compatibility in a mixed cell product.

FIG. 35 provides phenotypic characteristics around the characteristics of T cells to be tested for their compatibility in a mixed cell product.

FIGS. 36A-36F show data related the percentage of B2M-negative cells among live T-cells when T cells expressing an anti-CD19 CAR and engineered for immune evasion according to embodiments disclosed herein are cultured with NK cells at various NK:T cell ratios. FIG. 36A shows data at an NK:T ratio of 0:1; FIG. 36B shows an NK:T ratio of 1:4; FIG. 36C shows an NK:T ratio of 1:2; FIG. 36D shows an NK:T ratio of 1:1, FIG. 36E shows an NK:T ratio of 2:1, and FIG. 36F shows an NK:T ratio of 4:1.

FIGS. 36G-36L show data related the percentage of B2M-negative cells among live T-cells when T cells expressing an anti-CD19 CAR and engineered for immune evasion according to embodiments disclosed herein are cultured with NK cells also expressing an anti-CD19 CAR, at various NK:T cell ratios. FIG. 36G shows data at an NK:T ratio of 0:1; FIG. 36H shows an NK:T ratio of 1:4; FIG. 36I shows an NK:T ratio of 1:2; FIG. 36J shows an NK:T ratio of 1:1, FIG. 36K shows an NK:T ratio of 2:1, and FIG. 36L shows an NK:T ratio of 4:1.

FIGS. 37A-37F show data related the percentage of B2M-negative cells among live T-cells when T cells (collected from a first donor) expressing an anti-CD19 CAR and engineered for immune evasion according to embodiments disclosed herein are cultured with NK cells also expressing an anti-CD19 CAR, at various NK:T cell ratios. FIG. 37A shows data at an NK:T ratio of 0:1; FIG. 37B shows an NK:T ratio of 1:4; FIG. 37C shows an NK:T ratio of 1:2; FIG. 37D shows an NK:T ratio of 1:1, FIG. 37E shows an NK:T ratio of 2:1, and FIG. 37F shows an NK:T ratio of 4:1.

FIGS. 38A-38F show data related the percentage of B2M-negative cells among live T-cells when T cells (collected from a second donor) expressing an anti-CD19 CAR and engineered for immune evasion according to embodiments disclosed herein are cultured with NK cells also expressing an anti-CD19 CAR, at various NK:T cell ratios. FIG. 38A shows data at an NK:T ratio of 0:1; FIG. 38B shows an NK:T ratio of 1:4; FIG. 38C shows an NK:T ratio of 1:2; FIG. 38D shows an NK:T ratio of 1:1, FIG. 38E shows an NK:T ratio of 2:1, and FIG. 38F shows an NK:T ratio of 4:1.

FIGS. 39A-39F show data related the percentage of B2M-negative cells among live T-cells when T cells (collected from a third donor) expressing an anti-CD19 CAR and engineered for immune evasion according to embodiments disclosed herein are cultured with NK cells also expressing an anti-CD19 CAR, at various NK:T cell ratios. FIG. 39A shows data at an NK:T ratio of 0:1; FIG. 39B shows an NK:T ratio of 1:4; FIG. 39C shows an NK:T ratio of 1:2; FIG. 39D shows an NK:T ratio of 1:1, FIG. 39E shows an NK:T ratio of 2:1, and FIG. 39F shows an NK:T ratio of 4:1.

FIGS. 40A-40F show data related the percentage of B2M-negative cells among live T-cells when T cells (collected from a first donor) expressing an anti-CD19 CAR and engineered for immune evasion (either with a TCR knockout, a TCR knockout and B2M knockout, or A TCR knockout with a B2M knockout and a re-expression of an HLA) are cultured with NK cells also expressing an anti-CD19 CAR, at various NK:T cell ratios. FIG. 40A shows data at an NK:T ratio of 0:1; FIG. 40B shows an NK:T ratio of 1:4; FIG. 40C shows an NK:T ratio of 1:2; FIG. 40D shows an NK:T ratio of 1:1, FIG. 40E shows an NK:T ratio of 2:1, and FIG. 40F shows an NK:T ratio of 4:1.

FIGS. 41A-41F show data related the percentage of B2M-negative cells among live T-cells when T cells (collected from a first donor) expressing an anti-CD19 CAR and engineered for immune evasion (either with a TCR knockout, a TCR knockout and B2M knockout, or A TCR knockout with a B2M knockout and a re-expression of an HLA) are cultured with NK cells also expressing an anti-CD19 CAR, at various NK:T cell ratios. FIG. 41A shows data at an NK:T ratio of 0:1; FIG. 41B shows an NK:T ratio of 1:4; FIG. 41C shows an NK:T ratio of 1:2; FIG. 41D shows an NK:T ratio of 1:1, FIG. 41E shows an NK:T ratio of 2:1, and FIG. 41F shows an NK:T ratio of 4:1.

FIGS. 42A-42B show schematics of various approaches to reduce immune responses against engineered therapeutic cells. FIG. 42A depicts a schematic of a CAR comprising a hypoimmune domain (HYPO) and various peptides or HLA sequences that are used to reduce immune responses against engineered therapeutic cells. FIG. 42B depicts various peptides and combinations of peptides that are used to reduce immune responses against engineered therapeutic cells.

FIG. 43 shows a schematic of an experimental design that allows for the evaluation of the various hypoimmunity-inducing embodiments disclosed herein.

FIG. 44 shows cytotoxicity data to evaluate hypoimmunity-inducing constructs according to embodiments provided for herein.

FIGS. 45A-45C show additional cytotoxicity data collected while evaluating hypoimmunity-inducing constructs according to embodiments provided for herein. FIG. 45A shows cytotoxicity data using NK cells from a first donor against K562 cells expressing various hypoimmune constructs. FIG. 45B shows cytotoxicity data using NK cells from an additional donor against K562 cells expressing various hypoimmune constructs. FIG. 45C shows cytotoxicity data using immortalized NK92 cells against K562 cells expressing various hypoimmune constructs.

FIG. 46 depicts an analysis of NKG2A expression by donor NK cells.

FIGS. 47A-47R show experimental setup and data related to cytotoxicity of various immune constructs provided for in embodiments disclosed herein. FIG. 47A shows a non-limiting schematic of an assay procedure for assessing the immune evasion effects of various constructs provided for in embodiments herein. FIG. 47B shows cytotoxicity curves after hypoimmune modified K562 cells were co-cultured with activated NK cells from a first donor. FIG. 47C shows a histogram depicting the detection of GFP expressed by the modified K562 cells, representing the degree of expression of the indicated hypoimmune construct. FIG. 47D shows cytotoxicity curves after hypoimmune modified K562 cells were co-cultured with activated NK cells from a second donor. FIG. 47E shows a histogram depicting the detection of GFP expressed by the modified K562 cells, representing the degree of expression of the indicated hypoimmune construct. FIG. 47F shows cytotoxicity curves after hypoimmune modified K562 cells were co-cultured with activated NK cells from a third donor. FIG. 47G shows a histogram depicting the detection of GFP expressed by the modified K562 cells, representing the degree of expression of the indicated hypoimmune construct. FIG. 47H shows cytotoxicity curves after hypoimmune modified K562 cells were co-cultured with activated NK cells from a donor. FIG. 47I shows a histogram depicting the detection of GFP expressed by the modified K562 cells, representing the degree of expression of the indicated hypoimmune construct. FIG. 47J shows cytotoxicity curves after hypoimmune modified K562 cells were co-cultured with activated NK cells from a donor. FIG. 47K shows a histogram depicting the detection of GFP expressed by the modified K562 cells, representing the degree of expression of the indicated hypoimmune construct. FIG. 47L shows cytotoxicity curves after hypoimmune modified K562 cells were co-cultured with activated NK cells from a donor. FIG. 47M shows a histogram depicting the detection of GFP expressed by the modified K562 cells, representing the degree of expression of the indicated hypoimmune construct. FIG. 47N shows cytotoxicity curves after hypoimmune modified K562 cells were co-cultured with activated NK cells from a donor. FIG. 47O shows a histogram depicting the detection of GFP expressed by the modified K562 cells, representing the degree of expression of the indicated hypoimmune construct. FIG. 47P shows a summary tabulation of the screening of the indicated hypoimmune constructs. FIG. 47Q shows cytotoxicity curves after hypoimmune modified K562 cells were co-cultured with activated NK cells from a donor. FIG. 47R shows a histogram depicting the detection of GFP expressed by the modified K562 cells, representing the degree of expression of the indicated hypoimmune construct.

FIGS. 48A-48B show cytotoxicity data from a variety of hypoimmune constructs as provided for herein. FIG. 48A shows cytotoxicity curves for K562 cells modified to express the indicated immune evasion constructs that were co-cultured with activated donor NK cells. FIG. 48B shows a histogram depicting the detection of GFP expressed by the modified K562 cells, representing the degree of expression of the indicated hypoimmune construct.

FIGS. 49A-49C show data correlating the degree of expression of selected hypoimmune constructs provided for herein with immune protection. FIG. 49A shows cytotoxicity curves for high HLA-E expressing K562NR cells and low HLA-E expressing K562NR cells as well as a summary table reflecting the relative expression of the HLA-E construct by both sets of cells. FIG. 49B shows cytotoxicity curves for high CD47 expressing K562NR cells and low CD47 expressing K562NR cells as well as a summary table reflecting the relative expression of the CD47 construct by both sets of cells. FIG. 49C shows cytotoxicity curves for high CD43_TM expressing K562NR cells and low CD47_TM expressing K562NR cells as well as a summary table reflecting the relative expression of the CD47_TM construct by both sets of cells.

FIGS. 50A-50D show data from non-limiting embodiments of combinations of gene edits made to NK cells and the resultant cytotoxicity. FIG. 50A shows data with the indicated edits made in NK cells and an evaluation of cytotoxicity against target 786-O cells 14 days after the edits were made. FIG. 50B shows data corresponding to that shown in FIG. 50A, but in the presence of TGFb. FIG. 50C shows data with the indicated edits made in NK cells and an evaluation of cytotoxicity against target 786-O cells 21 days after the edits were made. FIG. 50D shows data corresponding to that shown in FIG. 9C, but in the presence of TGFb.

FIGS. 51A-51D show data from non-limiting embodiments of combinations of gene edits made to NK cells and the resultant cytotoxicity. FIG. 51A shows data with the indicated edits made in NK cells and an evaluation of cytotoxicity against target 786-O cells 14 days after the edits were made. FIG. 51B shows data corresponding to that shown in FIG. 51A, but in the presence of TGFb. FIG. 51C shows data with the indicated edits made in NK cells and an evaluation of cytotoxicity against target ACHN cells 21 days after the edits were made. FIG. 51D shows data corresponding to that shown in FIG. 51C, but in the presence of TGFb.

FIGS. 52A-52B show data related to the cytotoxicity of NK cells edited at the indicated genes against target cells. FIG. 52A shows data with the indicated edits made in NK cells and an evaluation of cytotoxicity against target 786-O cells 21 days after the edits were made. FIG. 52B shows data corresponding to that shown in FIG. 52A, but in the presence of adenosine.

FIGS. 53A-53D show data related to the cytotoxicity of NK cells edited at multiple gene targets. FIG. 53A shows data related to NK cells edited in the indicated manner and their cytotoxicity against target 786-O cells 14 days post-edit. FIG. 53B shows summary data of the traces in FIG. 53A. FIG. 53C shows data related to NK cells edited in the indicated manner and their cytotoxicity against target 786-O cells 14 days post-edit in the presence of TGFb. FIG. 53D shows summary data of the traces in FIG. 53C.

FIGS. 54A-54B show data related to the cytotoxicity of NK cells edited at multiple gene targets. FIG. 53A shows data related to NK cells edited in the indicated manner and their cytotoxicity against target ACHN cells 14 days post-edit. FIG. 54B shows data related to NK cells edited in the indicated manner and their cytotoxicity against target ACHN cells 14 days post-edit in the presence of TGFb

FIGS. 55A-55D show data related to the cytotoxicity of NK cells edited at multiple gene targets. FIG. 55A shows data related to NK cells edited in the indicated manner and their cytotoxicity against target 786-O cells 28 days post-edit. FIG. 55B shows summary data of the traces in FIG. 55A. FIG. 55C shows data related to NK cells edited in the indicated manner and their cytotoxicity against target 786-O cells 28 days post-edit in the presence of TGFb. FIG. 55D shows summary data of the traces in FIG. 55C.

FIGS. 56A-56D show data related to the cytotoxicity of NK cells edited at multiple gene targets. FIG. 56A shows data related to NK cells edited in the indicated manner and their cytotoxicity against target ACHN cells 28 days post-edit. FIG. 56B shows summary data of the traces in FIG. 56A. FIG. 56C shows data related to NK cells edited in the indicated manner and their cytotoxicity against target ACHN cells 28 days post-edit in the presence of TGFb. FIG. 56D shows summary data of the traces in FIG. 56C.

### DETAILED DESCRIPTION

Some embodiments of the methods and compositions provided herein relate to engineered immune cells and combinations of the same for use in immunotherapy. In several embodiments, the engineered cells are engineered in multiple ways, for example, to express a cytotoxicity-inducing receptor complex. As used herein, the term “cytotoxic receptor complexes” shall be given its ordinary meaning and shall also refer to (unless otherwise indicated), Chimeric Antigen Receptors (CAR), chimeric receptors (also called activating chimeric receptors in the case of NKG2D chimeric receptors). In several embodiments, the cells are further engineered to achieve a modification of the reactivity of the cells against non-tumor tissue. Several embodiments relate to the modification of T cells, through various genetic engineering methodologies, such that the resultant T cells have reduced and/or eliminated alloreactivity. Such non-alloreactive T cells can also be engineered to express a chimeric antigen receptor (CAR) that enables the non-alloreactive T cells to impart cytotoxic effects against tumor cells. In several embodiments, natural killer (NK) cells are also engineered to express a city-inducing receptor complex (e.g., a chimeric antigen receptor or chimeric receptor). In several embodiments, combinations of these engineered immune cell types are used in immunotherapy, which results in both a rapid (NK-cell based) and persistent (T-cell based) anti-tumor effect, all while advantageously having little to no graft versus host disease. Some embodiments include methods of use of the compositions or cells in immunotherapy.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, and/or amelioration of various physiological symptoms associated with the cancerous condition.

### Cell Types

Some embodiments of the methods and compositions provided herein relate to a cell such as an immune cell. For example, an immune cell, such as a T cell, may be engineered to include a chimeric receptor such as a CD19-directed chimeric receptor, or engineered to include a nucleic acid encoding said chimeric receptor as described herein. Additional embodiments relate to engineering a second set of cells to express another cytotoxic receptor complex, such as an NKG2D chimeric receptor complex as disclosed herein. Still additional embodiments relate to the further genetic manipulation of T cells (e.g., donor T cells) to reduce, disrupt, minimize and/or eliminate the ability of the donor T cell to be alloreactive against recipient cells (graft versus host disease).

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are provided for herein polynucleotides, polypeptides, and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. For example, some embodiments include a polynucleotide, polypeptide, or vector that encodes, for example a chimeric antigen receptor directed against a tumor marker, for example, CD19, CD123, CD70, Her2, mesothelin, Claudin 6, BCMA, EGFR, among others, to facilitate targeting of an immune cell to a cancer and exerting cytotoxic effects on the cancer cell. Also provided are engineered immune cells (e.g., T cells or NK cells) expressing such CARs. There are also provided herein, in several embodiments, polynucleotides, polypeptides, and vectors that encode a construct comprising an extracellular domain comprising two or more subdomains, e.g., first CD19-targeting subdomain comprising a CD19 binding moiety as disclosed herein and a second subdomain comprising a C-type lectin-like receptor and a cytotoxic signaling complex. Also provided are engineered immune cells (e.g., T cells or NK cells) expressing such bi-specific constructs. Methods of treating cancer and other uses of such cells for cancer immunotherapy are also provided for herein.

To facilitate cancer immunotherapies, there are also provided for herein polynucleotides, polypeptides, and vectors that encode chimeric receptors that comprise a target binding moiety (e.g., an extracellular binder of a ligand expressed by a cancer cell) and a cytotoxic signaling complex. For example, some embodiments include a polynucleotide, polypeptide, or vector that encodes, for example an activating chimeric receptor comprising an NKG2D extracellular domain that is directed against a tumor marker, for example, MICA, MICB, ULBP1, ULBP2, ULBP3, ULBP4, ULBP5, and ULBP6, among others, to facilitate targeting of an immune cell to a cancer and exerting cytotoxic effects on the cancer cell. Also provided are engineered immune cells (e.g., T cells or NK cells) expressing such chimeric receptors. There are also provided herein, in several embodiments, polynucleotides, polypeptides, and vectors that encode a construct comprising an extracellular domain comprising two or more subdomains, e.g., first and second ligand binding receptor and a cytotoxic signaling complex. Also provided are engineered immune cells (e.g., T cells or NK cells) expressing such bi-specific constructs (in some embodiments the first and second ligand binding domain target the same ligand). Methods of treating cancer and other uses of such cells for cancer immunotherapy are also provided for herein.

### Engineered Cells for Immunotherapy

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric receptor, such as an NKG2D chimeric receptor, and/or a CAR, such as a CD19-directed CAR, or a nucleic acid encoding the chimeric receptor or the CAR. In several embodiments, the cells are optionally engineered to co-express a membrane-bound interleukin 15 (mbIL15) co-stimulatory domain. As discussed in more detail below, in several embodiments, the cells, particularly T cells, are further genetically modified to reduce and/or eliminate the alloreactivity of the cells.

### Monocytes for Immunotherapy

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake of cellular material, or entire cells, followed by digestion and destruction of the engulfed cellular material. In several embodiments, monocytes are used in connection with one or more additional engineered cells as disclosed herein. Some embodiments of the methods and compositions described herein relate to a monocyte that includes a tumor-directed CAR, or a nucleic acid encoding the tumor-directed CAR. Several embodiments of the methods and compositions disclosed herein relate to monocytes engineered to express a CAR that targets a tumor marker, for example, CD19, CD123, CD70, Her2, mesothelin, Claudin 6, BCMA, EGFR, among any of the others disclosed herein, and a membrane-bound interleukin 15 (mbIL15) co-stimulatory domain. Several embodiments of the methods and compositions disclosed herein relate to monocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Lymphocytes for Immunotherapy

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Several embodiments of the methods and compositions disclosed herein relate to lymphocytes engineered to express a CAR that targets a tumor marker, for example, CD19, CD123, CD70, Her2, mesothelin, Claudin 6, BCMA, EGFR, among any of the others disclosed herein, and a membrane-bound interleukin 15 (mbIL15) co-stimulatory domain. Several embodiments of the methods and compositions disclosed herein relate to lymphocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### T Cells for Immunotherapy

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Several embodiments of the methods and compositions disclosed herein relate to T cells engineered to express a CAR that targets a tumor marker, for example, CD19, CD123, CD70, Her2, mesothelin, Claudin 6, BCMA, EGFR, among any of the others as disclosed herein, and a membrane-bound interleukin 15 (mbIL15) co-stimulatory domain. Several embodiments of the methods and compositions disclosed herein relate to T cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### NK Cells for Immunotherapy

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). Some embodiments of the methods and compositions described herein relate to NK cells engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to NK cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain. In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference. NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

### Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to hematopoietic stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Induced Pluripotent Stem Cells

In some embodiments, induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the cells are engineered to express a homing moiety and/or a cytotoxic receptor complex. In several embodiments, iPSCs are used in combination with one or more additional engineered cell type disclosed herein. Some embodiments of the methods and compositions described herein relate to a stem cell, such as a induced pluripotent stem cell engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Genetic Engineering of Immune Cells

As discussed above, a variety of cell types can be utilized in cellular immunotherapy. Further, as elaborated on in more detail below, and shown in the Examples, genetic modifications can be made to these cells in order to enhance one or more aspects of their efficacy (e.g., cytotoxicity) and/or persistence (e.g., active life span).

### Targets for Gene Editing

Additional cellular engineering strategies are provided for herein that serve to further enhance the persistence of allogeneic cellular therapy products, such as allogeneic CAR-T cells and/or allogeneic CAR-NK cells. There is provided for herein, in several embodiments, a population of genetically engineered immune cells for cancer immunotherapy where the genetically engineered immune cells are genetically modified (e.g., gene edited) at one, two, three or more gene loci to enhance the cytotoxic activity, persistence, or other feature of the cells, such as NK cells and/or T cells.

As discussed herein, in several embodiments NK cells are used for immunotherapy. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein. As discussed below, in several embodiments, gene editing is employed to reduce or knockout expression of target proteins, for example by disrupting the underlying gene encoding the protein. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). Unless indicated otherwise to the contrary, the sequences provided for guide RNAs that are recited using deoxyribonucleotides refer to the target DNA and shall be considered as also referencing those guides used in practice (e.g., employing ribonucleotides, where the ribonucleotide uracil is used in lieu of deoxyribonucleotide thymine or vice-versa where thymine is used in lieu of uracil, wherein both are complementary base pairs to adenine when reciting either an RNA or DNA sequence). For example, a gRNA with the sequence ATGCTCAATGCGTC (SEQ ID NO: 995) shall also refer to the following sequence AUGCUCAAUGCGUC (SEQ ID NO: 996) or a gRNA with sequence AUGCUCAAUGCGUC (SEQ ID NO: 996) shall also refer to the following sequence ATGCTCAATGCGTC (SEQ ID NO: 995).

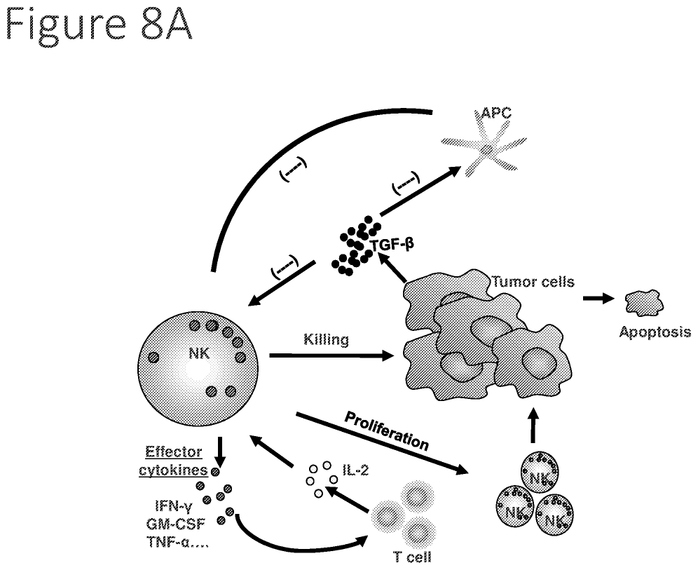

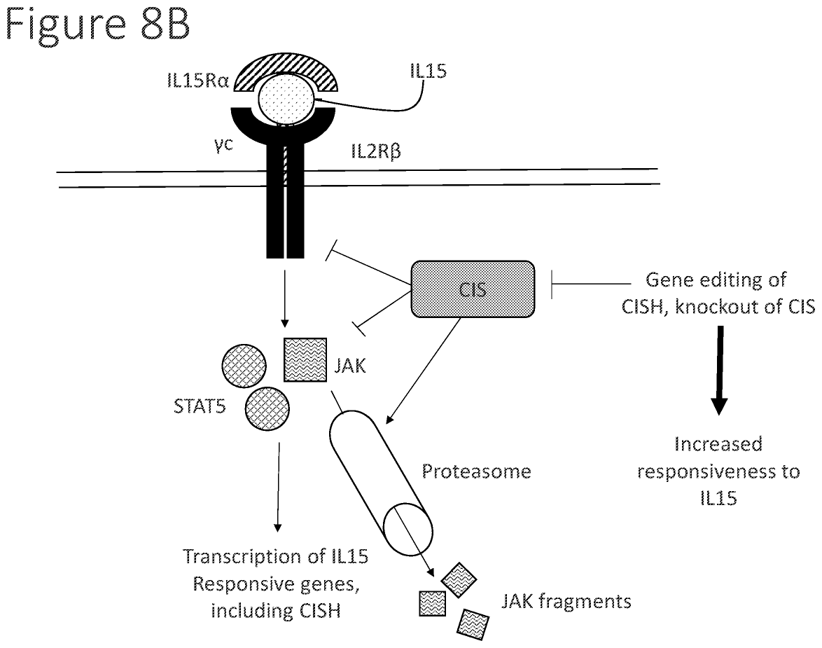

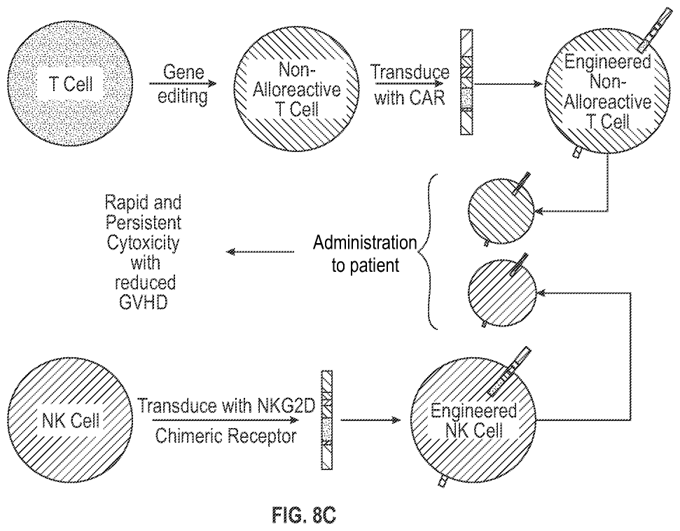

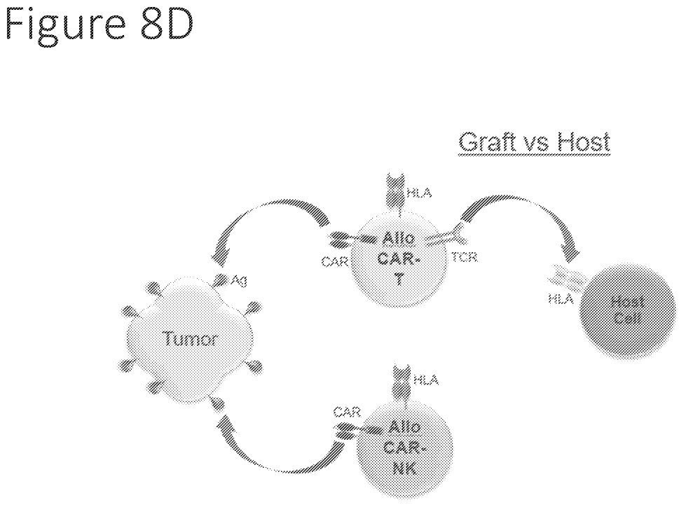

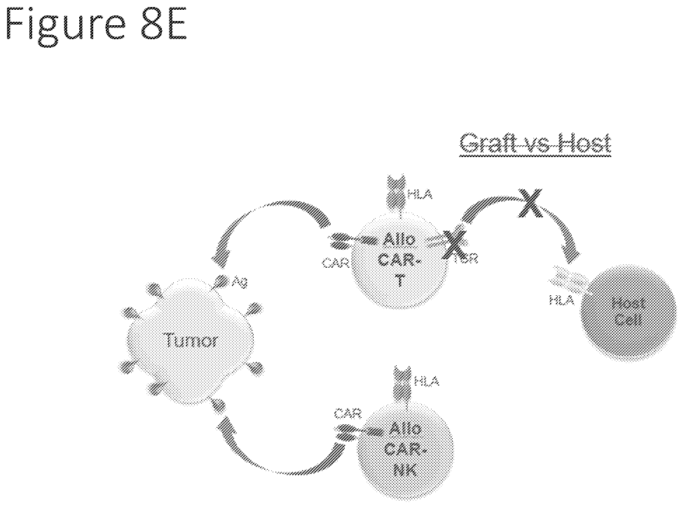

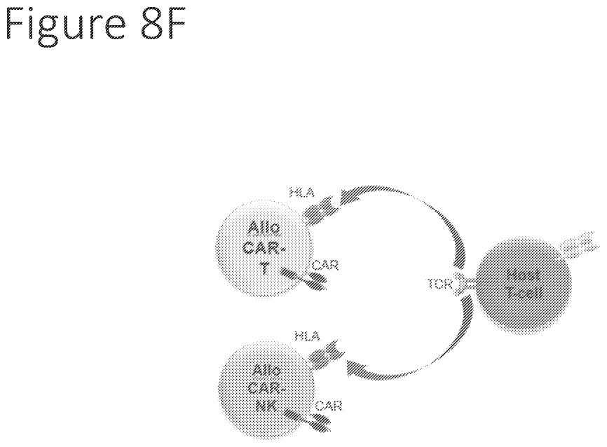

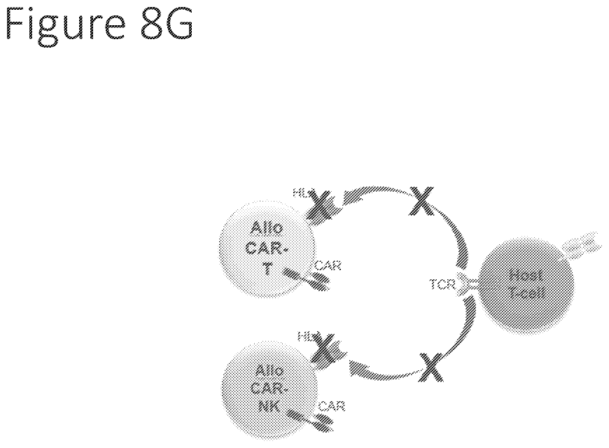

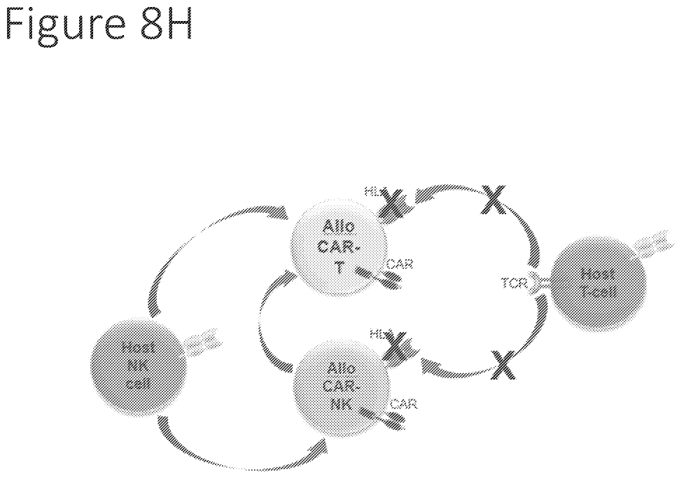

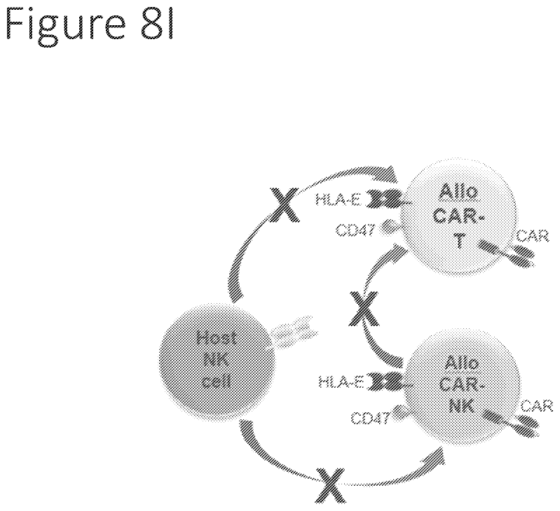

By way of non-limiting example, TGF-beta is one such cytokine released by tumor cells that results in immune suppression within the tumor microenvironment. That immune suppression reduces the ability of immune cells, even engineered CAR-immune cells is some cases, to destroy the tumor cells, thus allowing for tumor progression. In several embodiments, as discussed in detail below, immune checkpoint inhibitors are disrupted through gene editing. In several embodiments, blockers of immune suppressing cytokines in the tumor microenvironment are used, including blockers of their release or competitive inhibitors that reduce the ability of the signaling molecule to bind and inhibit an immune cell. Such signaling molecules include, but are not limited to TGF-beta, IL10, arginase, inducible NOS, reactive-NOS, Arg1, Indoleamine 2,3-dioxygenase (IDO), and PGE₂. However, in additional embodiments, there are provided immune cells, such as NK cells, wherein the ability of the NK cell (or other cell) to respond to a given immunosuppressive signaling molecule is disrupted and/or eliminated. For example, in several embodiments, in several embodiments, NK cells or T cells are genetically edited to become have reduced sensitivity to TGF-beta. TGF-beta is an inhibitor of NK cell function on at least the levels of proliferation and cytotoxicity. See, for example, FIG. 8A which schematically shows some of the inhibitory pathways by which TGF-beta reduces NK cell activity and/or proliferation. Thus, according to some embodiments, the expression of the TGF-beta receptor is knocked down or knocked out through gene editing, such that the edited NK is resistant to the immunosuppressive effects of TGF-beta in the tumor microenvironment. In several embodiments, the TGFB2 receptor is knocked down or knocked out through gene editing, for example, by use of CRISPR-Cas editing. Small interfering RNA, antisense RNA, TALENs or zinc fingers are used in other embodiments. Other isoforms of the TGF-beta receptor (e.g., TGF-beta 1 and/or TGF-beta 3) are edited in some embodiments. In some embodiments TGF-beta receptors in T cells are knocked down through gene editing.

In accordance with additional embodiments, other modulators of one or more aspects of NK cell (or T cell) function are modulated through gene editing. A variety of cytokines impart either negative (as with TGF-beta above) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL-15 signaling in NK cells. As discussed herein, because IL 5 biology impacts multiple aspects of NK cell functionality, including, but not limited to, proliferation/expansion, activation, cytotoxicity, persistence, homing, migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, the CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing.

In several embodiments, CISH gene editing endows an NK cell with enhanced ability to home to a target site. In several embodiments, CISH gene editing endows an NK cell with enhanced ability to migrate, e.g., within a tissue in response to, for example chemoattractants or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells and/or engineered T cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL 5-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Ra (CD25), but not IL-15Ra or IL-2/15RP, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

In several embodiments, gene editing of the immune cells can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of CISH expression in NK cells removes a potent negative regulator of I-15-mediated signaling in NK cells, disinhibits the NK cells and allows for one or more of enhanced NK cell homing, NK cell migration, activation of NK cells, expansion, cytotoxicity and/or persistence. Additionally, in several embodiments, the editing can enhance NK and/or T cell function in the otherwise suppressive tumor microenvironment. In several embodiments, CISH gene editing results in enhanced NK cell expansion, persistence and/or cytotoxicity without requiring Notch ligand being provided exogenously.

As discussed above, T cells that are engineered to express a CAR or chimeric receptor are employed in several embodiments. Also as mentioned above, T cells express a T Cell Receptor (TCR) on their surface. As disclosed herein, in several embodiments, autologous immune cells are transferred back into the original donor of the cells. In such embodiments, immune cells, such as NK cells or T cells are obtained from patients, expanded, genetically modified (e.g., with a CAR or chimeric receptor) and/or optionally further expanded and re-introduced into the patient. As disclosed herein, in several embodiments, allogeneic immune cells are transferred into a subject that is not the original donor of the cells. In such embodiments, immune cells, such as NK cells or T cells are obtained from a donor, expanded, genetically modified (e.g., with a CAR or chimeric receptor) and/or optionally further expanded and administered to the subject.

Allogeneic immunotherapy presents several hurdles to be overcome. In immune-competent hosts, the administered allogeneic cells are rapidly rejected, known as host versus graft rejection (HvG). This substantially limits the efficacy of the administered cells, particularly their persistence. In immune-incompetent hosts, allogeneic cells are able to engraft. However, if the administered cells comprise a T cell (several embodiments disclosed herein employ mixed populations of NK and T cells), the endogenous T cell receptor (TCR) specificities recognize the host tissue as foreign, resulting in graft versus host disease (GvHD). GvHD can lead to significant tissue damage in the host (cell recipient). Several embodiments disclosed herein address both of these hurdles, thereby allowing for effective and safe allogeneic immunotherapy. In several embodiments, gene edits can advantageously help to reduce and/or avoid graft vs. host disease (GvHD). A non-limiting embodiment of such an approach, using a mixed population of NK cell and T cells, is schematically illustrated in FIG. 8C, wherein the NK cells are engineered to express a CAR and the T cells are engineered to not only express a CAR, but also edited to render the T cells non-alloreactive. FIG. 8D schematically shows a mechanism by which graft v. host disease occurs. An allogeneic T cell and an allogeneic NK cell, both engineered to express a CAR that targets the tumor, are introduced into a host. However, the T cell still bears the native T-cell receptor (TCR). This TCR recognizes the HLA type of the host cell as “non-self” and can exert cytotoxicity against host cells. FIG. 8E shows a non-limiting embodiment of how graft v. host disease can be reduced or otherwise avoided through gene editing of the T cells. Briefly, as this approach is discussed in more detail below, gene editing can be performed in order to knockout the native TCR on T cells. Lacking a TCR, the allogeneic T cell cannot detect the “non-self” HLA of the host cells, and therefore is not triggered to exert cytotoxicity against host cells. Thus, in several embodiments T cells are subjected to gene editing to either reduce functionality of and/or reduce or eliminate expression of the native T cell. In several embodiments, CRISPR is used to knockout the TCR. These, and other, embodiments are discussed below.

T cell receptors (TCR) are cell surface receptors that participate in the activation of T cells in response to the presentation of an antigen. The TCR is made up of two different protein chains (it is a heterodimer). The majority of human T cells have TCRs that are made up of an alpha (α) chain and a beta (β) chain (encoded by separate genes). A small percentage of T cells have TCRs made up of gamma and delta (γ/δ) chains (the cells being known as gamma-delta T cells).

Rather than recognizing an intact antigen (as with immunoglobulins), T cells are activated by processed peptide fragments in association with an MHC molecule. This is known as MHC restriction. When the TCR recognizes disparities between the donor and recipient MHC, that recognition stimulates T cell proliferation and the potential development of GVHD. In some embodiments, the genes encoding either the TCRα, TCRβ, TCRγ, and/or the TCEδ are disrupted or otherwise modified to reduce the tendency of donor T cells to recognize disparities between donor and host MHC, thereby reducing recognition of alloantigen and GVHD.

T-cell mediated immunity involves a balance between co-stimulatory and inhibitory signals that serve to fine-tune the immune response. Inhibitory signals, also known as immune checkpoints, allow for avoidance of auto-immunity (e.g., self-tolerance) and also limit immune-mediated damage. Immune checkpoint protein expression are often altered by tumors, enhancing immune resistance in tumor cells and limiting immunotherapy efficacy. CTLA4 downregulates the amplitude of T cell activation. In contrast, PD1 limits T cell effector functions in peripheral tissue during an inflammatory response and also limits autoimmunity. Immune checkpoint blockade, in several embodiments, helps to overcome a barriers to activation of functional cellular immunity. In several embodiments, antagonistic antibodies specific for inhibitory ligands on T cells including Cytotoxic-T-lymphocyte-associated antigen 4 (CTLA-4; also known as CD152) and programmed cell death protein 1 (PD1 or PDCD1 also known as CD279) are used to enhance immunotherapy.

In several embodiments, there is provided genetically modified T cells that are non-alloreactive and highly active. In several embodiments, the T cells are further modified such that certain immune checkpoint genes are inactivated, and the immune checkpoint proteins are thus not expressed by the T cell. In several embodiments, this is done in the absence of manipulation or disruption of the CD3z signaling domain (e.g., the TCRs are still able initiate T cell signaling).

In several embodiments, genetic inactivation of TCRalpha and/or TCRbeta coupled with inactivation of immune checkpoint genes in T lymphocytes derived from an allogeneic donor significantly reduces the risk of GVHD. In several embodiments, this is done by eliminating at least a portion of one or more of the substituent protein chains (alpha, beta, gamma, and/or delta) responsible for recognition of MHC disparities between donor and recipient cells. In several embodiments, this is done while still allowing for T cell proliferation and activity.

In some embodiments wherein allogeneic cells are administered, the receiving subject may receive some other adjunct treatment to support or otherwise enhance the function of the administered immune cells. In several embodiments, the subject may be pre-conditioned (e.g., with radiation or chemotherapy). In some embodiments, the adjunct treatment comprises administration of lymphocyte growth factors (such as IL-2).

Moreover, in several embodiments, editing can improve persistence of administered cells (whether NK cells, T cells, or otherwise) for example, by masking cells to the host immune response. In some cases, a recipient's immune cells will attack donor cells, especially from an allogeneic donor, known as Host vs. Graft disease (HvG). FIG. 8F shows a schematic representation of HvG, where the host T cells, with a native/functional TCR identify HLA on donor T and/or donor NK cells as non-self. In such cases, the host T-cell TCR binding to allogeneic cell HLA leads to elimination of allogeneic cells, thus reducing the persistence of the donor engineered NK/T cells. Regarding HvG, to prevent rejection of administered allogeneic T cells, the subject receiving the cells requires suppression of their immune system In several embodiments, glucocorticoids are used, and include, but are not limited to beclomethasone, betamethasone, budesonide, cortisone, dexamethasone, hydrocortisone, methylprednisolone, prednisolone, prednisone, triamcinolone, among others. Activation of the glucocorticoid receptor in recipient's own T cells alters expression of genes involved in the immune response and results in reduced levels of cytokine production, which translates to T cell anergy and interference with T cell activation (in the recipient). Other embodiments relate to administration of antibodies that can deplete certain types of the recipients immune cells. One such target is CD52, which is expressed at high levels on T and B lymphocytes and lower levels on monocytes while being absent on granulocytes and bone marrow precursors. Treatment or pre-treatment of the recipient with Alemtuzumab, a humanized monoclonal antibody directed against CD52, has been shown to induce a rapid depletion of circulating lymphocytes and monocytes, thus lessening the probability of HvG, given the reduction in recipient immune cells. Immunosuppressive drugs may limit the efficacy of administered allogeneic engineered T cells. Therefore, as disclosed herein, several embodiments relate to genetically engineered allogeneic donor cells that are resistant to immunosuppressive treatment. In several embodiments, as discussed in more detail below, immune cells, such as NK cells and/or T cells are edited (in addition to being engineered to express a CAR) to extend their persistence by avoiding cytotoxic responses from host immune cells. In several embodiments, gene editing to remove one or more HLA molecules from the allogeneic NK and/or T cells reduce elimination by host T-cells. In several embodiments, the allogeneic NK and/or T cells are edited to knock out one or more of beta-2 microglobulin (an HLA Class I molecule) and CIITA (an HLA Class II molecule). FIG. 8G schematically depicts this approach.

In some embodiments of mixed allogeneic cell therapy, the populations of engineered cells actually target one another, for example when the therapeutic cells are edited to remove HLA molecules in order to avoid HvG. Such editing of, for example CAR T cells can result in the vulnerability of the edited allogeneic CAR T cells to cytotoxic attack by the CAR NK cells as well as elimination by host NK cells. This is caused by the missing “self” inhibitory signals generally presented by KIR molecules. FIG. 8H schematically depicts this process. In several embodiments, gene editing can be used to knock in expression of one or more “masking” molecules which mask the allogeneic cells from the host immune system and from fratricide by other administered engineered cells. FIG. 8I schematically depicts this approach. In several embodiments, proteins can be expressed on the surface of the allogeneic cells to inhibit targeting by NKs (both engineered NKs and host NKs), which advantageously prolongs persistence of both allogeneic CAR-Ts and CAR-NKs. In several embodiments, gene editing is used to knock in CD47, expression of which effectively functions as a “don't eat me” signal. In several embodiments, gene editing is used to knock in expression of HLA-E. HLA-E binds to both the inhibiting and activating receptors NKG2A and NKG2C, respectively that exist on the surface of NK cells. However, NKG2A is expressed to a greater degree in most human NK cells, thus, in several embodiments, expression of HLA-E on engineered cells results in an inhibitory effect of NK cells (both host and donor) against such cells edited to (or naturally expressing) HLA-E. In addition, in several embodiments, one or more viral HLA homologs are knocked in such that they are expressed by the engineered NK and/or T cells, thus conferring on the cells the ability of viruses to evade the host immune system. In several embodiments, these approaches advantageously prolong persistence of both allogeneic CAR-Ts and CAR-NKs.

In several embodiments, genetic editing (whether knock out or knock in) of any of the target genes (e.g., CISH, TGFBR, TCR, B2M, CIISH, CD47, HLA-E, or any other target gene disclosed herein), is accomplished through targeted introduction of DNA breakage, and subsequent DNA repair mechanism. In several embodiments, double strand breaks of DNA are repaired by non-homologous end joining (NHEJ), wherein enzymes are used to directly join the DNA ends to one another to repair the break. In several embodiments, however, double strand breaks are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of a TCR) within a sequence that is homologous to the flanking sequences of a double strand break. This will result in the desired change (e.g., insertion) being inserted at the site of the DSB.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in the TCR, or CISH, or any other target gene disclosed herein. Target sites in the TCR can readily be identified. Further information of target sites within a region of the TCR can be found in US Patent Publication No. 2018/0325955, and US Patent Publication No. 2015/0017136, each of which is incorporated by reference herein in its entirety. In several embodiments, two or more meganucleases, or functions fragments thereof, are fused to create a hybrid enzymes that recognize a desired target sequence within the target gene (e.g., CISH).

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the TCR (or an immune checkpoint inhibitor). The combined ZFNs are then fused with the catalytic domain(s) of an endonuclease, such as Fokl (optionally a Fokl heterodimer), in order to induce a targeted DNA break. Additional information on uses of ZFNs to edit the TCR and/or immune checkpoint inhibitors can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein. In several embodiments, CRISPR is used to manipulate the gene(s) encoding a target gene to be knocked out or knocked in, for example CISH, TGFBR2, TCR, B2M, CIITA, CD47, HLA-E, etc. In several embodiments, CRISPR is used to edit one or more of the TCRs of a T cell and/or the genes encoding one or more immune checkpoint inhibitors. In several embodiments, the immune checkpoint inhibitor is selected from one or more of CTLA4 and PD1. In several embodiments, CRISPR is used to truncate one or more of TCRα, TCRβ, TCRγ, and TCRδ. In several embodiments, a TCR is truncated without impacting the function of the CD3z signaling domain of the TCR. Depending on the embodiment and which target gene is to be edited, a Class 1 or Class 2 Cas is used. In several embodiments, a Class 1 Cas is used and the Cas type is selected from the following types: I, IA, IB, IC, ID, IE, IF, IU, III, IIIA, IIIB, IIIC, lID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, a Class 2 Cas is used and the Cas type is selected from the following types: II, IIA, IIB, IIC, V, VI, and combinations thereof. In several embodiments, the Cas is selected from the group consisting of Cas9, Csn2, Cas4, Cpf1, C2c1, C2c3, Cas13a (previously known as C2c2), Cas13b, Cas13c, CasX, CasY and combinations thereof. In some embodiments, class 2 CasX is used, wherein CasX is capable of forming a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

In several embodiments, as discussed above, editing of CISH advantageously imparts to the edited cells, particularly edited NK cells, enhanced expansion, cytotoxicity and/or persistence. Additionally, in several embodiments, the modification of the TCR comprises a modification to TCRα, but without impacting the signaling through the CD3 complex, allowing for T cell proliferation. In one embodiment, the TCRα is inactivated by expression of pre-Ta in the cells, thus restoring a functional CD3 complex in the absence of a functional alpha/beta TCR. As disclosed herein, the non-alloreactive modified T cells are also engineered to express a CAR to redirect the non-alloreactive T cells specificity towards tumor marker, but independent of MHC. Combinations of editing are used in several embodiments, such as knockout of the TCR and CISH in combination, or knock out of CISH and knock in of CD47, by way of non-limiting examples.

In several embodiments, the expression of ADORA2A (Adenosine 2a Receptor) is reduced and/or eliminated in order to increase overall activation in resultant T cells and/or NK cells, or other cell type provided for herein. In several embodiments, ADORA2A is disrupted and/or knocked out using one or more of the gene editing methods disclosed herein. In several embodiments, ADORA2A is disrupted and/or knocked out using a Crispr-Cas mediated approach (e.g., Cas9) as disclosed elsewhere herein, with the Cas nuclease guided by the use of one more of the following ADORA2A-specific guide RNAs: SEQ ID NO: 503-506. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). Loss of expression of ADORA2A induces decreased sensitivity to adenosine, a well-established immunosuppressant for T cells and NK cells. Thus, according to several embodiments, gene editing ADORA2A increases the cytotoxicity, persistence, immune avoidance or otherwise enhances the efficacy of engineered NK, T, or other cell as disclosed herein.

In several embodiments, the expression of TGFBR2 is reduced and/or eliminated in order to increase overall activation in resultant T cells and/or NK cells, or other cell type provided for herein. In several embodiments, TGFBR2 is disrupted and/or knocked out using one or more of the gene editing methods disclosed herein. In several embodiments, TGFBR2 is disrupted and/or knocked out using a Crispr-Cas mediated approach (e.g., Cas9) as disclosed elsewhere herein, with the Cas nuclease guided by the use of one more of the following TGFBR2-specific guide RNAs: SEQ ID NO: 544-547. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In NK cells, TGFBR2 is a potent checkpoint in NK cell-mediated tumor immunity, while for T cells, knockout of TGFBR2 rescues car T cell exhaustion induced by TGF-β1. Thus, according to several embodiments, gene editing TGFBR2 increases the cytotoxicity, persistence, immune avoidance or otherwise enhances the efficacy of engineered NK, T, or other cell as disclosed herein.

In several embodiments, the expression of NKG2A is reduced and/or eliminated in order to increase overall activation in resultant T cells and/or NK cells, or other cell type provided for herein. In several embodiments, NKG2A is disrupted and/or knocked out using one or more of the gene editing methods disclosed herein. In several embodiments, NKG2A is disrupted and/or knocked out using a Crispr-Cas mediated approach (e.g., Cas9) as disclosed elsewhere herein, with the Cas nuclease guided by the use of one more of the following NKG2A-specific guide RNAs: SEQ ID NO: 548-551. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). NKG2A binds to HLA-E and is recognized as an MHC-recognizing receptor. Since NKG2A is an inhibitor receptor, loss of expression of NKG2A induces increased activation of constituent cells. In NK and T cells, loss of NKG2A leads to increased activation and cytotoxicity against HLA-E expressing tumor cells. Thus, according to several embodiments, gene editing NKG2A increases the cytotoxicity, persistence, immune avoidance or otherwise enhances the efficacy of engineered NK, T, or other cell as disclosed herein.

In several embodiments, the expression of Cytokine Signaling 2 (SOCS2) is reduced and/or eliminated in order to increase overall activation in resultant T cells and/or NK cells, or other cell type provided for herein. In several embodiments, SOCS2 is disrupted and/or knocked out using one or more of the gene editing methods disclosed herein. In several embodiments, SOCS2 is disrupted and/or knocked out using a Crispr-Cas mediated approach (e.g., Cas9) as disclosed elsewhere herein, with the Cas nuclease guided by the use of one more of the following SOCS2-specific guide RNAs: SEQ ID NO: 556-561. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). SOCS proteins are negative regulators of cytokine responses, and SOCS2 specifically negatively regulates the development of NK cells through inhibiting JAK2 activity. Loss of expression of SOCS2 in NK cells induces increased NK cell development and overall cytotoxicity. Thus, according to several embodiments, gene editing SOCS2 increases the cytotoxicity, persistence, immune avoidance or otherwise enhances the efficacy of engineered NK, T, or other cell as disclosed herein.

In several embodiments, the expression of Casitas B-lineage lymphoma-b (Cbl-b) is reduced and/or eliminated in order to increase overall activation in resultant T cells and/or NK cells, or other cell type provided for herein. In several embodiments, Cbl-b is disrupted and/or knocked out using one or more of the gene editing methods disclosed herein. In several embodiments, Cbl-b is disrupted and/or knocked out using a Crispr-Cas mediated approach (e.g., Cas9) as disclosed elsewhere herein, with the Cas nuclease guided by the use of one more of the following Cbl-b-specific guide RNAs: In several embodiments, the expression of Casitas B-lineage lymphoma-b (Cbl-b) is reduced and/or eliminated in order to increase overall activation in resultant T cells and NK cells. In several embodiments, Cbl-b is disrupted and/or knocked out using a Crispr-Cas mediated approach (e.g., Cas9) as disclosed elsewhere herein, but with the use of one more of the following Cbl-b-specific guide RNAs: SEQ ID NO: 552-555. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). Cbl-b is an E3 ubiquitin ligase that negatively regulates T cell activation Loss of expression of Cbl-b in NK cells and T cells demonstrate increased antitumor immunity. Moreover, Cbl-b deficient T cells and NK cells are resistant to PD-L1/PD-1 mediated suppression. Thus, according to several embodiments, gene editing Cbl-b increases the cytotoxicity, persistence, immune avoidance or otherwise enhances the efficacy of engineered NK, T, or other cell as disclosed herein.

In several embodiments, the expression of Beta-2 Microglobulin (B2-microglobulin) is reduced and/or eliminated in order to increase overall activation in resultant T cells and/or NK cells, or other cell type provided for herein. In several embodiments, B2-microglobulin is disrupted and/or knocked out using one or more of the gene editing methods disclosed herein. In several embodiments, B2-microglobulin is disrupted and/or knocked out using a Crispr-Cas mediated approach (e.g., Cas9) as disclosed elsewhere herein, with the Cas nuclease guided by the use of one more of the following B2-microglobulin-specific guide RNAs: SEQ ID NO: 290-299. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). Loss of expression of B2-microglobulin induces greatly reduced levels of MHC class I molecules, and in both NK cells and T cells, reduction of B2-microglobulin can modulate overall cell recognition of autologous and allogenic cells. Thus, according to several embodiments, gene editing B2-microglobulin increases the cytotoxicity, persistence, immune avoidance or otherwise enhances the efficacy of engineered NK, T, or other cell as disclosed herein.

In several embodiments, the expression of T cell immunoreceptor with Ig and ITIM domains (TIGIT) is reduced and/or eliminated in order to increase overall activation in resultant T cells and/or NK cells, or other cell type provided for herein. In several embodiments, TIGIT is disrupted and/or knocked out using one or more of the gene editing methods disclosed herein. In several embodiments, TIGIT is disrupted and/or knocked out using a Crispr-Cas mediated approach (e.g., Cas9) as disclosed elsewhere herein, with the Cas nuclease guided by the use of one more of the following TIGIT-specific guide RNAs: SEQ ID NO: 507-510. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). TIGIT is a checkpoint receptor associated with T cell and NK cell exhaustion. Loss of expression of TIGIT in NK cells prevents NK cell exhaustion and promotes NK cell-dependent tumor immunity. Loss of expression of TIGIT in T cells can similarly lead to downstream activation of resultant T cells. Thus, according to several embodiments, gene editing TIGIT increases the cytotoxicity, persistence, immune avoidance or otherwise enhances the efficacy of engineered NK, T, or other cell as disclosed herein.

In several embodiments, the expression of Programmed cell death protein-1 (PD-1) is reduced and/or eliminated in order to increase overall activation in resultant T cells and/or NK cells, or other cell type provided for herein. In several embodiments, PD-1 is disrupted and/or knocked out using one or more of the gene editing methods disclosed herein. In several embodiments, PD-1 is disrupted and/or knocked out using a Crispr-Cas mediated approach (e.g., Cas9) as disclosed elsewhere herein, with the Cas nuclease guided by the use of one more of the following PD-1-specific guide RNAs: SEQ ID NO: 511-514. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). PD-1 plays an inhibitory role in immune regulation and down-regulates overall function by suppressing immune cell activity. Loss of expression of PD-1 in NK cells increases overall cytotoxicity due to increased secretion of interferon-gamma, granzyme B, and perforin. Similarly, T cells with loss of expression of PD-1 demonstrate increased cytotoxicity and overall caspase activation. Thus, according to several embodiments, gene editing PD-1 increases the cytotoxicity, persistence, immune avoidance or otherwise enhances the efficacy of engineered NK, T, or other cell as disclosed herein.

In several embodiments, the expression of T-cell immunoglobulin and mucin-domain containing-3 (TIM-3) is reduced and/or eliminated in order to increase overall activation in resultant T cells and/or NK cells, or other cell type provided for herein. In several embodiments, TIM-3 is disrupted and/or knocked out using one or more of the gene editing methods disclosed herein. In several embodiments, TIM-3 is disrupted and/or knocked out using a Crispr-Cas mediated approach (e.g., Cas9) as disclosed elsewhere herein, with the Cas nuclease guided by the use of one more of the following TIM-3-specific guide RNAs: SEQ ID NO:515-518. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). TIM-3 is an inhibitory receptor involved in immune checkpoint function. Loss of expression of TIM-3 increases overall cytotoxicity in engineered NK and T cells as well as decreased exhaustion of NK cells and T cells, leading to increased effector function of constituent cells lacking TIM-3 expression. Thus, according to several embodiments, gene editing TIM-3 increases the cytotoxicity, persistence, immune avoidance or otherwise enhances the efficacy of engineered NK, T, or other cell as disclosed herein.

In several embodiments, the expression of CD38 is reduced and/or eliminated in order to increase overall activation in resultant T cells and/or NK cells, or other cell type provided for herein. In several embodiments, CD38 is disrupted and/or knocked out using one or more of the gene editing methods disclosed herein. In several embodiments, CD38 is disrupted and/or knocked out using a Crispr-Cas mediated approach (e.g., Cas9) as disclosed elsewhere herein, with the Cas nuclease guided by the use of one more of the following CD38-specific guide RNAs: SEQ ID NO:519-522. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). CD38 plays a role in the maturation cycle of immune cells, and blood cancers can often present upregulated CD38. Loss of CD38 expression on constituent NK cells allows for greater cytotoxicity due to decreased fratricide. Wild-type NK cells self-express CD38, leading to downstream self-targeting effects in wild-type NK cells. For T cells, loss of CD38 expression for constituent T cells leads to increased cytotoxicity. Thus, according to several embodiments, gene editing CD38 increases the cytotoxicity, persistence, immune avoidance or otherwise enhances the efficacy of engineered NK, T, or other cell as disclosed herein.

In several embodiments, the expression of T cell receptor alpha (TCR a) is reduced and/or eliminated in order to increase overall activation in resultant T cells and/or NK cells, or other cell type provided for herein. In several embodiments, TCR a is disrupted and/or knocked out using one or more of the gene editing methods disclosed herein. In several embodiments, TCR a is disrupted and/or knocked out using a Crispr-Cas mediated approach (e.g., Cas9) as disclosed elsewhere herein, with the Cas nuclease guided by the use of one more of the following TCR a-specific guide RNAs: SEQ ID NO:566-569.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). T cell receptors (TCR) are protein complexes found on T cells responsible for recognizing MHC molecules. Loss of certain TCRs and preferential expression of other TCRs can lead to increased cytotoxicity in engineered cells due to increased selective targeting and recognition by constituent cells. Thus, according to several embodiments, gene editing TCRs increases the cytotoxicity, persistence, immune avoidance or otherwise enhances the efficacy of engineered NK, T, or other cell as disclosed herein.

In several embodiments, the expression of CISH is reduced and/or eliminated in order to increase overall activation in resultant T cells and/or NK cells, or other cell type provided for herein. In several embodiments, CISH is disrupted and/or knocked out using one or more of the gene editing methods disclosed herein. In several embodiments, CISH is disrupted and/or knocked out using a Crispr-Cas mediated approach (e.g., Cas9) as disclosed elsewhere herein, with the Cas nuclease guided by the use of one more of the following CISH-specific guide RNAs: SEQ ID NO: 562-565, or other guide disclosed herein. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In CD8+ T cells, CISH actively silences TCR signaling to maintain tumor tolerance, and CISH has been shown to be a downstream negative regulator of IL-15 receptor signaling. In NK and T cells, CISH plays a role in checkpoint maturation and proliferation. Thus, according to several embodiments, gene editing CISH increases the cytotoxicity, persistence, immune avoidance or otherwise enhances the efficacy of engineered NK, T, or other cell as disclosed herein.

In several embodiments, the expression of CEACAMI is reduced and/or eliminated in order to increase overall activation in resultant T cells and/or NK cells, or other cell type provided for herein. In several embodiments, CEACAMI is disrupted and/or knocked out using one or more of the gene editing methods disclosed herein. In several embodiments, CEACAMI is disrupted and/or knocked out using a Crispr-Cas mediated approach (e.g., Cas9) as disclosed elsewhere herein, with the Cas nuclease guided by the use of one more of the following CEACAMI-specific guide RNAs: SEQ ID NO: 497-499. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). CEACAMI is a checkpoint inhibitor for both NK and T cells and can inhibit lysis of CEACAMI-bearing tumor cell lines. Loss of expression of CEACAMI can increase overall cytotoxicity for NK and T cells. Thus, according to several embodiments, gene editing CEACAMI increases the cytotoxicity, persistence, immune avoidance or otherwise enhances the efficacy of engineered NK, T, or other cell as disclosed herein.

In several embodiments, the expression of DDIT4 is reduced and/or eliminated in order to increase overall activation in resultant T cells and/or NK cells, or other cell type provided for herein. In several embodiments, DDIT4 is disrupted and/or knocked out using one or more of the gene editing methods disclosed herein. In several embodiments, DDIT4 is disrupted and/or knocked out using a Crispr-Cas mediated approach (e.g., Cas9) as disclosed elsewhere herein, with the Cas nuclease guided by the use of one more of the following DDIT4-specific guide RNAs: SEQ ID NO: 500-502. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In NK and T cells, DDIT4 is a negative regulator of mTORC1, which itself enhances IL-15 mediated survival and proliferation of NK cells. Moreover, DDIT4 is upregulated by oxidative stress conditions as is common in tumor microenvironments. Loss of DDIT4 function in engineered cells may increase overall glucose metabolism leading to enhanced proliferation, as well as increasing overall NK or T cell cytotoxicity. Thus, according to several embodiments, gene editing DDIT4 increases the cytotoxicity, persistence, immune avoidance or otherwise enhances the efficacy of engineered NK, T, or other cell as disclosed herein.

In several embodiments, the expression of MAPKAP Kinase 3 (MAPKAPK3) is reduced and/or eliminated in order to increase overall activation in resultant T cells and/or NK cells, or other cell type provided for herein. In several embodiments, MAPKAPK3 is disrupted and/or knocked out using one or more of the gene editing methods disclosed herein. In several embodiments, MAPKAPK3 is disrupted and/or knocked out using a Crispr-Cas mediated approach (e.g., Cas9) as disclosed elsewhere herein, with the Cas nuclease guided by the use of one more of the following MAPKAPK3-specific guide RNAs: SEQ ID NO: 494-496. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). MAPKAP Kinase 3 in expressed in both NK and T cells. Loss of MAPKAPK3 in engineered cells is expected to increase cytotoxicity, cytokine secretion, and overall NK signaling. Thus, according to several embodiments, gene editing MAPKAPK3 increases the cytotoxicity, persistence, immune avoidance or otherwise enhances the efficacy of engineered NK, T, or other cell as disclosed herein.

In several embodiments, the expression of SMAD3 is reduced and/or eliminated in order to increase overall activation in resultant T cells and/or NK cells, or other cell type provided for herein. In several embodiments, SMAD3 is disrupted and/or knocked out using one or more of the gene editing methods disclosed herein. In several embodiments, SMAD3 is disrupted and/or knocked out using a Crispr-Cas mediated approach (e.g., Cas9) as disclosed elsewhere herein, with the Cas nuclease guided by the use of one more of the following SMAD3-specific guide RNAs: SEQ ID NO: 491-493. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). SMAD3 is a downstream mediator of TGF-Beta and Activin A signaling. Inhibition of activin A provides an effective downstream TGFBR knockout. Smad3 silenced NK cells demonstrate increased proliferation and differentiation, as well as increased cytotoxicity in engineered T and NK cells. Thus, according to several embodiments, gene editing SMAD3 increases the cytotoxicity, persistence, immune avoidance or otherwise enhances the efficacy of engineered NK, T, or other cell as disclosed herein.

As discussed above, genetically edited cells can be edited at a plurality of locations. For example in several embodiments, cells (e.g., NK cells or T cells, or a mixture thereof) are edited at two locations. In several embodiments, one of the gene edits is made at a target site in the CISH gene. In several embodiments, one of the gene edits is made at a target site in the CBLB gene. In several embodiments, one of the gene edits is made at a target site in the TGFBR2 gene. In several embodiments, one of the gene edits is made at a target site in the TIGIT gene. Any combination of such edits is also within the provided embodiments, for example dual TGFBR2/CBLB, dual TIGIT/TGFBR2, CISH/CBLB, CISH/TGFBR2, CISH/TIGIT, etc. Moreover, any combination of edits of any of the target genes for editing (e.g., by Crispr or other nuclease) can be made according to some embodiments. Additionally, to the extent necessary to achieve a desired amount of reduction in gene expression, multiple edits may be made within a single target gene, or genes.

In several embodiments, gene edits are made at a target site in a CISH gene and a target site in a CBLB gene. In some embodiments, a double edit, e.g., CISH/CBLB is made in NK cells and/or T cells for use in therapy. In several embodiments, a combination CISH/CBLB gene edit is made in an NK cell that does not include an edit at a CD70 gene. In several embodiments, a combination CISH/CBLB gene edit is made in an NK cell that does not include an edit at any additional gene. In several embodiments, a combination CISH/CBLB gene edit is made in an NK cell that does not express any one or combination of any of an anti-CD70 CAR, an anti-CD19 CAR, or an anti-NKG2D chimeric receptor. In some embodiments, a triple edit, e.g., CD70/CISH/CBLB is made in NK cells and/or T cells for use in therapy. In some embodiments, a triple edit, e.g., CD70/CISH/CBLB is made in NK cells and/or T cells that are engineered to express a tumor-targeting CAR.

In several embodiments, gene edits are made at a target site in a CISH gene and a target site in a TGFBR2 gene. In some embodiments, a double edit, e.g., CISH/TGFBR2 is made in NK cells and/or T cells for use in therapy. In several embodiments, a combination CISH/TGFBR2 gene edit is made in an NK cell that does not include an edit at a CD70 gene. In several embodiments, a combination CISH/TGFBR2 gene edit is made in an NK cell that does not include an edit at any additional gene. In several embodiments, a combination CISH/TGFBR2 gene edit is made in an NK cell that does not express any one or combination of any of an anti-CD70 CAR, an anti-CD19 CAR, or an anti-NKG2D chimeric receptor. In some embodiments, a triple edit, e.g., CD70/CISH/TGFBR2 is made in NK cells and/or T cells for use in therapy. In some embodiments, a triple edit, e.g., CD70/CISH/TGFBR2 is made in NK cells and/or T cells that are engineered to express a tumor-targeting CAR.

In several embodiments, gene edits are made at a target site in a CISH gene and a target site in a TIGIT gene. In some embodiments, a double edit, e.g., CISH/TIGIT is made in NK cells and/or T cells for use in therapy. In several embodiments, a combination CISH/TIGIT gene edit is made in an NK cell that does not include an edit at a CD70 gene. In several embodiments, a combination CISH/TIGIT gene edit is made in an NK cell that does not include an edit at any additional gene. In several embodiments, a combination CISH/TIGIT gene edit is made in an NK cell that does not express any one or combination of any of an anti-CD70 CAR, an anti-CD19 CAR, or an anti-NKG2D chimeric receptor. In some embodiments, a triple edit, e.g., CD70/CISH/TIGIT is made in NK cells and/or T cells for use in therapy. In some embodiments, a triple edit, e.g., CD70/CISH/TIGIT is made in NK cells and/or T cells that are engineered to express a tumor-targeting CAR.

### Engineering Enhanced Persistence Through Immunosuppressive Effector

Additional cellular engineering strategies are provided for herein that serve to further enhance the persistence of allogeneic cellular therapy products, such as allogeneic CAR-T cells and/or allogeneic CAR-NK cells. As discussed herein, there are various strategies that can be employed to reduce the tendency of an allogeneic cell therapy product to induce host cell-mediated graft rejection. For example, in several embodiments the expression of B2M is reduced and/or eliminated in order to reduce the host-mediated graft rejection. In several embodiments, B2M expression is disrupted and/or knocked out using a Crispr-Cas mediated approach (e.g., Cas9) as disclosed elsewhere herein, but with the use of one more of the following B2M-specific guide RNAs: SEQ ID NO: 290—CGCGAGCACAGCTAAGGCCA; SEQ ID NO: 291—GAGTAGCGCGAGCACAGCTA; SEQ ID NO: 292—GCTACTCTCTCTTTCTGGCC; SEQ ID 293—GGCCGAGATGTCTCGCTCCG; SEQ ID NO: 294—GGCCACGGAGCGAGACATCT; SEQ ID NO: 295—CACAGCCCAAGATAGTTAAG; SEQ ID NO: 296—AGTCACATGGTTCACACGGC; SEQ ID NO: 297—AAGTCAACTTCAATGTCGGA; SEQ ID NO: 298—ACTTGTCTTTCAGCAAGGAC; and SEQ ID NO: 299—TGGGCTGTGACAAAGTCACA. Loss of expression of B2M induces a complete loss of HLA expression, which can reduce, and in some embodiments, eliminate, the host T-cell mediated graft rejection. However, this can make also render the administered cells susceptible to host NK-cell mediated graft rejection (as well as to administered engineered NK cells, when a mixed NK/T cell population is used). This, as discussed above, results in loss of the KIR inhibitory signals (e.g., “missing self” signals). See FIGS. 8E-8I. In some embodiments various proteins can be expressed on the surface of a cell to be administered to an allogeneic recipient in order to inhibit host (or administered) NK cells from targeting the administered NK and or T cells. In several embodiments, approaches involve the expression of, for example HLA-E/HLA-G expression, CD47, or one or more viral peptide/proteins, and combinations of these (among other disclosed herein).

### Viral Immunosuppressive Peptides

Many viral infections occur, at least in part, due to the ability of a virus to evade an host immune system, either by camouflage or suppression of host immune reactivity. In vitro studies have shown that particular aspects of certain viruses have such immunosuppressive effects, for example, retroviral transmembrane envelope proteins, such as the p15E protein. Additionally, studies have identified conserved regions of sequences across multiple viral types, see for example Table 1, which includes, among others, a 17 amino acid conserved sequence, CKS-17 (SEQ ID NO: 199), and the nucleic acid encoding the same (SEQ ID NO: 216).

TABLE 1
Viral Peptides

| SEQ ID | | |
|---|---|---|
| NO: | Name | Sequence |
| 199 | CKS-17 | LQNRRGLDLLFLKEGGL |
| 200 | MoLV p15E | LQNRRGLDLLFLKEGGLCAALKEECCF |
| 201 | FLC p15E | LQNRRGLDLLFLKEGGLCAALKEECCF |
| 202 | AKV p15E | LQNRRGLDLLFLKEGGLCAALKEECCF |
| 203 | GLV p15E | LQNRRGLDLLFLKEGGLCAALKEECCF |
| 204 | MMCF p15E | LQNRRGLDLLFLKEGGLCAALKEECCF |
| 205 | AMCF p15E | LQNRRGLDLLFLKEGGLCAALKEECCF |
| 206 | FeLV p15E | LQNRRGLDLLFLQEGGLCAALKEECCF |
| 207 | MPMV gp20 | LQNRRGLDLLTAEQGGICLALQEKCCF |
| 208 | REV-A GP20 | LQNRRGLDLLTAEQGGICLALQEKCCF |
| 209 | HTLV-I gp21 | AQNRRGLDLLFWEQGGLCKALQEQCRF |
| 210 | HTLV-II gp21 | AQNRRGLDLLFWEQGGLCKAIQEQCCF |
| 211 | HIV-1 NEF | MTYKAAVDLSHFLKEKGGL |
| 212 | HIV-1 gp41 | LQARVLAVERYLKDQQL |
| 213 | B26 | LQNKRGLDLLFLX₁X₂GGL |
| | | (X₁ = K/E, X₂ = E/K) |
| 214 | BaEV | LQNRRGLDLLTAEQGGX₁ |
| | | (X₁ = L/I) |
| 215 | ERV-9, 4-1 | X₁QNRX₂X₃LX₄X₅X₆X₇AX₈X₉X₁₀GX₁₁ |
| | | (X₁ = L/Y, X₂ = */L, X₃ = G/A, X₄ = D/N, X₅ = L/Y, |
| | | X₆ = L/S, X₇ = L/T, X₈ = E/Q/A, X₉ = E/K, X₁₀ = G/E, |
| | | X₁₁ = L/V |
| 216 | CKS-17 | ctgcaaaacagaaggggattagacctgctttttcttaaagagggagggctc |

In several embodiments, one or more of such viral immunosuppressive peptides (also referred to as viral peptides) are used to confer resistance to inactivation of engineered immune cells by host NK cells. Various approaches can be undertaken depending on the embodiment and the cell type to be used.

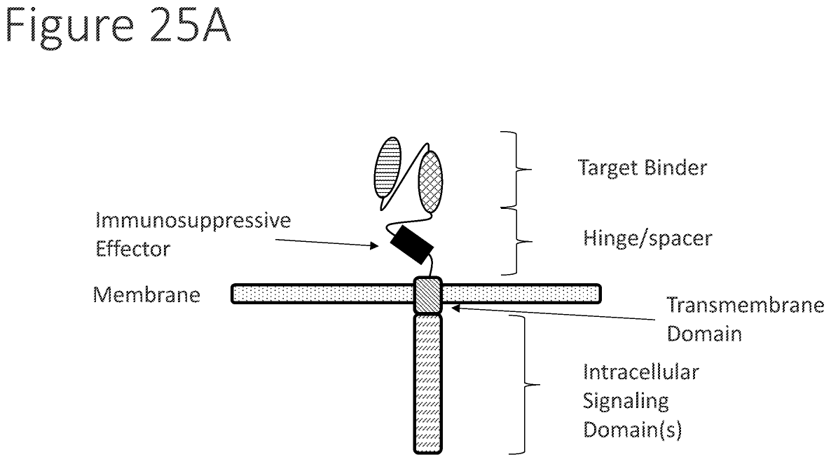

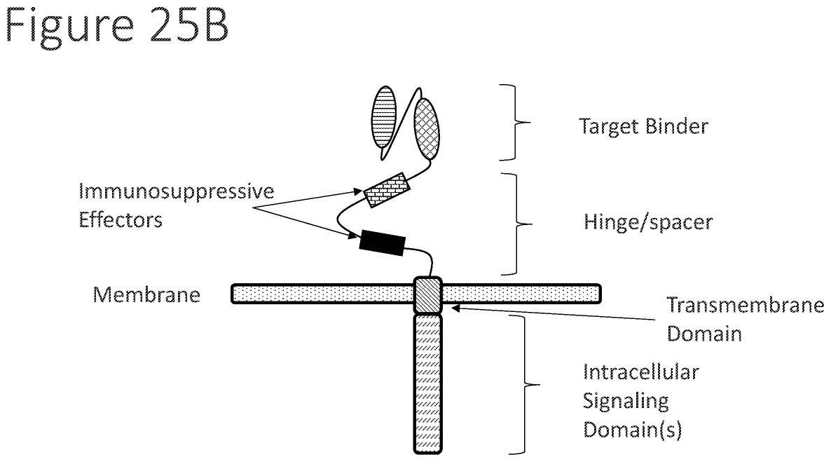

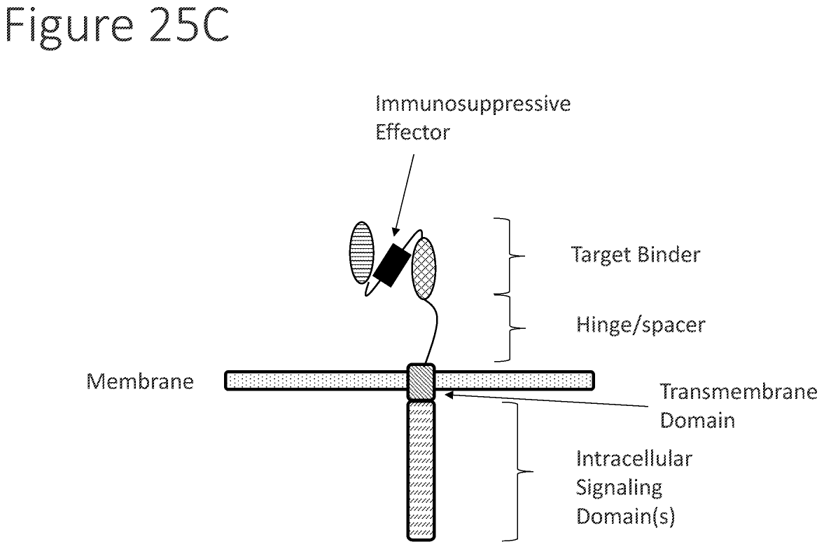

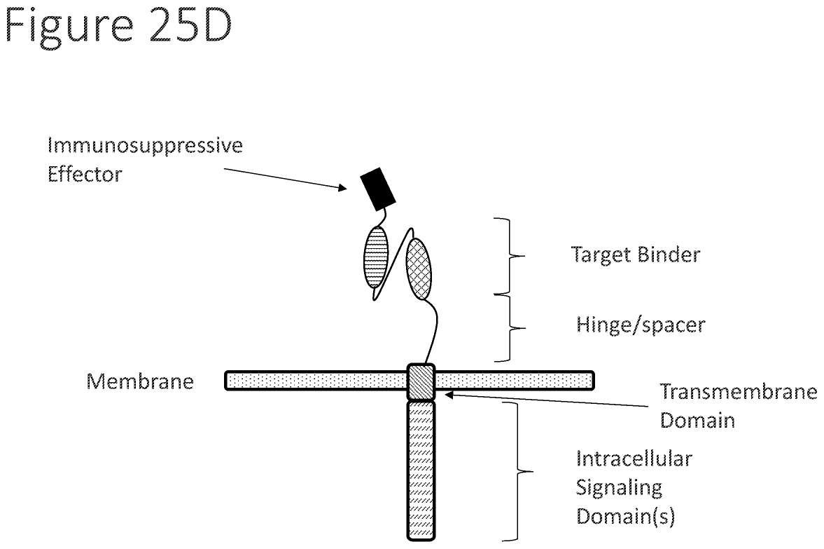

According to several embodiments, one or more viral immunosuppressive peptides are integrated into a chimeric antigen receptor that is then expressed by a population of immune cells to be used in treating a patient. In several embodiments, the immune cells are allogeneic to the patient. In several embodiments, a combination of NK cells and T are engineered to express one or more CARs that comprise one or more viral immunosuppressive peptide. FIG. 25A depicts a non-limiting embodiment of a viral immunosuppressive peptide that is incorporated into a CAR (identified generically as “Immunosuppressive effector Domain”, which shall be understood to refer to any of the viral immunosuppressive peptides or other immunosuppressive peptides/proteins disclosed herein, unless otherwise specified. Shown in FIG. 25A, the viral immunosuppressive peptide is integrated into the hinge/spacer domain of a CAR comprising a target binder (such as an scFv), a hinge (also referred to herein as a spacer), a transmembrane domain and one or more intracellular signaling domains. Depending on the embodiment, more than one (e.g., two, three, four, five, or more) viral immunosuppressive peptides can be introduced into the hinge/spacer region of the CAR. In several such embodiments, as schematically depicted in FIG. 25B, when two (or more) viral immunosuppressive peptides are used, they can be of a different sequence or type. For example, in several embodiments, a CKS-17 peptide is integrated into the hinge region of the CAR in conjunction with, for example, a REV-A peptide. In several embodiments, the inclusion of two or more viral immunosuppressive peptides creates a synergistic immunosuppressive effect.

Depending on the embodiment, the length of the hinge/spacer region can be altered. In several embodiments, a CD8alpha hinge/spacer region is use, but, in some embodiments, a longer or a shorter spacer is used. The spacer can be, depending on the embodiment, an IgG1, IgG2, IgG3, IgG4, or CD28 spacer domain or be derived from IgG1, IgG2, IgG3, IgG4, CD28, or can be a fully synthetic sequence. In several embodiments, IgG-based spacers are edited to reduce or eliminate the ability of the spacer to bring Fc-receptor bearing cells, which can advantageously reduce off-target activation of immune cells (such as those bearing the immunosuppressive effectors as disclosed herein). Non-limiting editing approaches include, but are not limited to, deletion of the heavy chain constant 2 (CH2) domain to abrogate binding to the Fc receptor, or mutating certain amino acids that are essential to Fc receptor binding. In several embodiments, a longer spacer advantageously allows for enhances targeting of certain membrane-proximal epitopes expressed by cancer cells and exposure of the immunosuppressive effector such that it can interact with host and/or administered immune cells to reduce unwanted suppression of the therapeutic cells. In several embodiments, a single hinge region can be made longer by including multiple hinge-encoding sequences, e.g., two, three, four, or more hinges. In several embodiments, wherein multiple hinge regions are used, they can be of the same type (e.g., three CD8α hinges) or can vary (e.g., one CD8αhinge, one CD28 hinge, and a IgG1 hinge). In several embodiments, a shorter hinge is used, wherein the shorter hinge limits the ability of host phosphatases (like CD45) to attenuate signaling of a CAR expressed by the engineered immune cell. In several embodiments, the hinge region comprises one or more of SEQ ID NOs: 479-487. In some embodiments, the hinge region comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence with any of SEQ ID NOs: 479-487.

FIG. 25C depicts an additional approach according to several embodiments disclosed herein, namely the engineering of the viral immunosuppressive peptide into the linker between the heavy chain and the light chain of an scFv. in additional embodiments, where a target binder that is not an scFv (e.g., a full antibody), the viral immunosuppressive peptide can be integrated into any region of that binder that allows for exposure of the vial immunosuppressive peptide as well as maintenance of target binding capability. As with integration into the hinge region, engineering the viral immunosuppressive peptide into the linker region can be done with a single peptide, or with multiple peptides. An additional approach is shown in FIG. 25D, with the viral immunosuppressive peptide engineered into the N-terminal region of the chimeric antigen receptor. As with the other approaches, in several embodiments multiple viral immunosuppressive peptides can be used. Additionally, combinations of these positions within the CAR can be used. For example, a viral immunosuppressive peptide (or more than one) can be positioned in the hinge region in combination with, for example a viral immunosuppressive peptide (or more than one) positioned in the linker region of an scFv and/or at the N-terminus of the CAR. In several embodiments, the positions allow the viral immunosuppressive peptides to be exposed such that they can interact with, and thus suppress, host immune cell activity that would otherwise reduce the efficacy of the engineered immune cell expressing the CAR.

In several embodiments, the engineered CAR comprises one or more copies of one or more of the following amino acid or DNA sequences: SEQ ID NO: 199-216, 220-221, 225-226, 230-231, 235-236, 240-241, 245-246, 250-251, 273-274, 278, 280, 288, or 289. As discussed above, those sequences (or individual sequence) can be positioned in the hinge region, the N-terminal region or within the target binder region (e.g., within the linker of an scFv). In several embodiments, the CAR comprises an amino acid sequence or DNA sequence with at least about 80%, at least about 85%, at least about 90%, at least about 94%, at least about 95%, at least about 96%, at least about 97%, at least about 98%, or at least about 99%, sequence identity, homology and/or functional equivalence with any of SEQ ID NOs: SEQ ID NO: 199-216, 220-221, 225-226, 230-231, 235-236, 240-241, 245-246, 250-251, 273-274, 278, 280, 288, or 289.

In several embodiments, the engineered cells provided for herein comprise a chimeric receptor that targets NKG2D, wherein the CAR comprises an amino acid of SEQ ID NO: 174 or 899, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 174 or 899 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 174 or 899. In addition to, or in place of the integrated viral immunosuppressive amino acids, the CAR optionally comprises an amino acid of SEQ ID NO: 174 or 899, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 174 or 899, with one or more of the viral immunosuppressive sequences expressed on the cell in a membrane-bound format, as discussed below.

In several embodiments, the engineered cells provided for herein comprise a CAR that targets CD19, wherein the CAR comprises an amino acid of SEQ ID NO: 178 or 901, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178 or 901 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 178. In addition to, or in place of the integrated viral immunosuppressive amino acid, the CAR optionally comprises an amino acid of SEQ ID NO: 178 or 901, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178 or 901, with one or more of the viral immunosuppressive sequences expressed on the cell in a membrane-bound format, as discussed below.

In several embodiments, the engineered cells provided for herein comprise a CAR that targets CD19, wherein the CAR is encoded by a nucleic acid sequence comprising SEQ ID NO: 466, or comprises an nucleic acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 466 with one or more copies of one or more of the following viral immunosuppressive amino acid sequences: SEQ ID NO: 199-216, 220, 225, 230, 235, 240, 245, 250, 273, 280, 288, or 289 integrated into the sequence encoded by SEQ ID NO: 466. In addition to, or in place of the integrated viral immunosuppressive amino acids, the CAR optionally comprises a CAR encoded by SEQ ID NO: 466, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to the sequence encoded by SEQ ID NO: 466, with one or more of the viral immunosuppressive sequences expressed on the cell in a membrane-bound format, as discussed below.

In several embodiments, the engineered cells provided for herein comprise a CAR that targets CD70, wherein the CAR comprises an amino acid of any of SEQ ID NOs: 383-465 or 912-994, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NO: 383-465 or 912-994 with one or more copies of one or more of the following viral immunosuppressive amino acid sequences: SEQ ID NO: 199-216, 220, 225, 230, 235, 240, 245, 250, 273, 280, 288, or 289 integrated into the sequence of any of SEQ ID NO: 383-465 or 912-994. In addition to, or in place of the integrated viral immunosuppressive amino acid, the CAR optionally comprises an amino acid of any of SEQ ID NO: 383-465 or 912-994, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 383-465 or 912-994, with one or more of the viral immunosuppressive sequences expressed on the cell in a membrane-bound format, as discussed below.

In addition to incorporation of the viral immunosuppressive peptides being positioned in the CAR, the peptides can be coupled to a domain that allows them to be expressed as a membrane bound viral immunosuppressive peptide.

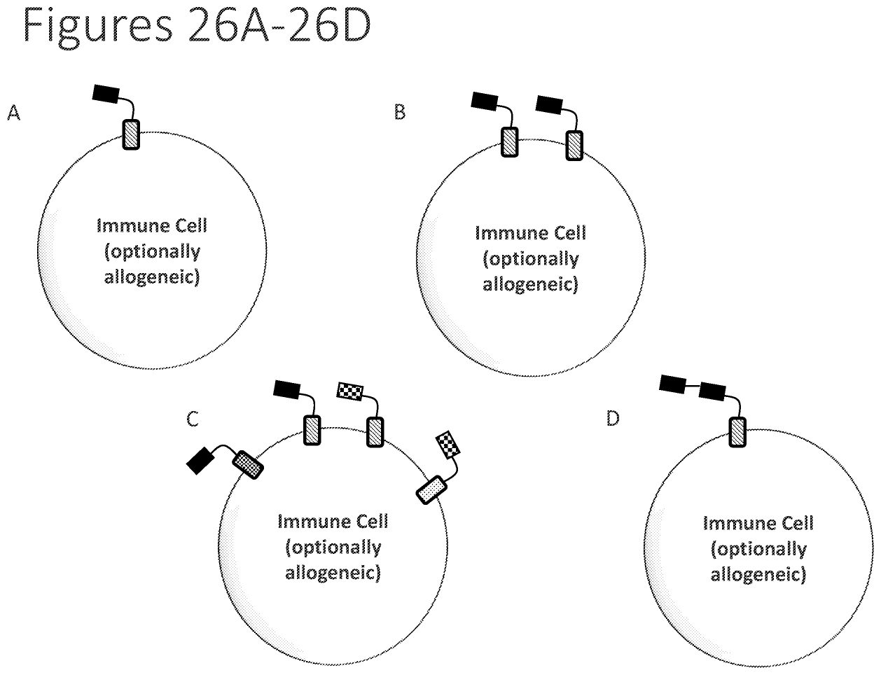

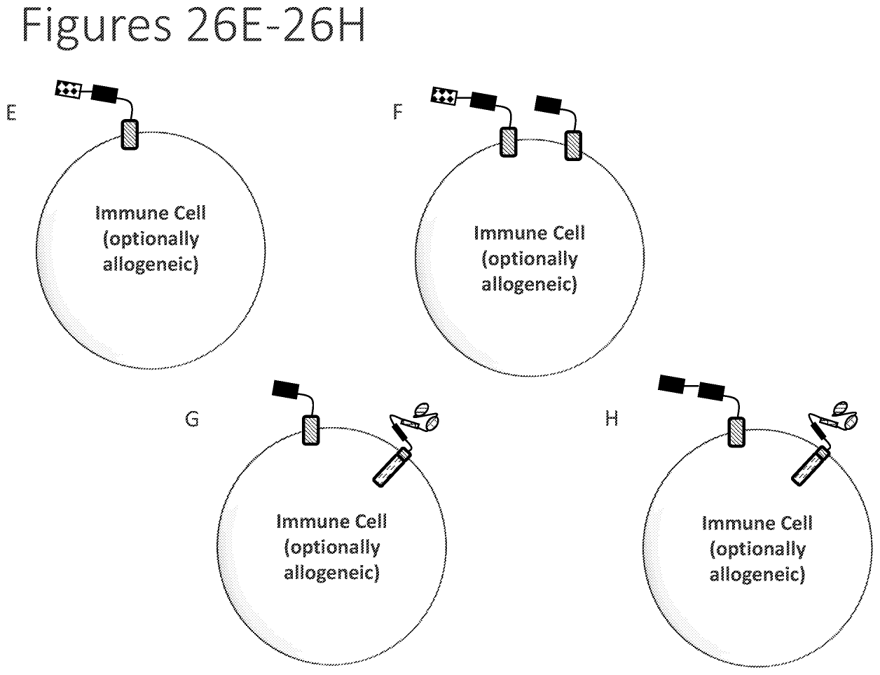

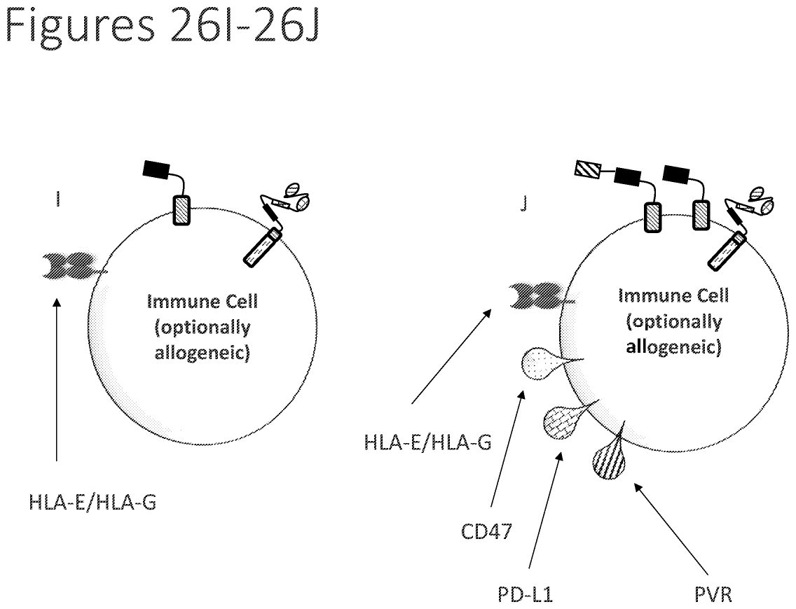

FIGS. 26A-26J show non-limiting schematic depictions of embodiments disclosed herein. FIG. 26A shows a single viral immunosuppressive peptide coupled to a transmembrane protein. FIG. 26B shows two individual viral immunosuppressive peptides, each coupled to a transmembrane protein. FIG. 26C shows a plurality of viral immunosuppressive peptides in a membrane-bound format. As shown, and described herein, both the viral immunosuppressive peptide and the transmembrane domain can vary, or can be the same. In other words, provided for herein are combinations such as viral immunosuppressive peptide (VIP) 1: transmembrane protein (TM) 1; VIP2:TM1; VIP2:TM2; VIPI:TM2; VIP3:TM1; VIPI:TM3, and the like, including VIPX:TMY.

FIG. 26D shows an additional non-limiting embodiment wherein multiple viral immunosuppressive peptides are coupled to a single transmembrane protein, with the viral immunosuppressive peptides being the same. FIG. 26E shows an additional non-limiting embodiment with wherein multiple viral immunosuppressive peptides are coupled to a single transmembrane protein, with the viral immunosuppressive peptides being distinct from one another. FIG. 26F shows a further non-limiting embodiment wherein multiple membrane-bound constructs are expressed on a single immune cell, one coupled to a single viral immunosuppressive peptide, and the other coupled to multiple viral immunosuppressive peptide. While not illustrated, it shall be appreciated that the transmembrane domains may differ from one another when multiple constructs are expressed by an individual cell.

Various transmembrane proteins can be used, such as one or more of CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. In several embodiments, a portion of one or more of these domains (e.g., a transmembrane domain) is used to anchor or otherwise tether the viral immunosuppressive peptide(s) to the immune cell surface. In several embodiments, the transmembrane protein comprises a CD8α transmembrane domain. In several embodiments, the CD8α transmembrane domain comprises the amino acid sequence of SEQ ID NO: 4, or a sequence with at least about 80%, at least about 85%, about 90%, about 95%, about 96%, about 97%, about 98% or about 99% sequence identity with SEQ ID NO: 4. In several embodiments, the CD8α transmembrane domain is encoded by the nucleic acid sequence of SEQ ID NO: 3, or a sequence with at least about 80%, at least about 85%, about 90%, about 95%, about 96%, about 97%, about 98% or about 99% sequence identity with SEQ ID NO: 3. In several embodiments, a hinge or other linker is used to couple the viral immunosuppressive peptide to the transmembrane protein. In several embodiments, a CD8α is used. In several embodiments, the CD8α hinge comprises the amino acid sequence of SEQ ID NO: 2, or a sequence with at least about 80%, at least about 85%, about 90%, about 95%, about 96%, about 97%, about 98% or about 99% sequence identity with SEQ ID NO: 2. In several embodiments, the CD8α hinge comprises the amino acid sequence of SEQ ID NO: 1, or a sequence with at least about 80%, at least about 85%, about 90%, about 95%, about 96%, about 97%, about 98% or about 99% sequence identity with SEQ ID NO: 1.

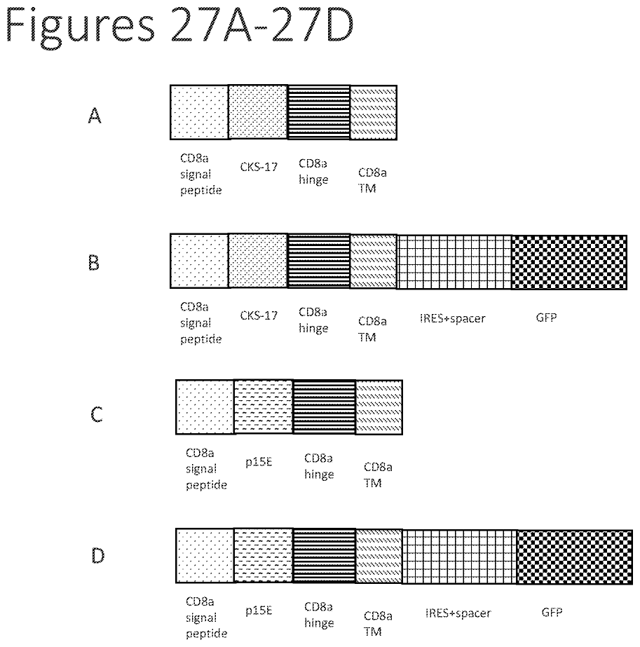

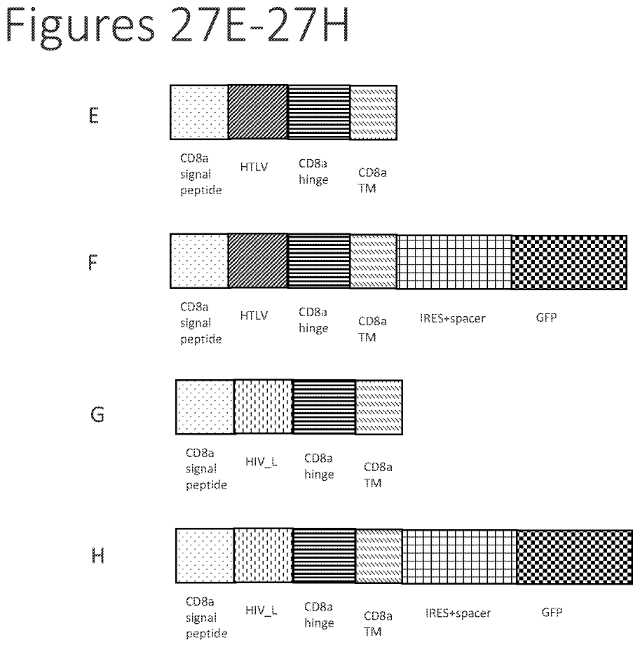

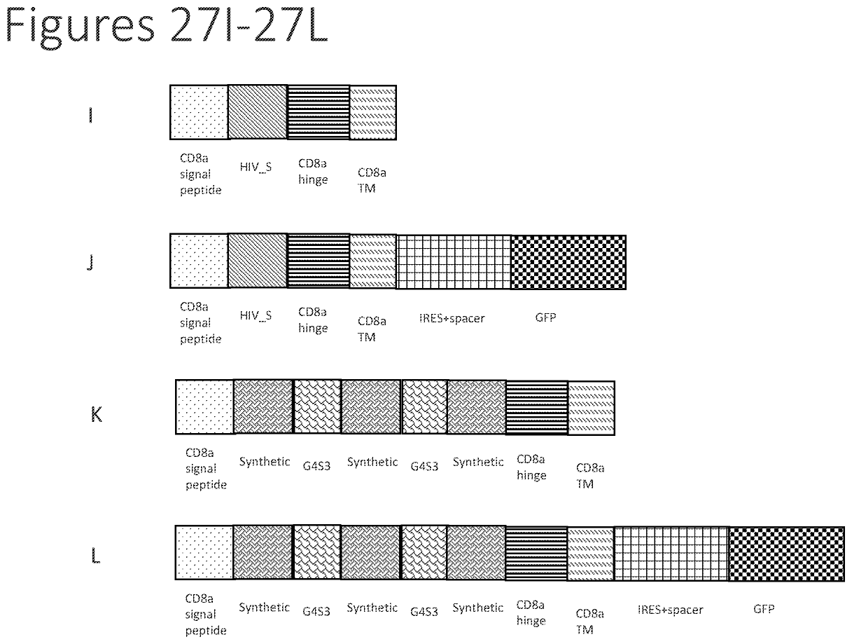

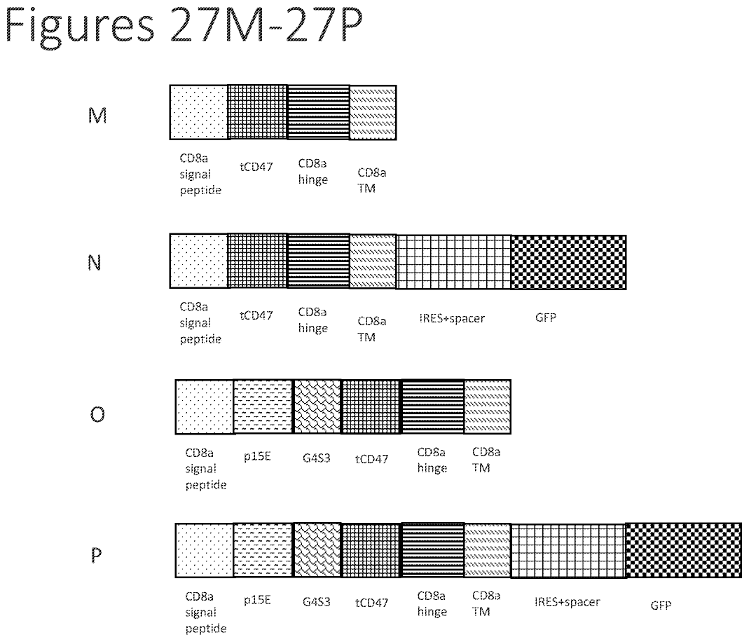

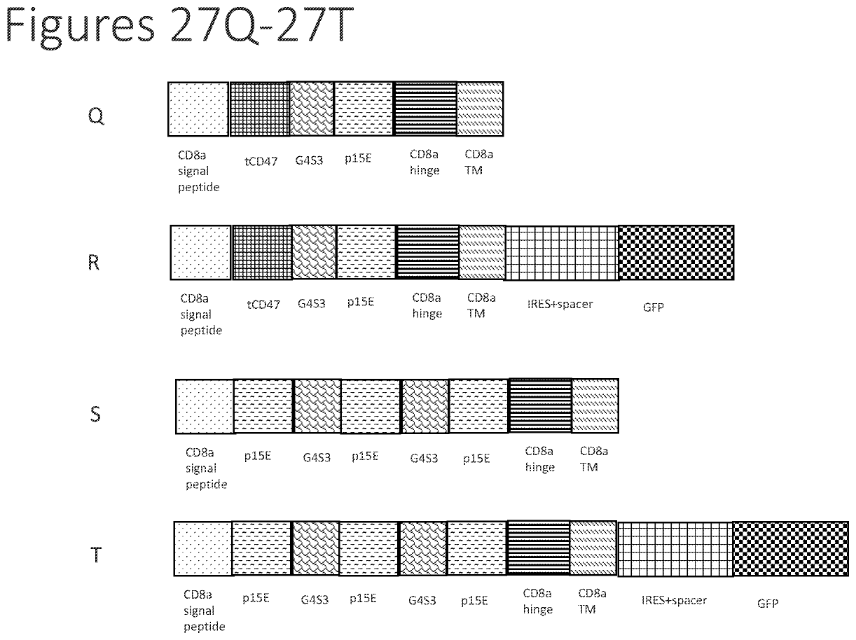

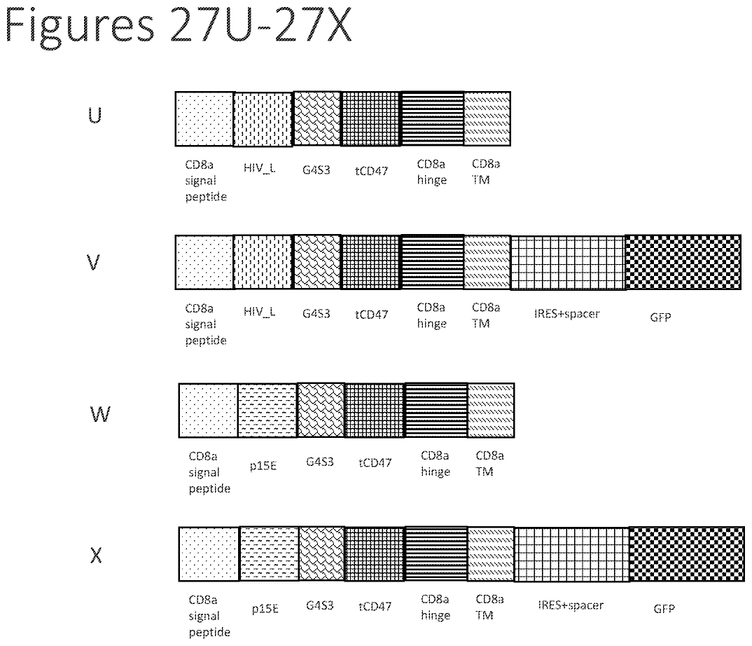

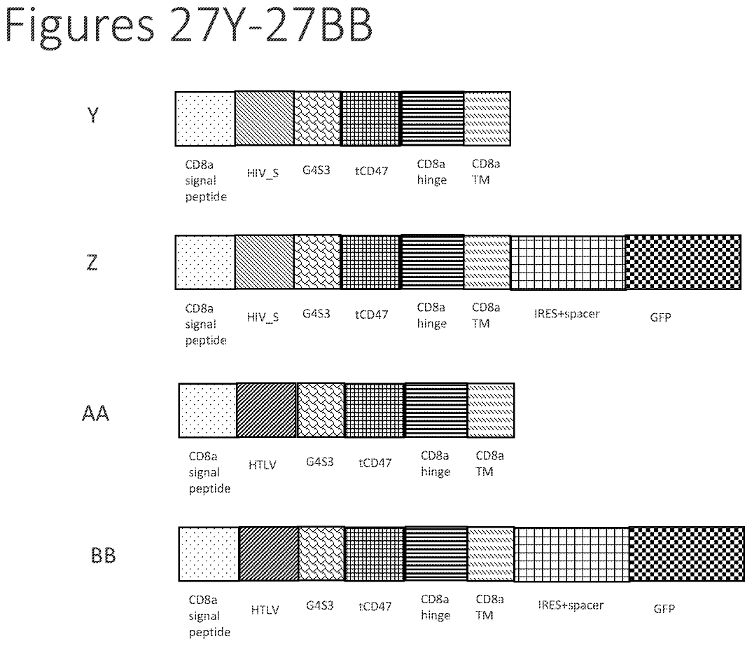

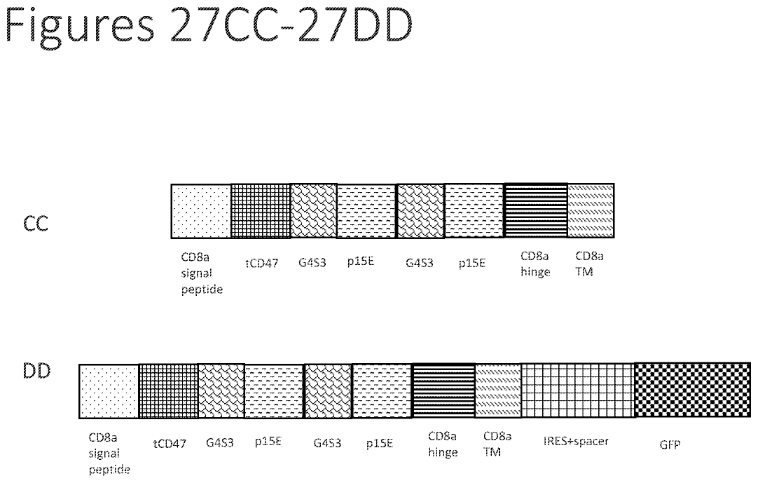

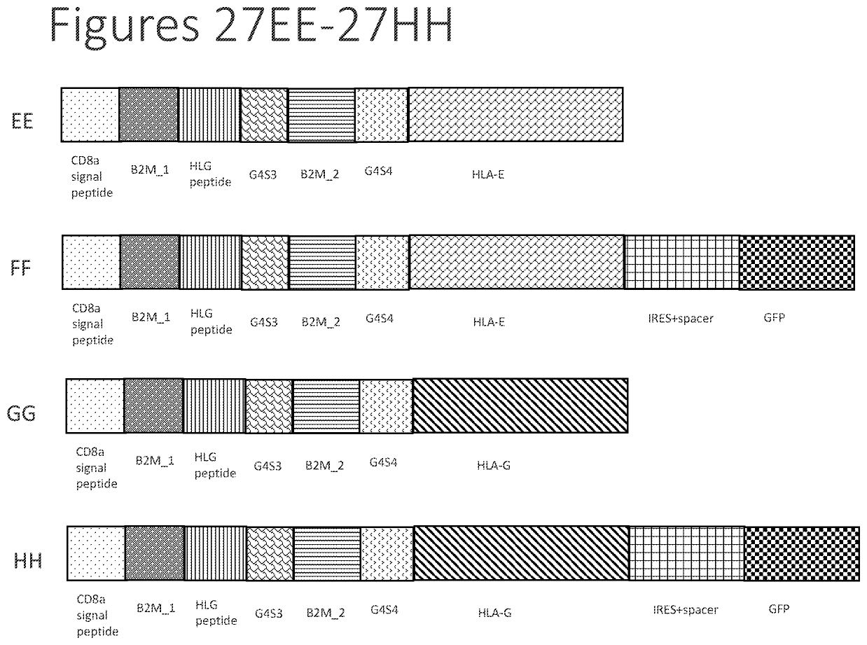

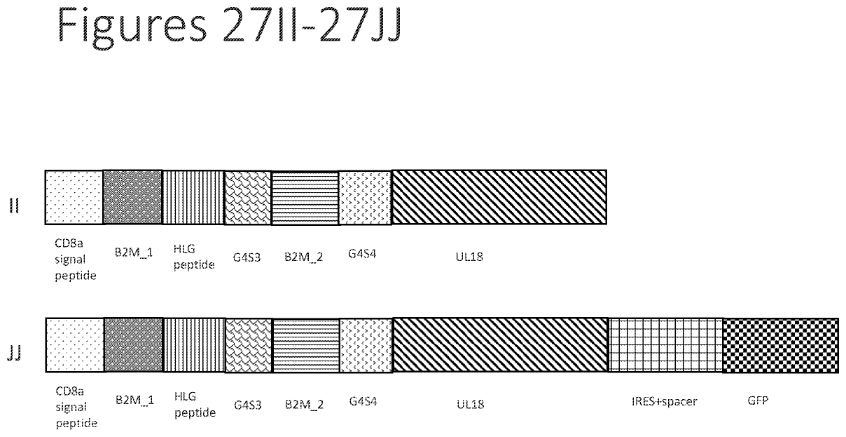

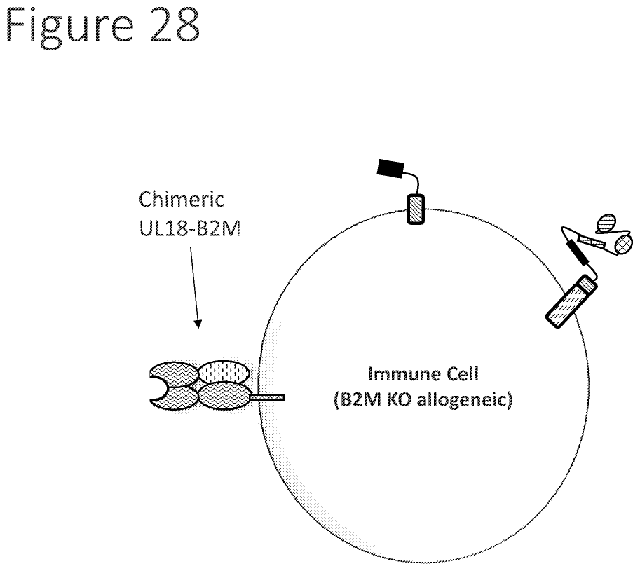

FIG. 26G shows an additional schematic of a non-limiting embodiment provided for herein. This embodiment comprises an immune cell engineered to express a CAR as well as a membrane-bound viral immunosuppressive peptide. FIG. 27G shows a non-limiting embodiment wherein the immune cell expresses a CAR along with a plurality of viral immunosuppressive peptides coupled to a transmembrane domain. Also provided for are immune cells (e.g., NK cells, T cells or combinations there) expressing a CAR and multiple membrane bound viral immunosuppressive peptides (e.g., as in FIG. 26F). In several embodiments, the CAR comprises one or more viral immunosuppressive peptides, while some embodiments involve expression of a CAR without a viral immunosuppressive peptide. As shown in these schematic figures, the immune cells engineered, in several embodiments, are allogeneic cells. In several embodiments, allogeneic NK cells are used. In several embodiments, allogeneic T cells are used. In several embodiments, combinations (e.g., a mixed population) of allogeneic NK cell and allogeneic T cells are used.

In several embodiments, there is provided a polynucleotide encoding a synthetic CKS-17 viral immunosuppressive peptide. In several embodiments, the polynucleotide encodes an amino acid sequence comprising SEQ ID NO: 199. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 199. In several embodiments, the polynucleotide comprises SEQ ID NO: 216 or shares at least about 80%, at least about 85%, at least about 90%, at least about 94%, at least about 95%, at least about 96%, at least about 97%, at least about 98%, or at least about 99%, sequence identity with SEQ ID NO: 216. In several embodiments, provided for is a membrane-bound synthetic CKS-17 viral immunosuppressive peptide. In several embodiments, the synthetic CKS-17 viral immunosuppressive peptide is coupled to a CD8α transmembrane protein and/or CD8α hinge domain. In several embodiments, the membrane-bound synthetic CKS-17 viral immunosuppressive peptide construct comprises one or more linker sequences. In several embodiments, the membrane-bound synthetic CKS-17 viral immunosuppressive peptide comprises a CD8α signal peptide, synthetic CKS-17, a CD8α hinge, and a CD8α transmembrane domain. In several embodiments, the membrane-bound synthetic CKS-17 viral immunosuppressive peptide construct is encoded by a polynucleotide that encodes an amino acid sequence comprising SEQ ID NO: 218 or SEQ ID NO: 693. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 218 or SEQ ID NO: 693. In several embodiments, the polynucleotide comprises SEQ ID NO: 219 (or SEQ ID NO: 694) or shares at least about 80%, at least about 85%, at least about 90%, at least about 94%, at least about 95%, at least about 96%, at least about 97%, at least about 98%, or at least about 99%, sequence identity with SEQ ID NO: 219 (or SEQ ID NO: 694). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound synthetic CKS-17 and GFP comprises SEQ ID NO: 217 or shares at least about 80%, at least about 85%, at least about 90%, at least about 94%, at least about 95%, at least about 96%, at least about 97%, at least about 98%, or at least about 99%, sequence identity with SEQ ID NO: 217. In several embodiments, the polynucleotide encoding membrane-bound synthetic CKS-17, a FLAG tag, and GFP comprises SEQ ID NO: 692 or shares at least about 80%, at least about 85%, at least about 90%, at least about 94%, at least about 95%, at least about 96%, at least about 97%, at least about 98%, or at least about 99%, sequence identity with SEQ ID NO: 692. In several embodiments, the polynucleotide encoding membrane-bound synthetic CKS-17, a FLAG tag, and GFP encodes the amino acid sequence of SEQ ID NO: 691 or an amino acid sequence that shares at least about 80%, at least about 85%, at least about 90%, at least about 94%, at least about 95%, at least about 96%, at least about 97%, at least about 98%, or at least about 99%, sequence identity with SEQ ID NO: 691.

In several embodiments, there is provided a polynucleotide encoding a p15E viral immunosuppressive peptide. In several embodiments, the polynucleotide encodes an amino acid sequence comprising SEQ ID NO: 220. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 220. In several embodiments, the polynucleotide comprises SEQ ID NO: 221 or shares at least about 80%, at least about 85%, at least about 90%, at least about 94%, at least about 95%, at least about 96%, at least about 97%, at least about 98%, or at least about 99%, sequence identity with SEQ ID NO: 221. In several embodiments, provided for is a membrane-bound p15E viral immunosuppressive peptide. In several embodiments, the p15E viral immunosuppressive peptide is coupled to a CD8α transmembrane protein and/or CD8α hinge domain. In several embodiments, the membrane-bound p15E viral immunosuppressive peptide construct comprises one or more linker sequences. In several embodiments, the membrane-bound p15E viral immunosuppressive peptide comprises a CD8α signal peptide, p15E, a CD8α hinge, and a CD8α transmembrane domain. In several embodiments, the membrane-bound p15E viral immunosuppressive peptide construct is encoded by a polynucleotide that encodes an amino acid sequence comprising SEQ ID NO: 223 (or SEQ ID NO: 697). In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO: 223 (or SEQ ID NO: 697). In several embodiments, the polynucleotide comprises SEQ ID NO: 224 (or SEQ ID NO: 698) or shares at least about 80%, at least about 85%, at least about 90%, at least about 94%, at least about 95%, at least about 96%, at least about 97%, at least about 98%, or at least about 99%, sequence identity with SEQ ID NO: 224 (or SEQ ID NO: 698). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding mbp15E and GFP comprises SEQ ID NO: 222 or shares at least about 80%, at least about 85%, at least about 90%, at least about 94%, at least about 95%, at least about 96%, at least about 97%, at least about 98%, or at least about 99%, sequence identity with SEQ ID NO: 222. In several embodiments, the polynucleotide encoding mbp15E, a FLAG tag, and GFP comprises SEQ ID NO: 696 or shares at least about 80%, at least about 85%, at least about 90%, at least about 94%, at least about 95%, at least about 96%, at least about 97%, at least about 98%, or at least about 99%, sequence identity with SEQ ID NO: 696. In several embodiments, the membrane-bound p15E viral immunosuppressive peptide construct including GFP and a FLAG tag is encoded by a polynucleotide that encodes an amino acid sequence comprising SEQ ID NO: 695). In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO: 695).

In several embodiments, there is provided a polynucleotide encoding a HTLV viral immunosuppressive peptide. In several embodiments, the polynucleotide encodes an amino acid sequence comprising SEQ ID NO: 225. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 225. In several embodiments, the polynucleotide comprises SEQ ID NO: 226 or shares at least about 80%, at least about 85%, at least about 90%, at least about 94%, at least about 95%, at least about 96%, at least about 97%, at least about 98%, or at least about 99%, sequence identity with SEQ ID NO: 226. In several embodiments, provided for is a membrane-bound HTLV viral immunosuppressive peptide. In several embodiments, the HTLV viral immunosuppressive peptide is coupled to a CD8α transmembrane protein and/or CD8α hinge domain. In several embodiments, the membrane-bound HTLV viral immunosuppressive peptide construct comprises one or more linker sequences. In several embodiments, the membrane-bound HTLV viral immunosuppressive peptide comprises a CD8α signal peptide, HTLV-1 (Gp21), a CD8α hinge, a and a CD8α transmembrane domain. In several embodiments, the membrane-bound HTLV viral immunosuppressive peptide construct is encoded by a polynucleotide that encodes an amino acid sequence comprising SEQ ID NO: 228 (or SEQ ID NO: 701). In several embodiments, the polynucleotide encodes an amino acid sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 228 (or SEQ ID NO: 701). In several embodiments, the polynucleotide comprises SEQ ID NO: 229 (or SEQ ID NO: 702) or shares at least about 90%, at least about 94%, at least about 95%, at least about 96%, at least about 97%, at least about 98%, or at least about 99%, sequence identity with SEQ ID NO: 229 (or SEQ ID NO: 702). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound HTLV and GFP comprises SEQ ID NO: 227 or shares at least about 80%, at least about 85%, at least about 90%, at least about 94%, at least about 95%, at least about 96%, at least about 97%, at least about 98%, or at least about 99%, sequence identity with SEQ ID NO: 227. In several embodiments, the polynucleotide encoding membrane-bound HTLV, a FLAG tag, and GFP comprises SEQ ID NO: 700 or shares at least about 80%, at least about 85%, at least about 90%, at least about 94%, at least about 95%, at least about 96%, at least about 97%, at least about 98%, or at least about 99%, sequence identity with SEQ ID NO: 700. In several embodiments, the membrane-bound HTLV, FLAG tag, GFP construct comprises the amino acid sequence of SEQ ID NO: 699 or shares at least about 80%, at least about 85%, at least about 90%, at least about 94%, at least about 95%, at least about 96%, at least about 97%, at least about 98%, or at least about 99%, sequence identity with SEQ ID NO: 699.

In several embodiments, there is provided a polynucleotide encoding a modified HIV Gp41 viral immunosuppressive peptide. In several embodiments, the polynucleotide encodes an amino acid sequence comprising SEQ ID NO: 230. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 230. In several embodiments, the polynucleotide comprises SEQ ID NO: 231 or shares at least about 80%, at least about 85%, at least about 90%, at least about 94%, at least about 95%, at least about 96%, at least about 97%, at least about 98%, or at least about 99%, sequence identity with SEQ ID NO: 231. In several embodiments, provided for is a membrane-bound modified HIV Gp41viral immunosuppressive peptide. In several embodiments, the modified HIV Gp41 viral immunosuppressive peptide is coupled to a CD8α transmembrane protein and/or CD8α hinge domain. In several embodiments, the membrane-bound modified HIV Gp41 viral immunosuppressive peptide construct comprises one or more linker sequences. In several embodiments, the membrane-bound modified HIV Gp41 viral immunosuppressive peptide comprises a CD8α signal peptide, modified HIV Gp41, a CD8α hinge, a and a CD8α transmembrane domain. In several embodiments, the membrane-bound modified HIV Gp41 viral immunosuppressive peptide construct is encoded by a polynucleotide that encodes an amino acid sequence comprising SEQ ID NO: 233 (or SEQ ID NO: 705). In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO: 233 (or SEQ ID NO: 705). In several embodiments, the polynucleotide comprises SEQ ID NO: 234 (or SEQ ID NO: 706) or shares at least about 80%, at least about 85%, at least about 90%, at least about 94%, at least about 95%, at least about 96%, at least about 97%, at least about 98%, or at least about 99%, sequence identity with SEQ ID NO: 234 (or SEQ ID NO: 706). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound modified HIV Gp41 and GFP comprises SEQ ID NO: 232 or shares at least about 80%, at least about 85%, at least about 90%, at least about 94%, at least about 95%, at least about 96%, at least about 97%, at least about 98%, or at least about 99%, sequence identity with SEQ ID NO: 232. In several embodiments, the polynucleotide encoding membrane-bound modified HIV Gp41, a FLAG tag, and GFP comprises SEQ ID NO: 704 or shares at least about 80%, at least about 85%, at least about 90%, at least about 94%, at least about 95%, at least about 96%, at least about 97%, at least about 98%, or at least about 99%, sequence identity with SEQ ID NO: 704. In several embodiments, the membrane-bound modified HIV Gp41, FLAG tag, GFP construct comprises the amino acid sequence of SEQ ID NO: 703 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 703.

In several embodiments, there is provided a polynucleotide encoding a truncated HIV Gp41 viral immunosuppressive peptide. In several embodiments, the polynucleotide encodes an amino acid sequence comprising SEQ ID NO: 235. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 235. In several embodiments, the polynucleotide comprises SEQ ID NO: 236 or shares at least about 80%, at least about 85%, at least about 90%, at least about 94%, at least about 95%, at least about 96%, at least about 97%, at least about 98%, or at least about 99%, sequence identity with SEQ ID NO: 236. In several embodiments, provided for is a membrane-bound HIV Gp41 viral immunosuppressive peptide. In several embodiments, the HIV Gp41 viral immunosuppressive peptide is coupled to a CD8α transmembrane protein and/or CD8α hinge domain. In several embodiments, the membrane-bound HIV Gp41 viral immunosuppressive peptide construct comprises one or more linker sequences. In several embodiments, the membrane-bound HIV Gp41 viral immunosuppressive peptide comprises a CD8α signal peptide, HIV Gp41, a CD8α hinge, a and a CD8αtransmembrane domain. In several embodiments, the membrane-bound HIV Gp41 viral immunosuppressive peptide construct is encoded by a polynucleotide that encodes an amino acid sequence comprising SEQ ID NO: 238 (or SEQ ID NO: 709). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 238 (or SEQ ID NO: 709). In several embodiments, the polynucleotide comprises SEQ ID NO: 239 (or SEQ ID NO: 710) or shares at least about 80%, at least about 85%, at least about 90%, at least about 94%, at least about 95%, at least about 96%, at least about 97%, at least about 98%, or at least about 99%, sequence identity with SEQ ID NO: 239 (or SEQ ID NO: 710). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound HIV Gp41 and GFP comprises SEQ ID NO: 237 or shares at least about 80%, at least about 85%, at least about 90%, at least about 94%, at least about 95%, at least about 96%, at least about 97%, at least about 98%, or at least about 99%, sequence identity with SEQ ID NO: 237. In several embodiments, the polynucleotide encoding membrane-bound HIV Gp41, a FLAG tag, and GFP comprises SEQ ID NO: 708 or shares at least about 80%, at least about 85%, at least about 90%, at least about 94%, at least about 95%, at least about 96%, at least about 97%, at least about 98%, or at least about 99%, sequence identity with SEQ ID NO: 708. In several embodiments, the membrane-bound truncated HIV Gp41, FLAG tag, GFP construct comprises the amino acid sequence of SEQ ID NO: 707 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 707.

In several embodiments, there is provided a polynucleotide encoding a synthetic viral immunosuppressive peptide. In several embodiments, the polynucleotide encodes an amino acid sequence comprising SEQ ID NO: 240. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 240. In several embodiments, the polynucleotide comprises SEQ ID NO: 241 or shares at least about 80%, at least about 85%, at least about 90%, at least about 94%, at least about 95%, at least about 96%, at least about 97%, at least about 98%, or at least about 99%, sequence identity with SEQ ID NO: 241. In several embodiments, provided for is a membrane-bound synthetic viral immunosuppressive peptide. In several embodiments, the synthetic viral immunosuppressive peptide is coupled to a CD8α transmembrane protein and/or CD8α hinge domain. In several embodiments, the membrane-bound synthetic viral immunosuppressive peptide construct comprises one or more linker sequences. In several embodiments, the membrane-bound synthetic viral immunosuppressive peptide comprises a CD8α signal peptide, a synthetic viral immunosuppressive peptide trimer, a CD8α hinge, a and a CD8α transmembrane domain. In several embodiments, the membrane-bound synthetic viral immunosuppressive peptide construct is encoded by a polynucleotide that encodes an amino acid sequence comprising SEQ ID NO: 243 (or SEQ ID NO: 713). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 243 (or SEQ ID NO: 713). In several embodiments, the polynucleotide comprises SEQ ID NO: 244 (or SEQ ID NO: 714) or shares at least about 80%, at least about 85%, at least about 90%, at least about 94%, at least about 95%, at least about 96%, at least about 97%, at least about 98%, or at least about 99%, sequence identity with SEQ ID NO: 244 (or SEQ ID NO: 714). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound synthetic viral immunosuppressive peptide and GFP comprises SEQ ID NO: 242 or shares at least about 80%, at least about 85%, at least about 90%, at least about 94%, at least about 95%, at least about 96%, at least about 97%, at least about 98%, or at least about 99%, sequence identity with SEQ ID NO: 242. In several embodiments, the polynucleotide encoding membrane-bound synthetic viral immunosuppressive peptide, a FLAG tag and GFP comprises SEQ ID NO: 712 or shares at least about 80%, at least about 85%, at least about 90%, at least about 94%, at least about 95%, at least about 96%, at least about 97%, at least about 98%, or at least about 99%, sequence identity with SEQ ID NO: 712. In several embodiments, membrane-bound synthetic viral immunosuppressive peptide, FLAG tag, GFP construct comprises the amino acid of SEQ ID NO: 712 or shares at least about 80%, at least about 85%, at least about 90%, at least about 94%, at least about 95%, at least about 96%, at least about 97%, at least about 98%, or at least about 99%, sequence identity with SEQ ID NO: 712.

In several embodiments, viral fusion peptides, or variants thereof, are used. By way of example, HIV initiates an immune evasive response by fusing to a target cell via a fusion peptide, a portion of which interacts with the T cell receptor on host T cells and suppresses their activation. In several embodiments, a portion of a viral fusion peptide is used. For example in several embodiments, an amino acid sequence comprising residues 5 to 13 of the HIV fusion peptide are used in an immunosuppressive effector as disclosed herein (e.g., incorporated into a CAR at one or more extracellular locations, or with one or more copies coupled to a transmembrane domain). In several embodiments, that amino acid sequence comprises SEQ ID NO: 467. In several embodiments, the amino acid sequence shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 467. In several embodiments, a modified viral fusion protein is used in an immunosuppressive effector as disclosed herein (e.g., incorporated into a CAR at one or more extracellular locations, or with one or more copies coupled to a transmembrane domain). In several embodiments, that amino acid sequence comprises SEQ ID NO: 468. In several embodiments, the amino acid sequence shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 468. In several embodiments, one or more consensus motifs derived from a viral fusion protein are used in an immunosuppressive effector as disclosed herein (e.g., incorporated into a CAR at one or more extracellular locations, or with one or more copies coupled to a transmembrane domain). In several embodiments, that amino acid consensus sequence comprises GXXXG (SEQ ID NO: 473) or AXXXG (SEQ ID NO: 474), where each X independently is any amino acid. In several embodiments, the amino acid sequence shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 473 or 474, while maintaining the consensus motif. In several embodiments, these motifs are particularly advantageous in that they are suppressive towards T-cells and not NKs. Thus, in several embodiments, these peptides allow engineered NK cells to be developed without gene editing to reduce/knock out B2M expression and still effect functional reduction in host versus graft rejection (e.g., through T cell suppression alone).

In several embodiments, there is provided a polynucleotide encoding a p15E viral immunosuppressive trimeric peptide. In several embodiments, the polynucleotide encodes an amino acid sequence comprising SEQ ID NO: 250. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 250. In several embodiments, the polynucleotide comprises SEQ ID NO: 251 or shares at least about 80%, at least about 85%, at least about 90%, at least about 94%, at least about 95%, at least about 96%, at least about 97%, at least about 98%, or at least about 99%, sequence identity with SEQ ID NO: 251. In several embodiments, provided for is a membrane-bound p15E viral immunosuppressive trimeric peptide. In several embodiments, the p15E viral immunosuppressive trimeric peptide is coupled to a CD8α transmembrane protein and/or CD8α hinge domain. In several embodiments, the membrane-bound p15E viral immunosuppressive trimeric peptide construct comprises one or more linker sequences (e.g., in between the peptide repeats). In several embodiments, the membrane-bound p15E viral immunosuppressive trimeric peptide comprises a CD8αsignal peptide, a first p15E peptide, a linker (e.g., a GS linker), a second p15E peptide, a linker (e.g., a second GS linker), a third p15E peptide, a CD8α hinge, and a CD8α transmembrane domain. In several embodiments, the membrane-bound p15E viral immunosuppressive peptide construct is encoded by a polynucleotide that encodes an amino acid sequence comprising SEQ ID NO: 253 (or SEQ ID NO: 721). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 253 (or SEQ ID NO: 721). In several embodiments, the polynucleotide comprises SEQ ID NO: 254 (or SEQ ID NO: 722) or shares at least about 80%, at least about 85%, at least about 90%, at least about 94%, at least about 95%, at least about 96%, at least about 97%, at least about 98%, or at least about 99%, sequence identity with SEQ ID NO: 254 (or SEQ ID NO: 722). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound trimeric p15E and GFP comprises SEQ ID NO: 252 or shares at least about 80%, at least about 85%, at least about 90%, at least about 94%, at least about 95%, at least about 96%, at least about 97%, at least about 98%, or at least about 99%, sequence identity with SEQ ID NO: 252. In several embodiments, the polynucleotide encoding membrane-bound trimeric p15E, a FLAG tag, and GFP comprises SEQ ID NO: 720 or shares at least about 80%, at least about 85%, at least about 90%, at least about 94%, at least about 95%, at least about 96%, at least about 97%, at least about 98%, or at least about 99%, sequence identity with SEQ ID NO: 720. In several embodiments, the membrane-bound trimeric p15E, FLAG tag, GFP construct comprises the amino acid sequence of SEQ ID NO: 719 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 719.

Chimeric proteins that include one or more viral immunosuppressive peptides are also used, in several embodiments. One such chimeric protein comprises the human cytomegalovirus class I MHC homolog, UL18. As described in more detail below, in several embodiments, UL18 is incorporated into a CAR, or otherwise expressed (e.g., through chimeric UL18-B2M or in a dimer/trimer construct) in order to evade host-NK cell cytotoxicity through the UL18 binding to the NK cell inhibitory receptor LIR-1.

Additionally, certain sequence domains or even particular residues within viral immunosuppressive peptides can facilitate further engineering of chimeric antigen receptors and their expression. For example, certain residues could be used to engineer CAR dimers, e.g., through disulfide bonding. Such an approach could be utilized to supplement, or replace, the use of bi-specific CARs. For example, rather than engineering a single CAR with, by way of example an anti-CD19 binder and a binder of NKG2D ligands, two separate CARs are engineered, each with cysteine residues positioned in a manner that allowed for the formation of di-sulfide bridges between the two CARs and thus the self-assembly of a dimerized CAR in vivo.

Advantageously, the use of the viral immunosuppressive peptides can not only serve to help mute the host NK cell response against administered engineered cells, but it can be used for other purposes as well. For example, in some embodiments, an antibody directed to a viral peptide is administered to a subject who has been dosed with a cell product expressing a CAR that comprises one or more viral immunosuppressive peptides. The antibody functions to bind the viral peptide and induce depletion of the CAR-expressing cells (e.g., via antibody-based immune response), thus serving as a safety mechanism or a route to end a treatment. Similarly, during manufacture of an immune cell comprising a CAR with an included viral immunosuppressive peptide, antibody-based detection of the viral immunosuppressive peptide can be used to determine CAR expression levels.

CD47 and Other Immunosuppressive Peptides, and Combinations with Viral Immunosuppressive Peptides

In addition to viral immunosuppressive peptides, other immunosuppressive peptides or polypeptides are provided for herein. Like the viral immunosuppressive peptides, these polypeptides may be included in one or more regions of a CAR, or can be expressed in a membrane-bound format. These additional immunosuppressive polypeptides can also be used in connection with one or more viral immunosuppressive peptides (see, e.g., FIGS. 25 and 26 and description of viral peptides above).

In several embodiments endogenous “self” signals are re-purposed to impart immune evasiveness to engineered immune cells. One such “self” protein is CD47, which impedes phagocytosis (e.g., by macrophages) through signaling through the phagocyte receptor CD172a. In several embodiments, one or more domains (or sub-domains) of CD47 are incorporated into a CAR and/or expressed in an immune cell in a membrane-bound configuration. In several embodiments, the expression of CD47 (in whole or in part) functions to impart to the engineered immune cell the ability to reduce or avoid phagocytosis by host immune cells, thereby enhancing the persistence (and thus functional life-span) of the engineered immune cells.

Other immunosuppressive peptides are also provided for herein. In several embodiments, PD-L1 (also known as CD274, PDL1, or PDCD1 L1) or an immunosuppressive portion thereof is expressed by an engineered immune cell through incorporation into a CAR and/or in a membrane-bound fashion. In several embodiments, one or more TIGIT ligands, including but not limited to PVR (also known as CD155, NECL5, or NECL-5) and CD113 (also known as PROM1 or prominin 1) or an immunosuppressive portion of is expressed by an engineered immune cell through incorporation into a CAR and/or in a membrane-bound fashion. In several embodiments, CD200 or an immunosuppressive portion thereof is expressed by an engineered immune cell through incorporation into a CAR and/or in a membrane-bound fashion. In several embodiments, CD276 (also known as B7-H3) or an immunosuppressive portion thereof is expressed by an engineered immune cell through incorporation into a CAR and/or in a membrane-bound fashion. In several embodiments, B7-H4 (also known as VTCN1, B7S1, or B7X)) or an immunosuppressive portion thereof is expressed by an engineered immune cell through incorporation into a CAR and/or in a membrane-bound fashion. In several embodiments, HVEM (also known as TNFSFi4, CD270 or ATAR) or an immunosuppressive portion thereof is expressed by an engineered immune cell through incorporation into a CAR and/or in a membrane-bound fashion. In several embodiments, CEACAM5 (also known as CEA) or an immunosuppressive portion thereof is expressed by an engineered immune cell through incorporation into a CAR and/or in a membrane-bound fashion. In several embodiments, Galectin-9 (also known as LGALS9) or an immunosuppressive portion thereof is expressed by an engineered immune cell through incorporation into a CAR and/or in a membrane-bound fashion. In several embodiments, the expression of these immunosuppressive proteins (in whole or in part) functions to impart to the engineered immune cell the ability to reduce or avoid immune clearance by host immune cells (or other engineered immune cells), thereby enhancing the persistence (and thus functional life-span) of the engineered immune cells.

As discussed above with respect to the viral immunosuppressive peptides, the engineered immune cells, in several embodiments, are allogeneic cells. In several embodiments, allogeneic NK cells are used. In several embodiments, allogeneic T cells are used. In several embodiments, combinations (e.g., a mixed population) of allogeneic NK cell and allogeneic T cells are used.

In several embodiments, the engineered CAR comprises one or more copies of one or more of the following amino acid sequences: SEQ ID NO: 245, 280, 285, 286, 288, or 289. In several embodiments, the CAR includes an immunosuppressive fragment of SEQ ID NO: 287. As discussed above, those sequences (or individual sequence) can be positioned in the hinge region, the N-terminal region or within the target binder region (e.g., within the linker of an scFv). In several embodiments, the CAR comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence with any of SEQ ID NOs: SEQ ID NO: 245, 280, 285, 286, 288, or 289 or an immunosuppressive fragment of SEQ ID NO: 287.

In addition to incorporation of the viral immunosuppressive peptides being positioned in the CAR, these non-viral immunosuppressive polypeptides can be coupled to a domain that allows them to be expressed as a membrane bound polypeptides.

Various transmembrane proteins can be used, such as one or more of CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. In several embodiments, a portion of one or more of these domains (e.g., a transmembrane domain) is used to anchor or otherwise tether the immunosuppressive peptide(s) to the immune cell surface. In several embodiments, the transmembrane protein comprises a CD8αtransmembrane domain. In several embodiments, the CD8α transmembrane domain comprises the amino acid sequence of SEQ ID NO: 4, or a sequence with at least about 80%, about 85%, about 90%, about 95%, about 96%, about 97%, about 98% or about 99% sequence identity with SEQ ID NO: 4. In several embodiments, the CD8α transmembrane domain is encoded by the nucleic acid sequence of SEQ ID NO: 3, or a sequence with at least about 80%, about 85%, about 90%, about 95%, about 96%, about 97%, about 98% or about 99% sequence identity with SEQ ID NO: 3. In several embodiments, a hinge or other linker is used to couple the immunosuppressive peptide to the transmembrane protein. In several embodiments, a CD8α is used. In several embodiments, the CD8α hinge comprises the amino acid sequence of SEQ ID NO: 2, or a sequence with at least about 80%, about 85%, about 90%, about 95%, about 96%, about 97%, about 98% or about 99% sequence identity with SEQ ID NO: 2. In several embodiments, the CD8α hinge comprises the amino acid sequence of SEQ ID NO: 1, or a sequence with at least about 80%, about 85%, about 90%, about 95%, about 96%, about 97%, about 98% or about 99% sequence identity with SEQ ID NO: 1.

In several embodiments, there is provided a polynucleotide encoding a truncated CD47 immunosuppressive peptide. In several embodiments, the polynucleotide encodes an amino acid sequence comprising SEQ ID NO: 245. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 245. In several embodiments, the polynucleotide comprises SEQ ID NO: 246 or shares at least about 80%, at least about 85%, at least about 90%, at least about 94%, at least about 95%, at least about 96%, at least about 97%, at least about 98%, or at least about 99%, sequence identity with SEQ ID NO: 246. In several embodiments, provided for is a membrane-bound truncated CD47 immunosuppressive peptide. In several embodiments, the truncated CD47 immunosuppressive peptide is coupled to a CD8α transmembrane protein and/or CD8α hinge domain. In several embodiments, the membrane-bound truncated CD47 immunosuppressive peptide construct comprises one or more linker sequences. In several embodiments, the membrane-bound truncated CD47 immunosuppressive peptide comprises a CD8α signal peptide, a truncated CD47 peptide (e.g., positions 110-130 of the extracellular domain), a CD8α hinge, and a CD8α transmembrane domain. In several embodiments, the membrane-bound synthetic truncated CD47 immunosuppressive peptide construct is encoded by a polynucleotide that encodes an amino acid sequence comprising SEQ ID NO: 248 (or SEQ ID NO: 717). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 248 (or SEQ ID NO: 717). In several embodiments, the polynucleotide comprises SEQ ID NO: 249 (or SEQ ID NO: 718) or shares at least about 80%, at least about 85%, at least about 90%, at least about 94%, at least about 95%, at least about 96%, at least about 97%, at least about 98%, or at least about 99%, sequence identity with SEQ ID NO: 249 (or SEQ ID NO: 718). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound truncated CD47 and GFP comprises SEQ ID NO: 247 or shares at least about 80%, at least about 85%, at least about 90%, at least about 94%, at least about 95%, at least about 96%, at least about 97%, at least about 98%, or at least about 99%, sequence identity with SEQ ID NO: 247. In several embodiments, the polynucleotide encoding membrane-bound truncated CD47, a FLAG tag and GFP comprises SEQ ID NO: 716 or shares at least about 80%, at least about 85%, at least about 90%, at least about 94%, at least about 95%, at least about 96%, at least about 97%, at least about 98%, or at least about 99%, sequence identity with SEQ ID NO: 716. In several embodiments, the membrane-bound truncated CD47, FLAG tag, GFP construct comprises the amino acid sequence of SEQ ID NO: 715 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 715.

As discussed above, any of the viral immunosuppressive peptides may be used in combination with one or more non-viral immunosuppressive peptides.

In several embodiments, there is provided a polynucleotide encoding a p15E viral immunosuppressive peptide truncated CD47 construct (p15E_tCD47). In several embodiments, provided for is a membrane-bound p15E_tCD47 construct. In several embodiments, the truncated CD47 peptide comprises a peptide corresponding to positions 110-130 of the extracellular domain of CD47. In several embodiments, the p15E_tCD47 construct is coupled to a CD8α transmembrane protein and/or CD8α hinge domain. In several embodiments, the membrane-bound p15E_tCD47 construct comprises one or more linker sequences. In several embodiments, the membrane-bound p15E_tCD47 construct comprises a CD8α signal peptide, a p15E peptide, a linker (e.g., a GS linker), a truncated CD47 peptide, CD8α hinge, and a CD8α transmembrane domain. In several embodiments, the membrane-bound p15E_tCD47 construct is encoded by a polynucleotide that encodes an amino acid sequence comprising SEQ ID NO: 256 (or SEQ ID NO: 725). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 256 (or SEQ ID NO: 725). In several embodiments, the polynucleotide comprises SEQ ID NO: 257 (or SEQ ID NO: 726) or shares at least about 80%, at least about 85%, at least about 90%, at least about 94%, at least about 95%, at least about 96%, at least about 97%, at least about 98%, or at least about 99%, sequence identity with SEQ ID NO: 257 (or SEQ ID NO: 726). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound p15E_tCD47 construct and GFP comprises SEQ ID NO: 255 or shares at least about 80%, at least about 85%, at least about 90%, at least about 94%, at least about 95%, at least about 96%, at least about 97%, at least about 98%, or at least about 99%, sequence identity with SEQ ID NO: 255. In several embodiments, the polynucleotide encoding the membrane-bound p15E_tCD47 construct, a FLAG tag, and GFP comprises SEQ ID NO: 724 or shares at least about 80%, at least about 85%, at least about 90%, at least about 94%, at least about 95%, at least about 96%, at least about 97%, at least about 98%, or at least about 99%, sequence identity with SEQ ID NO: 724. In several embodiments, the membrane-bound p15E_tCD47, FLAG tag, GFP construct comprises the amino acid sequence of SEQ ID NO: 723 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 723.

In several embodiments, there is provided a polynucleotide encoding a p15E viral immunosuppressive peptide truncated CD47 construct (tCD47_p15E), a swap of the peptides of the construct just described. In several embodiments, provided for is a membrane-bound tCD47_p15E construct. In several embodiments, the truncated CD47 peptide comprises a peptide corresponding to positions 110-130 of the extracellular domain of CD47. In several embodiments, the tCD47_p15E construct is coupled to a CD8α transmembrane protein and/or CD8α hinge domain. In several embodiments, the membrane-bound tCD47_p15E construct comprises one or more linker sequences. In several embodiments, the membrane-bound tCD47_p15E construct comprises a CD8α signal peptide, a truncated CD47 peptide, a linker (e.g., a GS linker), a p15E peptide, a CD8α hinge, and a CD8α transmembrane domain. In several embodiments, the membrane-bound tCD47_p15E construct is encoded by a polynucleotide that encodes an amino acid sequence comprising SEQ ID NO: 259 (or SEQ ID NO: 729). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 259 (or SEQ ID NO: 729). In several embodiments, the polynucleotide comprises SEQ ID NO: 260 (or SEQ ID NO: 730) or shares at least about 80%, at least about 85%, at least about 90%, at least about 94%, at least about 95%, at least about 96%, at least about 97%, at least about 98%, or at least about 99%, sequence identity with SEQ ID NO: 260 (or SEQ ID NO: 730). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound tCD47_p15E construct and GFP comprises SEQ ID NO: 258 or shares at least about 80%, at least about 85%, at least about 90%, at least about 94%, at least about 95%, at least about 96%, at least about 97%, at least about 98%, or at least about 99%, sequence identity with SEQ ID NO: 258. In several embodiments, the polynucleotide encoding the membrane-bound tCD47_p15E construct, a FLAG tag, and GFP comprises SEQ ID NO: 728 or shares at least about 80%, at least about 85%, at least about 90%, at least about 94%, at least about 95%, at least about 96%, at least about 97%, at least about 98%, or at least about 99%, sequence identity with SEQ ID NO: 728. In several embodiments, the membrane-bound tCD47_p15E, FLAG tag, GFP construct comprises the amino acid sequence of SEQ ID NO: 727 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 727.

In several embodiments, there is provided a polynucleotide encoding a modified HIV Gp41 viral immunosuppressive peptide truncated CD47 construct (HIV_L_tCD47). In several embodiments, provided for is a membrane-bound HIV_L_tCD47 construct. In several embodiments, the truncated CD47 peptide comprises a peptide corresponding to positions 110-130 of the extracellular domain of CD47. In several embodiments, the HIV_L_tCD47 construct is coupled to a CD8α transmembrane protein and/or CD8α hinge domain. In several embodiments, the membrane-bound HIV_L_tCD47 construct comprises one or more linker sequences. In several embodiments, the membrane-bound HIV_L_tCD47 construct comprises a CD8α signal peptide, a modified HIV Gp41 viral immunosuppressive peptide, a linker (e.g., a GS linker), a truncated CD47 peptide, a CD8α hinge, and a CD8α transmembrane domain. In several embodiments, the membrane-bound HIV_L_tCD47 construct is encoded by a polynucleotide that encodes an amino acid sequence comprising SEQ ID NO: 262 (or SEQ ID NO: 733). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 262 (or SEQ ID NO: 733). In several embodiments, the polynucleotide comprises SEQ ID NO: 263 (or SEQ ID NO: 734) or shares at least about 80%, at least about 85%, at least about 90%, at least about 94%, at least about 95%, at least about 96%, at least about 97%, at least about 98%, or at least about 99%, sequence identity with SEQ ID NO: 263 (or SEQ ID NO: 734). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound HIV_L_tCD47 construct and GFP comprises SEQ ID NO: 261 or shares at least about 80%, at least about 85%, at least about 90%, at least about 94%, at least about 95%, at least about 96%, at least about 97%, at least about 98%, or at least about 99%, sequence identity with SEQ ID NO: 261. In several embodiments, the polynucleotide encoding the membrane-bound HIV_L_tCD47 construct, a FLAG tag, and GFP comprises SEQ ID NO: 732 or shares at least about 80%, at least about 85%, at least about 90%, at least about 94%, at least about 95%, at least about 96%, at least about 97%, at least about 98%, or at least about 99%, sequence identity with SEQ ID NO: 732. In several embodiments, the membrane-bound HIV_L_tCD47, FLAG tag, GFP construct comprises the amino acid sequence of SEQ ID NO: 731 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 731.

In several embodiments, there is provided a polynucleotide encoding an HIV Gp41 viral immunosuppressive peptide truncated CD47 construct (HIV_S_tCD47). In several embodiments, provided for is a membrane-bound HIV_S_tCD47 construct. In several embodiments, the truncated CD47 peptide comprises a peptide corresponding to positions 110-130 of the extracellular domain of CD47. In several embodiments, the HIV_S_tCD47 construct is coupled to a CD8α transmembrane protein and/or CD8αhinge domain. In several embodiments, the membrane-bound HIV_S_tCD47 construct comprises one or more linker sequences. In several embodiments, the membrane-bound HIV_S_tCD47 construct comprises a CD8α signal peptide, an HIV Gp41 viral immunosuppressive peptide, a linker (e.g., a GS linker), a truncated CD47 peptide, a CD8α hinge, and a CD8α transmembrane domain. In several embodiments, the membrane-bound HIV_S_tCD47 construct is encoded by a polynucleotide that encodes an amino acid sequence comprising SEQ ID NO: 265 (or SEQ ID NO: 737). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 265 (or SEQ ID NO: 737). In several embodiments, the polynucleotide comprises SEQ ID NO: 266 (or SEQ ID NO: 738) or shares at least about 80%, at least about 85%, at least about 90%, at least about 94%, at least about 95%, at least about 96%, at least about 97%, at least about 98%, or at least about 99%, sequence identity with SEQ ID NO: 266 (or SEQ ID NO: 738). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound HIV_S_tCD47 construct and GFP comprises SEQ ID NO: 264 or shares at least about 80%, at least about 85%, at least about 90%, at least about 94%, at least about 95%, at least about 96%, at least about 97%, at least about 98%, or at least about 99%, sequence identity with SEQ ID NO: 264. In several embodiments, the polynucleotide encoding the membrane-bound HIV_S_tCD47 construct, a FLAG tag, and GFP comprises SEQ ID NO: 736 or shares at least about 80%, at least about 85%, at least about 90%, at least about 94%, at least about 95%, at least about 96%, at least about 97%, at least about 98%, or at least about 99%, sequence identity with SEQ ID NO: 736. In several embodiments, the membrane-bound HIV_S_tCD47, FLAG tag, GFP construct comprises the amino acid sequence of SEQ ID NO: 735 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 735.

In several embodiments, there is provided a polynucleotide encoding an HTLV viral immunosuppressive peptide truncated CD47 construct (HTLV_tCD47). In several embodiments, provided for is a membrane-bound HTLV_tCD47 construct. In several embodiments, the truncated CD47 peptide comprises a peptide corresponding to positions 110-130 of the extracellular domain of CD47. In several embodiments, the HTLV_tCD47 construct is coupled to a CD8α transmembrane protein and/or CD8α hinge domain. In several embodiments, the membrane-bound HTLV_tCD47 construct comprises one or more linker sequences. In several embodiments, the membrane-bound HTLV_tCD47 construct comprises a CD8α signal peptide, an HTLV viral immunosuppressive peptide, a linker (e.g., a GS linker), a truncated CD47 peptide, a CD8α hinge, and a CD8α transmembrane domain. In several embodiments, the membrane-bound HTLV_tCD47 construct is encoded by a polynucleotide that encodes an amino acid sequence comprising SEQ ID NO: 268 (or SEQ ID NO: 741). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 268 (or SEQ ID NO: 741). In several embodiments, the polynucleotide comprises SEQ ID NO: 269 (or SEQ ID NO: 742) or shares at least about 80%, at least about 85%, at least about 90%, at least about 94%, at least about 95%, at least about 96%, at least about 97%, at least about 98%, or at least about 99%, sequence identity with SEQ ID NO: 269 (or SEQ ID NO: 742). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound HTLV_tCD47 construct and GFP comprises SEQ ID NO: 267 or shares at least about 80%, at least about 85%, at least about 90%, at least about 94%, at least about 95%, at least about 96%, at least about 97%, at least about 98%, or at least about 99%, sequence identity with SEQ ID NO: 267. In several embodiments, the polynucleotide encoding the membrane-bound HTLV_tCD47 construct, a FLAG tag, and GFP comprises SEQ ID NO: 740 or shares at least about 80%, at least about 85%, at least about 90%, at least about 94%, at least about 95%, at least about 96%, at least about 97%, at least about 98%, or at least about 99%, sequence identity with SEQ ID NO: 740. In several embodiments, the membrane-bound HTLV_tCD47, FLAG tag, GFP construct comprises the amino acid sequence of SEQ ID NO: 739 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 739.

In several embodiments, there is provided a polynucleotide encoding a truncated CD47-p15E-dimer viral immunosuppressive peptide truncated CD47 construct (tCD47_p15Ex2). In several embodiments, provided for is a membrane-bound tCD47_p15Ex2 construct. In several embodiments, the truncated CD47 peptide comprises a peptide corresponding to positions 110-130 of the extracellular domain of CD47. In several embodiments, the tCD47_p15Ex2 construct is coupled to a CD8α transmembrane protein and/or CD8α hinge domain. In several embodiments, the membrane-bound tCD47_p15Ex2 construct comprises one or more linker sequences. In several embodiments, the membrane-bound tCD47_p15Ex2 construct comprises a CD8α signal peptide, a truncated CD47 peptide, a linker (e.g., a GS linker), a first p15E peptide, a linker (e.g., a GS linker), a second p15E peptide, a CD8α hinge, and a CD8αtransmembrane domain. In several embodiments, the membrane-bound tCD47_p15Ex2 construct is encoded by a polynucleotide that encodes an amino acid sequence comprising SEQ ID NO: 271 (or SEQ ID NO: 745). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 271 (or SEQ ID NO: 745). In several embodiments, the polynucleotide comprises SEQ ID NO: 272 (or SEQ ID NO: 746) or shares at least about 80%, at least about 85%, at least about 90%, at least about 94%, at least about 95%, at least about 96%, at least about 97%, at least about 98%, or at least about 99%, sequence identity with SEQ ID NO: 272 (or SEQ ID NO: 746). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound tCD47_p15Ex2 construct and GFP comprises SEQ ID NO: 270 or shares at least about 80%, at least about 85%, at least about 90%, at least about 94%, at least about 95%, at least about 96%, at least about 97%, at least about 98%, or at least about 99%, sequence identity with SEQ ID NO: 270. In several embodiments, the polynucleotide encoding the membrane-bound tCD47_p15Ex2 construct, a FLAG tag, and GFP comprises SEQ ID NO: 744 or shares at least about 80%, at least about 85%, at least about 90%, at least about 94%, at least about 95%, at least about 96%, at least about 97%, at least about 98%, or at least about 99%, sequence identity with SEQ ID NO: 744. In several embodiments, the membrane-bound tCD47_p15Ex2, FLAG tag, GFP construct comprises the amino acid sequence of SEQ ID NO: 743 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 743.

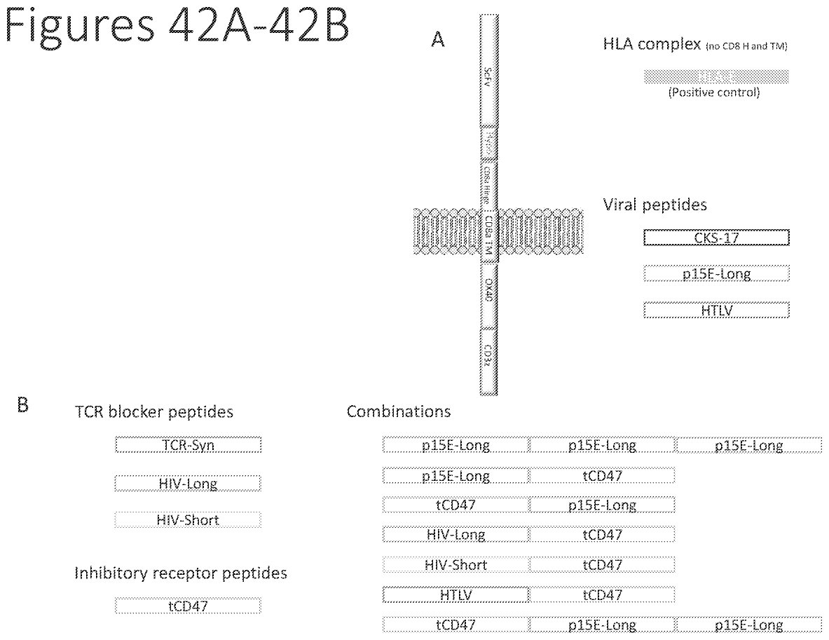

Additionally, according to several embodiments, combinations and/or replicates of various viral peptides are provided for. See FIGS. 42A and 42B, for example. FIG. 42A shows a non-limiting schematic of construct according to embodiments disclosed herein, in which a domain designed to reduce cytotoxic effects on a cell expressing such a construct (also referred to as a hypoimmune domain) is coupled to at least a transmembrane domain or (as shown in the non-limiting schematic), in some embodiments, integrated into a CAR construct. In several embodiments, viral peptides such as those disclosed herein are integrated into the CAR (e.g., CKS-17, p15E, including p15E-long, and/or HTLV). As shown in FIG. 42B, multimeric formats are provided for, in several embodiments, such as doublet or triplet of p15E (including p15E-long). Viral and non-viral peptides may also be used in combination, such as any of a synthetic TCR blocking peptide, HIV TCR blocking peptides (including truncated formats), an inhibitory receptor peptide (such as tCD47) with any of the other viral peptides disclosed herein. Non-limiting examples include p15E-tCD47, tCD47-p15E, HIV-long-TCD47, HIV-short-tCD47, HTLV-tCD47, and tCD47-p15E (doublet or triplet, optionally long format).

In several embodiments, there is provided a polynucleotide encoding a synthetic CKS-17 viral immunosuppressive peptide. In several embodiments, provided for is a membrane-bound synthetic CKS-17 viral immunosuppressive peptide. In several embodiments, the synthetic CKS-17 viral immunosuppressive peptide is coupled to a CD8α transmembrane protein and/or CD8α hinge domain. In several embodiments, the membrane-bound synthetic CKS-17 viral immunosuppressive peptide construct comprises one or more linker sequences. In several embodiments, the membrane-bound synthetic CKS-17 viral immunosuppressive peptide comprises a CD8α signal peptide, synthetic CKS-17, a CD8α hinge, and a CD8α transmembrane domain. In several embodiments, the membrane-bound synthetic CKS-17 viral immunosuppressive peptide construct is encoded by a polynucleotide that encodes an amino acid sequence comprising SEQ ID NO: 749. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 749. In several embodiments, the polynucleotide comprises SEQ ID NO: 750 or shares at least about 80%, at least about 85%, at least about 90%, at least about 94%, at least about 95%, at least about 96%, at least about 97%, at least about 98%, or at least about 99%, sequence identity with SEQ ID NO: 750. In several embodiments, the polynucleotide encoding membrane-bound synthetic CKS-17 also encodes GFP (without an IRES or a linker) and comprises SEQ ID NO: 748 or shares at least about 80%, at least about 85%, at least about 90%, at least about 94%, at least about 95%, at least about 96%, at least about 97%, at least about 98%, or at least about 99%, sequence identity with SEQ ID NO: 748. In several embodiments, the membrane-bound synthetic CKS-17-GFP construct comprises SEQ ID NO: 747 or shares at least about 80%, at least about 85%, at least about 90%, at least about 94%, at least about 95%, at least about 96%, at least about 97%, at least about 98%, or at least about 99%, sequence identity with SEQ ID NO: 747.

In several embodiments, a truncated CD47 immunosuppressive peptide is employed. In several embodiments, there is provided a polynucleotide that encodes an amino acid sequence comprising SEQ ID NO: 754. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 754. In several embodiments, the polynucleotide comprises SEQ ID NO: 753 or shares at least about 80%, at least about 85%, at least about 90%, at least about 94%, at least about 95%, at least about 96%, at least about 97%, at least about 98%, or at least about 99%, sequence identity with SEQ ID NO: 753. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding truncated CD47 and GFP comprises SEQ ID NO: 752 or shares at least about 80%, at least about 85%, at least about 90%, at least about 94%, at least about 95%, at least about 96%, at least about 97%, at least about 98%, or at least about 99%, sequence identity with SEQ ID NO: 752. In several embodiments, the truncated CD47-GFP construct comprises the amino acid sequence of SEQ ID NO: 751 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 751.

In several embodiments, there is provided a polynucleotide encoding an HIV Gp41 viral immunosuppressive peptide construct that includes a native HIV transmembrane domain (fGP41_HIVtm). In several embodiments, the HIV Gp41 viral immunosuppressive peptide is coupled to a CD8α signal peptide (e.g., for expression purposes, the sequence of which is MALPVTALLLPLALLLHAARP (SEQ ID NO 895) at the amino acid level and ATGGCACTCCCCGTAACTGCTCTGCTGCTG CCGTTGGCATTGCTCCTGCACGCCGCACGCCCG (SEQ ID NO 896) at the nucleotide level). In several embodiments, the HIV Gp41 viral immunosuppressive construct comprises a CD8α signal peptide and HIV Gp41 and is encoded by a polynucleotide that encodes an amino acid sequence comprising SEQ ID NO:757. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 757. In several embodiments, the polynucleotide comprises SEQ ID NO: 758 or shares at least about 80%, at least about 85%, at least about 90%, at least about 94%, at least about 95%, at least about 96%, at least about 97%, at least about 98%, or at least about 99%, sequence identity with SEQ ID NO: 758. Optionally, the polynucleotide encodes a detectable tag (e.g., a FLAG tag) and/or can comprise an internal ribosome entry site (IRES) that allows for expression of an additional protein, such as a detectable tag (e.g., GFP). n several embodiments, a linker (e.g., a GS linker) is positioned between the Gp41 domain and the tag. In several embodiments, the polynucleotide encoding HIV Gp41 and GFP comprises SEQ ID NO: 756 or shares at least about 80%, at least about 85%, at least about 90%, at least about 94%, at least about 95%, at least about 96%, at least about 97%, at least about 98%, or at least about 99%, sequence identity with SEQ ID NO: 756. In several embodiments, the fGP41_HIVtm-GFP construct comprises that amino acid sequence of SEQ ID NO: 755 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 755.

In several embodiments, there is provided a polynucleotide encoding a p15E transmembrane domain immunosuppressive peptide (p15Etm_sf). In several embodiments, the p15E transmembrane domain is coupled to a CD8α signal peptide (e.g., for expression purposes, the sequence of which is provided for separately herein). In several embodiments, the p15E transmembrane domain immunosuppressive construct comprises a CD8α signal peptide and the p15E transmembrane domain and is encoded by a polynucleotide that encodes an amino acid sequence comprising SEQ ID NO: 761. In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO:761. In several embodiments, the polynucleotide comprises SEQ ID NO: 762 or shares at least about 80%, at least about 85%, at least about 90%, at least about 94%, at least about 95%, at least about 96%, at least about 97%, at least about 98%, or at least about 99%, sequence identity with SEQ ID NO: 762. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the p15E transmembrane domain and the tag. In several embodiments, the polynucleotide encoding the p15E transmembrane domain and GFP comprises SEQ ID NO: 760 or shares at least about 80%, at least about 85%, at least about 90%, at least about 94%, at least about 95%, at least about 96%, at least about 97%, at least about 98%, or at least about 99%, sequence identity with SEQ ID NO: 760. In several embodiments, the p15E transmembrane domain-GFP construct comprises the amino acid sequence of SEQ ID NO: 759 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 759.

In several embodiments, there is provided a polynucleotide encoding a CD43-derived immunosuppressive peptide (fCD43_TM). In several embodiments, the CD43 peptide is coupled to a CD8αsignal peptide (e.g., for expression purposes, the sequence of which is provided for separately herein). In several embodiments, the CD43 immunosuppressive construct comprises a CD8α signal peptide and the CD43 peptide and is encoded by a polynucleotide that encodes an amino acid sequence comprising SEQ ID NO: 765. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 765. In several embodiments, the polynucleotide comprises SEQ ID NO: 766 or shares at least about 80%, at least about 85%, at least about 90%, at least about 94%, at least about 95%, at least about 96%, at least about 97%, at least about 98%, or at least about 99%, sequence identity with SEQ ID NO: 766. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD43 and the tag. In several embodiments, the polynucleotide encoding fCD43 and GFP comprises SEQ ID NO: 764 or shares at least about 80%, at least about 85%, at least about 90%, at least about 94%, at least about 95%, at least about 96%, at least about 97%, at least about 98%, or at least about 99%, sequence identity with SEQ ID NO: 764. In several embodiments, the CD43-GFP construct comprises the amino acid sequence of SEQ ID NO: 763 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 763.

In several embodiments, there is provided a polynucleotide encoding a latent membrane protein 1 (LMP1) peptide of the Epstein-Barr Virus (LMP1_TM). In several embodiments, the LMP1 peptide is coupled to a CD8α signal peptide (e.g., for expression purposes, the sequence of which is provided for separately herein). In several embodiments, the LMP1 immunosuppressive construct comprises a CD8αsignal peptide and the LMP1 peptide and is encoded by a polynucleotide that encodes an amino acid sequence comprising SEQ ID NO: 769. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 769. In several embodiments, the polynucleotide comprises SEQ ID NO: 770 or shares at least about 80%, at least about 85%, at least about 90%, at least about 94%, at least about 95%, at least about 96%, at least about 97%, at least about 98%, or at least about 99%, sequence identity with SEQ ID NO: 770. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LMP1 and the tag. In several embodiments, the polynucleotide encoding fLMP1 and GFP comprises SEQ ID NO: 768 or shares at least about 80%, at least about 85%, at least about 90%, at least about 94%, at least about 95%, at least about 96%, at least about 97%, at least about 98%, or at least about 99%, sequence identity with SEQ ID NO: 768. In several embodiments, the LMP1-GFP construct comprises the amino acid sequence of SEQ ID NO: 767 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 767.

In several embodiments, there is provided a polynucleotide encoding a glycoprotein D (gD) of the Herpes Simplex Virus (fgD_TM). In several embodiments, the gD peptide is coupled to a CD8αsignal peptide (e.g., for expression purposes, the sequence of which is provided for separately herein). In several embodiments, the gD immunosuppressive construct comprises a CD8α signal peptide and the gD peptide and is encoded by a polynucleotide that encodes an amino acid sequence comprising SEQ ID NO: 773. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 773. In several embodiments, the polynucleotide comprises SEQ ID NO: 774 or shares at least about 80%, at least about 85%, at least about 90%, at least about 94%, at least about 95%, at least about 96%, at least about 97%, at least about 98%, or at least about 99%, sequence identity with SEQ ID NO: 774. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the gD and the tag. In several embodiments, the polynucleotide encoding gD and GFP comprises SEQ ID NO: 772 or shares at least about 80%, at least about 85%, at least about 90%, at least about 94%, at least about 95%, at least about 96%, at least about 97%, at least about 98%, or at least about 99%, sequence identity with SEQ ID NO: 772. In several embodiments, the gD-GFP construct comprises the amino acid sequence of SEQ ID NO: 771 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 771.

In several embodiments, there is provided a polynucleotide encoding a lectin-like transcript 1 (LLT1), which, when interacting with CD161, inhibits Natural Killer cell activation and contributes to tumor cell immunosuppressive properties. In several embodiments, the LLT1 peptide is coupled to a CD8α signal peptide (e.g., for expression purposes, the sequence of which is provided for separately herein). In several embodiments, the LLT1 immunosuppressive construct comprises a CD8α signal peptide and the LLT1 peptide and is encoded by a polynucleotide that encodes an amino acid sequence comprising SEQ ID NO: 777. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 777. In several embodiments, the polynucleotide comprises SEQ ID NO: 778 or shares at least about 80%, at least about 85%, at least about 90%, at least about 94%, at least about 95%, at least about 96%, at least about 97%, at least about 98%, or at least about 99%, sequence identity with SEQ ID NO: 778. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LLT1 and the tag. In several embodiments, the polynucleotide encoding LLT1 and GFP comprises SEQ ID NO: 776 or shares at least about 80%, at least about 85%, at least about 90%, at least about 94%, at least about 95%, at least about 96%, at least about 97%, at least about 98%, or at least about 99%, sequence identity with SEQ ID NO: 776. In several embodiments, the LLT1-GFP construct comprises the amino acid sequence of SEQ ID NO: 775 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 775.

In several embodiments, there is provided a polynucleotide encoding at least a portion of the extracellular and transmembrane domains of CD47 (CD47tm162). In several embodiments, the CD47 domains are coupled to a CD8α signal peptide (e.g., for expression purposes, the sequence of which is provided for separately herein). In several embodiments, the CD47 immunosuppressive construct comprises a CD8α signal peptide and the CD47 extracellular and transmembrane domains (positions 19 to 162 of UniProtKB—Q08722) and is encoded by a polynucleotide that encodes an amino acid sequence comprising SEQ ID NO: 781. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 781. In several embodiments, the polynucleotide comprises SEQ ID NO: 782 or shares at least about 80%, at least about 85%, at least about 90%, at least about 94%, at least about 95%, at least about 96%, at least about 97%, at least about 98%, or at least about 99%, sequence identity with SEQ ID NO: 782. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD47 domains and the tag. In several embodiments, the polynucleotide encoding CD47 and GFP comprises SEQ ID NO: 780 or shares at least about 80%, at least about 85%, at least about 90%, at least about 94%, at least about 95%, at least about 96%, at least about 97%, at least about 98%, or at least about 99%, sequence identity with SEQ ID NO: 780. In several embodiments, the CD47tm162-GFP construct comprises the amino acid sequence of SEQ ID NO: 779 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 779.

In several embodiments, there is provided a polynucleotide encoding at least a portion of the latent membrane protein (LMP) oncogene of the Epstein-Barr virus (LMP_L_CD8HTM). In several embodiments, the LMP domain is coupled to a CD8α signal peptide (e.g., for expression purposes, the sequence of which is provided for separately herein). In several embodiments, the LMP immunosuppressive construct comprises a CD8α signal peptide, the LMP_L domain, a CD8α hinge, and a CD8α transmembrane domain and is encoded by a polynucleotide that encodes an amino acid sequence comprising SEQ ID NO: 785. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 785. In several embodiments, the polynucleotide comprises SEQ ID NO: 786 or shares at least about 80%, at least about 85%, at least about 90%, at least about 94%, at least about 95%, at least about 96%, at least about 97%, at least about 98%, or at least about 99%, sequence identity with SEQ ID NO: 786. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD47 domains and the tag. In several embodiments, the polynucleotide encoding the LMP_L domain and GFP comprises SEQ ID NO: 784 or shares at least about 80%, at least about 85%, at least about 90%, at least about 94%, at least about 95%, at least about 96%, at least about 97%, at least about 98%, or at least about 99%, sequence identity with SEQ ID NO: 784. In several embodiments, the LMP_L_CD8HTM-GFP construct comprises the amino acid sequence of SEQ ID NO: 783 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 783.

In several embodiments, there is provided a polynucleotide encoding a synthetic immunosuppressive construct comprising a multimeric repeat (e.g., dimer, trimer, quatramer, pentamer, etc.) of a peptide with immunosuppressive properties (LALLFWLx5-CD8HTM). In several embodiments, the synthetic domain is coupled to a CD8α signal peptide (e.g., for expression purposes, the sequence of which is provided for separately herein). In several embodiments, the synthetic immunosuppressive construct comprises a CD8α signal peptide, the five repeats of the synthetic peptide, a CD8α hinge, and a CD8α transmembrane domain and is encoded by a polynucleotide that encodes an amino acid sequence comprising SEQ ID NO: 789. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 789. In several embodiments, the polynucleotide comprises SEQ ID NO: 790 or shares at least about 80%, at least about 85%, at least about 90%, at least about 94%, at least about 95%, at least about 96%, at least about 97%, at least about 98%, or at least about 99%, sequence identity with SEQ ID NO: 790. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the transmembrane domain and the tag. In several embodiments, the polynucleotide encoding the synthetic immunosuppressive peptide and GFP comprises SEQ ID NO: 788 or shares at least about 80%, at least about 85%, at least about 90%, at least about 94%, at least about 95%, at least about 96%, at least about 97%, at least about 98%, or at least about 99%, sequence identity with SEQ ID NO: 788. In several embodiments, the synthetic immunosuppressive peptide-GFP construct comprises the amino acid sequence of SEQ ID NO: 787 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 787.

In several embodiments, there is provided a polynucleotide encoding at least a portion of the N-terminal fusion peptide (FP) of the human immunodeficiency virus (HIV)-1 envelope glycoprotein (Env) gp41 (GP41fp). In several embodiments, the GP41fp is coupled to a CD8α signal peptide (e.g., for expression purposes, the sequence of which is provided for separately herein). In several embodiments, the GP41fp immunosuppressive construct comprises a CD8α signal peptide, the GP41fp peptide, and at least a portion of the GP41 transmembrane domain and is encoded by a polynucleotide that encodes an amino acid sequence comprising SEQ ID NO: 793. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 793. In several embodiments, the polynucleotide comprises SEQ ID NO: 794 or shares at least about 80%, at least about 85%, at least about 90%, at least about 94%, at least about 95%, at least about 96%, at least about 97%, at least about 98%, or at least about 99%, sequence identity with SEQ ID NO: 794. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the transmembrane domain and the tag. In several embodiments, the polynucleotide encoding the GP41fp and GFP comprises SEQ ID NO: 792 or shares at least about 80%, at least about 85%, at least about 90%, at least about 94%, at least about 95%, at least about 96%, at least about 97%, at least about 98%, or at least about 99%, sequence identity with SEQ ID NO: 792. In several embodiments, the GP41fp-GFP construct comprises the amino acid sequence of SEQ ID NO: 791 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 791.

In several embodiments, there is provided a polynucleotide encoding a CKS-17 viral immunosuppressive peptide. In several embodiments, provided for is a membrane-bound CKS-17 viral immunosuppressive peptide. In several embodiments, the CKS-17 viral immunosuppressive peptide is coupled to a CD8α transmembrane protein and/or CD8α hinge domain. In several embodiments, the membrane-bound CKS-17 viral immunosuppressive peptide construct comprises one or more linker sequences. In several embodiments, the membrane-bound CKS-17 viral immunosuppressive peptide comprises a CD8α signal peptide, synthetic CKS-17, a CD8α hinge, and a CD8α transmembrane domain. In several embodiments, the membrane-bound CKS-17 viral immunosuppressive peptide construct is encoded by a polynucleotide that encodes an amino acid sequence comprising SEQ ID NO: 797. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 797. In several embodiments, the polynucleotide comprises SEQ ID NO: 798 or shares at least about 80%, at least about 85%, at least about 90%, at least about 94%, at least about 95%, at least about 96%, at least about 97%, at least about 98%, or at least about 99%, sequence identity with SEQ ID NO: 798. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound synthetic CKS-17 and GFP comprises SEQ ID NO: 796 or shares at least about 80%, at least about 85%, at least about 90%, at least about 94%, at least about 95%, at least about 96%, at least about 97%, at least about 98%, or at least about 99%, sequence identity with SEQ ID NO: 796. In several embodiments, the membrane-bound synthetic CKS-17-GFP construct comprises SEQ ID NO: 795 or shares at least about 80%, at least about 85%, at least about 90%, at least about 94%, at least about 95%, at least about 96%, at least about 97%, at least about 98%, or at least about 99%, sequence identity with SEQ ID NO: 795.

In several embodiments, there is provided a polynucleotide encoding a chimeric immunosuppressive construct comprising a CKS-17 domain and an HTLV-1 (Gp21) domain. In several embodiments, provided for is a membrane-bound immunosuppressive construct comprising a CKS-17 domain and a an HTLV-1 (Gp21) domain (CKS_HTLV-8aTM). In several embodiments, the CKS_HTLV immunosuppressive peptide is coupled to a CD8α transmembrane protein and/or CD8α hinge domain. In several embodiments, the membrane-bound CKS_HTLV viral immunosuppressive peptide construct comprises one or more linker sequences. In several embodiments, the membrane-bound CKS_HTLV viral immunosuppressive peptide comprises a CD8α signal peptide, a CKS-17 peptide, an HTLV(Gp21) peptide, a CD8α hinge, and a CD8α transmembrane domain. In several embodiments, the membrane-bound CKS_HTLV immunosuppressive peptide construct is encoded by a polynucleotide that encodes an amino acid sequence comprising SEQ ID NO: 801. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 801. In several embodiments, the polynucleotide comprises SEQ ID NO: 802 or shares at least about 80%, at least about 85%, at least about 90%, at least about 94%, at least about 95%, at least about 96%, at least about 97%, at least about 98%, or at least about 99%, sequence identity with SEQ ID NO: 802. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound CKS_HTLV and GFP comprises SEQ ID NO: 800 or shares at least about 80%, at least about 85%, at least about 90%, at least about 94%, at least about 95%, at least about 96%, at least about 97%, at least about 98%, or at least about 99%, sequence identity with SEQ ID NO: 800. In several embodiments, the membrane-bound CKS_HTLV-GFP construct comprises an amino acid sequence of SEQ ID NO: 799 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 799.

In several embodiments, there is provided a polynucleotide encoding a chimeric immunosuppressive construct comprising a CKS-17 domain and an LMP domain. In several embodiments, provided for is a membrane-bound immunosuppressive construct comprising a CKS-17 domain and a an LMP domain (CKS_LMP-8aTM). In several embodiments, the CKS_LMP immunosuppressive peptide is coupled to a CD8α transmembrane protein and/or CD8α hinge domain. In several embodiments, the membrane-bound CKS_LMP viral immunosuppressive peptide construct comprises one or more linker sequences. In several embodiments, the membrane-bound CKS_LMP viral immunosuppressive construct comprises a CD8α signal peptide, a CKS-17 peptide, an LMP peptide, a CD8α hinge, and a CD8αtransmembrane domain. In several embodiments, the membrane-bound CKS_LMP immunosuppressive construct is encoded by a polynucleotide that encodes an amino acid sequence comprising SEQ ID NO: 805. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 805. In several embodiments, the polynucleotide comprises SEQ ID NO: 806 or shares at least about 80%, at least about 85%, at least about 90%, at least about 94%, at least about 95%, at least about 96%, at least about 97%, at least about 98%, or at least about 99%, sequence identity with SEQ ID NO: 806. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound CKS_LMP and GFP comprises SEQ ID NO: 804 or shares at least about 80%, at least about 85%, at least about 90%, at least about 94%, at least about 95%, at least about 96%, at least about 97%, at least about 98%, or at least about 99%, sequence identity with SEQ ID NO: 804. In several embodiments, the membrane-bound CKS_LMP-GFP construct comprises an amino acid sequence of SEQ ID NO: 803 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 803.

In several embodiments, there is provided a polynucleotide encoding a chimeric immunosuppressive construct comprising a CKS-17 domain and a GP41fp domain. In several embodiments, provided for is a membrane-bound immunosuppressive construct comprising a CKS-17 domain and an GP41fp domain (CKS_GP41fptm). In several embodiments, the membrane-bound CKS_GP41fptm viral immunosuppressive peptide construct comprises one or more linker sequences. In several embodiments, the membrane-bound CKS_GP41fptm viral immunosuppressive construct comprises a CD8α signal peptide, a first portion of the GP41fp (amino acids 1-16), a CKS-17 peptide, a second portion of the GP41fp (amino acids 17-70), and a gp41 transmembrane domain. In several embodiments, the membrane-bound CKS_GP41fptm immunosuppressive construct is encoded by a polynucleotide that encodes an amino acid sequence comprising SEQ ID NO: 809. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 809. In several embodiments, the polynucleotide comprises SEQ ID NO: 810 or shares at least about 80%, at least about 85%, at least about 90%, at least about 94%, at least about 95%, at least about 96%, at least about 97%, at least about 98%, or at least about 99%, sequence identity with SEQ ID NO: 810. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound CKS_GP41fptm and GFP comprises SEQ ID NO: 808 or shares at least about 80%, at least about 85%, at least about 90%, at least about 94%, at least about 95%, at least about 96%, at least about 97%, at least about 98%, or at least about 99%, sequence identity with SEQ ID NO: 808. In several embodiments, the membrane-bound CKS_GP41fptm-GFP construct comprises an amino acid sequence of SEQ ID NO: 807 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 807.

In several embodiments, there is provided a polynucleotide encoding a truncated portion of the latent membrane protein 1 (LMP1) peptide of the Epstein-Barr Virus (LMP_S). In several embodiments, the LMP_S peptide is coupled to a CD8α signal peptide (e.g., for expression purposes, the sequence of which is provided for separately herein). In several embodiments, provided for is a membrane-bound LMP_S immunosuppressive construct comprising an LMP_S peptide coupled to a CD8αtransmembrane protein and/or CD8α hinge domain. In several embodiments, the membrane-bound LMP_S immunosuppressive construct comprises one or more linker sequences. In several embodiments, the membrane-bound LMP_S immunosuppressive construct comprises a CD8α signal peptide, an LMP_S peptide, an HTLV(Gp21) peptide, a CD8α hinge, and a CD8α transmembrane domain and is encoded by a polynucleotide that encodes an amino acid sequence comprising SEQ ID NO: 813. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 813. In several embodiments, the polynucleotide comprises SEQ ID NO: 814 or shares at least about 80%, at least about 85%, at least about 90%, at least about 94%, at least about 95%, at least about 96%, at least about 97%, at least about 98%, or at least about 99%, sequence identity with SEQ ID NO: 814. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LMP_S and the tag. In several embodiments, the polynucleotide encoding LMP_S and GFP comprises SEQ ID NO: 812 or shares at least about 80%, at least about 85%, at least about 90%, at least about 94%, at least about 95%, at least about 96%, at least about 97%, at least about 98%, or at least about 99%, sequence identity with SEQ ID NO: 812. In several embodiments, the LMP_S-GFP construct comprises the amino acid sequence of SEQ ID NO: 811 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 811.

In several embodiments, there is provided a polynucleotide encoding a chimeric immunosuppressive construct comprising at least a portion of the latent membrane protein 1 (LMP1) peptide of the Epstein-Barr Virus (LMP_L) coupled with at least a portion of the extracellular and transmembrane domains of CD47 (CD47tm162), together referred to as LMP_CD47tm162. In several embodiments, the LMP_CD47tm162 is coupled to a CD8α signal peptide (e.g., for expression purposes, the sequence of which is provided for separately herein). In several embodiments, the LMP_CD47tm162 construct is membrane-bound based on the portion of the CD47 transmembrane domain and comprises one or more linker sequences. In several embodiments, the membrane-bound LMP_CD47tm162 immunosuppressive construct comprises a CD8α signal peptide, an LMP_L peptide, and a CD47tm162 peptide, and is encoded by a polynucleotide that encodes an amino acid sequence comprising SEQ ID NO: 817. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 817. In several embodiments, the polynucleotide comprises SEQ ID NO: 818 or shares at least about 80%, at least about 85%, at least about 90%, at least about 94%, at least about 95%, at least about 96%, at least about 97%, at least about 98%, or at least about 99%, sequence identity with SEQ ID NO: 818. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LMP_CD47tm162 and the tag. In several embodiments, the polynucleotide encoding LMP_CD47tm162 and GFP comprises SEQ ID NO: 816 or shares at least about 80%, at least about 85%, at least about 90%, at least about 94%, at least about 95%, at least about 96%, at least about 97%, at least about 98%, or at least about 99%, sequence identity with SEQ ID NO: 816. In several embodiments, the LMP_CD47tm162-GFP construct comprises the amino acid sequence of SEQ ID NO: 815 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 815.

In several embodiments, there is provided a polynucleotide encoding a chimeric immunosuppressive construct comprising at least a portion of a p15E viral peptide coupled with at least a portion of the extracellular and transmembrane domains of CD47 (CD47tm162), together referred to as p15E_CD47tm162. In several embodiments, the p15E_CD47tm162 is coupled to a CD8α signal peptide (e.g., for expression purposes, the sequence of which is provided for separately herein). In several embodiments, the p15E_CD47tm162 construct is membrane-bound based on the portion of the CD47 transmembrane domain and comprises one or more linker sequences. In several embodiments, the membrane-bound p15E_CD47tm162 immunosuppressive construct comprises a CD8α signal peptide, a p15E peptide, and a CD47tm162 peptide, and is encoded by a polynucleotide that encodes an amino acid sequence comprising SEQ ID NO: 821. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 821. In several embodiments, the polynucleotide comprises SEQ ID NO: 822 or shares at least about 80%, at least about 85%, at least about 90%, at least about 94%, at least about 95%, at least about 96%, at least about 97%, at least about 98%, or at least about 99%, sequence identity with SEQ ID NO: 822. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the p15E_CD47tm162 and the tag. In several embodiments, the polynucleotide encoding p15E_CD47tm162 and GFP comprises SEQ ID NO: 820 or shares at least about 80%, at least about 85%, at least about 90%, at least about 94%, at least about 95%, at least about 96%, at least about 97%, at least about 98%, or at least about 99%, sequence identity with SEQ ID NO: 820. In several embodiments, the p15E_CD47tm162-GFP construct comprises the amino acid sequence of SEQ ID NO: 819 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 819.

In several embodiments, there is provided a polynucleotide encoding a chimeric immunosuppressive construct comprising at least one domain derived from HIV Gp41 and at least one domain from CD47 (CD47_GP41fptm). In several embodiments, the CD47_GP41fptm is coupled to a CD8α signal peptide (e.g., for expression purposes, the sequence of which is provided for separately herein). In several embodiments, the CD47_GP41fptm construct is membrane-bound based on a gp41 transmembrane domain. In several embodiments, the membrane-bound CD47_GP41fptm immunosuppressive construct comprises a CD8α signal peptide, a first GP41 domain (amino acids 1-16 of the extracellular domain), a CD47 domain (amino acids 2-141), a second GP41 domain (amino acids 17-70 of the extracellular domain), and a GP41 transmembrane domain and is encoded by a polynucleotide that encodes an amino acid sequence comprising SEQ ID NO: 825. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 825. In several embodiments, the polynucleotide comprises SEQ ID NO: 826 or shares at least about 80%, at least about 85%, at least about 90%, at least about 94%, at least about 95%, at least about 96%, at least about 97%, at least about 98%, or at least about 99%, sequence identity with SEQ ID NO: 826. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD47_GP41fptm and the tag. In several embodiments, the polynucleotide encoding CD47_GP41fptm and GFP comprises SEQ ID NO: 824 or shares at least about 80%, at least about 85%, at least about 90%, at least about 94%, at least about 95%, at least about 96%, at least about 97%, at least about 98%, or at least about 99%, sequence identity with SEQ ID NO: 824. In several embodiments, the CD47_GP41fptm-GFP construct comprises the amino acid sequence of SEQ ID NO: 823 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 823.

In several embodiments, there is provided a polynucleotide encoding an immunosuppressive construct comprising at least a portion of an antibody that targets a signal-regulatory protein α (SIRPα) (anti-SIRPa agonist_vHL), which is an innate immune checkpoint expressed on dendritic cells, macrophages, monocytes and neutrophils. In several embodiments, the constructs are engineered to be membrane-bound. In several embodiments, the anti-SIRPa agonist_vHL is coupled to a CD8α signal peptide (e.g., for expression purposes, the sequence of which is provided for separately herein). In several embodiments, the anti-SIRPa agonist_vHL construct is membrane-bound based on a CD8α hinge region and/or a CD8α transmembrane domain. In several embodiments, the membrane-bound anti-SIRPa agonist_vHL immunosuppressive construct comprises a CD8α signal peptide, an anti-SIRPa heavy chain, a linker and anti-SIRPa light chain, a CD8α hinge region and, a CD8α transmembrane domain and is encoded by a polynucleotide that encodes an amino acid sequence comprising SEQ ID NO: 833. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 833. In several embodiments, the polynucleotide comprises SEQ ID NO: 834 or shares at least about 80%, at least about 85%, at least about 90%, at least about 94%, at least about 95%, at least about 96%, at least about 97%, at least about 98%, or at least about 99%, sequence identity with SEQ ID NO: 834. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the anti-SIRPa agonist_vHL and the tag. In several embodiments, the polynucleotide encoding anti-SIRPa agonist_vHL and GFP comprises SEQ ID NO: 832 or shares at least about 80%, at least about 85%, at least about 90%, at least about 94%, at least about 95%, at least about 96%, at least about 97%, at least about 98%, or at least about 99%, sequence identity with SEQ ID NO: 832. In several embodiments, the anti-SIRPa agonist_vHL-GFP construct comprises the amino acid sequence of SEQ ID NO: 831 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 831.

In several embodiments, there is provided a polynucleotide encoding an immunosuppressive construct comprising at least one domain derived from HTLV GP21 (HTLV1_GP21). In several embodiments, the HTLV1_GP21 is coupled to a CD8α signal peptide (e.g., for expression purposes, the sequence of which is provided for separately herein). In several embodiments, the HTLV1_GP21 comprises a CD8α signal peptide and a GP21 transmembrane domain and is encoded by a polynucleotide that encodes an amino acid sequence comprising SEQ ID NO: 841. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 841. In several embodiments, the polynucleotide comprises SEQ ID NO: 842 or shares at least about 80%, at least about 85%, at least about 90%, at least about 94%, at least about 95%, at least about 96%, at least about 97%, at least about 98%, or at least about 99%, sequence identity with SEQ ID NO: 842. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HTLV1_GP21 and the tag. In several embodiments, the polynucleotide encoding HTLV1_GP21 and GFP comprises SEQ ID NO: 840 or shares at least about 80%, at least about 85%, at least about 90%, at least about 94%, at least about 95%, at least about 96%, at least about 97%, at least about 98%, or at least about 99%, sequence identity with SEQ ID NO: 840. In several embodiments, the HTLV1_fGP62-GFP construct comprises the amino acid sequence of SEQ ID NO: 839 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 839.

In several embodiments, there is provided a polynucleotide encoding an immunosuppressive construct comprising at least one domain derived from a Lassa virus membrane glycoprotein 2 (LASV_fGP2). In several embodiments, the LASV_fGP2 is coupled to a CD8α signal peptide (e.g., for expression purposes, the sequence of which is provided for separately herein). In several embodiments, the LASV_fGP2 comprises a CD8α signal peptide and at least a portion of a Lassa Virus GP2 domain, including a transmembrane region and is encoded by a polynucleotide that encodes an amino acid sequence comprising SEQ ID NO: 845. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 845. In several embodiments, the polynucleotide comprises SEQ ID NO: 846 or shares at least about 80%, at least about 85%, at least about 90%, at least about 94%, at least about 95%, at least about 96%, at least about 97%, at least about 98%, or at least about 99%, sequence identity with SEQ ID NO: 846. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HTLV1_GP21 and the tag. In several embodiments, the polynucleotide encoding LASV_fGP2 and GFP comprises SEQ ID NO: 844 or shares at least about 80%, at least about 85%, at least about 90%, at least about 94%, at least about 95%, at least about 96%, at least about 97%, at least about 98%, or at least about 99%, sequence identity with SEQ ID NO: 844. In several embodiments, the LASV_fGP2-GFP construct comprises the amino acid sequence of SEQ ID NO: 843 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 843.

In several embodiments, there is provided a polynucleotide encoding an immunosuppressive construct comprising at least one domain derived from a Sudan ebolavirus envelope glycoprotein (SEBOV_fGP). In several embodiments, the SEBOV_fGP is coupled to a CD8α signal peptide (e.g., for expression purposes, the sequence of which is provided for separately herein). In several embodiments, the SEBOV_fGP comprises a CD8α signal peptide and at least two domains from the ebolavirus envelope. In several embodiments, the SEBOV_fGP comprises a full GP1 glycoprotein sequence followed by a truncated form of GP1 (known as GP2, resulting from proteolysis of the GP1 domain). In several embodiments, the GP2 domain includes a transmembrane domain. In several embodiments, the SEBOV_fGP is encoded by a polynucleotide that encodes an amino acid sequence comprising SEQ ID NO: 849. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 849. In several embodiments, the polynucleotide comprises SEQ ID NO: 850 or shares at least about 80%, at least about 85%, at least about 90%, at least about 94%, at least about 95%, at least about 96%, at least about 97%, at least about 98%, or at least about 99%, sequence identity with SEQ ID NO: 850. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the SEBOV_fGP and the tag. In several embodiments, the polynucleotide encoding SEBOV_fGP and GFP comprises SEQ ID NO: 848 or shares at least about 80%, at least about 85%, at least about 90%, at least about 94%, at least about 95%, at least about 96%, at least about 97%, at least about 98%, or at least about 99%, sequence identity with SEQ ID NO: 848. In several embodiments, the SEBOV_fGP-GFP construct comprises the amino acid sequence of SEQ ID NO: 847 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 847.

In several embodiments, there is provided a polynucleotide encoding an immunosuppressive construct comprising at least one domain derived from a Sudan ebolavirus envelope glycoprotein (SEBOV_GP2). In several embodiments, the SEBOV_GP2 is coupled to a CD8α signal peptide (e.g., for expression purposes, the sequence of which is provided for separately herein). In several embodiments, the SEBOV_GP2 comprises a CD8α signal peptide and a GP2 domain, resulting from proteolysis of the GP1 domain. In several embodiments, the GP2 domain includes a transmembrane domain. In several embodiments, the SEBOV_GP2 is encoded by a polynucleotide that encodes an amino acid sequence comprising SEQ ID NO: 853. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 853. In several embodiments, the polynucleotide comprises SEQ ID NO: 854 or shares at least about 80%, at least about 85%, at least about 90%, at least about 94%, at least about 95%, at least about 96%, at least about 97%, at least about 98%, or at least about 99%, sequence identity with SEQ ID NO: 854. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the SEBOV_GP2 and the tag. In several embodiments, the polynucleotide encoding SEBOV_GP2 and GFP comprises SEQ ID NO: 852 or shares at least about 80%, at least about 85%, at least about 90%, at least about 94%, at least about 95%, at least about 96%, at least about 97%, at least about 98%, or at least about 99%, sequence identity with SEQ ID NO: 852. In several embodiments, the SEBOV_GP2-GFP construct comprises the amino acid sequence of SEQ ID NO: 851 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 851.

In several embodiments, there is provided a polynucleotide encoding an immunosuppressive construct comprising at least one domain derived from a Sars-CoV-2 spike protein (SCoV_S2). In several embodiments, the SCoV_S2 is coupled to a CD8α signal peptide (e.g., for expression purposes, the sequence of which is provided for separately herein). In several embodiments, the SCoV_S2 comprises a CD8α signal peptide and a an S2 spike protein, including a transmembrane domain. In several embodiments, the SCoV_S2 is encoded by a polynucleotide that encodes an amino acid sequence comprising SEQ ID NO: 857. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 857. In several embodiments, the polynucleotide comprises SEQ ID NO: 858 or shares at least about 80%, at least about 85%, at least about 90%, at least about 94%, at least about 95%, at least about 96%, at least about 97%, at least about 98%, or at least about 99%, sequence identity with SEQ ID NO: 858. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the SCoV_S2 and the tag. In several embodiments, the polynucleotide encoding SCoV_S2and GFP comprises SEQ ID NO: 856 or shares at least about 80%, at least about 85%, at least about 90%, at least about 94%, at least about 95%, at least about 96%, at least about 97%, at least about 98%, or at least about 99%, sequence identity with SEQ ID NO: 856. In several embodiments, the SCoV_S2-GFP construct comprises the amino acid sequence of SEQ ID NO: 855 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 855.

In several embodiments, there is provided a polynucleotide encoding an immunosuppressive construct comprising at least a portion of a Galectin-3-binding protein (LGALS3BP). In several embodiments, the constructs are engineered to be membrane-bound. In several embodiments, the LGALS3BP is coupled to a CD8α signal peptide (e.g., for expression purposes, the sequence of which is provided for separately herein). In several embodiments, LGALS3BP construct is membrane-bound based on a CD8α hinge region and/or a CD8α transmembrane domain. In several embodiments, the membrane-bound LGALS3BP immunosuppressive construct comprises a CD8α signal peptide, a Galectin 3 binding protein, and a CD8α transmembrane domain and is encoded by a polynucleotide that encodes an amino acid sequence comprising SEQ ID NO: 861. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 861. In several embodiments, the polynucleotide comprises SEQ ID NO: 862 or shares at least about 80%, at least about 85%, at least about 90%, at least about 94%, at least about 95%, at least about 96%, at least about 97%, at least about 98%, or at least about 99%, sequence identity with SEQ ID NO: 862. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LGALS3BP and the tag. In several embodiments, the polynucleotide encoding LGALS3BP and GFP comprises SEQ ID NO: 860 or shares at least about 80%, at least about 85%, at least about 90%, at least about 94%, at least about 95%, at least about 96%, at least about 97%, at least about 98%, or at least about 99%, sequence identity with SEQ ID NO: 860. In several embodiments, the LGALS3BP-GFP construct comprises the amino acid sequence of SEQ ID NO: 859 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 859.

In several embodiments, there is provided a polynucleotide encoding an immunosuppressive construct comprising at least a portion of CD24. In several embodiments, the constructs are engineered to be membrane-bound. In several embodiments, the CD24 construct comprises a CD24 signal peptide and a CD24 protein and is encoded by a polynucleotide that encodes an amino acid sequence comprising SEQ ID NO: 865. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 865. In several embodiments, the polynucleotide comprises SEQ ID NO: 866 or shares at least about 80%, at least about 85%, at least about 90%, at least about 94%, at least about 95%, at least about 96%, at least about 97%, at least about 98%, or at least about 99%, sequence identity with SEQ ID NO: 866. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a T2A domain is positioned between the CD24 and the tag. In several embodiments, the polynucleotide encoding CD24 and GFP comprises SEQ ID NO: 864 or shares at least about 80%, at least about 85%, at least about 90%, at least about 94%, at least about 95%, at least about 96%, at least about 97%, at least about 98%, or at least about 99%, sequence identity with SEQ ID NO: 864. In several embodiments, the CD24-GFP construct comprises the amino acid sequence of SEQ ID NO: 863 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 863.

In several embodiments, there is provided a polynucleotide encoding an immunosuppressive construct comprising at least a portion of a Hepatitis C envelope glycoprotein (HCV_E2). In several embodiments, the HCV_E2 is coupled to a CD8α signal peptide (e.g., for expression purposes, the sequence of which is provided for separately herein). In several embodiments, the HCV_E2 immunosuppressive construct comprises a CD8α signal peptide, and a E2 protein of HCV and is encoded by a polynucleotide that encodes an amino acid sequence comprising SEQ ID NO: 869. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 869. In several embodiments, the polynucleotide comprises SEQ ID NO: 870 or shares at least about 80%, at least about 85%, at least about 90%, at least about 94%, at least about 95%, at least about 96%, at least about 97%, at least about 98%, or at least about 99%, sequence identity with SEQ ID NO: 870. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HCV_E2 and the tag. In several embodiments, the polynucleotide encoding HCV_E2 and GFP comprises SEQ ID NO: 868 or shares at least about 80%, at least about 85%, at least about 90%, at least about 94%, at least about 95%, at least about 96%, at least about 97%, at least about 98%, or at least about 99%, sequence identity with SEQ ID NO: 868. In several embodiments, the HCV_E2-GFP construct comprises the amino acid sequence of SEQ ID NO: 867 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 867.

In several embodiments, there is provided a polynucleotide encoding an immunosuppressive construct comprising at least a portion of an antibody that targets a signal-regulatory protein α (SIRPa) (anti-SIRPa agonist_vLH). In several embodiments, the constructs are engineered to be membrane-bound. In several embodiments, the anti-SIRPa agonist_vLH is coupled to a CD8α signal peptide (e.g., for expression purposes, the sequence of which is provided for separately herein). In several embodiments, the anti-SIRPa agonist_vLH construct is membrane-bound based on a CD8α hinge region and/or a CD8α transmembrane domain. In several embodiments, the membrane-bound anti-SIRPa agonist_vLH immunosuppressive construct comprises a CD8α signal peptide, an anti-SIRPa light chain, a linker, anti-SIRPa heavy chain, a CD8α hinge region and, a CD8α transmembrane domain and is encoded by a polynucleotide that encodes an amino acid sequence comprising SEQ ID NO: 873. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 873. In several embodiments, the polynucleotide comprises SEQ ID NO: 874 or shares at least about 80%, at least about 85%, at least about 90%, at least about 94%, at least about 95%, at least about 96%, at least about 97%, at least about 98%, or at least about 99%, sequence identity with SEQ ID NO: 874. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the anti-SIRPa agonist_vLH and the tag. In several embodiments, the polynucleotide encoding anti-SIRPa agonist_vLH and GFP comprises SEQ ID NO: 872 or shares at least about 80%, at least about 85%, at least about 90%, at least about 94%, at least about 95%, at least about 96%, at least about 97%, at least about 98%, or at least about 99%, sequence identity with SEQ ID NO: 872. In several embodiments, the anti-SIRPa agonist_vLH-GFP construct comprises the amino acid sequence of SEQ ID NO: 871 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 871.

In several embodiments, there is provided a polynucleotide encoding an immunosuppressive construct comprising at least CEA Cell Adhesion Molecule 1-derived domain (CEACAMI). In several embodiments, the constructs are engineered to be membrane-bound. In several embodiments, the CEACAMI is coupled to a CD8α signal peptide (e.g., for expression purposes, the sequence of which is provided for separately herein). In several embodiments, the CEACAMI immunosuppressive construct comprises a CD8α signal peptide and a CEACAMI-derived protein. In several embodiments, the CEACAMI immunosuppressive construct is encoded by a polynucleotide that encodes an amino acid sequence comprising SEQ ID NO: 877. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 877. In several embodiments, the polynucleotide comprises SEQ ID NO: 878 or shares at least about 80%, at least about 85%, at least about 90%, at least about 94%, at least about 95%, at least about 96%, at least about 97%, at least about 98%, or at least about 99%, sequence identity with SEQ ID NO: 878. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CEACAMI and the tag. In several embodiments, the polynucleotide encoding the CEACAMI and GFP comprises SEQ ID NO: 876 or shares at least about 80%, at least about 85%, at least about 90%, at least about 94%, at least about 95%, at least about 96%, at least about 97%, at least about 98%, or at least about 99%, sequence identity with SEQ ID NO: 876. In several embodiments, the CEACAMI-GFP construct comprises the amino acid sequence of SEQ ID NO: 875 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 875.

In several embodiments, there is provided a polynucleotide encoding an immunosuppressive construct comprising CD155 transmembrane domain (CD155tm_3M). In several embodiments, the CD155tm_3M is coupled to a CD8α signal peptide (e.g., for expression purposes, the sequence of which is provided for separately herein). In several embodiments, the CD155tm_3Mimmunosuppressive construct comprises a CD8α signal peptide and a CD155-derived protein. In several embodiments, the CD155tm_3M immunosuppressive construct is encoded by a polynucleotide that encodes an amino acid sequence comprising SEQ ID NO: 881. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 881. In several embodiments, the polynucleotide comprises SEQ ID NO: 882 or shares at least about 80%, at least about 85%, at least about 90%, at least about 94%, at least about 95%, at least about 96%, at least about 97%, at least about 98%, or at least about 99%, sequence identity with SEQ ID NO: 882. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD155tm_3M and the tag. In several embodiments, the polynucleotide encoding the CD155tm_3M and GFP comprises SEQ ID NO: 880 or shares at least about 80%, at least about 85%, at least about 90%, at least about 94%, at least about 95%, at least about 96%, at least about 97%, at least about 98%, or at least about 99%, sequence identity with SEQ ID NO: 880. In several embodiments, the CD155tm_3M-GFP construct comprises the amino acid sequence of SEQ ID NO: 879 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 879.

In several embodiments, there is provided a polynucleotide encoding an immunosuppressive construct comprising CD31 transmembrane domain (CD31tm). In several embodiments, the CD31tm is coupled to a CD8α signal peptide (e.g., for expression purposes, the sequence of which is provided for separately herein). In several embodiments, the CD31tm immunosuppressive construct comprises a CD8α signal peptide and a CD31-derived protein. In several embodiments, the CD31tm immunosuppressive construct is encoded by a polynucleotide that encodes an amino acid sequence comprising SEQ ID NO: 885. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 885. In several embodiments, the polynucleotide comprises SEQ ID NO: 886 or shares at least about 80%, at least about 85%, at least about 90%, at least about 94%, at least about 95%, at least about 96%, at least about 97%, at least about 98%, or at least about 99%, sequence identity with SEQ ID NO: 886. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD31tm and the tag. In several embodiments, the polynucleotide encoding the CD31tm and GFP comprises SEQ ID NO: 884 or shares at least about 80%, at least about 85%, at least about 90%, at least about 94%, at least about 95%, at least about 96%, at least about 97%, at least about 98%, or at least about 99%, sequence identity with SEQ ID NO: 884. In several embodiments, the CD31tm-GFP construct comprises the amino acid sequence of SEQ ID NO: 883 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 883.

In several embodiments, there is provided a polynucleotide encoding an immunosuppressive construct comprising CD111 transmembrane domain (CD111tm). In several embodiments, the CD111tm is coupled to a CD8α signal peptide (e.g., for expression purposes, the sequence of which is provided for separately herein). In several embodiments, the CD111tm immunosuppressive construct comprises a CD8α signal peptide and a CD111tm-derived protein. In several embodiments, the CD111 tm immunosuppressive construct is encoded by a polynucleotide that encodes an amino acid sequence comprising SEQ ID NO: 889. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 889. In several embodiments, the polynucleotide comprises SEQ ID NO: 890 or shares at least about 80%, at least about 85%, at least about 90%, at least about 94%, at least about 95%, at least about 96%, at least about 97%, at least about 98%, or at least about 99%, sequence identity with SEQ ID NO: 890. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD111tm and the tag. In several embodiments, the polynucleotide encoding the CD111tm and GFP comprises SEQ ID NO: 888 or shares at least about 80%, at least about 85%, at least about 90%, at least about 94%, at least about 95%, at least about 96%, at least about 97%, at least about 98%, or at least about 99%, sequence identity with SEQ ID NO: 888. In several embodiments, the CD111tm-GFP construct comprises the amino acid sequence of SEQ ID NO: 887 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 887.

In several embodiments, there is provided a polynucleotide encoding an immunosuppressive construct comprising CD200 transmembrane domain (CD200tm). In several embodiments, the CD200tm is coupled to a CD8α signal peptide (e.g., for expression purposes, the sequence of which is provided for separately herein). In several embodiments, the CD200tm immunosuppressive construct comprises a CD8α signal peptide and a CD200tm-derived protein. In several embodiments, the CD200tm immunosuppressive construct is encoded by a polynucleotide that encodes an amino acid sequence comprising SEQ ID NO: 893. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 893. In several embodiments, the polynucleotide comprises SEQ ID NO: 894 or shares at least about 80%, at least about 85%, at least about 90%, at least about 94%, at least about 95%, at least about 96%, at least about 97%, at least about 98%, or at least about 99%, sequence identity with SEQ ID NO: 894. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD200tm and the tag. In several embodiments, the polynucleotide encoding the CD200tm and GFP comprises SEQ ID NO: 892 or shares at least about 80%, at least about 85%, at least about 90%, at least about 94%, at least about 95%, at least about 96%, at least about 97%, at least about 98%, or at least about 99%, sequence identity with SEQ ID NO: 892. In several embodiments, the CD200tm-GFP construct comprises the amino acid sequence of SEQ ID NO: 891 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 891.

In several embodiments, the engineered cells provided for herein comprise a chimeric receptor that targets NKG2D, wherein the CAR comprises an amino acid of SEQ ID NO: 174, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 174 and one or more copies of one or more of the following membrane-bound immunosuppressive amino acid sequences: SEQ ID NO: 218, 223, 228, 233, 238, 243, 248, 253, 256, 259, 262, 265, 268, and 271.

In several embodiments, the engineered cells provided for herein comprise a CAR that targets CD19, wherein the CAR comprises an amino acid of SEQ ID NO: 178, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178 and one or more copies of one or more of the following membrane-bound immunosuppressive amino acid sequences: SEQ ID NO: 218, 223, 228, 233, 238, 243, 248, 253, 256, 259, 262, 265, 268, and 271.

In several embodiments, the engineered cells provided for herein comprise a CAR that targets CD19, wherein the CAR comprises is encoded by SEQ ID NO: 466, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to the sequence encoded by SEQ ID NO: 466 and one or more copies of one or more of the following membrane-bound immunosuppressive amino acid sequences: SEQ ID NO: 218, 223, 228, 233, 238, 243, 248, 253, 256, 259, 262, 265, 268, and 271.

In several embodiments, the engineered cells provided for herein comprise a CAR that targets CD70, wherein the CAR comprises an amino acid of any of SEQ ID NOs: 383-465, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NO: 383-465 and one or more copies of one or more of the following membrane-bound immunosuppressive amino acid sequences: SEQ ID NO: 218, 223, 228, 233, 238, 243, 248, 253, 256, 259, 262, 265, 268, and 271.

### HLA-Modification

In several embodiments, immune cells are engineered to alter their HLA expression profile, order to interact with one or more inhibitory receptors on host immune cells. In several embodiments, the alteration of the HLA expression profile of engineered cells functions to impart to the engineered immune cell the ability to reduce or avoid cytotoxicity or other immune clearance by host immune cells (or other engineered immune cells), thereby enhancing the persistence (and thus functional life-span) of the engineered immune cells.

Human leukocyte antigen-E (HLA-E) is a major ligand for the natural killer inhibitory receptor CD94/NKG2A that is expressed on NK cells. When HLA-E is bound by the CD94/NKG2A complex, an inhibitory signaling cascade is initiated, resulting in reduced NK cell activity. Thus, expression of HLA-E can dampen the cytotoxic effects of host NK cells against an engineered immune cell (e.g., expressing a CAR and/or immunosuppressive peptides). Likewise, HLA-G also plays an important role in inhibiting natural killer (NK) cell function, not only in the maintenance of fetal-maternal immune tolerance but also in the context of organ or tissue transplantation. It also plays a role in the immune escape of tumors from host immune cells. HLA-G can inhibit the function of many immune cells such as NK cells, CD4+ and CD8+ T cells, and dendritic cells by binding to cell surface-expressed receptors, including immunoglobulin-like transcript 2 (ILT2), ILT4 and killer cell immunoglobulin-like receptor 2DL4 (KIR2DL4). In several embodiments, immune cells as disclosed herein are engineered to express HLA-E and/or HLA-G in order to suppress host NK cell (or other engineered NK cells administered) against the engineered immune cells.

Non-limiting embodiments of such an approach are schematically depicted in FIGS. 261-26J. FIG. 261 shows an engineered immune cell according to embodiments disclosed herein that expresses a CAR comprising an immunosuppressive domain as well as a membrane-bound immunosuppressive construct. In some embodiments, expression of HLA-E and/or HLA-G is in connection with a CAR and not a membrane-bound immunosuppressive construct, or optionally with a CAR that does not include a immunosuppressive domain. FIG. 26J schematically depicts another non-limiting embodiment wherein HLA-E and/or HLA-G are co-expressed with one or more membrane-bound immunosuppressive constructs, optionally a CAR (with or without an integrated immunosuppressive domain) and optionally one or more additional immunosuppressive proteins (non-limiting examples of with include, but are not limited to CD47, PD-L1, and the poliovirus receptor (PVR, also known as CD155). As discussed above, each of CD47, PD-L1 and CD155 can operate to reduce activity of immune cells (e.g., host immune cells and/or other administered engineered immune cells). Any combination of these molecules, or any others disclosed herein can be used.

In several embodiments, such as when B2M expression is reduced or knocked out, endogenous HLA expression is lost, and in several embodiments, specific re-expression of HLA-E and or HLA-G can aid in reduce NK cell activity against immune cells engineered to express HLA-E and or HLA-G. In several embodiments, this re-expression is coupled with gene editing to reduce NKG2A expression on NK cells to be administered, which limits the suppressive effect of HLA-E on the therapeutic cells themselves. In several embodiments, HLA-E expression is specifically introduced only on T cells. In several embodiments, those T cells operate to suppress NK cell activity via interaction with the NKG2A receptor on NK cells. In some embodiments, the activity of the engineered allogeneic NK cells is suppressed temporarily. In several embodiments, the temporary suppression of engineered allogeneic NK cell activity reduces the risk of NK cell exhaustion, which prolongs the persistence of the engineered allogeneic NK cells.

In several embodiments, there is provided a polynucleotide encoding HLA-E. In several embodiments, the polynucleotide encodes an HLA-E amino acid sequence comprising SEQ ID NO: 273. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 273. In several embodiments, the polynucleotide comprises SEQ ID NO: 274 or shares at least about 90%, at least about 94%, at least about 95%, at least about 96%, at least about 97%, at least about 98%, or at least about 99%, sequence identity with SEQ ID NO: 274. In several embodiments, the encoded HLA-E is an HLA-E single chain trimer (SCT) composed of a canonical HLA-E binding peptide, mature human beta2-microglobulin, and mature HLA-E heavy chain (HLA-E trimer_SS). In several embodiments, the construct comprises a CD8α signal peptide, a first B2M sequence, an HLG peptide leader sequence, a linker (e.g., a GS linker), a second B2M sequence, a linker (e.g., a second GS linker), and an HLA-E sequence. In several embodiments, the HLA-E trimer_SS construct is encoded by a polynucleotide that encodes an amino acid sequence comprising SEQ ID NO: 276 or SEQ ID NO: 689. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 276 or SEQ ID NO: 689. In several embodiments, the polynucleotide comprises SEQ ID NO: 277 (or SEQ ID NO: 690) or shares at least about 80%, at least about 85%, at least about 90%, at least about 94%, at least about 95%, at least about 96%, at least about 97%, at least about 98%, or at least about 99%, sequence identity with SEQ ID NO: 277 or SEQ ID NO: 690. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the HLA-E trimer_SS construct and GFP comprises SEQ ID NO: 275 or shares at least about 80%, at least about 85%, at least about 90%, at least about 94%, at least about 95%, at least about 96%, at least about 97%, at least about 98%, or at least about 99%, sequence identity with SEQ ID NO: 275. In several embodiments, the polynucleotide encoding the HLA-E trimer_SS construct, a FLAG tag, and GFP comprises SEQ ID NO: 688 or shares at least about 80%, at least about 85%, at least about 90%, at least about 94%, at least about 95%, at least about 96%, at least about 97%, at least about 98%, or at least about 99%, sequence identity with SEQ ID NO: 688. In several embodiments, the HLA-E trimer_SS construct with a FLAG tag and GFP is encoded by a polynucleotide that encodes an amino acid sequence comprising SEQ ID NO: 687, or having at least about 80%, at least about 85%, at least about 90%, at least about 94%, at least about 95%, at least about 96%, at least about 97%, at least about 98%, or at least about 99%, sequence identity with SEQ ID NO: 687.

In several embodiments, similar constructs are provided for HLA-G wherein the HLA-G trimer_SS construct comprises a CD8α signal peptide, a first B2M sequence, an HLG peptide leader sequence, a linker (e.g., a GS linker), a second B2M sequence, a linker (e.g., a second GS linker), and an HLA-G sequence. In several embodiments, the HLA-G trimer_SS construct is encoded by a polynucleotide that encodes an amino acid sequence comprising SEQ ID NO: 279. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 279. In several embodiments, the HLA-G polypeptide comprises a functional portion of SEQ ID NO: 278.

In the context of viral infection, MHC class I-restricted CD8+ cytotoxic T lymphocytes are recruited to control viral infections. These cytotoxic T lymphocytes recognize and lyse virus-infected cells through engagement of the lymphocyte T cell receptor with MHC class I molecules that present viral antigens on the surface of infected cells. MHC class I heavy chain associates with beta-2 microglobulin (B2M) to form a heterodimer, which constitutes part of the MHC class I peptide-loading complex. Human cytomegalovirus has evolved several gene products of the unique short region protein, US2, US3, US6, and US11, which interfere with antigen presentation and cell surface expression of MHC class I molecules. While interference in antigen presentation and MHC class I down-regulation on the cell surface allows infected cells to evade virus-specific cytotoxic T lymphocytes, the down-regulation of MHC class I molecules renders the virally infected cells more susceptible to host NK cells. To counteract this, human cytomegalovirus encodes multiple genes that function to evade NK-mediated cell lysis of infected cells, one of which is UL18. UL18 binds LIR-1, an NK cell inhibitory receptor. UL18 shares a high level of amino acid sequence identity with MHC class I and therefore UL18 can act as an MHCI surrogate and associate with B2M. Thus, in several embodiments, a chimeric UL18-B2M construct (see a non-limiting schematic at FIG. 27) is expressed on engineered cells as disclosed herein, enabling UL18 interaction with the LIR-1 receptor on NK cells (either host or administered) and reduce the cytotoxic activity of those NK cells against the engineered UL18-expressing immune cell.

In several embodiments, there is provided a polynucleotide encoding UL18. In several embodiments, the UL18-encoding polynucleotide encodes an amino acid sequence comprising SEQ ID NO: 280. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 280. In several embodiments, the polynucleotide comprises SEQ ID NO: 281 or shares at least about 80%, at least about 85%, at least about 90%, at least about 94%, at least about 95%, at least about 96%, at least about 97%, at least about 98%, or at least about 99%, sequence identity with SEQ ID NO: 281. In several embodiments, the encoded UL18 is an chimeric UL18-B2M single chain trimer (SCT) composed of a canonical HLA-E binding peptide, mature human beta2-microglobulin, and UL18. In several embodiments, the chimeric UL18-B2M construct comprises a CD8α signal peptide, a first B2M sequence, an HLG peptide leader sequence, a linker (e.g., a GS linker), a second B2M sequence, a linker (e.g., a second GS linker), an a UL18 sequence. In several embodiments, the chimeric UL18-B2M construct is encoded by a polynucleotide that encodes an amino acid sequence comprising SEQ ID NO: 283. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 283 or SEQ ID NO: 686. In several embodiments, the polynucleotide comprises SEQ ID NO: 284 (or SEQ ID NO: 685) or shares at least about 80%, at least about 85%, at least about 90%, at least about 94%, at least about 95%, at least about 96%, at least about 97%, at least about 98%, or at least about 99%, sequence identity with SEQ ID NO: 284 (or SEQ ID NO: 685). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the chimeric UL18-B2M construct and GFP comprises SEQ ID NO: 292 or shares at least about 80%, at least about 85%, at least about 90%, at least about 94%, at least about 95%, at least about 96%, at least about 97%, at least about 98%, or at least about 99%, sequence identity with SEQ ID NO: 292. In several embodiments, the polynucleotide encoding the chimeric UL18-B2M construct, a FLAG tag and GFP comprises SEQ ID NO: 684 or shares at least about 80%, at least about 85%, at least about 90%, at least about 94%, at least about 95%, at least about 96%, at least about 97%, at least about 98%, or at least about 99%, sequence identity with SEQ ID NO: 684. In additional embodiments, the polynucleotide encoding the chimeric UL18-B2M construct with a FLAG tag and GFP encodes the amino acid sequence of SEQ ID NO: 684 or a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 684. In additional embodiments, the polynucleotide encoding the chimeric UL18-B2M construct encodes the amino acid sequence of SEQ ID NO: 285 or a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 285. In several embodiments, the polynucleotide comprises SEQ ID NO: 281 or shares at least about 80%, at least about 85%, at least about 90%, at least about 94%, at least about 95%, at least about 96%, at least about 97%, at least about 98%, or at least about 99%, sequence identity with SEQ ID NO: 285.

In several embodiments, the engineered cells provided for herein comprise a chimeric receptor that targets NKG2D, wherein the CAR comprises an amino acid of SEQ ID NO: 174, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 174 and one or more copies of one or more of the following membrane-bound immunosuppressive constructs: SEQ ID NO: 273, 276, 279, 280, or 283.

In several embodiments, the engineered cells provided for herein comprise a CAR that targets CD19, wherein the CAR comprises an amino acid of SEQ ID NO: 178, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178 and one or more copies of one or more of the following membrane-bound immunosuppressive constructs: SEQ ID NO: 273, 276, 279, 280, or 283.

In several embodiments, the engineered cells provided for herein comprise a CAR that targets CD19, wherein the CAR is encoded by SEQ ID NO: 466, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to the sequence encoded by SEQ ID NO: 466 and one or more copies of one or more of the following membrane-bound immunosuppressive constructs: SEQ ID NO: 273, 276, 279, 280, or 283.

In several embodiments, the engineered cells provided for herein comprise a CAR that targets CD70, wherein the CAR comprises an amino acid of any of SEQ ID NOs: 383-465, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NO: 383-465 and one or more copies of one or more of the following membrane-bound immunosuppressive constructs: SEQ ID NO: 273, 276, 279, 280, or 283.

As discussed herein, in several embodiments, knockout of B2M expression results in loss of MHC expression on the edited cell. However, in several embodiments, HLA can be re-expressed, for example HLA-E or HLA-G. In several embodiments, the re-expression of the HLA is accomplished using a disulfilde trap single chain trimer (dtSCT) is used to express HLA-E and/or HLA-G and an immunosuppressive peptide, as well as B2M (HLA-E_STE20 or HLA-E_STE15). Provided for herein, in several embodiments, is a polynucleotide encoding a chimeric immunosuppressive construct comprising an HLA-G peptide, mature B2M and mature HLA-E. In several embodiments, such a construct comprises one or more linkers. In several embodiments, the immunosuppressive construct comprises a B2M signal peptide, an HLA-G peptide (amino acids 3-11 of HLA-G), a disulfide-bridge containing linker (e.g., a GS linker comprising at least two cysteine residues), a mature B2M domain, an additional linker (e.g., a GS linker), and a mature HLA-E domain. In several embodiments, the HLA-E_STE20 is encoded by a polynucleotide that encodes an amino acid sequence comprising SEQ ID NO: 829. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 829. In several embodiments, the polynucleotide comprises SEQ ID NO: 830 or shares at least about 80%, at least about 85%, at least about 90%, at least about 94%, at least about 95%, at least about 96%, at least about 97%, at least about 98%, or at least about 99%, sequence identity with SEQ ID NO: 830. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HLA-E_STE20 and the tag. In several embodiments, the polynucleotide encoding HLA-E_STE20 and GFP comprises SEQ ID NO: 828 or shares at least about 80%, at least about 85%, at least about 90%, at least about 94%, at least about 95%, at least about 96%, at least about 97%, at least about 98%, or at least about 99%, sequence identity with SEQ ID NO: 828. In several embodiments, the HLA-E_STE20-GFP construct comprises the amino acid sequence of SEQ ID NO: 827 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 827.

### Extracellular Domains (Tumor Binder)

Some embodiments of the compositions and methods described herein relate to a chimeric antigen receptor that includes an extracellular domain that comprises a tumor-binding domain (also referred to as an antigen-binding protein or antigen-binding domain) as described herein. The tumor binding domain, depending on the embodiment, targets, for example CD19, CD123, CD70, Her2, mesothelin, Claudin 6, BCMA, EGFR, among others. Several embodiments of the compositions and methods described herein relate to a chimeric receptor that includes an extracellular domain that comprises a ligand binding domain that binds a ligand expressed by a tumor cell (also referred to as an activating chimeric receptor) as described herein. The ligand binding domain, depending on the embodiment, targets for example MICA, MICB, ULBP1, ULBP2, ULBP3, ULBP4, ULBP5, and ULBP6 (among others).

In some embodiments, the antigen-binding domain is derived from or comprises wild-type or non-wild-type sequence of an antibody, an antibody fragment, an scFv, a Fv, a Fab, a (Fab′)2, a single domain antibody (SDAB), a vH or vL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the tumor-binding domain contains more than one antigen binding domain. In embodiments, the antigen-binding domain is operably linked directly or via an optional linker to the NH2-terminal end of a TCR domain (e.g. constant chains of TCR-alpha, TCR-betal, TCR-beta2, preTCR-alpha, pre-TCR-alpha-Del48, TCR-gamma, or TCR-delta).

### Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen (e.g., CD19) or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Nonlimiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g. enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (A) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (p), delta (A), gamma (γ), alpha (a), and epsilon (c), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3)(see U.S. Pat. No. 6,703,199, which describes fibronectin polypeptide mini bodies). An antibody fragment may include a Fab, Fab′, F(ab′)2, and/or Fv fragment that contains at least one CDR of an immunoglobulin that is sufficient to confer specific antigen binding to a cancer antigen (e.g., CD19). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it an antigen-binding protein.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

There is also provided, in some embodiments, antigen-binding proteins with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets. In several embodiments, this is particularly advantageous, as a bispecific chimeric antigen receptor can impart to an engineered cell the ability to target multiple tumor markers. For example, CD19 and an additional tumor marker, such as CD123, CD70, Her2, mesothelin, Claudin 6, BCMA, EGFR, MICA, MICB, ULBP1, ULBP2, ULBP3, ULBP4, ULBP5, and ULBP6, among others, or any other marker disclosed herein or appreciated in the art as a tumor specific antigen or tumor associated antigen can be bound by a bispecific antibody.

As used herein, the term “chimeric antibody” shall be given its ordinary meaning, and shall also refer to an antibody that contains one or more regions from one antibody and one or more regions from one or more other antibodies. In some embodiments, one or more of the CDRs are derived from an anti-cancer antigen (e.g., CD19, CD123, CD70, Her2, mesothelin, PD-L1, Claudin 6, BCMA, EGFR, etc.) antibody. In several embodiments, all of the CDRs are derived from an anti-cancer antigen antibody (such as an anti-CD19 antibody). In some embodiments, the CDRs from more than one anti-cancer antigen antibodies are mixed and matched in a chimeric antibody. For instance, a chimeric antibody may comprise a CDR1 from the light chain of a first anti-cancer antigen antibody, a CDR2 and a CDR3 from the light chain of a second anti-cancer antigen antibody, and the CDRs from the heavy chain from a third anti-cancer antigen antibody. Further, the framework regions of antigen-binding proteins disclosed herein may be derived from one of the same anti-cancer antigen (e.g., CD19, CD123, CD70, Her2, mesothelin, Claudin 6, BCMA, EGFR, etc.) antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also provided herein are fragments of such antibodies that exhibit the desired biological activity.

In several embodiments, an antigen binding protein is directed against CD38 (also known as ADP-ribosyl cyclase 1, cADPr hydrolase 1, Cyclic ADP-ribose hydrolase 1, or T10). According to one embodiment, the CD38 antigen binding protein is an antigen binding domain of a CAR which comprises a transmembrane domain, a signaling domain, and optionally a co-stimulatory domain as disclosed herein. In several embodiments, the antigen binding protein binds to an epitope of the human CD38, and in particular to an epitope of the extracellular domain of the human CD38. In several embodiments, the CD38 binding protein comprises an scFv comprising a vL and vH domain. In several embodiments, the anti-CD38 vL domain comprises the sequence of SEQ ID NO: 523, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 523. In several embodiments, the anti-CD38 vH domain comprises the sequence of SEQ ID NO: 524, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 524. In several embodiments, the anti-CD38 binding protein is an scFv that comprises the sequence of SEQ ID NO: 532, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 532. In several embodiments, the anti-CD38 CAR comprises the sequence of SEQ ID NO: 525, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 525. In several embodiments, the anti-CD38 binding protein comprises at least one CDR from SEQ ID NO: 526-531 or a CDR having at least about 95%, at least about 96%, at least about 97%, at least about 98%, or at least about 99%, sequence identity, homology and/or functional equivalence to any of SEQ ID NO: 526-531. In several embodiments, the antigen binding protein is affinity matured to enhance binding to CD38. In several embodiments, the nucleotide sequence encoding the antigen binding protein is codon-optimized to enhance expression and/or stability of the protein.

In several embodiments, an antigen binding protein is directed against GPRCSD. According to one embodiment, the GPRCSD antigen binding protein is an antigen binding domain of a CAR which comprises a transmembrane domain, a signaling domain, and optionally a co-stimulatory domain as disclosed herein. In several embodiments, the antigen binding protein binds to an epitope of the human GPRCSD. In several embodiments, the anti-GPRCSD is an scFv comprising the amino acid sequence of any one of SEQ ID NOs: 621-630, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NOs: 620-628. In several embodiments, the antigen binding protein is affinity matured to enhance binding to GPRCSD. In several embodiments, the nucleotide sequence encoding the antigen binding protein is codon-optimized to enhance expression and/or stability of the protein.

In several embodiments, an antigen binding protein is directed against CD138. In several embodiments, the anti-CD138 binding protein comprises a vL and/or vH chain. In several embodiments, the vL chain comprises the amino acid sequence of SEQ ID NO: 533, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 533. In several embodiments, the vH chain comprises the amino acid sequence of SEQ ID NO: 534, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 534. In several embodiments, the anti-CD138 binding protein comprises at least one CDR from SEQ ID NO: 536-541 or a CDR having at least about 95%, at least about 96%, at least about 97%, at least about 98%, or at least about 99%, sequence identity, homology and/or functional equivalence to any of SEQ ID NO: 536-541. In several embodiments, the anti-CD138 binding protein is an scFv comprising the amino acid sequence of SEQ ID NO: 542, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 542. In several embodiments, the anti-CD138 binding protein is integrated into a CAR which comprises a transmembrane domain, a signaling domain, and optionally a co-stimulatory domain as disclosed herein comprising the amino acid sequence of SEQ ID NO: 535 or 543, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 535 or 543. In several embodiments, the antigen binding protein is affinity matured to enhance binding to CD138. In several embodiments, the nucleotide sequence encoding the antigen binding protein is codon-optimized to enhance expression and/or stability of the protein.

In several embodiments, an antigen binding protein is directed against DLL3. In several embodiments, the anti-DLL3 binding protein is an antigen binding domain of a CAR which comprises a transmembrane domain, a signaling domain, and optionally a co-stimulatory domain as disclosed herein. In several embodiments, the anti-DLL3 antigen binding protein comprises a vH chain comprising the amino acid sequence of any of SEQ ID NOs: 570-581, or a sequence having at least about 95%, at least about 96%, at least about 97%, at least about 98%, or at least about 99%, sequence identity, homology and/or functional equivalence to any of SEQ ID NO: 570-581. In several embodiments, the anti-DLL3 antigen binding protein comprises a vL chain comprising the amino acid sequence of any of SEQ ID NOs: 582-593, or a sequence having at least about 95%, at least about 96%, at least about 97%, at least about 98%, or at least about 99%, sequence identity, homology and/or functional equivalence to any of SEQ ID NO: 582-593. In several embodiments, the anti-DLL3 binding protein comprises a polypeptide that targets DLL3 and comprises the amino acid sequence of any of SEQ ID NO: 594-595, or a sequence having at least about 95%, at least about 96%, at least about 97%, at least about 98%, or at least about 99%, sequence identity, homology and/or functional equivalence to any of SEQ ID NO: 594-595. In several embodiments, the anti-DLL3 binding protein comprises an scFv comprising the sequence of any of SEQ ID NO: 596-599, or a sequence having at least about 95%, at least about 96%, at least about 97%, at least about 98%, or at least about 99%, sequence identity, homology and/or functional equivalence to any of SEQ ID NO: 596-599. In several embodiments, the antigen binding protein is affinity matured to enhance binding to DLL3. In several embodiments, the nucleotide sequence encoding the antigen binding protein is codon-optimized to enhance expression and/or stability of the protein.

In several embodiments, an antigen binding protein is directed against the epidermal growth factor receptor EGFR. In several embodiments, the anti-EGFR binding protein is an antigen binding domain of a CAR which comprises a transmembrane domain, a signaling domain, and optionally a co-stimulatory domain as disclosed herein. In several embodiments, the anti-EGFR binding protein comprises a vH chain comprising the amino acid sequence of any of SEQ ID NO: 600, 606-607, or a sequence having at least about 95%, at least about 96%, at least about 97%, at least about 98%, or at least about 99%, sequence identity, homology and/or functional equivalence to any of SEQ ID NO: 600, 606-607. In several embodiments, the anti-EGFR binding protein comprises a vL chain comprising the amino acid sequence of any of SEQ ID NO: 601, 608-609, or a sequence having at least about 95%, at least about 96%, at least about 97%, at least about 98%, or at least about 99%, sequence identity, homology and/or functional equivalence to any of SEQ ID NO: 601, 608-609. In several embodiments, the anti-EGFR binding protein is an scFv comprising the amino acid sequence of any of SEQ ID NOs: 610-620, or a sequence having at least about 95%, at least about 96%, at least about 97%, at least about 98%, or at least about 99%, sequence identity, homology and/or functional equivalence to any of SEQ ID NO: 610-620. In several embodiments, the anti-EGFR binding protein is incorporated into a CAR having the sequence of any of SEQ ID NOs: 602-605, or a sequence having at least about 95%, at least about 96%, at least about 97%, at least about 98%, or at least about 99%, sequence identity, homology and/or functional equivalence to any of SEQ ID NO: 602-605. In several embodiments, the antigen binding protein is affinity matured to enhance binding to the EGFR. In several embodiments, the nucleotide sequence encoding the antigen binding protein is codon-optimized to enhance expression and/or stability of the protein.

In several embodiments, an antigen binding protein is directed against PSMA. In several embodiments, the anti-PSMA binding protein is an antigen binding domain of a CAR which comprises a transmembrane domain, a signaling domain, and optionally a co-stimulatory domain as disclosed herein. In several embodiments, the anti-PSMA binding protein comprises a vL chain comprising the amino acid sequence of SEQ ID NO: 634, or a sequence having at least about 95%, at least about 96%, at least about 97%, at least about 98%, or at least about 99%, sequence identity, homology and/or functional equivalence to SEQ ID NO: 634. In several embodiments, the anti-PSMA binding protein comprises a vH chain comprising the amino acid sequence of SEQ ID NO: 635, or a sequence having at least about 95%, at least about 96%, at least about 97%, at least about 98%, or at least about 99%, sequence identity, homology and/or functional equivalence to SEQ ID NO: 635. In several embodiments, the anti-PSMA binding protein comprises an scFv comprising the amino acid sequence of SEQ ID NO: 632 or 633, or a sequence having at least about 95%, at least about 96%, at least about 97%, at least about 98%, or at least about 99%, sequence identity, homology and/or functional equivalence to SEQ ID NO: 632 or 633. In several embodiments, the anti-PSMA binding protein comprises an antibody comprising the amino acid sequence of SEQ ID NO: 631, or a sequence having at least about 95%, at least about 96%, at least about 97%, at least about 98%, or at least about 99%, sequence identity, homology and/or functional equivalence to SEQ ID NO: 631. In several embodiments, the antigen binding protein is affinity matured to enhance binding to PSMA. In several embodiments, the nucleotide sequence encoding the antigen binding protein is codon-optimized to enhance expression and/or stability of the protein.

In several embodiments, an antigen binding protein is directed against FLT3. In several embodiments, the anti-FLT3 binding protein is an antigen binding domain of a CAR which comprises a transmembrane domain, a signaling domain, and optionally a co-stimulatory domain as disclosed herein. In several embodiments, the anti-FLT3 binding protein comprises one or more CDRs from the vL and/or vH chain selected from SEQ ID NOs: 636-644, or a o CDR having at least about 95%, at least about 96%, at least about 97%, at least about 98%, or at least about 99%, sequence identity, homology and/or functional equivalence to any of SEQ ID NO: 636-644. In several embodiments, the anti-FLT3 binding protein comprises a vL chain comprising the amino acid sequence of SEQ ID NO: 645, or a sequence having at least about 95%, at least about 96%, at least about 97%, at least about 98%, or at least about 99%, sequence identity, homology and/or functional equivalence to SEQ ID NO: 645. In several embodiments, the anti-FLT3 binding protein comprises a vH chain comprising the amino acid sequence of SEQ ID NO: 646, or a sequence having at least about 95%, at least about 96%, at least about 97%, at least about 98%, or at least about 99%, sequence identity, homology and/or functional equivalence to SEQ ID NO: 646. In several embodiments, the antigen binding protein is affinity matured to enhance binding to FLT3. In several embodiments, the nucleotide sequence encoding the antigen binding protein is codon-optimized to enhance expression and/or stability of the protein.

In several embodiments, an antigen binding protein is directed against KREMEN2. In several embodiments, the anti-KREMEN2 binding protein is an antigen binding domain of a CAR which comprises a transmembrane domain, a signaling domain, and optionally a co-stimulatory domain as disclosed herein. In several embodiments, the anti-KREMEN2 binding protein comprises a vL chain comprising the amino acid sequence of any of SEQ ID NOs: 647-651, or a sequence having at least about 95%, at least about 96%, at least about 97%, at least about 98%, or at least about 99%, sequence identity, homology and/or functional equivalence to any of SEQ ID NOs: 647-651. In several embodiments, the anti-KREMEN2 binding protein comprises a vH chain comprising the amino acid sequence of any of SEQ ID NOs: 652-656, or a sequence having at least about 95%, at least about 96%, at least about 97%, at least about 98%, or at least about 99%, sequence identity, homology and/or functional equivalence to any of SEQ ID NOs: 652-656. In several embodiments, the antigen binding protein is affinity matured to enhance binding to KREMEN2. In several embodiments, the nucleotide sequence encoding the antigen binding protein is codon-optimized to enhance expression and/or stability of the protein.

In several embodiments, an antigen binding protein is directed against ALPPL2. In several embodiments, the anti-ALPPL2 binding protein is an antigen binding domain of a CAR which comprises a transmembrane domain, a signaling domain, and optionally a co-stimulatory domain as disclosed herein. In several embodiments, the anti-ALPPL2 binding protein comprises a vL chain comprising the amino acid sequence of any of SEQ ID NOs: 657-659, or a sequence having at least about 95%, at least about 96%, at least about 97%, at least about 98%, or at least about 99%, sequence identity, homology and/or functional equivalence to any of SEQ ID NOs: 657-659. In several embodiments, the anti-ALPPL2 binding protein comprises a vH chain comprising the amino acid sequence of any of SEQ ID NOs: 660-662, or a sequence having at least about 95%, at least about 96%, at least about 97%, at least about 98%, or at least about 99%, sequence identity, homology and/or functional equivalence to any of SEQ ID NOs: 660-662. In several embodiments, the anti-ALPPL2 binding protein is an antibody, or scFv, containing one or more combinations of the vL and vH domains comprising the amino acid sequence of any of SEQ ID NOs: 657-662, or a sequence having at least about 95%, at least about 96%, at least about 97%, at least about 98%, or at least about 99%, sequence identity, homology and/or functional equivalence to any of SEQ ID NO: 657-662. In several embodiments, the antigen binding protein is affinity matured to enhance binding to ALPPL2. In several embodiments, the nucleotide sequence encoding the antigen binding protein is codon-optimized to enhance expression and/or stability of the protein.

In several embodiments, an antigen binding protein is directed against CLDN4. In several embodiments, the anti-CLDN4 binding protein is an antigen binding domain of a CAR which comprises a transmembrane domain, a signaling domain, and optionally a co-stimulatory domain as disclosed herein. In several embodiments, the anti-CLDN4 binding protein comprises a vL chain comprising the amino acid sequence of any of SEQ ID NOs: 663, 664, or 667, or a sequence having at least about 95%, at least about 96%, at least about 97%, at least about 98%, or at least about 99%, sequence identity, homology and/or functional equivalence to any of SEQ ID NOs: 663, 664, or 667. In several embodiments, the anti-CLDN4 binding protein comprises a vH chain comprising the amino acid sequence of any of SEQ ID NOs: 665, 666, or 668, or a sequence having at least about 95%, at least about 96%, at least about 97%, at least about 98%, or at least about 99%, sequence identity, homology and/or functional equivalence to any of SEQ ID NOs: 665, 666, or 668. In several embodiments, the anti-CLDN4 binding protein is an antibody, or scFv, containing one or more combinations of the vL and vH domains comprising the amino acid sequence of any of SEQ ID NOs: 663-668, or a sequence having at least about 95%, at least about 96%, at least about 97%, at least about 98%, or at least about 99%, sequence identity, homology and/or functional equivalence to any of SEQ ID NOs: 663-668. In several embodiments, the antigen binding protein is affinity matured to enhance binding to CLDN4. In several embodiments, the nucleotide sequence encoding the antigen binding protein is codon-optimized to enhance expression and/or stability of the protein. In several embodiments, the antigen binding protein binds to CLDN4, but not to other claudins.

In several embodiments, an antigen binding protein is directed against CLDN6. In several embodiments, the anti-CLDN6 binding protein is an antigen binding domain of a CAR which comprises a transmembrane domain, a signaling domain, and optionally a co-stimulatory domain as disclosed herein. In several embodiments, the anti-CLDN6 binding protein comprises a vL chain comprising the amino acid sequence of any of SEQ ID NOs: 669-678, or a sequence having at least about 95%, at least about 96%, at least about 97%, at least about 98%, or at least about 99%, sequence identity, homology and/or functional equivalence to any of SEQ ID NOs: 669-678. In several embodiments, the anti-CLDN6 binding protein comprises a vH chain comprising the amino acid sequence of any of SEQ ID NOs: 679-682, or a sequence having at least about 95%, at least about 96%, at least about 97%, at least about 98%, or at least about 99%, sequence identity, homology and/or functional equivalence to any of SEQ ID NOs: 679-682. In several embodiments, the anti-CLDN6 binding protein is an antibody, or scFv, containing one or more combinations of the vL and vH domains comprising the amino acid sequence of any of SEQ ID NOs: 669-682, or a sequence having at least about 95%, at least about 96%, at least about 97%, at least about 98%, or at least about 99%, sequence identity, homology and/or functional equivalence to any of SEQ ID NOs: 669-682. In several embodiments, the antigen binding protein is affinity matured to enhance binding to CLDN6. In several embodiments, the nucleotide sequence encoding the antigen binding protein is codon-optimized to enhance expression and/or stability of the protein. In several embodiments, the antigen binding protein binds to CLDN6, but not to other claudins.

In some embodiments, an antigen-binding protein is provided comprising a heavy chain variable domain having at least 90% identity to the VH domain amino acid sequence set forth in SEQ ID NO: 33. In some embodiments, the antigen-binding protein comprises a heavy chain variable domain having at least 95% identity to the VH domain amino acid sequence set forth in SEQ ID NO: 33. In some embodiments, the antigen-binding protein comprises a heavy chain variable domain having at least 96, 97, 98, or 99% identity to the VH domain amino acid sequence set forth in SEQ ID NO: 33. In several embodiments, the heavy chain variable domain may have one or more additional mutations (e.g., for purposes of humanization) in the VH domain amino acid sequence set forth in SEQ ID NO: 33, but retains specific binding to a cancer antigen (e.g., CD19). In several embodiments, the heavy chain variable domain may have one or more additional mutations in the VH domain amino acid sequence set forth in SEQ ID NO: 33, but has improved specific binding to a cancer antigen (e.g., CD19).

In some embodiments, the antigen-binding protein comprises a light chain variable domain having at least 90% identity to the VL domain amino acid sequence set forth in SEQ ID NO: 32. In some embodiments, the antigen-binding protein comprises a light chain variable domain having at least 95% identity to the VL domain amino acid sequence set forth in SEQ ID NO: 32. In some embodiments, the antigen-binding protein comprises a light chain variable domain having at least 96, 97, 98, or 99% identity to the VL domain amino acid sequence set forth in SEQ ID NO: 32. In several embodiments, the light chain variable domain may have one or more additional mutations (e.g., for purposes of humanization) in the VL domain amino acid sequence set forth in SEQ ID NO: 32, but retains specific binding to a cancer antigen (e.g., CD19). In several embodiments, the light chain variable domain may have one or more additional mutations in the VL domain amino acid sequence set forth in SEQ ID NO: 32, but has improved specific binding to a cancer antigen (e.g., CD19).

In some embodiments, the antigen-binding protein comprises a heavy chain variable domain having at least 90% identity to the VH domain amino acid sequence set forth in SEQ ID NO: 33, and a light chain variable domain having at least 90% identity to the VL domain amino acid sequence set forth in SEQ ID NO: 32. In some embodiments, the antigen-binding protein comprises a heavy chain variable domain having at least 95% identity to the VH domain amino acid sequence set forth in SEQ ID NO: 33, and a light chain variable domain having at least 95% identity to the VL domain amino acid sequence set forth in SEQ ID NO: 32. In some embodiments, the antigen-binding protein comprises a heavy chain variable domain having at least 96, 97, 98, or 99% identity to the VH domain amino acid sequence set forth in SEQ ID NO: 33, and a light chain variable domain having at least 96, 97, 98, or 99% identity to the VL domain amino acid sequence set forth in SEQ ID NO: 32.

In some embodiments, the antigen-binding protein comprises a heavy chain variable domain having the VH domain amino acid sequence set forth in SEQ ID NO: 33, and a light chain variable domain having the VL domain amino acid sequence set forth in SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 33.

In some embodiments, the light chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the polynucleotide sequence SEQ ID NO: 32.

In some embodiments, the light chain variable domain comprises a sequence of amino acids that is encoded by a polynucleotide that hybridizes under moderately stringent conditions to the complement of a polynucleotide that encodes a light chain variable domain in accordance with the sequence in SEQ ID NO: 32. In some embodiments, the light chain variable domain comprises a sequence of amino acids that is encoded by a polynucleotide that hybridizes under stringent conditions to the complement of a polynucleotide that encodes a light chain variable domain in accordance with the sequence in SEQ ID NO: 32.

In some embodiments, the heavy chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with the sequence of SEQ ID NO: 33. In some embodiments, the heavy chain variable domain comprises a sequence of amino acids that is encoded by a polynucleotide that hybridizes under moderately stringent conditions to the complement of a polynucleotide that encodes a heavy chain variable domain in accordance with the sequence of SEQ ID NO: 33. In some embodiments, the heavy chain variable domain comprises a sequence of amino acids that is encoded by a polynucleotide that hybridizes under stringent conditions to the complement of a polynucleotide that encodes a heavy chain variable domain in accordance with the sequence of SEQ ID NO: 33.

In several embodiments, additional anti-CD19 binding constructs are provided. For example, in several embodiments, there is provided an scFv that targets CD19 wherein the scFv comprises a heavy chain variable region comprising the sequence of SEQ ID NO. 35. In some embodiments, the antigen-binding protein comprises a heavy chain variable domain having at least 95% identity to the HCV domain amino acid sequence set forth in SEQ ID NO: 35. In some embodiments, the antigen-binding protein comprises a heavy chain variable domain having at least 96, 97, 98, or 99% identity to the HCV domain amino acid sequence set forth in SEQ ID NO: 35. In several embodiments, the heavy chain variable domain may have one or more additional mutations (e.g., for purposes of humanization) in the HCV domain amino acid sequence set forth in SEQ ID NO: 35, but retains specific binding to a cancer antigen (e.g., CD19). In several embodiments, the heavy chain variable domain may have one or more additional mutations in the HCV domain amino acid sequence set forth in SEQ ID NO: 35, but has improved specific binding to a cancer antigen (e.g., CD19).

Additionally, in several embodiments, an scFv that targets CD19 comprises a light chain variable region comprising the sequence of SEQ ID NO. 36. In some embodiments, the antigen-binding protein comprises a light chain variable domain having at least 95% identity to the LCV domain amino acid sequence set forth in SEQ ID NO: 36. In some embodiments, the antigen-binding protein comprises a light chain variable domain having at least 96, 97, 98, or 99% identity to the LCV domain amino acid sequence set forth in SEQ ID NO: 36. In several embodiments, the light chain variable domain may have one or more additional mutations (e.g., for purposes of humanization) in the LCV domain amino acid sequence set forth in SEQ ID NO: 36, but retains specific binding to a cancer antigen (e.g., CD19). In several embodiments, the light chain variable domain may have one or more additional mutations in the LCV domain amino acid sequence set forth in SEQ ID NO: 36, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 37. In several embodiments, the LC CDR1 comprises an amino acid sequence with at least about 85%, about 90%, about 95%, or about 98% sequence identity to the sequence of SEQ NO. 37. In several embodiments, the LC CDR2 comprises the sequence of SEQ ID NO. 38. In several embodiments, the LC CDR2 comprises an amino acid sequence with at least about 85%, about 90%, about 95%, or about 98% sequence identity to the sequence of SEQ NO. 38. In several embodiments, the LC CDR3 comprises the sequence of SEQ ID NO. 39. In several embodiments, the LC CDR3 comprises an amino acid sequence with at least about 85%, about 90%, about 95%, or about 98% sequence identity to the sequence of SEQ NO. 39. In several embodiments, the HC CDR1 comprises the sequence of SEQ ID NO. 40. In several embodiments, the HC CDR1 comprises an amino acid sequence with at least about 85%, about 90%, about 95%, or about 98% sequence identity to the sequence of SEQ NO. 40. In several embodiments, the HC CDR2 comprises the sequence of SEQ ID NO. 41, 42, or 43. In several embodiments, the HC CDR2 comprises an amino acid sequence with at least about 85%, about 90%, about 95%, or about 98% sequence identity to the sequence of SEQ NO. 41, 42, or 43. In several embodiments, the HC CDR3 comprises the sequence of SEQ ID NO. 44. In several embodiments, the HC CDR3 comprises an amino acid sequence with at least about 85%, about 90%, about 95%, or about 98% sequence identity to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48. In several embodiments, the VL region comprises an amino acid sequence with at least about 85%, about 90%, about 95%, or about 98% sequence identity to the sequence of SEQ NO. 45, 46, 47, or 48. In several embodiments, the VH region comprises the sequence of SEQ ID NO. 49, 50, 51 or 52. In several embodiments, the VH region comprises an amino acid sequence with at least about 85%, about 90%, about 95%, or about 98% sequence identity to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53. In several embodiments, the LC CDR1 comprises an amino acid sequence with at least about 85%, about 90%, about 95%, or about 98% sequence identity to the sequence of SEQ NO. 53. In several embodiments, the LC CDR2 comprises the sequence of SEQ ID NO. 54. In several embodiments, the LC CDR2 comprises an amino acid sequence with at least about 85%, about 90%, about 95%, or about 98% sequence identity to the sequence of SEQ NO. 54. In several embodiments, the LC CDR3 comprises the sequence of SEQ ID NO. 55. In several embodiments, the LC CDR3 comprises an amino acid sequence with at least about 85%, about 90%, about 95%, or about 98% sequence identity to the sequence of SEQ NO. 55. In several embodiments, the HC CDR1 comprises the sequence of SEQ ID NO. 56. In several embodiments, the HC CDR1 comprises an amino acid sequence with at least about 85%, about 90%, about 95%, or about 98% sequence identity to the sequence of SEQ NO. 56. In several embodiments, the HC CDR2 comprises the sequence of SEQ ID NO. 57. In several embodiments, the HC CDR2 comprises an amino acid sequence with at least about 85%, about 90%, about 95%, or about 98% sequence identity to the sequence of SEQ NO. 57. In several embodiments, the HC CDR3 comprises the sequence of SEQ ID NO. 58. In several embodiments, the HC CDR3 comprises an amino acid sequence with at least about 85%, about 90%, about 95%, or about 98% sequence identity to the sequence of SEQ NO. 58.

In some embodiments, the antigen-binding protein comprises a heavy chain variable region comprising the amino acid sequence of SEQ ID NO: 104. In some embodiments, the antigen-binding protein comprises a heavy chain variable region having at least 90% identity to the VH domain amino acid sequence set forth in SEQ ID NO: 104. In some embodiments, the antigen-binding protein comprises a heavy chain variable domain having at least 95% sequence identity to the VH domain amino acid sequence set forth in SEQ ID NO: 104. In some embodiments, the antigen-binding protein comprises a heavy chain variable domain having at least 96, 97, 98, or 99% sequence identity to the VH domain amino acid sequence set forth in SEQ ID NO: 104. In several embodiments, the heavy chain variable domain may have one or more additional mutations (e.g., for purposes of humanization) in the VH domain amino acid sequence set forth in SEQ ID NO: 104, but retains specific binding to a cancer antigen (e.g., CD19). In several embodiments, the heavy chain variable domain may have one or more additional mutations in the VH domain amino acid sequence set forth in SEQ ID NO: 104, but has improved specific binding to a cancer antigen (e.g., CD19).

In some embodiments, the antigen-binding protein comprises a light chain variable region comprising the amino acid sequence of SEQ ID NO: 105. In some embodiments, the antigen-binding protein comprises a light chain variable region having at least 90% sequence identity to the VL domain amino acid sequence set forth in SEQ ID NO: 105. In some embodiments, the antigen-binding protein comprises a light chain variable domain having at least 95% sequence identity to the VL domain amino acid sequence set forth in SEQ ID NO: 105. In some embodiments, the antigen-binding protein comprises a light chain variable domain having at least 96, 97, 98, or 99% sequence identity to the VL domain amino acid sequence set forth in SEQ ID NO: 105. In several embodiments, the light chain variable domain may have one or more additional mutations (e.g., for purposes of humanization) in the VL domain amino acid sequence set forth in SEQ ID NO: 105, but retains specific binding to a cancer antigen (e.g., CD19). In several embodiments, the light chain variable domain may have one or more additional mutations in the VL domain amino acid sequence set forth in SEQ ID NO: 105, but has improved specific binding to a cancer antigen (e.g., CD19).

In some embodiments, the antigen-binding protein comprises a heavy chain variable domain having the VH domain amino acid sequence set forth in SEQ ID NO: 104, and a light chain variable domain having the VL domain amino acid sequence set forth in SEQ ID NO: 105. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 105. In some embodiments, the heavy-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 104.

In some embodiments, the antigen-binding protein comprises a heavy chain variable comprising the amino acid sequence of SEQ ID NO: 106. In some embodiments, the antigen-binding protein comprises a heavy chain variable having at least 90% sequence identity to the VH amino acid sequence set forth in SEQ ID NO: 106. In some embodiments, the antigen-binding protein comprises a heavy chain variable having at least 95% sequence identity to the VH amino acid sequence set forth in SEQ ID NO: 106. In some embodiments, the antigen-binding protein comprises a heavy chain variable having at least 96, 97, 98, or 99% identity to the VH amino acid sequence set forth in SEQ ID NO: 106. In several embodiments, the heavy chain variable may have one or more additional mutations (e.g., for purposes of humanization) in the VH amino acid sequence set forth in SEQ ID NO: 106, but retains specific binding to a cancer antigen (e.g., CD19). In several embodiments, the heavy chain variable may have one or more additional mutations in the VH amino acid sequence set forth in SEQ ID NO: 106, but has improved specific binding to a cancer antigen (e.g., CD19).

In some embodiments, the antigen-binding protein comprises a light chain variable comprising the amino acid sequence of SEQ ID NO: 107. In some embodiments, the antigen-binding protein comprises a light chain variable region having at least 90% sequence identity to the VL amino acid sequence set forth in SEQ ID NO: 107. In some embodiments, the antigen-binding protein comprises a light chain variable having at least 95% sequence identity to the VL amino acid sequence set forth in SEQ ID NO: 107. In some embodiments, the antigen-binding protein comprises a light chain variable having at least 96, 97, 98, or 99% identity to the VL amino acid sequence set forth in SEQ ID NO: 107. In several embodiments, the light chain variable may have one or more additional mutations (e.g., for purposes of humanization) in the VL amino acid sequence set forth in SEQ ID NO: 107, but retains specific binding to a cancer antigen (e.g., CD19). In several embodiments, the light chain variable may have one or more additional mutations in the VL amino acid sequence set forth in SEQ ID NO: 107, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 108. In several embodiments, the LC CDR1 comprises an amino acid sequence with at least about 85%, about 90%, about 95%, or about 98% sequence identity to the sequence of SEQ NO. 108. In several embodiments, the LC CDR2 comprises the sequence of SEQ ID NO. 109. In several embodiments, the LC CDR2 comprises an amino acid sequence with at least about 85%, about 90%, about 95%, or about 98% sequence identity to the sequence of SEQ NO. 109. In several embodiments, the LC CDR3 comprises the sequence of SEQ ID NO. 110. In several embodiments, the LC CDR3 comprises an amino acid sequence with at least about 85%, about 90%, about 95%, or about 98% sequence identity to the sequence of SEQ NO. 110. In several embodiments, the HC CDR1 comprises the sequence of SEQ ID NO. 111. In several embodiments, the HC CDR1 comprises an amino acid sequence with at least about 85%, about 90%, about 95%, or about 98% sequence identity to the sequence of SEQ NO. 111. In several embodiments, the HC CDR2 comprises the sequence of SEQ ID NO. 112, 113, or 114. In several embodiments, the HC CDR2 comprises an amino acid sequence with at least about 85%, about 90%, about 95%, or about 98% sequence identity to the sequence of SEQ NO. 112, 113, or 114. In several embodiments, the HC CDR3 comprises the sequence of SEQ ID NO. 115. In several embodiments, the HC CDR3 comprises an amino acid sequence with at least about 85%, about 90%, about 95%, or about 98% sequence identity to the sequence of SEQ NO. 115. In several embodiments, the anti-CD19 binding moiety comprises SEQ ID NO: 116, or is sequence with at least about 85%, about 90%, about 95%, or about 98% sequence identity to the sequence of SEQ NO. 116.

In some embodiments, the antigen-binding protein comprises a light chain variable comprising the amino acid sequence of SEQ ID NO: 117, 118, or 119. In some embodiments, the antigen-binding protein comprises a light chain variable region having at least 90% identity to the VL amino acid sequence set forth in SEQ ID NO: 117, 118, or 119. In some embodiments, the antigen-binding protein comprises a light chain variable having at least 95% identity to the VL amino acid sequence set forth in SEQ ID NO: 117, 118, or 119. In some embodiments, the antigen-binding protein comprises a light chain variable having at least 96, 97, 98, or 99% identity to the VL amino acid sequence set forth in SEQ ID NO: 117, 118, or 119. In several embodiments, the light chain variable may have one or more additional mutations (e.g., for purposes of humanization) in the VL amino acid sequence set forth in SEQ ID NO: 117, 118, or 119, but retains specific binding to a cancer antigen (e.g., CD19). In several embodiments, the light chain variable may have one or more additional mutations in the VL amino acid sequence set forth in SEQ ID NO: 117, 118, or 119, but has improved specific binding to a cancer antigen (e.g., CD19).

In some embodiments, the antigen-binding protein comprises a heavy chain variable comprising the amino acid sequence of SEQ ID NO: 120,121, 122, or 123. In some embodiments, the antigen-binding protein comprises a heavy chain variable having at least 90% identity to the VH amino acid sequence set forth in SEQ ID NO: 120,121, 122, or 123. In some embodiments, the antigen-binding protein comprises a heavy chain variable having at least 95% identity to the VH amino acid sequence set forth in SEQ ID NO: 120,121, 122, or 123. In some embodiments, the antigen-binding protein comprises a heavy chain variable having at least 96, 97, 98, or 99% identity to the VH amino acid sequence set forth in SEQ ID NO: 120,121, 122, or 123. In several embodiments, the heavy chain variable may have one or more additional mutations (e.g., for purposes of humanization) in the VH amino acid sequence set forth in SEQ ID NO: 120,121, 122, or 123, but retains specific binding to a cancer antigen (e.g., CD19). In several embodiments, the heavy chain variable may have one or more additional mutations in the VH amino acid sequence set forth in SEQ ID NO: 120,121, 122, or 123, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 124, 127, or 130. In several embodiments, the LC CDR1 comprises an amino acid sequence with at least about 85%, about 90%, about 95%, or about 98% sequence identity to the sequence of SEQ NO. 124, 127, or 130. In several embodiments, the LC CDR2 comprises the sequence of SEQ ID NO. 125, 128, or 131. In several embodiments, the LC CDR2 comprises an amino acid sequence with at least about 85%, about 90%, about 95%, or about 98% sequence identity to the sequence of SEQ NO. 125, 128, or 131. In several embodiments, the LC CDR3 comprises the sequence of SEQ ID NO. 126, 129, or 132. In several embodiments, the LC CDR3 comprises an amino acid sequence with at least about 85%, about 90%, about 95%, or about 98% sequence identity to the sequence of SEQ NO. 126, 129, or 132. In several embodiments, the HC CDR1 comprises the sequence of SEQ ID NO. 133, 136, 139, or 142. In several embodiments, the HC CDR1 comprises an amino acid sequence with at least about 85%, about 90%, about 95%, or about 98% sequence identity to the sequence of SEQ NO. 133, 136, 139, or 142. In several embodiments, the HC CDR2 comprises the sequence of SEQ ID NO. 134, 137, 140, or 143. In several embodiments, the HC CDR2 comprises an amino acid sequence with at least about 85%, about 90%, about 95%, or about 98% sequence identity to the sequence of SEQ NO. 134, 137, 140, or 143. In several embodiments, the HC CDR3 comprises the sequence of SEQ ID NO. 135, 138, 141, or 144. In several embodiments, the HC CDR3 comprises an amino acid sequence with at least about 85%, about 90%, about 95%, or about 98% sequence identity to the sequence of SEQ NO. 135, 138, 141, or 144.

Additional anti-CD19 binding moieties are known in the art, such as those disclosed in, for example, U.S. Pat. No. 8,399,645, US Patent Publication No. 2018/0153977, US Patent Publication No. 2014/0271635, US Patent Publication No. 2018/0251514, US Patent Publication No. 2018/0312588, and WO 2020/180882, the entirety of each of which is incorporated by reference herein.

Several embodiments relate to CARs that are directed to Claudin 6, and show little or no binding to Claudin 3, 4, or 9 (or other Claudins). In some embodiments, the antigen-binding protein comprises a heavy chain variable comprising the amino acid sequence of SEQ ID NO: 88. In some embodiments, the antigen-binding protein comprises a heavy chain variable having at least 90% identity to the VH amino acid sequence set forth in SEQ ID NO: 88. In some embodiments, the antigen-binding protein comprises a heavy chain variable having at least 95% identity to the VH amino acid sequence set forth in SEQ ID NO: 88. In some embodiments, the antigen-binding protein comprises a heavy chain variable having at least 96, 97, 98, or 99% identity to the VH amino acid sequence set forth in SEQ ID NO: 88. In several embodiments, the heavy chain variable may have one or more additional mutations (e.g., for purposes of humanization) in the VH amino acid sequence set forth in SEQ ID NO: 88, but retains specific binding to a cancer antigen (e.g., CLDN6). In several embodiments, the heavy chain variable may have one or more additional mutations in the VH amino acid sequence set forth in SEQ ID NO: 88, but has improved specific binding to a cancer antigen (e.g., CLDN6).

In some embodiments, the antigen-binding protein comprises a light chain variable comprising the amino acid sequence of SEQ ID NO: 89, 90 or 91. In some embodiments, the antigen-binding protein comprises a light chain variable region having at least 90% identity to the VL amino acid sequence set forth in SEQ ID NO: 89, 90 or 91. In some embodiments, the antigen-binding protein comprises a light chain variable having at least 95% identity to the VL amino acid sequence set forth in SEQ ID NO: 89, 90 or 91. In some embodiments, the antigen-binding protein comprises a light chain variable having at least 96, 97, 98, or 99% identity to the VL amino acid sequence set forth in SEQ ID NO: 89, 90 or 91. In several embodiments, the light chain variable may have one or more additional mutations (e.g., for purposes of humanization) in the VL amino acid sequence set forth in SEQ ID NO: 89, 90 or 91, but retains specific binding to a cancer antigen (e.g., CLDN6). In several embodiments, the light chain variable may have one or more additional mutations in the VL amino acid sequence set forth in SEQ ID NO: 89, 90 or 91, but has improved specific binding to a cancer antigen (e.g., CLDN6).

In several embodiments, there is also provided an anti-CLDN6 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 95, 98, or 101. In several embodiments, the LC CDR1 comprises an amino acid sequence with at least about 85%, about 90%, about 95%, or about 98% sequence identity to the sequence of SEQ NO. 95, 98, or 101. In several embodiments, the LC CDR2 comprises the sequence of SEQ ID NO. 96, 99, or 102. In several embodiments, the LC CDR2 comprises an amino acid sequence with at least about 85%, about 90%, about 95%, or about 98% sequence identity to the sequence of SEQ NO. 96, 99, or 102. In several embodiments, the LC CDR3 comprises the sequence of SEQ ID NO. 97, 100, or 103. In several embodiments, the LC CDR3 comprises an amino acid sequence with at least about 85%, about 90%, about 95%, or about 98% sequence identity to the sequence of SEQ NO. 97, 100, or 103. In several embodiments, the HC CDR1 comprises the sequence of SEQ ID NO. 92. In several embodiments, the HC CDR1 comprises an amino acid sequence with at least about 85%, about 90%, about 95%, or about 98% sequence identity to the sequence of SEQ NO. 92. In several embodiments, the HC CDR2 comprises the sequence of SEQ ID NO. 93. In several embodiments, the HC CDR2 comprises an amino acid sequence with at least about 85%, about 90%, about 95%, or about 98% sequence identity to the sequence of SEQ NO. 93. In several embodiments, the HC CDR3 comprises the sequence of SEQ ID NO. 94. In several embodiments, the HC CDR3 comprises an amino acid sequence with at least about 85%, about 90%, about 95%, or about 98% sequence identity to the sequence of SEQ NO. 94. In several embodiments, the antigen-binding protein does not bind claudins other than CLDN6.

Natural Killer Group Domains that Bind Tumor Ligands

In several embodiments, engineered immune cells such as NK cells are leveraged for their ability to recognize and destroy tumor cells. For example, an engineered NK cell may include a CD19-directed chimeric antigen receptor or a nucleic acid encoding said chimeric antigen receptor (or a CAR directed against, for example, one or more of CD123, CD70, Her2, mesothelin, Claudin 6, BCMA, EGFR, etc.). NK cells express both inhibitory and activating receptors on the cell surface. Inhibitory receptors bind self-molecules expressed on the surface of healthy cells (thus preventing immune responses against “self” cells), while the activating receptors bind ligands expressed on abnormal cells, such as tumor cells. When the balance between inhibitory and activating receptor activation is in favor of activating receptors, NK cell activation occurs and target (e.g., tumor) cells are lysed.

Natural killer Group 2 member D (NKG2D) is an NK cell activating receptor that recognizes a variety of ligands expressed on cells. The surface expression of various NKG2D ligands is generally low in healthy cells but is upregulated upon, for example, malignant transformation. Non-limiting examples of ligands recognized by NKG2D include, but are not limited to, MICA, MICB, ULBP1, ULBP2, ULBP3, ULBP4, ULBP5, and ULBP6, as well as other molecules expressed on target cells that control the cytolytic or cytotoxic function of NK cells. In several embodiments, T cells are engineered to express an extracellular domain to binds to one or more tumor ligands and activate the T cell. For example, in several embodiments, T cells are engineered to express an NKG2D receptor as the binder/activation moiety. In several embodiments, engineered cells as disclosed herein are engineered to express another member of the NKG2 family, e.g., NKG2A, NKG2C, and/or NKG2E. Combinations of such receptors are engineered in some embodiments. Moreover, in several embodiments, other receptors are expressed, such as the Killer-cell immunoglobulin-like receptors (KIRs).

In several embodiments, cells are engineered to express a cytotoxic receptor complex comprising a full length NKG2D as an extracellular component to recognize ligands on the surface of tumor cells (e.g., liver cells). In one embodiment, full length NKG2D has the nucleic acid sequence of SEQ ID NO: 27. In several embodiments, the full length NKG2D, or functional fragment thereof is human NKG2D. Additional information about chimeric receptors for use in the presently disclosed methods and compositions can be found in PCT Patent Publication No. WO/2018/183385, which is incorporated in its entirety by reference herein.

In several embodiments, cells are engineered to express a cytotoxic receptor complex comprising a functional fragment of NKG2D as an extracellular component to recognize ligands on the surface of tumor cells or other diseased cells. In one embodiment, the functional fragment of NKG2D has the nucleic acid sequence of SEQ ID NO: 25. In several embodiments, the fragment of NKG2D is at least 70%, at least 75%, at least 80%, at least 85%, at least 90%, or at least 95% homologous with full-length wild-type NKG2D. In several embodiments, the fragment may have one or more additional mutations from SEQ ID NO: 25, but retains, or in some embodiments, has enhanced, ligand-binding function. In several embodiments, the functional fragment of NKG2D comprises the amino acid sequence of SEQ ID NO: 26. In several embodiments, the NKG2D fragment is provided as a dimer, trimer, or other concatameric format, such embodiments providing enhanced ligand-binding activity. In several embodiments, the sequence encoding the NKG2D fragment is optionally fully or partially codon optimized. In one embodiment, a sequence encoding a codon optimized NKG2D fragment comprises the sequence of SEQ ID NO: 28. Advantageously, according to several embodiments, the functional fragment lacks its native transmembrane or intracellular domains but retains its ability to bind ligands of NKG2D as well as transduce activation signals upon ligand binding. A further advantage of such fragments is that expression of DAP10 to localize NKG2D to the cell membrane is not required. Thus, in several embodiments, the cytotoxic receptor complex encoded by the polypeptides disclosed herein does not comprise DAP10. In several embodiments, immune cells, such as NK or T cells (e.g., non-alloreactive T cells engineered according to embodiments disclosed herein), are engineered to express one or more chimeric receptors that target, for example CD19, CD123, CD70, Her2, mesothelin, Claudin 6, BCMA, EGFR, and an NKG2D ligand, such as MICA, MICB, ULBP1, ULBP2, ULBP3, ULBP4, ULBP5, and/or ULBP6. Such cells, in several embodiments, also co-express mbIL15.

In several embodiments, the cytotoxic receptor complexes are configured to dimerize. Dimerization may comprise homodimers or heterodimers, depending on the embodiment. In several embodiments, dimerization results in improved ligand recognition by the cytotoxic receptor complexes (and hence the NK cells expressing the receptor), resulting in a reduction in (or lack) of adverse toxic effects. In several embodiments, the cytotoxic receptor complexes employ internal dimers, or repeats of one or more component subunits. For example, in several embodiments, the cytotoxic receptor complexes may optionally comprise a first NKG2D extracellular domain coupled to a second NKG2D extracellular domain, and a transmembrane/signaling region (or a separate transmembrane region along with a separate signaling region).

In several embodiments, the various domains/subdomains are separated by a linker such as, a GS3 linker (SEQ ID NO: 15 and 16, nucleotide and protein, respectively) is used (or a GSn linker). Other linkers used according to various embodiments disclosed herein include, but are not limited to those encoded by SEQ ID NO: 17, 19, 21 or 23. This provides the potential to separate the various component parts of the receptor complex along the polynucleotide, which can enhance expression, stability, and/or functionality of the receptor complex.

### Cytotoxic Signaling Complex

Some embodiments of the compositions and methods described herein relate to a chimeric receptor, such as a chimeric antigen receptor (e.g., a CAR directed to CD19, CD123, CD70, Her2, mesothelin, Claudin 6, BCMA, or EGFR (among others), or a chimeric receptor directed against an NKG2D ligand, such as MICA, MICB, ULBP1, ULBP2, ULBP3, ULBP4, ULBP5, and/or ULBP6) that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells.

In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. Moreover, in some embodiments, a domain may serve multiple functions, for example, a transmembrane domain may also serve to provide signaling function.

### Transmembrane Domains

Some embodiments of the compositions and methods described herein relate to chimeric receptors (e.g., tumor antigen-directed CARs and/or ligand-directed chimeric receptors) that comprise a transmembrane domain. Some embodiments include a transmembrane domain from NKG2D or another transmembrane protein. In several embodiments in which a transmembrane domain is employed, the portion of the transmembrane protein employed retains at least a portion of its normal transmembrane domain.

In several embodiments, however, the transmembrane domain comprises at least a portion of CD8, a transmembrane glycoprotein normally expressed on both T cells and NK cells. In several embodiments, the transmembrane domain comprises CD8a. In several embodiments, the transmembrane domain is referred to as a “hinge”. In several embodiments, the “hinge” of CD8α has the nucleic acid sequence of SEQ ID NO: 1. In several embodiments, the CD8α hinge is truncated or modified and is at least 70%, at least 75%, at least 80%, at least 85%, at least 90%, at least 95% homologous with the CD8αhaving the sequence of SEQ ID NO: 1. In several embodiments, the “hinge” of CD8α comprises the amino acid sequence of SEQ ID NO: 2. In several embodiments, the CD8α can be truncated or modified, such that it is at least 70%, at least 75%, at least 80%, at least 85%, at least 90%, at least 95% homologous with the sequence of SEQ ID NO: 2.

In several embodiments, the transmembrane domain comprises a CD8α transmembrane region. In several embodiments, the CD8α transmembrane domain has the nucleic acid sequence of SEQ ID NO: 3. In several embodiments, the CD8α hinge is truncated or modified and is at least 70%, at least 75%, at least 80%, at least 85%, at least 90%, at least 95% homologous with the CD8α having the sequence of SEQ ID NO: 3. In several embodiments, the CD8α transmembrane domain comprises the amino acid sequence of SEQ ID NO: 4. In several embodiments, the CD8α hinge is truncated or modified and is at least 70%, at least 75%, at least 80%, at least 85%, at least 90%, at least 95% homologous with the CD8αhaving the sequence of SEQ ID NO: 4.

Taken together in several embodiments, the CD8 hinge/transmembrane complex is encoded by the nucleic acid sequence of SEQ ID NO: 13. In several embodiments, the CD8 hinge/transmembrane complex is truncated or modified and is at least 70%, at least 75%, at least 80%, at least 85%, at least 90%, at least 95% homologous with the CD8 hinge/transmembrane complex having the sequence of SEQ ID NO: 13. In several embodiments, the CD8 hinge/transmembrane complex comprises the amino acid sequence of SEQ ID NO: 14. In several embodiments, the CD8 hinge/transmembrane complex hinge is truncated or modified and is at least 70%, at least 75%, at least 80%, at least 85%, at least 90%, at least 95% homologous with the CD8 hinge/transmembrane complex having the sequence of SEQ ID NO: 14.

In some embodiments, the transmembrane domain comprises a CD28 transmembrane domain or a fragment thereof. In several embodiments, the CD28 transmembrane domain comprises the amino acid sequence of SEQ ID NO: 30. In several embodiments, the CD28 transmembrane domain complex hinge is truncated or modified and is at least 70%, at least 75%, at least 80%, at least 85%, at least 90%, at least 95% homologous with the CD28 transmembrane domain having the sequence of SEQ ID NO: 30.

### Co-Stimulatory Domains

Some embodiments of the compositions and methods described herein relate to chimeric receptors (e.g., tumor antigen-directed CARs and/or tumor ligand-directed chimeric receptors) that comprise a co-stimulatory domain. In addition the various the transmembrane domains and signaling domain (and the combination transmembrane/signaling domains), additional co-activating molecules can be provided, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such co-stimulatory domains are engineered to be membrane bound, acting as autocrine stimulatory molecules (or even as paracrine stimulators to neighboring cells). In several embodiments, NK cells are engineered to express membrane-bound interleukin 15 (mbIL15). In such embodiments, mbIL15 expression on the NK enhances the cytotoxic effects of the engineered NK cell by enhancing the proliferation and/or longevity of the NK cells. In several embodiments, T cells, such as the genetically engineered non-alloreactive T cells disclosed herein are engineered to express membrane-bound interleukin 15 (mbIL15). In such embodiments, mbIL15 expression on the T cell enhances the cytotoxic effects of the engineered T cell by enhancing the activity and/or propagation (e.g., longevity) of the engineered T cells. In several embodiments, mbIL15 has the nucleic acid sequence of SEQ ID NO: 11. In several embodiments, mbIL15 can be truncated or modified, such that it is at least 70%, at least 75%, at least 80%, at least 85%, at least 90%, at least 95% homologous with the sequence of SEQ ID NO: 11. In several embodiments, the mbIL15 comprises the amino acid sequence of SEQ ID NO: 12. In several embodiments, the mbIL15 is truncated or modified and is at least 70%, at least 75%, at least 80%, at least 85%, at least 90%, at least 95% homologous with the mbIL15 having the sequence of SEQ ID NO: 12.

In some embodiments, the tumor antigen-directed CARs and/or tumor ligand-directed chimeric receptors are encoded by a polynucleotide that includes one or more cytosolic protease cleavage sites, for example a T2A cleavage site, a P2A cleavage site, an E2A cleavage site, and/or a F2A cleavage site. Such sites are recognized and cleaved by a cytosolic protease, which can result in separation (and separate expression) of the various component parts of the receptor encoded by the polynucleotide. As a result, depending on the embodiment, the various constituent parts of an engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site has the nucleic acid sequence of SEQ ID NO: 9. In several embodiments, T2A cleavage site can be truncated or modified, such that it is at least 70%, at least 75%, at least 80%, at least 85%, at least 90%, at least 95% homologous with the sequence of SEQ ID NO: 9. In several embodiments, the T2A cleavage site comprises the amino acid sequence of SEQ ID NO: 10. In several embodiments, the T2A cleavage site is truncated or modified and is at least 70%, at least 75%, at least 80%, at least 85%, at least 90%, at least 95% homologous with the T2A cleavage site having the sequence of SEQ ID NO: 10.

### Signaling Domains

Some embodiments of the compositions and methods described herein relate to a chimeric receptor (e.g., tumor antigen-directed CARs and/or tumor ligand-directed chimeric receptors) that includes a signaling domain. For example, immune cells engineered according to several embodiments disclosed herein may comprise at least one subunit of the CD3 T cell receptor complex (or a fragment thereof). In several embodiments, the signaling domain comprises the CD3 zeta subunit. In several embodiments, the CD3 zeta is encoded by the nucleic acid sequence of SEQ ID NO: 7. In several embodiments, the CD3 zeta can be truncated or modified, such that it is at least 70%, at least 75%, at least 80%, at least 85%, at least 90%, at least 95% homologous with the CD3 zeta having the sequence of SEQ ID NO: 7. In several embodiments, the CD3 zeta domain comprises the amino acid sequence of SEQ ID NO: 8. In several embodiments, the CD3 zeta domain is truncated or modified and is at least 70%, at least 75%, at least 80%, at least 85%, at least 90%, at least 95% homologous with the CD3 zeta domain having the sequence of SEQ ID NO: 8.

In several embodiments, unexpectedly enhanced signaling is achieved through the use of multiple signaling domains whose activities act synergistically. For example, in several embodiments, the signaling domain further comprises an OX40 domain. In several embodiments, the OX40 domain is an intracellular signaling domain. In several embodiments, the OX40 intracellular signaling domain has the nucleic acid sequence of SEQ ID NO: 5. In several embodiments, the OX40 intracellular signaling domain can be truncated or modified, such that it is at least 70%, at least 75%, at least 80%, at least 85%, at least 90%, at least 95% homologous with the OX40 having the sequence of SEQ ID NO: 5. In several embodiments, the OX40 intracellular signaling domain comprises the amino acid sequence of SEQ ID NO: 6. In several embodiments, the OX40 intracellular signaling domain is truncated or modified and is at least 70%, at least 75%, at least 80%, at least 85%, at least 90%, at least 95% homologous with the OX40 intracellular signaling domain having the sequence of SEQ ID NO: 6. In several embodiments, OX40 is used as the sole transmembrane/signaling domain in the construct, however, in several embodiments, OX40 can be used with one or more other domains. For example, combinations of OX40 andCD3zeta are used in some embodiments. By way of further example, combinations of CD28, OX40, 4-1 BB, and/or CD3zeta are used in some embodiments.

In several embodiments, the signaling domain comprises a 4-1 BB domain. In several embodiments, the 4-1 BB domain is an intracellular signaling domain. In several embodiments, the 4-1 BB intracellular signaling domain comprises the amino acid sequence of SEQ ID NO: 29. In several embodiments, the 4-1 BB intracellular signaling domain is truncated or modified and is at least 70%, at least 75%, at least 80%, at least 85%, at least 90%, at least 95% homologous with the 4-1BB intracellular signaling domain having the sequence of SEQ ID NO: 29. In several embodiments, 4-1 BB is used as the sole transmembrane/signaling domain in the construct, however, in several embodiments, 4-1BB can be used with one or more other domains. For example, combinations of 4-1 BB andCD3zeta are used in some embodiments. By way of further example, combinations of CD28, OX40, 4-1 BB, and/or CD3zeta are used in some embodiments.

In several embodiments, the signaling domain comprises a CD28 domain. In several embodiments the CD28 domain is an intracellular signaling domain. In several embodiments, the CD28 intracellular signaling domain comprises the amino acid sequence of SEQ ID NO: 31. In several embodiments, the CD28 intracellular signaling domain is truncated or modified and is at least 70%, at least 75%, at least 80%, at least 85%, at least 90%, at least 95% homologous with the CD28 intracellular signaling domain having the sequence of SEQ ID NO: 31. In several embodiments, CD28 is used as the sole transmembrane/signaling domain in the construct, however, in several embodiments, CD28 can be used with one or more other domains. For example, combinations of CD28 andCD3zeta are used in some embodiments. By way of further example, combinations of CD28, OX40, 4-1 BB, and/or CD3zeta are used in some embodiments.

### Cytotoxic Receptor Complex Constructs

Some embodiments of the compositions and methods described herein relate to chimeric antigen receptors, such as a CD19-directed chimeric receptor, as well as chimeric receptors, such as an activating chimeric receptor (ACR) that targets ligands of NKG2D. The expression of these cytotoxic receptors complexes in immune cells, such as genetically modified non-alloreactive T cells and/or NK cells, allows the targeting and destruction of particular target cells, such as cancerous cells. Non-limiting examples of such cytotoxic receptor complexes are discussed in more detail below.

### Chimeric Antigen Receptor Cytotoxic Receptor Complex Constructs

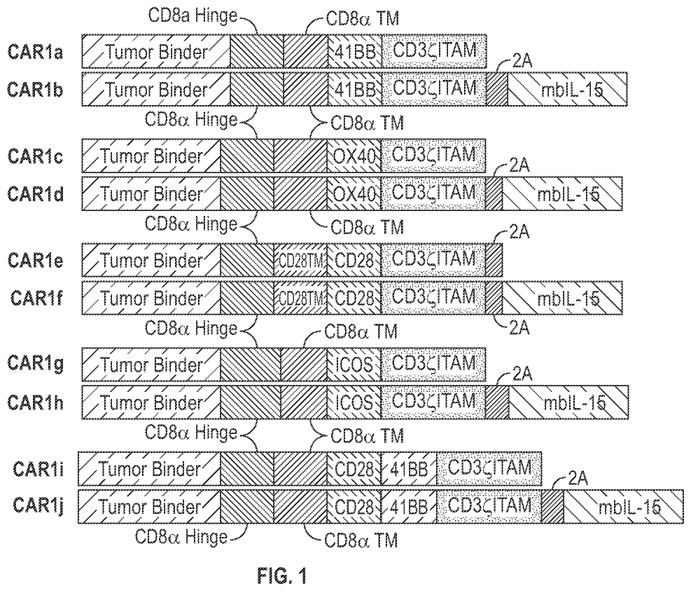

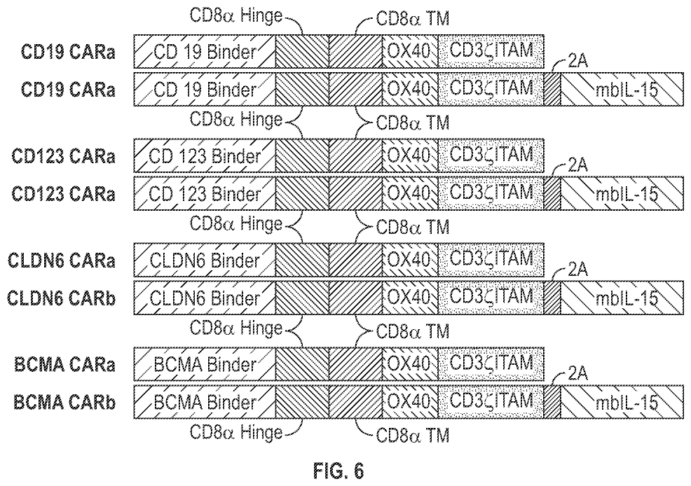

In several embodiments, there are provided for herein a variety of cytotoxic receptor complexes (also referred to as cytotoxic receptors) are provided for herein with the general structure of a chimeric antigen receptor. FIGS. 1-7 schematically depict non-limiting schematics of constructs that include an tumor binding moiety that binds to tumor antigens or tumor-associated antigens expressed on the surface of cancer cells and activates the engineered cell expressing the chimeric antigen receptor. FIG. 6 shows a schematic of a chimeric receptor complex, with an NKG2D activating chimeric receptor as a non-limiting example (see NKG2D ACRa and ACRb). FIG. 6 shows a schematic of a bispecific CAR/chimeric receptor complex, with an NKG2D activating chimeric receptor as a non-limiting example (see Bi-spec CAR/ACRa and CAR/ACRb).

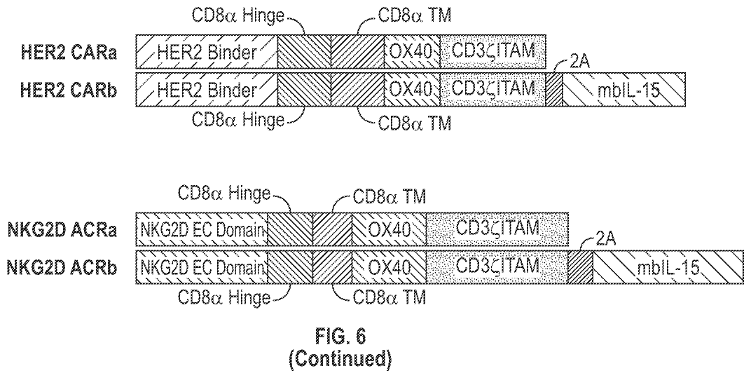

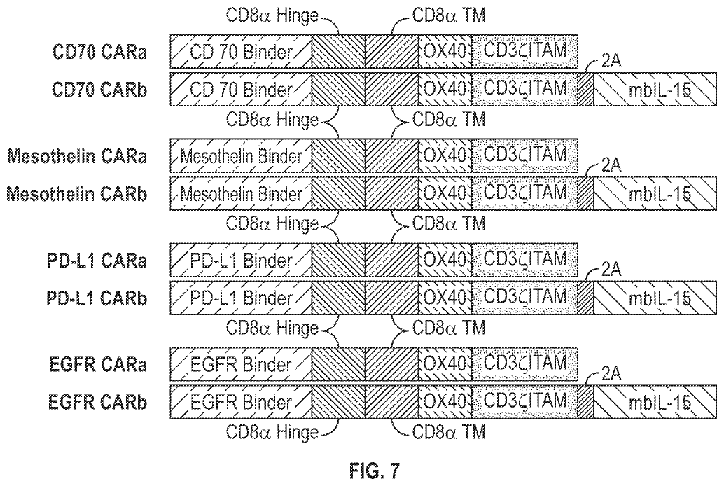

As shown in the figures, several embodiments of the chimeric receptor include an anti-tumor binder, a CD8α hinge domain, an Ig4 SH domain (or hinge), a CD8α transmembrane domain, a CD28 transmembrane domain, an OX40 domain, a 4-1BB domain, a CD28 domain, a CD3(ITAM domain or subdomain, a CD3zeta domain, an NKp80 domain, a CD16 IC domain, a 2A cleavage site, and a membrane-bound IL-15 domain (though, as above, in several embodiments soluble IL-15 is used). In several embodiments, the binding and activation functions are engineered to be performed by separate domains. Several embodiments relate to complexes with more than one tumor binder moiety or other binder/activation moiety. In some embodiments, the binder/activation moiety targets other markers besides CD19, such as a cancer target described herein. For example, FIGS. 6 and 7 depict schematics of non-limiting examples of CAR constructs that target different antigens, such as CD123, CLDN6, BCMA, HER2, CD70, Mesothelia, PD-L1, and EGFR. In several embodiments, the general structure of the chimeric antigen receptor construct includes a hinge and/or transmembrane domain. These may, in some embodiments, be fulfilled by a single domain, or a plurality of subdomains may be used, in several embodiments. The receptor complex further comprises a signaling domain, which transduces signals after binding of the homing moiety to the target cell, ultimately leading to the cytotoxic effects on the target cell. In several embodiments, the complex further comprises a co-stimulatory domain, which operates, synergistically, in several embodiments, to enhance the function of the signaling domain. Expression of these complexes in immune cells, such as T cells and/or NK cells, allows the targeting and destruction of particular target cells, such as cancerous cells that express a given tumor marker. Some such receptor complexes comprise an extracellular domain comprising an anti-CD19 moiety, or CD19-binding moiety, that binds CD19 on the surface of target cells and activates the engineered cell. The CD3zeta ITAM subdomain may act in concert as a signaling domain. The IL-15 domain, e.g., mblL-15 domain, may act as a co-stimulatory domain. The IL-15 domain, e.g. mblL-15 domain, may render immune cells (e.g., NK or T cells) expressing it particularly efficacious against target tumor cells. It shall be appreciated that the IL-15 domain, such as an mblL-15 domain, can, in accordance with several embodiments, be encoded on a separate construct. Additionally, each of the components may be encoded in one or more separate constructs. In some embodiments, the cytotoxic receptor or CD19-directed receptor comprises a sequence of amino acids that is at least 70%, 75%, 80%, 85%, 90%, 91%, 92%, 93%, 94%, 95%, 96%, 97%, 98%, 99%, or more, or a range defined by any two of the aforementioned percentages, identical to the sequence of SEQ ID NO: 34.

Depending on the embodiment, various binders can be used to target CD19. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CD19. In several embodiments, an scFv is provided that is specific for CD19. In several embodiments, the antibody or scFv specific for CD19 comprises a heavy chain variable comprising the amino acid sequence of SEQ ID NO: 104 or 106. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 104 or 106. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 104 or 106. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 104 or 106.

In several embodiments, the antibody or scFv specific for CD19 comprises a light chain variable comprising the amino acid sequence of any of SEQ ID NO. 105 or 107. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 105 or 107. In some embodiments, the light chain variable comprises a sequence of amino acids that is encoded by a polynucleotide that hybridizes under moderately stringent conditions to the complement of a polynucleotide that encodes a light chain variable of SEQ ID NO. 105 or 107. In some embodiments, the light chain variable domain comprises a sequence of amino acids that is encoded by a polynucleotide that hybridizes under stringent conditions to the complement of a polynucleotide that encodes a light chain variable domain of SEQ ID NO. 105 or 107.

In several embodiments, the anti-CD19 antibody or scFv comprises one, two, or three heavy chain complementarity determining region (CDR) and one, two, or three light chain CDRs. In several embodiments, a first heavy chain CDR has the amino acid sequence of SEQ ID NO: 111. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 111. In several embodiments, a second heavy chain CDR has the amino acid sequence of SEQ ID NO: 112, 113, or 114. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 112, 113, or 114. In several embodiments, a third heavy chain CDR has the amino acid sequence of SEQ ID NO: 115. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 115.

In several embodiments, a first light chain CDR has the amino acid sequence of SEQ ID NO: 108. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 108. In several embodiments, a second light chain CDR has the amino acid sequence of SEQ ID NO: 109. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 109. In several embodiments, a third light chain CDR has the amino acid sequence of SEQ ID NO: 110. In some embodiments, the third light chain CDR comprises a sequence of amino acids that is at least 70%, 75%, 80%, 85%, 90%, 91%, 92%, 93%, 94%, 95%, 96%, 97%, 98%, 99% or more, identical to the sequence of SEQ ID NO. 110.

In several embodiments, there is provided an anti-CD19 CAR comprising the amino acid sequence of SEQ ID NO. 116. In some embodiments, there is provided an anti-CD19 CAR comprising a sequence of amino acids that is at least 70%, 75%, 80%, 85%, 90%, 91%, 92%, 93%, 94%, 95%, 96%, 97%, 98%, 99% or more, identical to the sequence of SEQ ID NO. 116.

In one embodiment, there is provided a polynucleotide encoding a Tumor Binder/CD8hinge-CD8TM/OX40/CD3zeta chimeric antigen receptor complex (see FIG. 1, CAR1c). The polynucleotide comprises or is composed of tumor binder, a CD8α hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a tumor binder/CD8hinge-CD8TM/OX40/CD3zeta/2A/mIL-15 chimeric antigen receptor complex (see FIG. 1, CAR 1d). The polynucleotide comprises or is composed of a Tumor Binder, a CD8α hinge, a CD8α transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

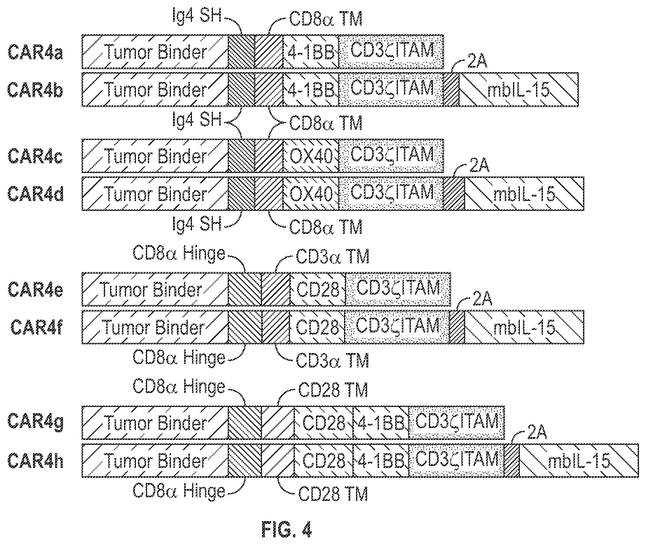

In several embodiments, there is provided a polynucleotide encoding a Tumor Binder/Ig4SH-CD8TM/4-1 BB/CD3zeta chimeric antigen receptor complex (see FIG. 4,CAR4a). The polynucleotide comprises or is composed of a Tumor Binder, an Ig4 SH domain, a CD8α transmembrane domain, a 4-1 BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/4-1 BB/CD3zeta/2A/mIL-15 chimeric antigen receptor complex (see FIG. 4, CAR4b). The polynucleotide comprises or is composed of a Tumor Binder, a Ig4 SH domain, a CD8α transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/CD3zeta chimeric antigen receptor complex (see FIG. 1, CAR1e). The polynucleotide comprises or is composed of a Tumor Binder, a CD8α hinge, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/CD3zeta/2A/mIL-15 chimeric antigen receptor complex (see FIG. 1, CAR1f). The polynucleotide comprises or is composed of a Tumor Binder, a CD8α hinge, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

It shall be appreciated that certain sequence variability, extensions, and/or truncations of the disclosed sequences may result when combining sequences, as a result of, for example, ease or efficiency in cloning (e.g., for creation of a restriction site).

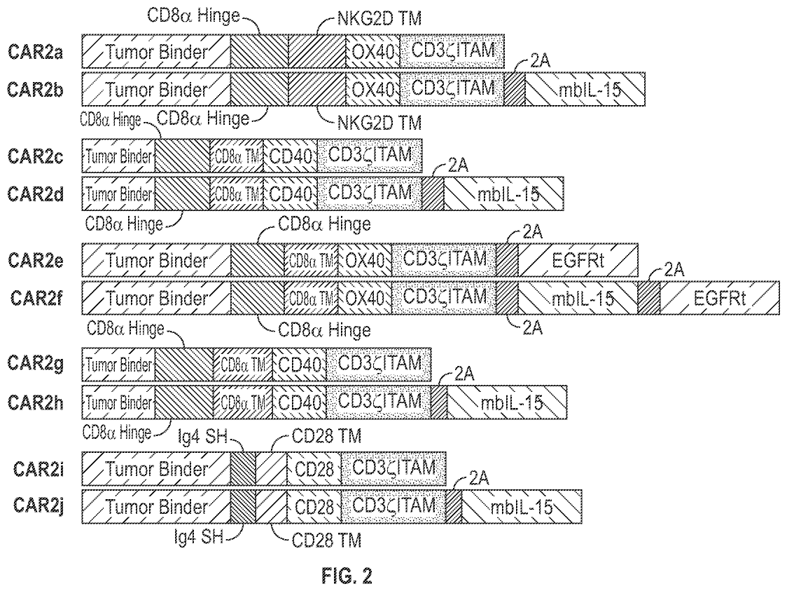

In several embodiments, there is provided a polynucleotide encoding a Tumor Binder/Ig4SH-CD28TM/CD28/CD3zeta chimeric antigen receptor complex (see FIG. 2, CAR2i). The polynucleotide comprises or is composed of a Tumor Binder, an Ig4 SH domain, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD28TM/CD28/CD3zeta/2A/mIL-15 chimeric antigen receptor complex (see FIG. 2, CAR2j). The polynucleotide comprises or is composed of a Tumor Binder, an Ig4 SH domain, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/OX40/CD3zeta chimeric antigen receptor complex (see FIG. 4, CAR4c). The polynucleotide comprises or is composed of a Tumor Binder, a Ig4 SH domain, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/OX40/CD3zeta/2A/mIL-15 chimeric antigen receptor complex (see FIG. 4, CAR4d). The polynucleotide comprises or is composed of a Tumor Binder, a Ig4 SH domain, a CD8α transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD3aTM/CD28/CD3zeta chimeric antigen receptor complex (see FIG. 4, CAR4e). The polynucleotide comprises or is composed of a Tumor Binder, a CD8α hinge, a CD3a transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD3aTM/CD28/CD3zeta/2A/mIL-15 chimeric antigen receptor complex (see FIG. 4, CAR4f). The polynucleotide comprises or is composed of a Tumor Binder, a CD8α hinge, a CD3a transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/4-1 BB/CD3zeta chimeric antigen receptor complex (see FIG. 4, CAR 4g). The polynucleotide comprises or is composed of a Tumor Binder, a CD8α hinge, a CD28 transmembrane domain, a CD28 domain, a 4-1 BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/4-1 BB/CD3zeta/2A/mIL-15 chimeric antigen receptor complex (see FIG. 4, CAR 4h). The polynucleotide comprises or is composed of a Tumor Binder, a CD8α hinge, a CD28 transmembrane domain, a CD28 domain, a 4-1 BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

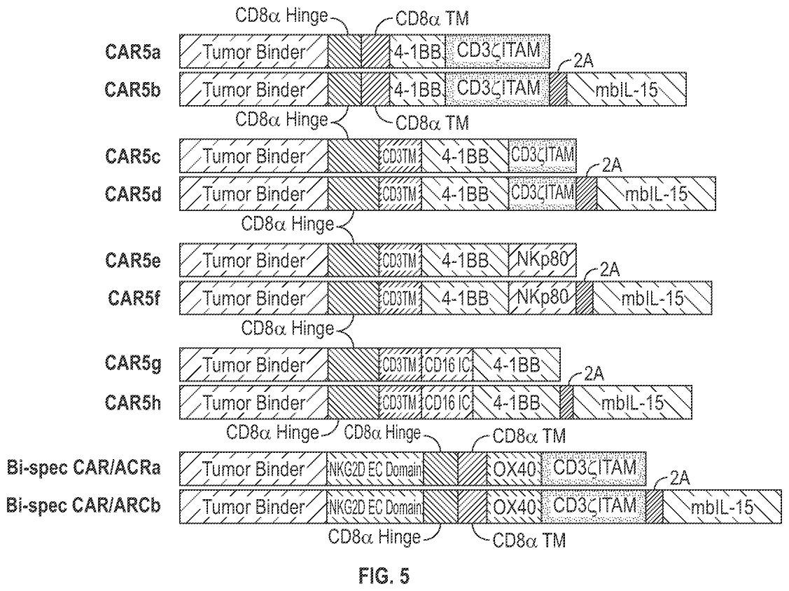

In several embodiments, there is provided a polynucleotide encoding a Tumor Binder/CD8 alpha hinge/CD8 alpha TM/4-1 BB/CD3zeta chimeric antigen receptor complex (see FIG. 5, CAR5a). The polynucleotide comprises or is composed of an anti-CD19 moiety, a CD8α hinge, a CD8α transmembrane domain, a 4-1 BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD8 alpha TM/4-1BB/CD3zeta/2A/mIL-15 chimeric antigen receptor complex (see FIG. 5, CAR 5b). The polynucleotide comprises or is composed of a Tumor Binder, a CD8α hinge, a CD8αtransmembrane domain, a 4-1 BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1 BB/CD3zeta chimeric antigen receptor complex (see FIG. 5, CAR5c). The polynucleotide comprises or is composed of a Tumor Binder, a CD8α hinge, a CD3 transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1 BB/CD3zeta/2A/mIL-15 chimeric antigen receptor complex (see FIG. 5, CAR5d). The polynucleotide comprises or is composed of a Tumor Binder, a CD8α hinge, a CD8α transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/NKp80 chimeric antigen receptor complex (see FIG. 5,CAR5e). The polynucleotide comprises or is composed of a Tumor Binder, a CD8α hinge, a CD3 transmembrane domain, a 4-1BB domain, and an NKp80 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/NKp80/2A/mIL-15 chimeric antigen receptor complex (see FIG. 5, CAR5f). The polynucleotide comprises or is composed of a Tumor Binder, a CD8α hinge, a CD8α transmembrane domain, a 4-1BB domain, an NKp80 domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/CD16 intracellular domain/4-1BB chimeric antigen receptor complex (see FIG. 5, CAR5g). The polynucleotide comprises or is composed of a Tumor Binder, a CD8α hinge, a CD3 transmembrane domain, CD16 intracellular domain, and a 4-1BB domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/CD16/4-1BB/2A/mIL-15 chimeric antigen receptor complex (see FIG. 5, CAR5h). The polynucleotide comprises or is composed of a Tumor Binder, a CD8α hinge, a CD8α transmembrane domain, a CD16 intracellular domain, a 4-1BB domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/NKG2D Extracellular Domain/CD8hinge-CD8TM/OX40/CD3zeta chimeric antigen receptor complex (see FIG. 5, Bi-spec CAR/ACRa). The polynucleotide comprises or is composed of a Tumor Binder, an NKG2D extracellular domain (either full length or a fragment), a CD8α hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/NKG2D EC Domain/CD8hinge-CD8TM/OX40/CD3zeta/2A/mIL-15 chimeric antigen receptor complex (see FIG. 5, Bi-spec CAR/ACRb). The polynucleotide comprises or is composed of a Tumor Binder, an NKG2D extracellular domain (either full length or a fragment), a CD8α hinge, a CD8αtransmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8TM/4-1 BB/CD3zeta chimeric antigen receptor complex (see FIG. 1, CAR1a). The polynucleotide comprises or is composed of a Tumor Binder, a CD8α hinge, a CD8α transmembrane domain, a 4-1 BB domain, and a CD3zeta domain. By way of non-limiting embodiment, there is provided herein an anti-CD19/CD8hinge/CD8TM/4-1BB/CD3zeta chimeric antigen receptor complex. In several embodiments, this receptor complex is encoded by a nucleic acid molecule having the sequence of SEQ ID NO: 85. In several embodiments, a nucleic acid sequence encoding an CAR1a chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 85. In several embodiments, the chimeric receptor comprises the amino acid sequence of SEQ ID NO: 86. In several embodiments, a CAR1a chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 86. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided an CAR1a construct that further comprises mbIL15, as disclosed herein (see e.g., FIG. 1 CAR1 b).

In several embodiments, there is provided a polynucleotide encoding a Tumor Binder/CD8hinge/CD8TM/OX40/CD3zeta chimeric antigen receptor complex (see FIG. 1, CAR1c). The polynucleotide comprises or is composed of a Tumor Binder, a CD8α hinge, a CD8α transmembrane domain, an OX40 domain, and a CD3zeta domain. In several embodiments, the chimeric antigen receptor further comprises mbIL15 (see FIG. 1, CAR1d). By way of non-limiting embodiment, there is provided herein an anti CD19/CD8hinge/CD8TM/OX40/CD3zeta/2A/mIL-15 chimeric antigen receptor. In such embodiments, the polynucleotide comprises or is composed of an anti-CD19 scFv, a CD8α hinge, a CD8αtransmembrane domain, an OX40 domain, a CD3zeta domain, a 2A cleavage site, and an mblL-15 domain as described herein. In several embodiments, this receptor complex is encoded by a nucleic acid molecule having the sequence of SEQ ID NO: 59. In several embodiments, a nucleic acid sequence encoding an CAR1d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 59. In several embodiments, the chimeric receptor comprises the amino acid sequence of SEQ ID NO: 60. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 60. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD28TM/CD28/CD3zeta chimeric antigen receptor complex (see FIG. 1, CAR1e). The polynucleotide comprises or is composed of a Tumor Binder, a CD8α hinge, a CD28 transmembrane domain, CD28 signaling domain, and a CD3zeta domain. In several embodiments, the chimeric antigen receptor further comprises mbIL15 (see FIG. 1, CAR1d). By way of non-limiting embodiment, there is provided herein an anti-CD19moiety/CD8hinge/CD28TM/CD28/CD3zeta/2A/mIL15 chimeric antigen receptor complex. In such embodiments, the polynucleotide comprises or is composed of an anti-CD19 scFv, a CD8α hinge, a CD28 transmembrane domain, CD28 signaling domain, a CD3zeta domain a 2A cleavage site, and an mblL-15 domain as described herein. In several embodiments, this receptor complex is encoded by a nucleic acid molecule having the sequence of SEQ ID NO: 61. In several embodiments, a nucleic acid sequence encoding an CAR1d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 61. In several embodiments, the chimeric receptor comprises the amino acid sequence of SEQ ID NO: 62. In several embodiments, a CAR1d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 62. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/ICOS/CD3zeta chimeric antigen receptor complex (see FIG. 1, CAR1g). The polynucleotide comprises or is composed of a Tumor Binder, a CD8α hinge, a CD8α transmembrane domain, inducible costimulator (ICOS) signaling domain, and a CD3zeta domain. In several embodiments, the chimeric antigen receptor further comprises mbIL15 (see 1, CAR1 h). By way of non-limiting embodiment, there is provided herein an anti-CD19moiety/CD8hinge/CD8aTM/ICOS/CD3zeta/2A/mIL15 chimeric antigen receptor complex. In such embodiments, the polynucleotide comprises or is composed of an anti-CD19 scFv, a CD8α hinge, a CD8α transmembrane domain, inducible costimulator (ICOS) signaling domain, a CD3zeta domain, a 2A cleavage site, and an mblL-15 domain as described herein. In several embodiments, this receptor complex is encoded by a nucleic acid molecule having the sequence of SEQ ID NO: 63. In several embodiments, a nucleic acid sequence encoding an CAR1 h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 63. In several embodiments, the chimeric receptor comprises the amino acid sequence of SEQ ID NO: 64. In several embodiments, a CAR1 h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 64. In several embodiments, the CAR1 h scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

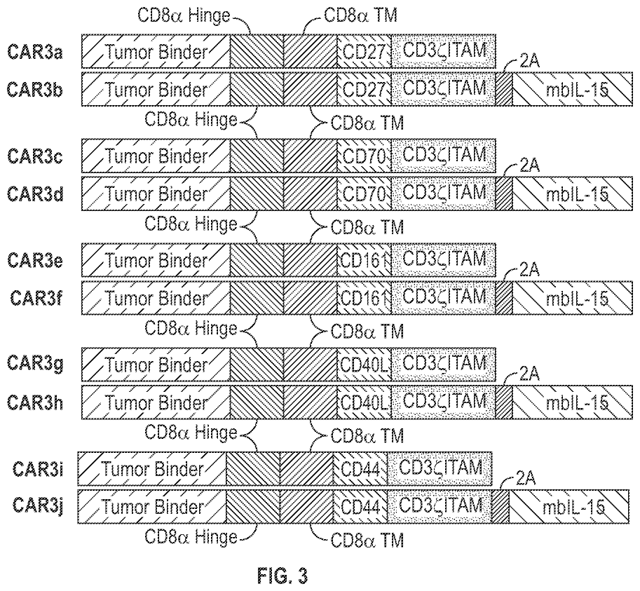

In several embodiments, there is provided a polynucleotide encoding a Tumor Binder/CD8hinge/CD8aTM/CD28/4-1 BB/CD3zeta chimeric antigen receptor complex (see FIG. 1, CAR1 i). The polynucleotide comprises or is composed of a Tumor Binder, a CD8α hinge, a CD8α transmembrane domain, a CD28 signaling domain, a 4-1BB signaling domain, and a CD3zeta domain. In several embodiments, the chimeric antigen receptor further comprises mbIL15 (see FIG. 3A, NK19-4b). By way of non-limiting embodiment, there is provided herein an anti-CD19moiety/CD8hinge/CD8aTM/CD28/4-1 BB/CD3zeta/2A/mIL-15. In such embodiments, the polynucleotide comprises or is composed of an anti-CD19 scFv, a CD8α hinge, a CD8α transmembrane domain, a CD28 signaling domain, a 4-1 BB signaling domain, a CD3zeta domain, a 2A cleavage site, and an mblL-15 domain as described herein. In several embodiments, this receptor complex is encoded by a nucleic acid molecule having the sequence of SEQ ID NO: 65. In several embodiments, a nucleic acid sequence encoding an CAR1 h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 65. In several embodiments, the chimeric receptor comprises the amino acid sequence of SEQ ID NO: 66. In several embodiments, a CAR1 h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 66. In several embodiments, the CAR1 h scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/NKG2DTM/OX40/CD3zeta chimeric antigen receptor complex (see FIG. 2, CAR2a). The polynucleotide comprises or is composed of a Tumor Binder, a CD8α hinge, a NKG2D transmembrane domain, an OX40 signaling domain, and a CD3zeta domain. In several embodiments, the chimeric antigen receptor further comprises mbIL15 (see FIG. 2, CAR2b). By way of non-limiting embodiment, there is provided herein an anti-CD19moiety/CD8hinge/NKG2DTM/OX40/CD3zeta/2A/mIL-15 chimeric antigen receptor complex. In such embodiments, the polynucleotide comprises or is composed of an anti-CD19 scFv, a CD8α hinge, a NKG2D transmembrane domain, an OX40 signaling domain, a CD3zeta domain, a 2A cleavage site, and an mblL-15 domain as described herein. In several embodiments, this receptor complex is encoded by a nucleic acid molecule having the sequence of SEQ ID NO: 67. In several embodiments, a nucleic acid sequence encoding an CAR2b chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 67. In several embodiments, the chimeric receptor comprises the amino acid sequence of SEQ ID NO: 68. In several embodiments, a CAR2b chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 68. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40/CD3zeta chimeric antigen receptor complex (see Figure CAR2c). The polynucleotide comprises or is composed of Tumor Binder, a CD8α hinge, a CD8α transmembrane domain, a CD40 signaling domain, and a CD3zeta domain. In several embodiments, the chimeric antigen receptor further comprises mbIL15 (see FIG. 1, CAR2d). By way of non-limiting embodiment, there is provided herein an anti-CD19moiety/CD8hinge/CD8aTM/CD40/CD3zeta/2A/mIL-15 chimeric antigen receptor complex. In such embodiments, the polynucleotide comprises or is composed of an anti-CD19 scFv variable heavy chain, a CD8α hinge, a CD8α transmembrane domain, a CD40 signaling domain, a CD3zeta domain, a 2A cleavage site, and an mblL-15 domain as described herein. In several embodiments, this receptor complex is encoded by a nucleic acid molecule having the sequence of SEQ ID NO: 69. In several embodiments, a nucleic acid sequence encoding an CAR2d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 69. In several embodiments, the chimeric receptor comprises the amino acid sequence of SEQ ID NO: 70. In several embodiments, a CAR2d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 70. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/OX40/CD3zeta/2A/EGFRt chimeric antigen receptor complex (see FIG. 2, CAR2e). The polynucleotide comprises or is composed of a Tumor Binder, a CD8α hinge, a CD8α transmembrane domain, an OX40 signaling domain, a CD3zeta domain, a 2A cleavage side, and a truncated version of the epidermal growth factor receptor (EGFRt). In several embodiments, the chimeric antigen receptor further comprises mbIL15 (see FIG. 2, CAR2f). By way of non-limiting embodiment, there is provided herein an anti-CD19moiety/CD8hinge/CD8aTM/OX40/CD3zeta/2A/mIL-15/2A/EGFRt chimeric antigen receptor complex. In such embodiments, the polynucleotide comprises or is composed of an anti-CD19 scFv, a CD8α hinge, a CD8α transmembrane domain, an OX40 signaling domain, a CD3zeta domain, a 2A cleavage side, a truncated version of the epidermal growth factor receptor (EGFRt), an additional 2A cleavage site, and an mblL-15 domain as described herein. In several embodiments, this receptor complex is encoded by a nucleic acid molecule having the sequence of SEQ ID NO: 71. In several embodiments, a nucleic acid sequence encoding an CAR2f chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 71. In several embodiments, the chimeric receptor comprises the amino acid sequence of SEQ ID NO: 72. In several embodiments, a CAR2f chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 72. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40/CD3zeta chimeric antigen receptor complex (see FIG. 2, CAR2g). The polynucleotide comprises or is composed of a Tumor Binder, a CD8α hinge, a CD8α transmembrane domain, a CD40 signaling domain, and a CD3zeta domain. In several embodiments, the chimeric antigen receptor further comprises mbIL15 (see FIG. 2, CAR2h). By way of non-limiting embodiment, there is provided herein an anti-CD19moiety/CD8hinge/CD8aTM/CD40/CD3zeta/2A/mIL-15 chimeric antigen receptor complex. In such embodiments, the polynucleotide comprises or is composed of an anti-CD19 scFv variable light chain, a CD8α hinge, a CD8α transmembrane domain, a CD40 signaling domain, a CD3zeta domain, a 2A cleavage site, and an mblL-15 domain as described herein. In several embodiments, this receptor complex is encoded by a nucleic acid molecule having the sequence of SEQ ID NO: 73. In several embodiments, a nucleic acid sequence encoding an CAR2h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 73. In several embodiments, the chimeric receptor comprises the amino acid sequence of SEQ ID NO: 74. In several embodiments, a CAR2h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 74. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD27/CD3zeta chimeric antigen receptor complex (see FIG. 3, CAR3a). The polynucleotide comprises or is composed of a Tumor Binder, a CD8α hinge, a CD8α transmembrane domain, a CD27 signaling domain, and a CD3zeta domain. In several embodiments, the chimeric antigen receptor further comprises mbIL15 (see FIG. 3, CAR3b). By way of non-limiting embodiment, there is provided herein an anti-CD19moiety/CD8hinge/CD8aTM/CD27/CD3zeta/2A/mIL-15 chimeric antigen receptor complex. In such embodiments, the polynucleotide comprises or is composed of an anti-CD19 scFv, a CD8α hinge, a CD8α transmembrane domain, a CD27 signaling domain, a CD3zeta domain, a 2A cleavage site, and an mblL-15 domain as described herein. In several embodiments, this receptor complex is encoded by a nucleic acid molecule having the sequence of SEQ ID NO: 75. In several embodiments, a nucleic acid sequence encoding an CAR3b chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 75. In several embodiments, the chimeric receptor comprises the amino acid sequence of SEQ ID NO: 76. In several embodiments, a CAR3b chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 76. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD70/CD3zeta chimeric antigen receptor complex (see FIG. 3, CAR3c). The polynucleotide comprises or is composed of a Tumor Binder, a CD8α hinge, a CD8α transmembrane domain, a CD70 signaling domain, and a CD3zeta domain. In several embodiments, the chimeric antigen receptor further comprises mbIL15 (see FIG. 3, CAR3d). By way of non-limiting embodiment, there is provided herein an anti-CD19moiety/CD8hinge/CD8aTM/CD70/CD3zeta/2A/mIL-15 chimeric antigen receptor complex. In such embodiments, the polynucleotide comprises or is composed of an anti-CD19 scFv, a CD8α hinge, a CD8α transmembrane domain, a CD70 signaling domain, a CD3zeta domain, a 2A cleavage site, and an mblL-15 domain as described herein. In several embodiments, this receptor complex is encoded by a nucleic acid molecule having the sequence of SEQ ID NO: 77. In several embodiments, a nucleic acid sequence encoding an CAR3d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 77. In several embodiments, the chimeric receptor comprises the amino acid sequence of SEQ ID NO: 78. In several embodiments, a CAR3d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 78. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD161/CD3zeta chimeric antigen receptor complex (see FIG. 3, CAR3e). The polynucleotide comprises or is composed of a Tumor Binder, a CD8α hinge, a CD8α transmembrane domain, a CD161 signaling domain, and a CD3zeta domain. In several embodiments, the chimeric antigen receptor further comprises mbIL15 (see FIG. 3, CAR3f). By way of non-limiting embodiment, there is provided herein an anti-CD19moiety/CD8hinge/CD8aTM/CD161/CD3zeta/2A/mIL-15 chimeric antigen receptor complex. In such embodiments, the polynucleotide comprises or is composed of an anti-CD19 scFv, a CD8α hinge, a CD8α transmembrane domain, a CD161 signaling domain, a CD3zeta domain, a 2A cleavage site, and an mblL-15 domain as described herein. In several embodiments, this receptor complex is encoded by a nucleic acid molecule having the sequence of SEQ ID NO: 79. In several embodiments, a nucleic acid sequence encoding an CAR3f chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 79. In several embodiments, the chimeric receptor comprises the amino acid sequence of SEQ ID NO: 80. In several embodiments, a CAR3f chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 80. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40L/CD3zeta chimeric antigen receptor complex (see FIG. 3, CAR3g). The polynucleotide comprises or is composed of a Tumor Binder, a CD8α hinge, a CD8α transmembrane domain, a CD40L signaling domain, and a CD3zeta domain. In several embodiments, the chimeric antigen receptor further comprises mbIL15 (see FIG. 3, CAR3h). By way of non-limiting embodiment, there is provided herein an anti-CD19moiety/CD8hinge/CD8aTM/CD40L/CD3zeta/2A/mIL-15 chimeric antigen receptor complex. In such embodiments, the polynucleotide comprises or is composed of an anti-CD19 scFv, a CD8α hinge, a CD8α transmembrane domain, a CD40L signaling domain, a CD3zeta domain, a 2A cleavage site, and an mblL-15 domain as described herein. In several embodiments, this receptor complex is encoded by a nucleic acid molecule having the sequence of SEQ ID NO: 81. In several embodiments, a nucleic acid sequence encoding an CAR3h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 81. In several embodiments, the chimeric receptor comprises the amino acid sequence of SEQ ID NO: 82. In several embodiments, a CAR3h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 82. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD44/CD3zeta chimeric antigen receptor complex (see FIG. 3, CAR3i). The polynucleotide comprises or is composed of a Tumor Binder, a CD8α hinge, a CD8α transmembrane domain, a CD44 signaling domain, and a CD3zeta domain. In several embodiments, the chimeric antigen receptor further comprises mbIL15 (see FIG. 3, CAR3j). By way of non-limiting embodiment, there is provided herein an anti-CD19moiety/CD8hinge/CD8aTM/CD44/CD3zeta/2A/mIL-15 chimeric antigen receptor complex. In such embodiments, the polynucleotide comprises or is composed of an anti-CD19 scFv, a CD8α hinge, a CD8α transmembrane domain, a CD44 signaling domain, a CD3zeta domain, a 2A cleavage site, and an mblL-15 domain as described herein. In several embodiments, this receptor complex is encoded by a nucleic acid molecule having the sequence of SEQ ID NO: 83. In several embodiments, a nucleic acid sequence encoding an CAR3j chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 83. In several embodiments, the chimeric receptor comprises the amino acid sequence of SEQ ID NO: 84. In several embodiments, a CAR3j chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 84. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding an anti CD123/CD8αhinge/CD8α transmembrane domain/OX40/CD3zeta chimeric antigen receptor complex (see FIG. 6, CD123 CARa). The polynucleotide comprises or is composed of an anti CD123 moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided an CD123 CAR construct that further comprises mbIL15, as disclosed herein (see e.g., FIG. 6, CD123 CARb).

In several embodiments, there is provided a polynucleotide encoding an anti CLDN6/CD8αhinge/CD8α transmembrane domain/OX40/CD3zeta chimeric antigen receptor complex (see FIG. 6, CLDN6 CARa). The polynucleotide comprises or is composed of an anti CLDN6 binding moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a CLDN6 CAR construct that further comprises mbIL15, as disclosed herein (see e.g., FIG. 6, CLDN6 CARb).

Depending on the embodiment, various binders can be used to target CLDN6. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CLDN6. In several embodiments, an scFv is provided that is specific for CLDN6. In several embodiments, the antibody or scFv specific for CLDN6 comprises a heavy chain variable comprising the amino acid sequence of SEQ ID NO. 88. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 88. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 88. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 88.

In several embodiments, the antibody or scFv specific for CLDN6 comprises a light chain variable comprising the amino acid sequence of any of SEQ ID NO. 89, 90, or 91. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 89, 90, or 91. In some embodiments, the light chain variable comprises a sequence of amino acids that is encoded by a polynucleotide that hybridizes under moderately stringent conditions to the complement of a polynucleotide that encodes a light chain variable of SEQ ID NO. 89, 90, or 91. In some embodiments, the light chain variable domain comprises a sequence of amino acids that is encoded by a polynucleotide that hybridizes under stringent conditions to the complement of a polynucleotide that encodes a light chain variable domain of SEQ ID NO. 89, 90, or 91.

In several embodiments, the anti-CLDN6 antibody or scFv comprises one, two, or three heavy chain complementarity determining region (CDR) and one, two, or three light chain CDRs. In several embodiments, a first heavy chain CDR has the amino acid sequence of SEQ ID NO: 92. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 92. In several embodiments, a second heavy chain CDR has the amino acid sequence of SEQ ID NO: 93. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 93. In several embodiments, a third heavy chain CDR has the amino acid sequence of SEQ ID NO: 94. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 94.

In several embodiments, a first light chain CDR has the amino acid sequence of SEQ ID NO: 95, 98, or 101. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 95, 98, or 101. In several embodiments, a second light chain CDR has the amino acid sequence of SEQ ID NO: 96, 99, or 102. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 96, 99, or 102. In several embodiments, a third light chain CDR has the amino acid sequence of SEQ ID NO: 97, 100, or 103. In some embodiments, the third light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 97, 100, or 103.

Advantageously, in several embodiments, the CLDN6 CARs are highly specific to CLDN6 and do not substantially bind to any of CLDN3, 4, or 9.

In several embodiments, there is provided a polynucleotide encoding an anti BCMA/CD8αhinge/CD8α transmembrane domain/OX40/CD3zeta chimeric antigen receptor complex (see FIG. 6, BCMA CARa). The polynucleotide comprises or is composed of an anti BCMA binding moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a BCMA CAR construct that further comprises mbIL15, as disclosed herein (see e.g., FIG. 6, BCMA CARb). Additional information about anti-BCMA CARs for use in the presently disclosed methods and compositions can be found in U.S. Provisional Patent Application No. 62/960,285, which is incorporated in its entirety by reference herein.

In several embodiments, there is provided a polynucleotide encoding an anti HER2/CD8αhinge/CD8α transmembrane domain/OX40/CD3zeta chimeric antigen receptor complex (see FIG. 6, HER2 CARa). The polynucleotide comprises or is composed of an anti HER2 binding moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a HER2 CAR construct that further comprises mbIL15, as disclosed herein (see e.g., FIG. 6, HER2 CARb).

In several embodiments, there is provided a polynucleotide encoding an NKG2D/CD8αhinge/CD8α transmembrane domain/OX40/CD3zeta activating chimeric receptor complex (see FIG. 6, NKG2D ACRa). The polynucleotide comprises or is composed of a fragment of the NKG2D receptor capable of binding a ligand of the NKG2D receptor, a CD8alpha hinge, a CD8α transmembrane domain, an OX40 domain, and a CD3zeta domain as described herein. In several embodiments, this receptor complex is encoded by a nucleic acid molecule comprising the nucleic acid sequence of SEQ ID NO: 145. In yet another embodiment, this chimeric receptor is encoded by the amino acid sequence of SEQ ID NO: 174. In some embodiments, the sequence of the chimeric receptor may vary from SEQ ID NO. 145, but remains, depending on the embodiment, at least 70%, at least 75%, at least 80%, at least 85%, at least 90%, or at least 95% homologous with SEQ ID NO. 145. In several embodiments, while the chimeric receptor may vary from SEQ ID NO. 145, the chimeric receptor retains, or in some embodiments, has enhanced, NK cell activating and/or cytotoxic function. Additionally, in several embodiments, this construct can optionally be co-expressed with mbIL15 (FIG. 7, NKG2D ACRb). Additional information about chimeric receptors for use in the presently disclosed methods and compositions can be found in PCT Patent Publication No. WO/2018/183385, which is incorporated in its entirety by reference herein.

In several embodiments, there is provided a polynucleotide encoding an anti CD70/CD8αhinge/CD8α transmembrane domain/OX40/CD3zeta chimeric antigen receptor complex (see FIG. 7, CD70 CARa). The polynucleotide comprises or is composed of an anti CD70 binding moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a CD70 CAR construct that further comprises mbIL15, as disclosed herein (see e.g., FIG. 7, CD70 CARb). Additional information about anti-CD70 CAR for use in the presently disclosed methods and compositions can be found in U.S. Provisional Patent Application Nos. 63/038,645 and 63/090,041, which are each incorporated in their entirety by reference herein.

In several embodiments, there is provided a polynucleotide encoding an anti mesothelin/CD8α hinge/CD8α transmembrane domain/OX40/CD3zeta chimeric antigen receptor complex (see FIG. 7, Mesothelin CARa). The polynucleotide comprises or is composed of an anti mesothelin binding moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a Mesothelin CAR construct that further comprises mblL15, as disclosed herein (see e.g., FIG. 7, Mesothelin CARb).

In several embodiments, there is provided a polynucleotide encoding an anti PD-L1/CD8αhinge/CD8α transmembrane domain/OX40/CD3zeta chimeric antigen receptor complex (see FIG. 7, PD-L1 CARa). The polynucleotide comprises or is composed of an anti PD-L1 binding moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a PD-L1 CAR construct that further comprises mbIL15, as disclosed herein (see e.g., FIG. 7, PD-L1 CARb).

In several embodiments, there is provided a polynucleotide encoding an anti EGFR/CD8αhinge/CD8α transmembrane domain/OX40/CD3zeta chimeric antigen receptor complex (see FIG. 7, EGFR CARa). The polynucleotide comprises or is composed of an anti EGFR binding moiety, a CD8alpha hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a EGFR CAR construct that further comprises mbIL15, as disclosed herein (see e.g., FIG. 7, EGFR CARb).

In several embodiments, an expression vector, such as a MSCV-IRES-GFP plasmid, a non-limiting example of which is provided in SEQ ID NO: 87, is used to express any of the chimeric antigen receptors provided for herein.

### Methods of Treatment

Some embodiments relate to a method of treating, ameliorating, inhibiting, or preventing cancer with a cell or immune cell comprising a chimeric antigen receptor and/or an activating chimeric receptor, as disclosed herein. In some embodiments, the method includes treating or preventing cancer. In some embodiments, the method includes administering a therapeutically effective amount of immune cells expressing a tumor-directed chimeric antigen receptor and/or tumor-directed chimeric receptor as described herein. Examples of types of cancer that may be treated as such are described herein.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Advantageously, the non-alloreactive engineered T cells disclosed herein further enhance one or more of the above. Administration can be by a variety of routes, including, without limitation, intravenous, intra-arterial, subcutaneous, intramuscular, intrahepatic, intraperitoneal and/or local delivery to an affected tissue.

### Administration and Dosing

Further provided herein are methods of treating a subject having cancer, comprising administering to the subject a composition comprising immune cells (such as NK and/or T cells) engineered to express a cytotoxic receptor complex as disclosed herein. For example, some embodiments of the compositions and methods described herein relate to use of a tumor-directed chimeric antigen receptor and/or tumor-directed chimeric receptor, or use of cells expressing a tumor-directed chimeric antigen receptor and/or tumor-directed chimeric receptor, for treating a cancer patient. Uses of such engineered immune cells for treating cancer are also provided.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein. Advantageously, the non-alloreactive engineered T cells disclosed herein further enhance one or more of the above.

Administration can be by a variety of routes, including, without limitation, intravenous, intra-arterial, subcutaneous, intramuscular, intrahepatic, intraperitoneal and/or local delivery to an affected tissue. Doses of immune cells such as NK and/or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg. Depending on the embodiment, various types of cancer can be treated. In several embodiments, hepatocellular carcinoma is treated. Additional embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-174 (or combinations of two or more of SEQ ID NOS: 1-174) and that also exhibit one or more of the functions as compared with the respective SEQ ID NOS. 1-174 (or combinations of two or more of SEQ ID NOS: 1-174) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein. Additionally, there are provided herein compositions comprising a mixture of engineered immune cells (such as NK cells and/or engineered T cells), each population comprising the polynucleotide, vector, or cytotoxic receptor complexes as disclosed herein and the T cell population having been genetically modified to reduce/eliminate gvHD and/or HvD. In some embodiments, the NK cells and the T cells are from the same donor. In some embodiments, the NK cells and the T cells are from different donors.

Doses of immune cells such as NK cells or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of NK cells is administered, for example between about 1×10⁶ cells/kg to about 1×10⁸ cells/kg. Depending on the embodiment, various types of cancer or infection disease can be treated.

### Cancer Types

Some embodiments of the compositions and methods described herein relate to administering immune cells comprising a tumor-directed chimeric antigen receptor and/or tumor-directed chimeric receptor to a subject with cancer. Various embodiments provided for herein include treatment or prevention of the following non-limiting examples of cancers. Examples of cancer include, but are not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

### Cancer Targets

Some embodiments of the compositions and methods described herein relate to immune cells comprising a chimeric receptor that targets a cancer antigen. Non-limiting examples of target antigens include: CD19; TNF receptor family member B cell maturation (BCMA); CD38; DLL3; G protein coupled receptor class C group 5, member D (GPRC5D); epidermal growth factor receptor (EGFR) CD138; prostate-specific membrane antigen (PSMA); Fms Like Tyrosine Kinase 3 (FLT3); KREMEN2 (Kringle Containing Transmembrane Protein 2); ALPPL2 (Alkaline phosphatase, placental-like 2); CLDN4 (Claudin 4); CLDN6 (Claudin 6); CD123; CD22; CD30; CD171; CS1 (also referred to as CD2 subset 1, CRACC, SLAMF7, CD319, and 19A24); CD5, C-type lectin-like molecule-1 (CLL-1 or CLECLi); CD33; epidermal growth factor receptor variant III (EGFRviii); ganglioside G2 (GD2); ganglioside GD3 (aNeu5Ac(2-8)aNeu5Ac(2-3)bDGalp(I-4)bDGlcp(I-I)Cer); Tn antigen ((Tn Ag) or (GalNAca-Ser/Thr)); Receptor tyrosine kinase-like orphan receptor 1 (ROR1); Tumor-associated glycoprotein 72 (TAG72); CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(I-4)bDGlcp(I-l)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRi); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator oflmprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMI, PTK7, gpNMB, CDH1-CD324, CD276/B7H3, ILI IRa, IL13Ra2, CD179b-IGLII, TCRgamma-delta, NKG2D, CD32 (FCGR2A), Tn ag, Timl-/HVCR1, CSF2RA (GM-CSFR-alpha), TGFbetaR2, Lews Ag, TCR-betal chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVI-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudinl 8.2 (CLD18A2 or CLDN18A.2)), P-glycoprotein, STEAPI, Livl, Nectin-4, Cripto, gpA33, BST1/CD157, low conductance chloride channel, and the antigen recognized by TNT antibody.

### EXAMPLES

The following are non-limiting descriptions of experimental methods and materials that were used in examples disclosed below.

### Example 1—Gene Editing of Immune Cells to Alter Cell Function

To further build on various embodiments disclosed herein, several genes that mediate NK function through different pathways were selected in order to evaluate the impact of reducing/eliminating their expression through gene editing techniques. These initial targets represent non-limiting examples of the type of gene that can be edited according to embodiments disclosed herein to enhance one or more aspect of immune cell-mediated immunotherapy, whether utilizing engineered NK cells, engineered T cells, or combinations thereof. The tumor microenvironment (TME), as suggested with the nomenclature, is the environment around a tumor, which includes the surrounding blood vessels and capillaries, immune cells circulating through or retained in the area, fibroblasts, various signaling molecules related by the tumor cells, the immune cells or other cells in the area, as well as the surrounding extracellular matrix. Various mechanisms are employed by tumors to evade detection and/or destruction by host immune cells, including modification of the TME. Tumors may alter the TME by releasing extracellular signals, promoting tumor angiogenesis or even inducing immune tolerance, in part by limiting immune cell entry in the TME and/or limiting reproduction/expansion of immune cells in the TME. The tumor can also modify the ECM, which can allow pathways to develop for tumor extravasation to new sites. Transforming Growth-Factor beta (TGFb) has beneficial effects when reducing inflammation and preventing autoimmunity. However, it can also function to inhibit anti-tumor immune responses, and thus, upregulated expression of TGFb has been implicated in tumor progression and metastasis. TGFb signaling can inhibit the cytotoxic function of NK cells by interacting with the TGFb receptor expressed by NK cells, for example the TGFb receptor isoform II (TGFBR2). In accordance with several embodiments disclosed herein, the reduction or elimination of expression of TGFBR2 through gene editing (e.g., by CRISPr/Cas9 guided by a TGFBR2 guide RNA) interrupts the inhibitory effect of TGFb on NK cells.

As discussed above, the CRISPR/Cas9 system was used to specifically target and reduce the expression of the TGFBR2 by NK cells. Various non-limiting examples of guide RNAs were tested, which are summarized below.

TABLE 2
TGFb Receptor Type 2 Isoform Guide RNAs

| SEQ ID NO: | Name | Sequence | Target |
|---|---|---|---|
| 147 | TGFBR2-1 | CCCCTACCATGACTTTATTC | Exon 4 |
| 148 | TGFBR2-2 | ATTGCACTCATCAGAGCTAC | Exon 4 |
| 149 | TGFBR2-3 | AGTCATGGTAGGGGAGCTTG | Exon 4 |
| 150 | TGFBR2-4 | TGCTGGCGATACGCGTCCAC | Exon 1 |
| 151 | TGFBR2-5 | GTGAGCAATCCCCCGGGCGA | Exon 4 |
| 152 | TGFBR2-6 | AACGTGCGGTGGGATCGTGC | Exon 1 |

Briefly, cryopreserved purified NK cells were thawed on Day 0 and subject to electroporation with CRISPr/Cas9 and a single (or two) guide RNA (using established commercially available transfection guidelines) and were then subsequently cultured in 400 IU/ml IL-2 media for 1 day, followed by 40 IU/ml IL-2 culture with feeder cells (e.g., modified K562 cells expressing, for example, 4-1 BBL and/or mbIL15). At Day 7, knockout efficiency was determined and NK cells were transduced with a virus encoding the NK19-1 CAR construct (as a non-limiting example of a CAR). At Day 14, the knockout efficiency was determined by flow cytometry or other means and cytotoxicity of the resultant NK cells was evaluated.

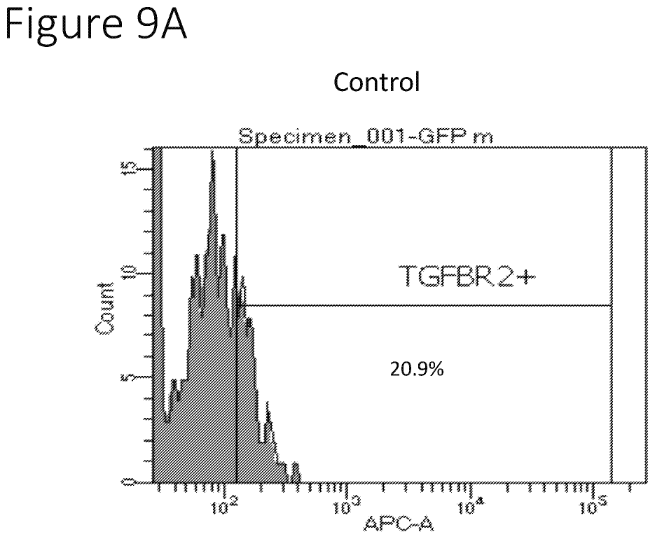

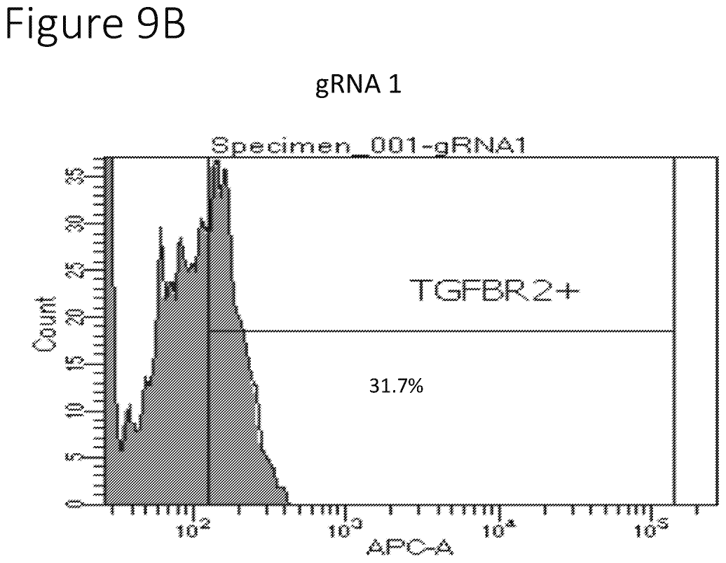

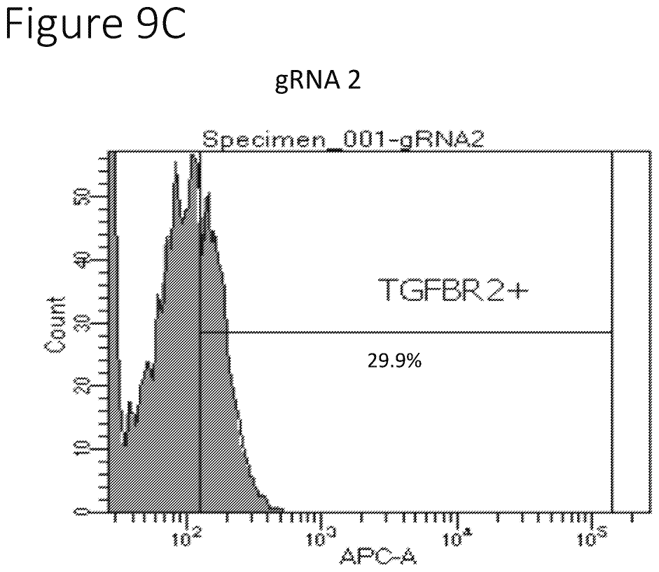

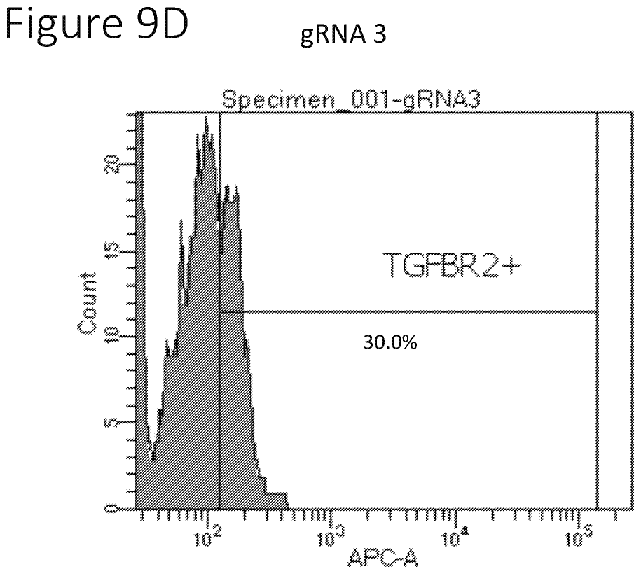

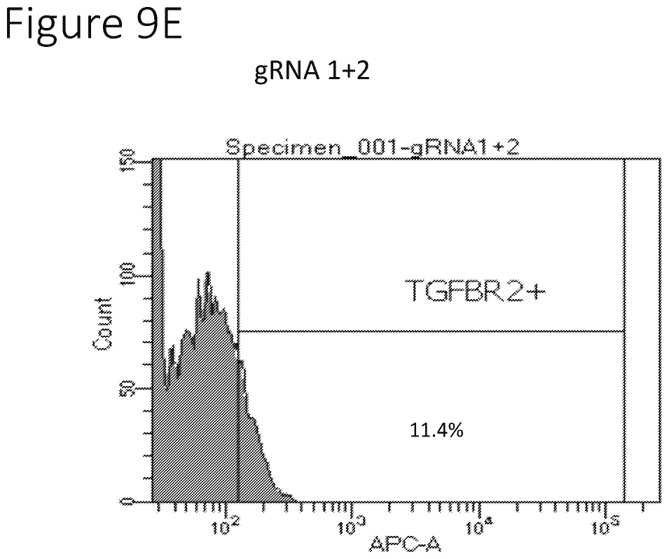

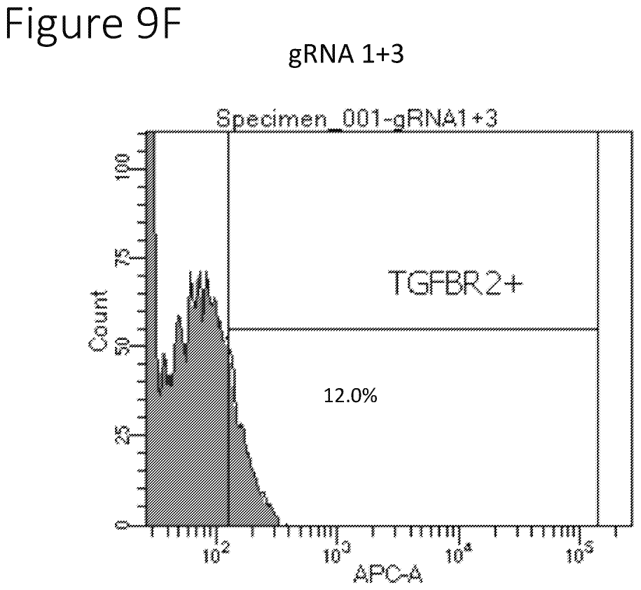

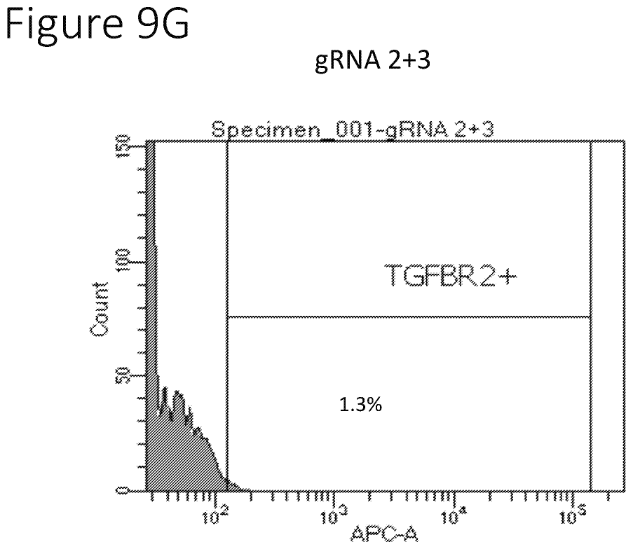

Flow cytometry analysis of TGFBR2 expression is shown in FIGS. 9A-9G. FIG. 9A shows control data in which NK cells were exposed to mock CRISPr/Cas9 gene editing conditions (nonsense or missing guide RNA). As shown, about 21% of the NK cells are positive for TGFBR2 expression. When the CRISPr/Cas9 machinery was guided using guide RNA 1 (SEQ ID NO. 147) TGFBR2 expression was not reduced (see FIG. 9B). Similar results are shown in FIGS. 9C and 9D, where guide RNA 2 (SEQ ID NO. 148) and guide RNA 3 (SEQ ID NO. 149) used individually had limited impact on TGFRB2 expression. In contrast, combinations of guide RNAs resulted in reduced TGFBR2 expression.

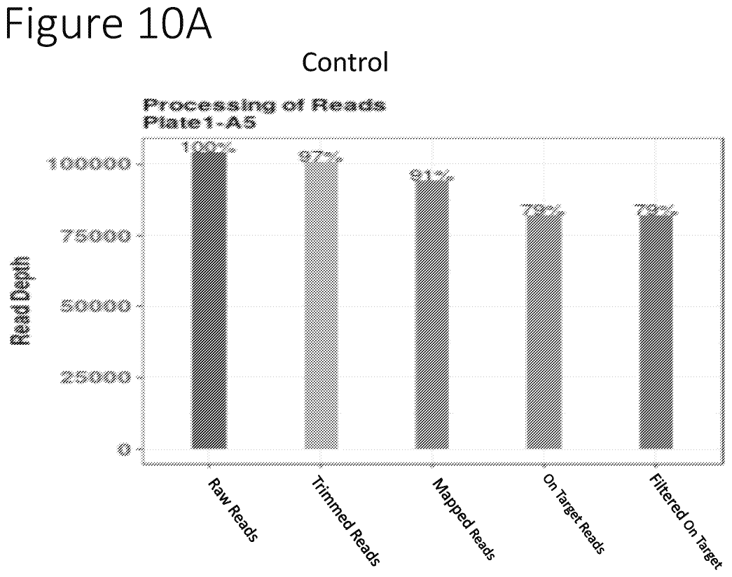

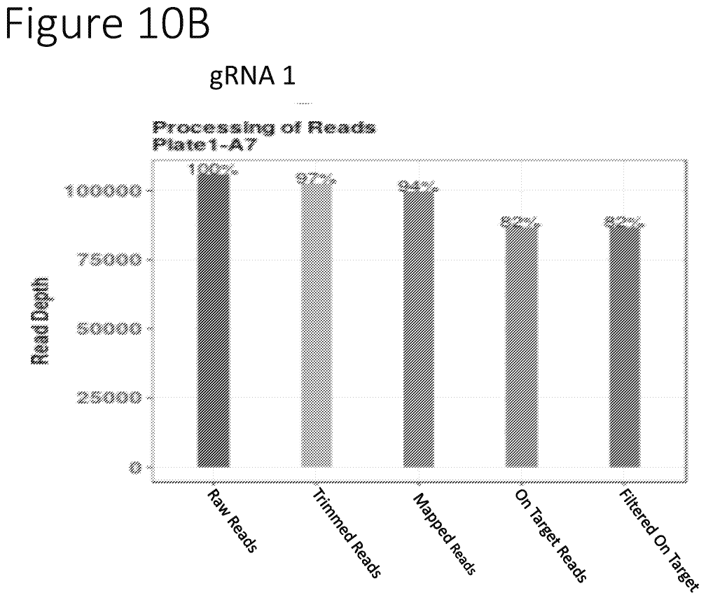

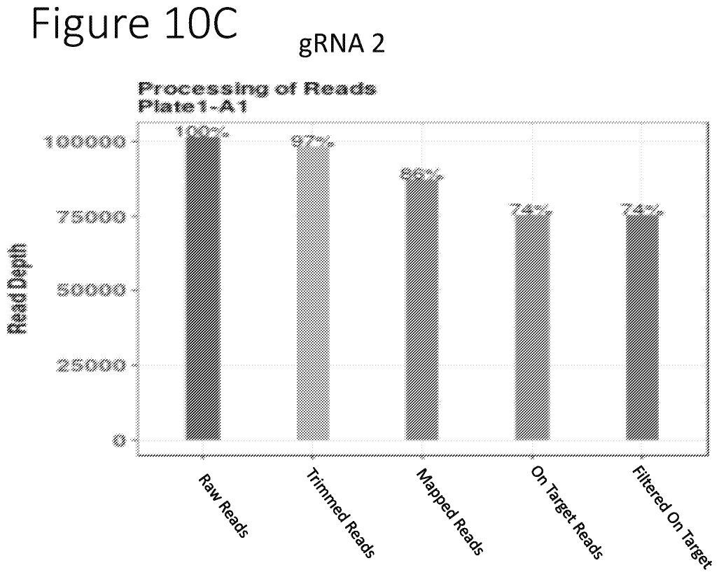

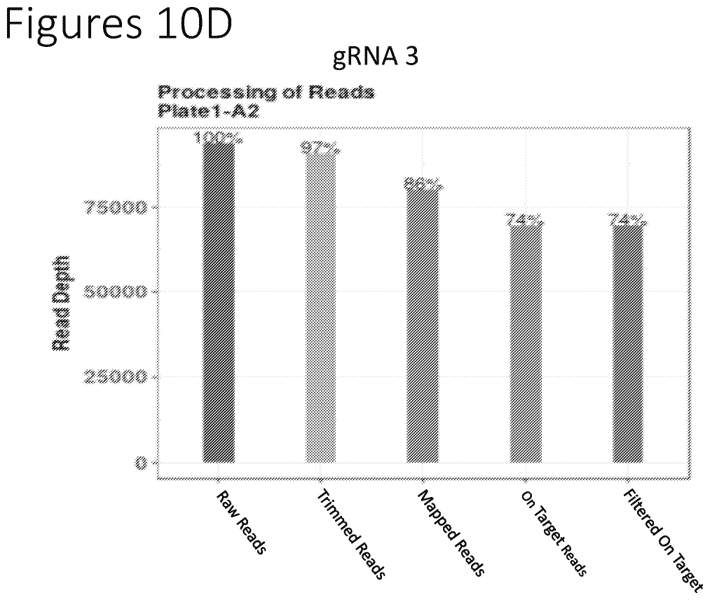

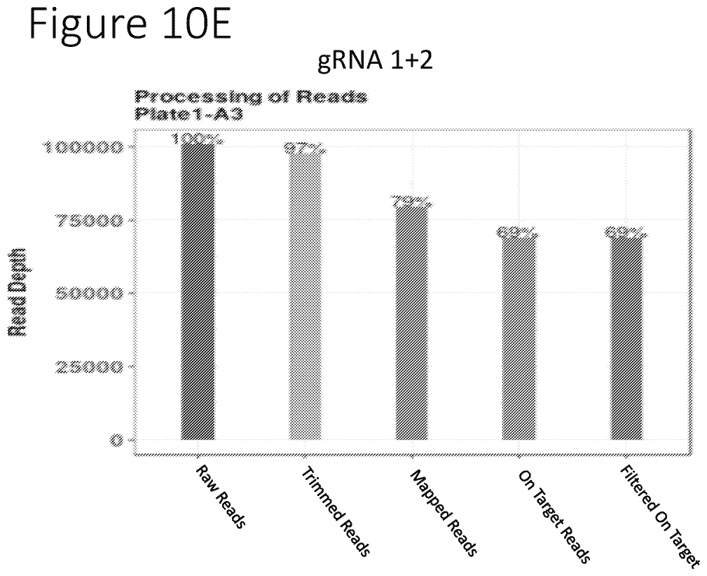

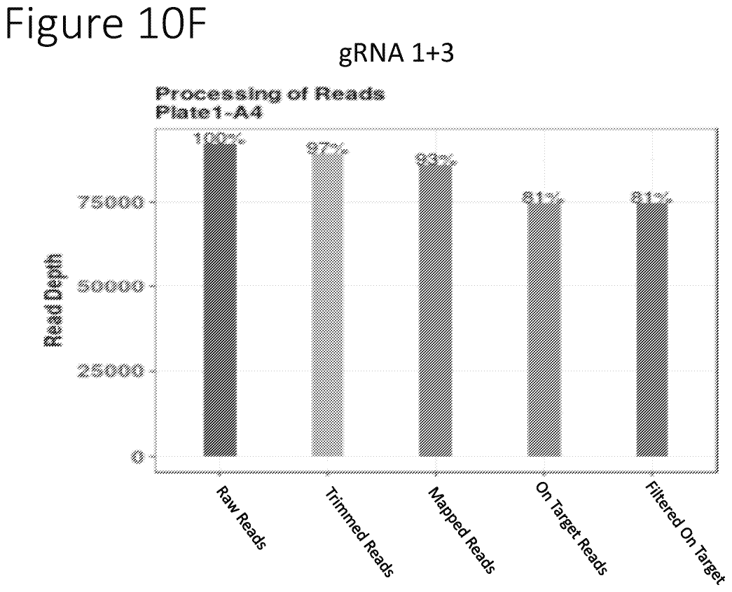

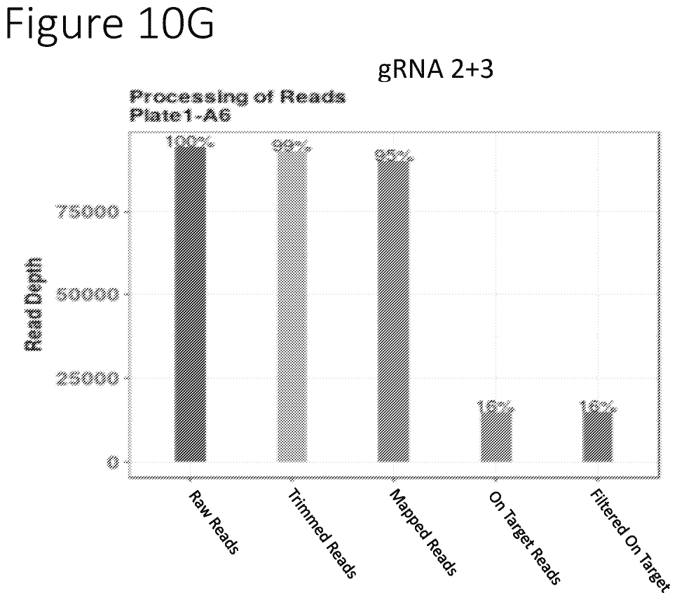

FIG. 9E shows results from the combination of guide RNA 1 (SEQ ID NO. 147) and guide RNA 2 (SEQ ID NO. 148) and FIG. 9F shows expression of TGFBR2 after use of the combination of guide RNA 1 (SEQ ID NO. 147) and guide RNA 3 (SEQ ID NO. 149). In each case, TGFBR2 expression was reduced by ˜50% as compared to the use of the guide RNAs alone (˜11-12% expression). FIG. 9G shows a marked reduction in TGFBR2 expression when both guide RNA 2 (SEQ ID NO. 148) and guide RNA 3 (SEQ ID NO. 149), with only ˜1% of the NK cells expressing TGFBR2. Next Generation Sequencing was used to confirm the flow cytometry expression analysis. These data are shown in FIGS. 10A-10G, which correspond to the respective guide RNAs in FIGS. 9A-9G. These data confirm that guide RNAs used with a CRISPr/Cas system can reduce expression of a specific target molecule, such as TGFBR2 on NK cells. According to several embodiments, a combination of guide RNAs, such as TGFBR guide RNA 2 and guide RNA 3 work synergistically together to essentially eliminate expression of the TGFBR2 by NK cells.

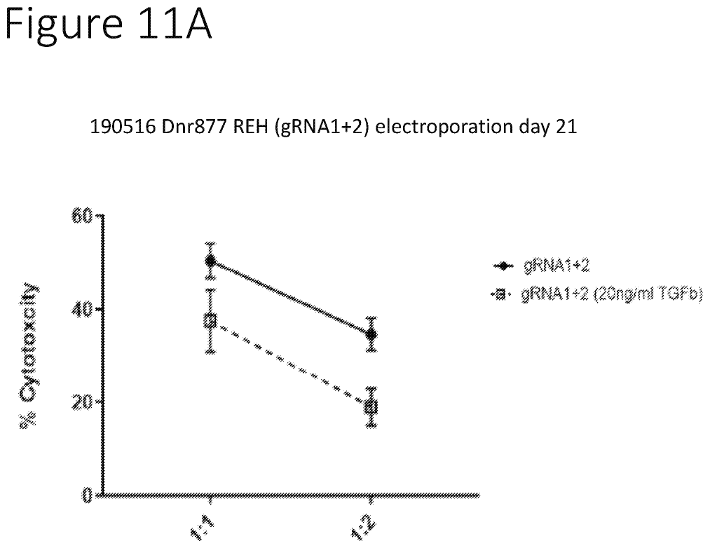

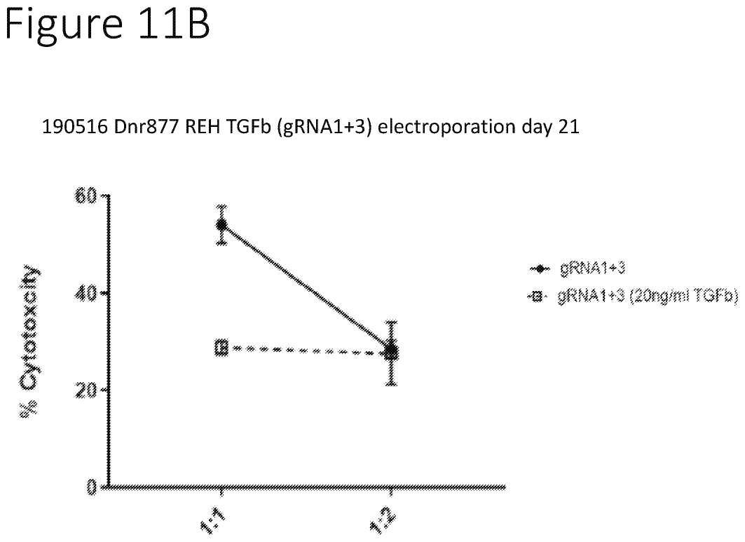

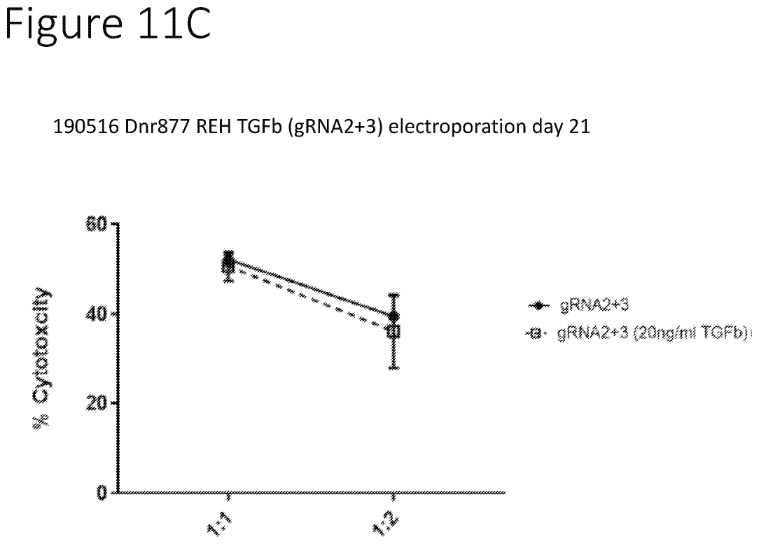

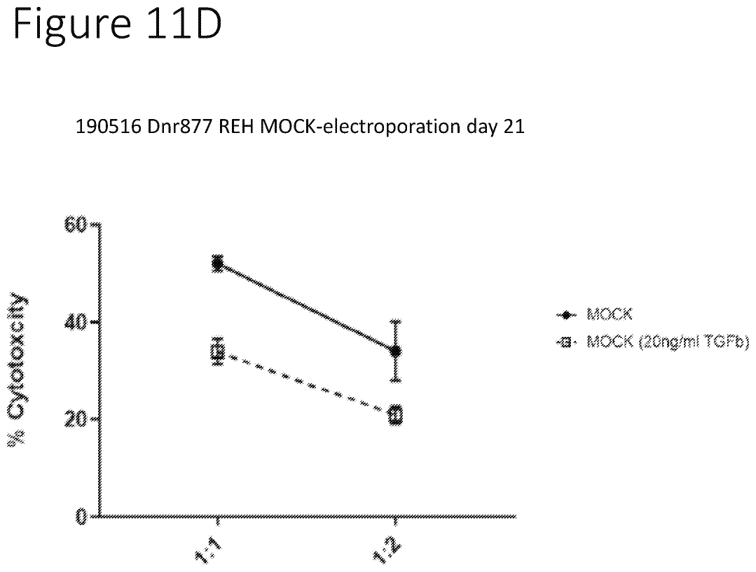

Building on these expression knockout experiments, the ability of TGFb to inhibit the cytotoxicity of TGFBR2 knockout NK cells was evaluated. To do so, NK cells were subject to TGFBR2 gene editing as discussed above, and at 21 days post-electroporation with the gene editing machinery, the cytotoxicity of the resultant cells was evaluated against REH tumor cells at 1:1 and 1:2 effector:target ratios and in the absence (closed circles) or presence of TGFb (20 ng/mL; open squares). Data are summarized in FIGS. 11A-11D. FIG. 11A shows data related to the combination of guide RNA 1 and 2. As evidenced by the decrease in the detected percent cytotoxicity at both 1:1 and 1:2 ratios with the addition of TGFb, these data are in line with the expression data discussed above, in that the presence of TGFBR2 (due to limited reduction in the expression of the receptor) allows TGFb to inhibit the cytotoxic activity of the NK cells. FIG. 11D shows mock results, with a similar cytotoxicity pattern to that shown in FIG. 11A. FIG. 11B shows similar data in that the presence of TGFb reduced the cytotoxicity of NK cells at a 1:1 target ratio when guide RNAs 1 and 3 were used to knock down TGFBR2 expression. At a 1:2 target ratio, the NK cells exhibited the same degree of cytotoxicity (reduced as compared to TGFBR2 knock down NK cells alone) whether TGFb was present or not. In contrast to the other experimental conditions, and in line with the expression data, FIG. 11C shows the cytotoxicity of NK cells edited with CRISPr using both guide RNAs 2 and 3. Despite the presence of TGFb at concentrations that reduced the cytotoxicity of the other NK cells tested, these NK cells that essentially lack TGFBR2 expression due to the gene editing show negligible fall off in cytotoxicity. These data show that, according to several embodiments, disclosed herein, use of gene editing techniques to disrupt, for example, expression of a negative regulator of immune cell activity results in an enhanced cytotoxicity and/or persistence of immune cells as disclosed herein.

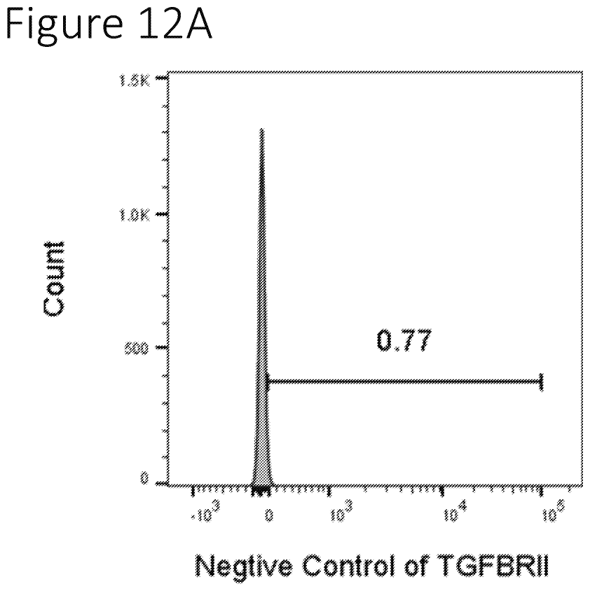

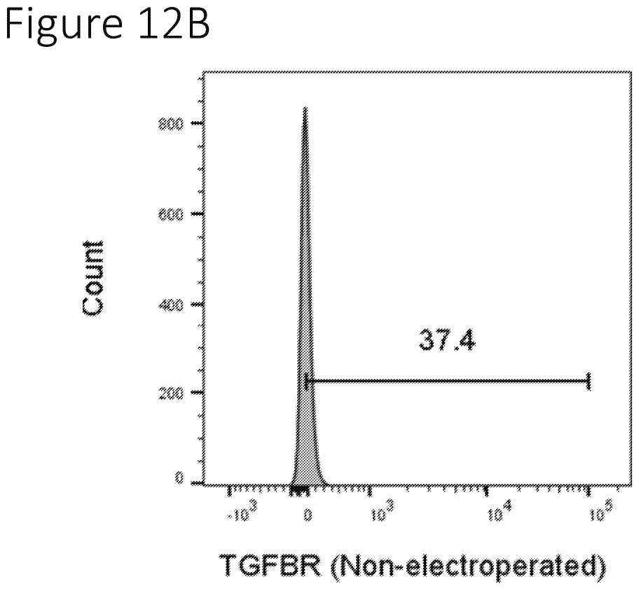

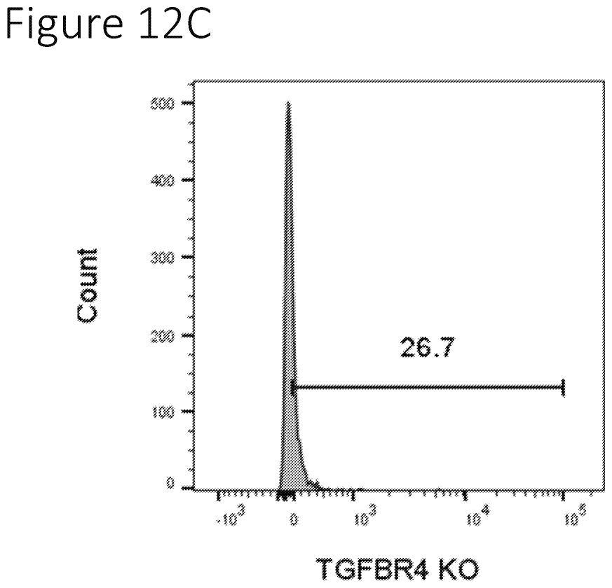

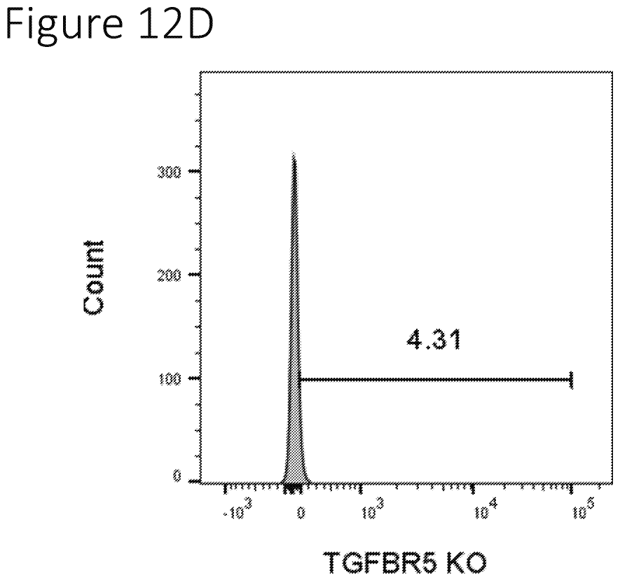

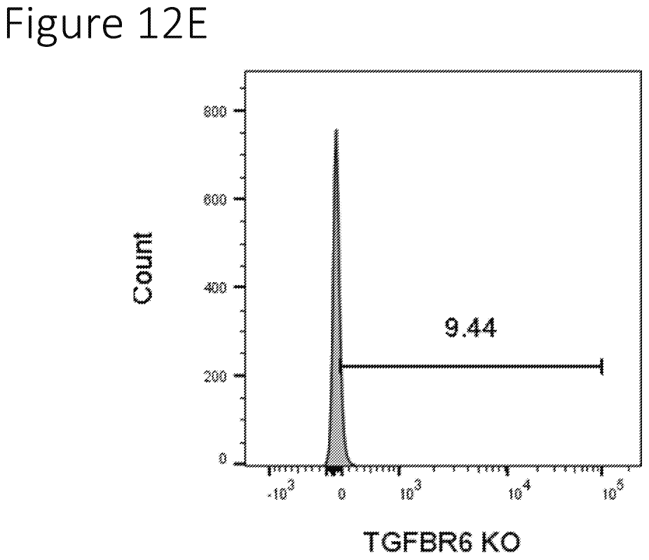

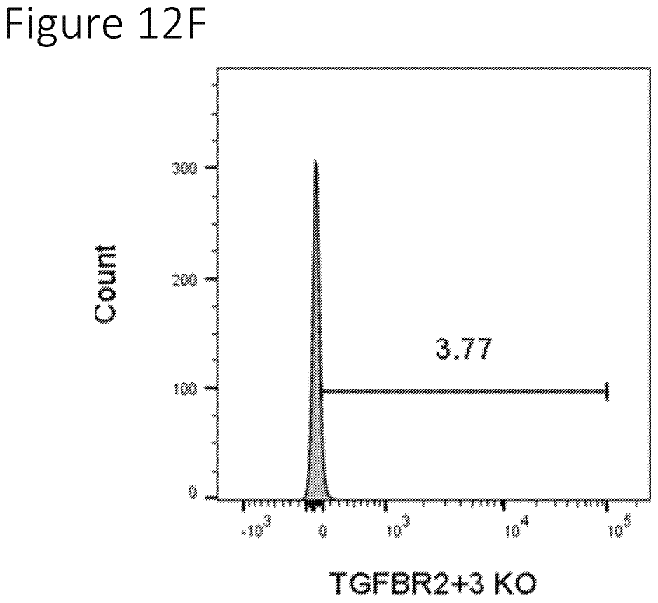

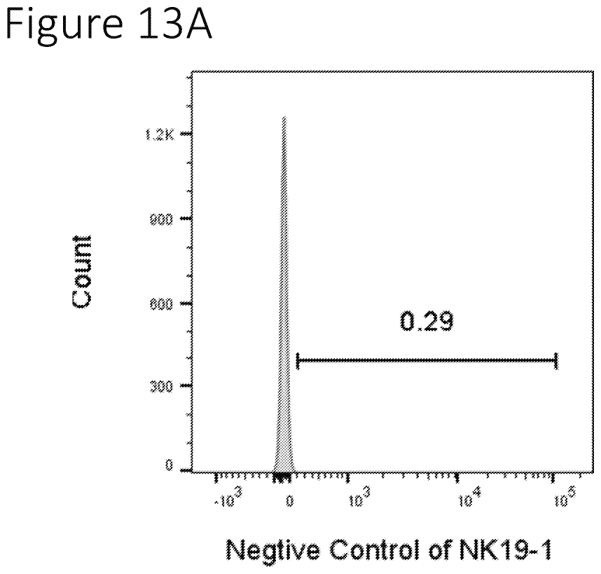

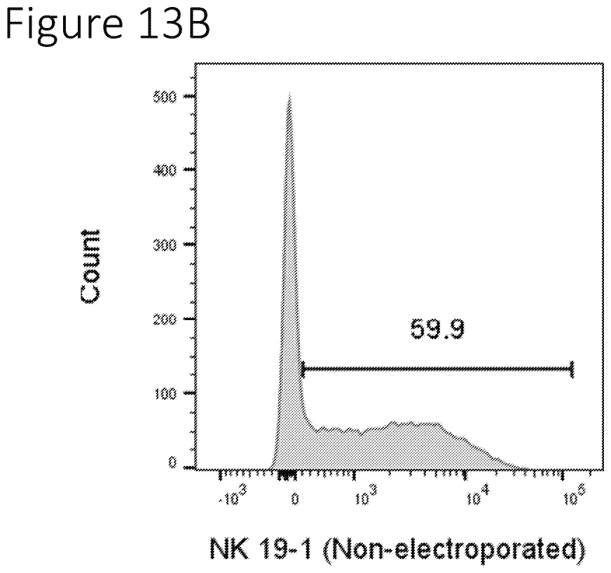

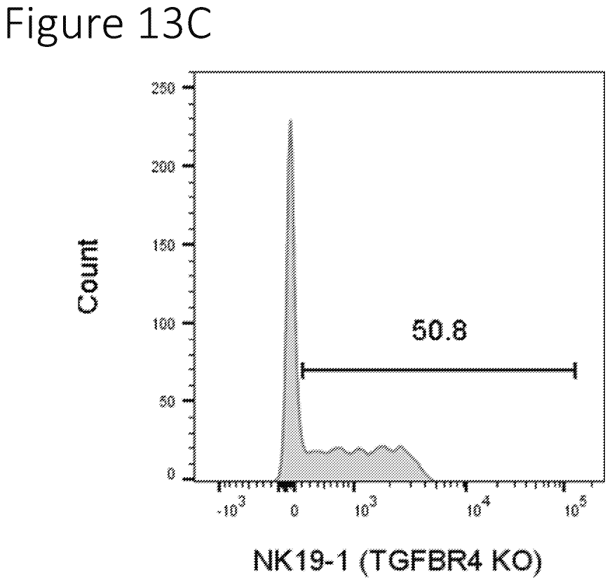

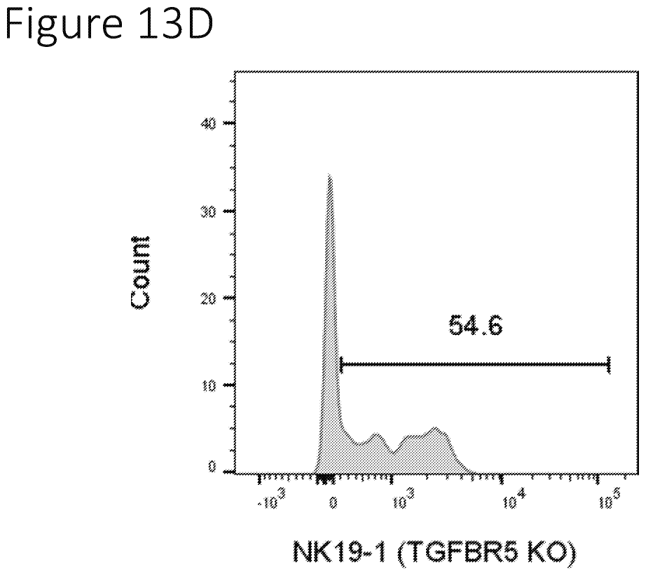

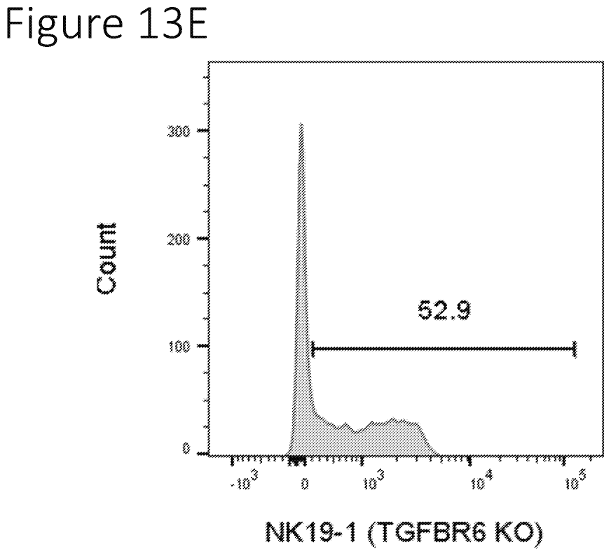

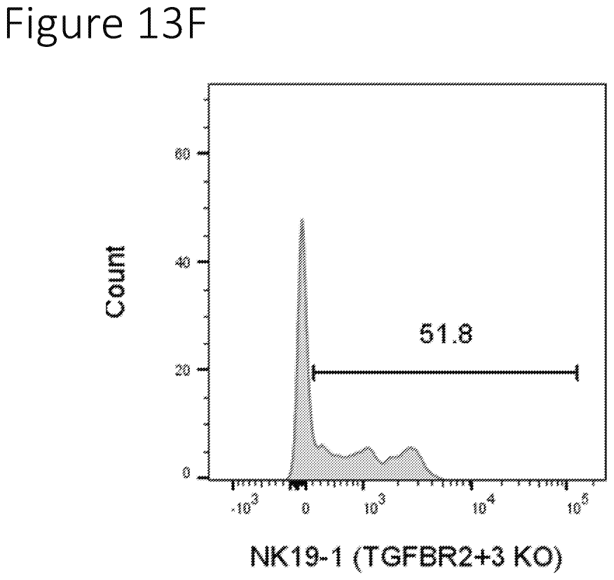

FIGS. 12A-12F present flow cytometry data related to additional guide RNAs directed against TGFBR2 (see Table 2). FIG. 12A shows negative control evaluation of expression of TGFBR2 by NK cells (e.g., NK cells not expressing TGFBR2). FIG. 12B shows positive control data for NK cells that were not electroporated with CRISPr/Cas9 gene editing machinery, thus resulting in ˜37% expression of TGFBR2 by the NK cells. FIGS. 12C, 12D and 12E show TGFBR2 expression by NK cells that were subject to CRISPr/Cas9 editing and guided by guide RNA 4 (SEQ ID NO. 150), guide RNA 5 (SEQ ID NO. 151), or guide RNA 6 (SEQ ID NO. 152), respectively. Guide RNA 4 resulted in modest knock down of TGFBR2 expression (˜10% reduced compared to positive control). In contrast, guide RNA 5 and guide RNA 6 each reduced TGFBR2 expression significantly, by about 33% and 28%, respectively. These two single guide RNAs were on par the with the reduction seen (discussed above) with the combination of guide RNA 2 and guide RNA 3 (additional data shown in FIG. 12F. In accordance with several embodiments discussed herein, engineered immune cells are subjected to gene editing, such that the resultant immune cell is engineered to express a chimeric construct that imparts enhanced cytotoxicity to the engineered cell. In addition, such cells are genetically modified, for example to dis-inhibit the immune cells by disrupting at least a portion of an inhibitory pathway that functions to decrease the activity or persistence of the immune cell. To confirm that gene editing and expression of cytotoxic constructs are compatible, as disclosed herein, expression of a non-limiting example of a chimeric antigen receptor construct targeting CD19 (here identified as NK19-1) was evaluated subsequent to gene editing to knock down TGFBR2 expression. These data are shown in FIGS. 13A-13F

FIG. 13A shows a negative control assessment of expression of a non-limiting example of an anti-CD19 directed CAR (NK19-1). Here, NK cells were not transduced with the NK19-1 construct. In contrast, FIG. 13B shows positive control expression of NK19-1 by non-electroporated NK cells (as a control to account for lack of processing through a CRISPr gene-editing protocol. FIG. 13C shows the expression of NK19-1 by NK cells that were subject to TGFBR2 knock down through the use of CRISPr/Cas9 and guide RNA 4. As shown, there is only a nominal reduction in NK19-1 expression after gene editing with CRISPr. According to some embodiments, depending on the guide RNA and/or the mechanism for gene editing (e.g., CRISPr vs. TALEN), the slight change in CAR expression is reduced and/or eliminated. This can be seen, for example, in FIG. 13D, wherein the use of guide RNA 5 resulted in an even smaller change in NK19-1 expression by the NK cells. FIGS. 13E and 13F show data for guide RNA 6 alone, as well as guide RNA 2+3 (respectively). Taken together, these data indicate that the two approaches that are used in accordance with several embodiments disclosed herein, namely gene editing and genetic modification to induce expression of a chimeric receptor, are compatible with one another in that the process of editing the immune cell to reduce/remove expression of a negative regulator of immune cell function does not prevent the robust expression of a chimeric receptor construct. In fact, in several embodiments, gene editing and engineering of the immune cells results in a more efficacious, potent and/or longer lasting cytotoxic immune cell.

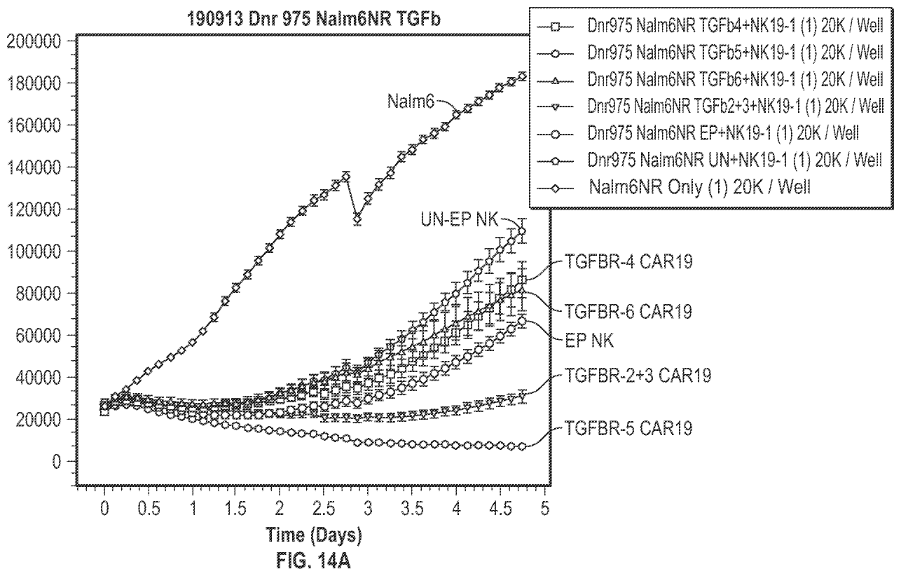

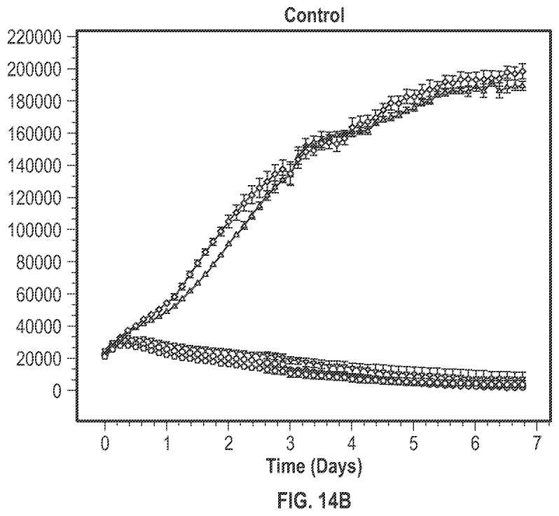

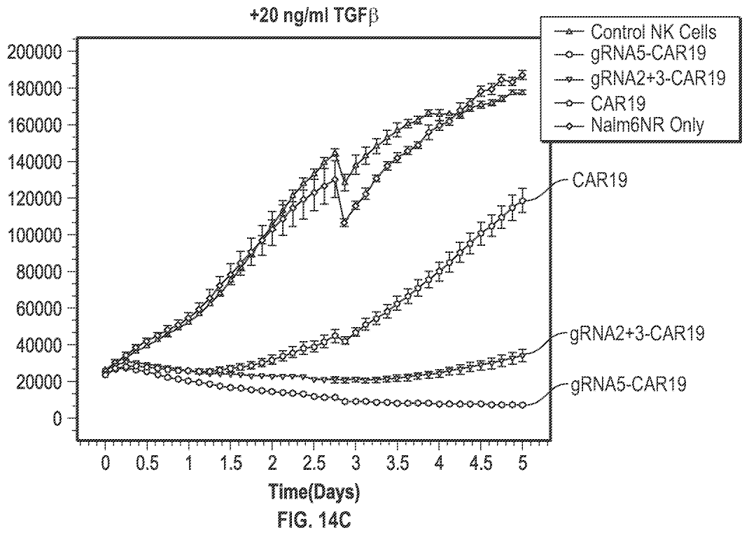

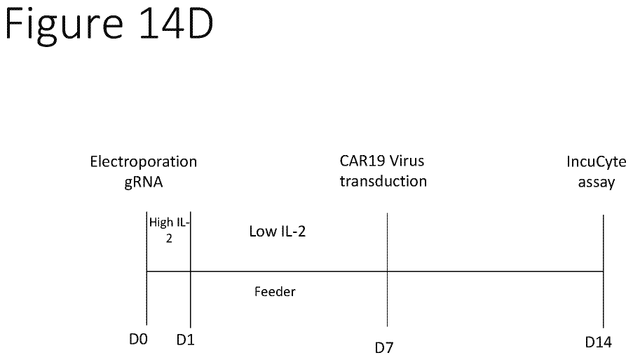

FIGS. 14A-14D show the methods and the results of an assessment of the cytotoxicity of NK cells that are subjected to gene editing (e.g., gene knockout) and/or genetic engineering (e.g., CAR expression) and their respective controls. Starting first with FIG. 14D, at Day 0, NK cells were subject to electroporation with the CRISPr/Cas9 components for gene editing, along with one (or a combination of) the indicated guide RNAs. NK cells were cultured in high-IL2 media for one day, followed by 6 additional days in culture with low IL2 and feeder cells (as discussed above). At Day 7, NK cells were transduced with the indicated anti-CD19 CAR viruses. Seven days later, the Incucyte cytotoxicity assay was performed in the presence of 20 ng/mL TGF-beta. As discussed above, TGF-beta is a potent immune suppressor that is released from the tumor cells and permeates the tumor microenvironment in vivo, in an attempt to decrease the effectiveness of immune cells in eliminating the tumor. Results are shown in FIG. 14A. As shown, in the top trace, Nalm6 cells grown alone expand robustly over the duration of the experiment. NK cells that were not electroporated (no gene editing or CAR expression; UN-EP NK) caused reduction in Nalm6 expansion. Reducing Nalm6 proliferation even further were NK cells that were subject to both gene editing and engineered CAR expression (TGFBR-4 CAR19 and TGFBR-6 CAR19). These results firstly demonstrate that these two techniques (e.g., editing and engineering) are compatible with one another and show that cytotoxicity can be enhanced in the resultant immune cells, in particular by engendering a resistance in the cells to immune suppressors in the tumor microenvironment, like TGF-beta. NK cells that were subject to electroporation, but not engineered to express a CAR (EP NK) reduced Nalm6 growth. Most notable, however, were the dramatic inhibition of Nalm6 expansion resulting from the use of NK cells engineered to express CAR19-1 (as a non-limiting example of a CAR) and which were also subject to knockout of TGFBR2 expression through either the combination of guide RNA 2 and guide RNA 3 (TGFBR-2+3 CAR19) or through the use of the single guide RNA, guide RNA 5 (TGFBR-5 CAR19). These data further evidence that, according to several embodiments disclosed herein, there a robust enhancement of the cytotoxicity of immune cells can be realized through a synergistic combination of reducing an inhibitory pathway (e.g., reduction in the inhibitory effects of TGFb by knockout of the TGFBR2 on immune cells through gene editing) and introducing a cytotoxic signaling complex (e.g., through engineering of the cells to express a CAR). FIGS. 14B and 14C show control data and selected data from FIG. 14A, respectively. FIG. 14B shows the significant cytotoxic effects of all constructs tested against Nalm6 cells alone (e.g., not recapitulating the immune suppressive effect of the tumor microenvironment). Each construct tested effectively eliminated tumor cell growth. In FIG. 14C, the tumor challenge experiments were performed in the presence of 20 ng/mL of TGF-beta to recapitulate the tumor microenvironment. FIG. 14C is selected data from 14A, to show the effects of gene editing to knockout the TGFB2 receptor more clearly. Cells engineered to express NK19-1 (as a non-limiting example of a CAR) showed the ability to reduce tumor growth as compared to controls. However, NK cells expressing NK19-1 and engineered (through CRISPR/Cas9 gene editing and the use of the non-limiting examples of guide RNAs) showed even more significant reductions in growth of tumor cells. Thus, according to several embodiments, leading to results such as those shown in FIG. 14A (and 14C), these gene editing techniques can be used to enhance the cytotoxicity of NK cells, even in the immune suppressive tumor microenvironment. In several embodiments, analogous techniques can be used on T cells. Additionally, in several embodiments, analogous approaches are used on both NK cells and T cells. Further, in additional embodiments, gene editing is used to engender edited cells, whether NK cells, T cell, or otherwise, resistance to one or more immune suppressors found in a tumor microenvironment.

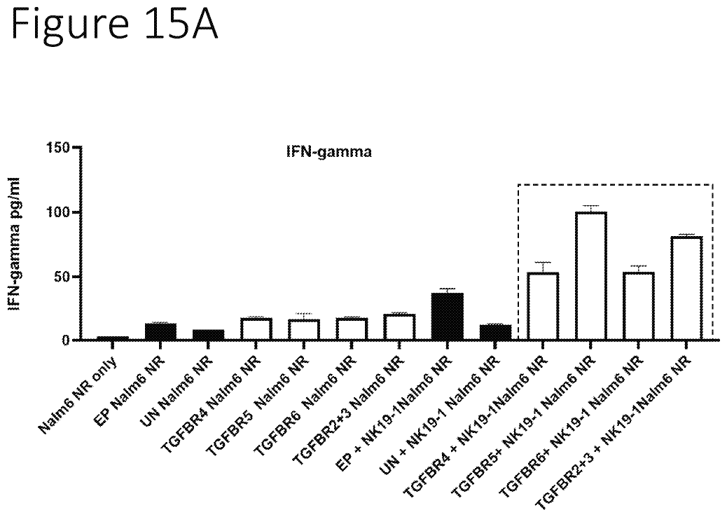

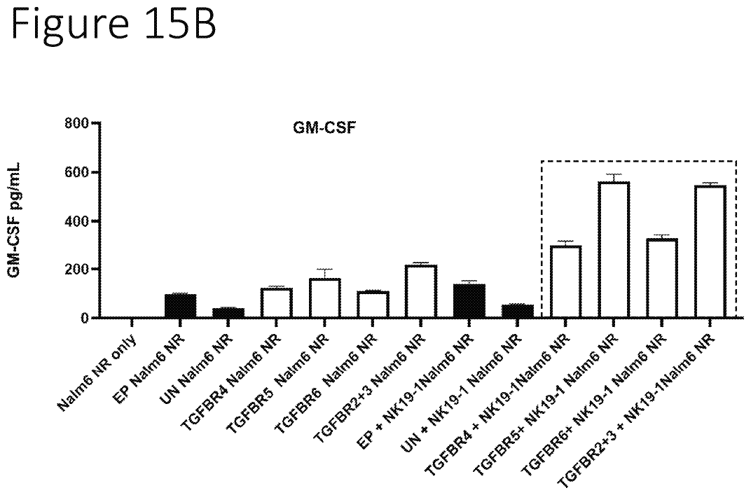

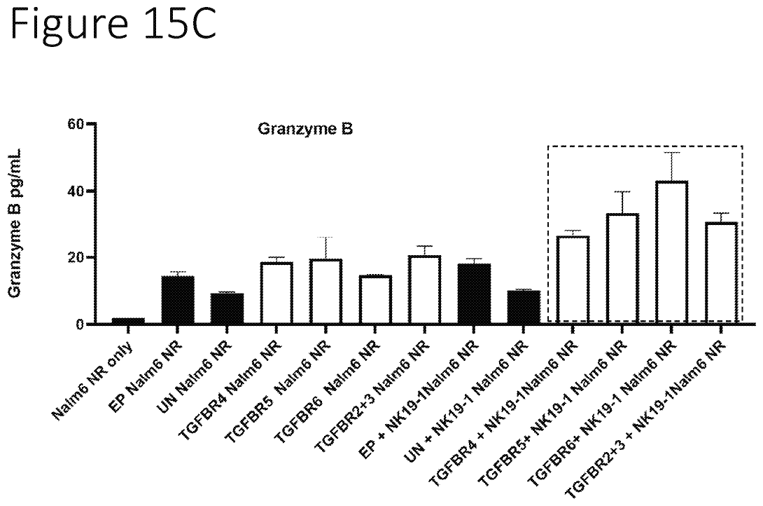

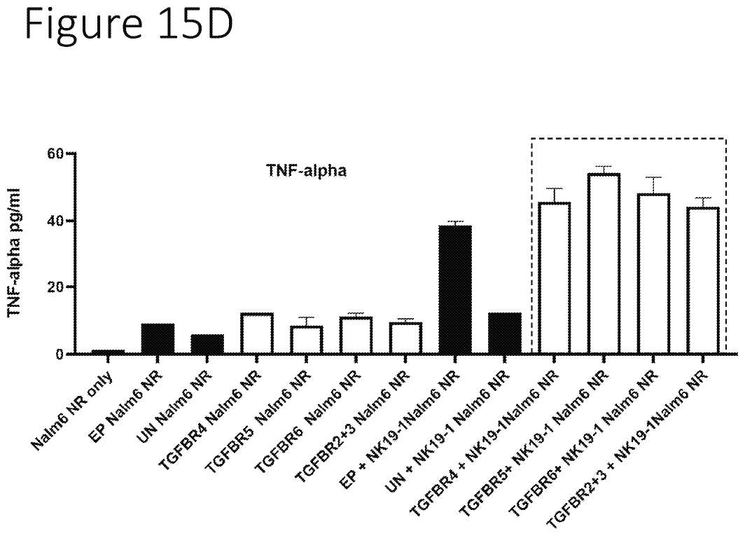

To evaluate the potential mechanisms by which the modified immune cells exert their increased cytotoxic activity the cytokine release profile of each of the types of cells tested was evaluated, the data being shown in FIGS. 15A-15D. In brief, each of the NK cell groups were treated with TGFb 1 at a concentration of 20 ng/mL overnight prior inception of the cytotoxicity assay. The NK cells were washed to remove TGFb prior to co-culture of the NK cells with Nalm6 tumor cells. NK cells were co-cultured with Nalm6 tumor cells expressing nuclear red fluorescent protein (Nalm6-NR) at an E:T ratio of 1:1 (2×10⁴ effector: 2×10⁴ target cells). Cytokines were measured by Luminex assay. As shown in FIG. 15A, there was a modest increase in the release of IFNg when TGFBR2 expression was reduced by gene editing (see for example the histogram bar for “TGFBR2+3 Nalm6 NR”). Introduction of the anti-CD19 CAR induced a substantial increase in IFNg production (EP+NK19-1 Nalm6-NR). Most notably, however, are the last four groups shown in FIG. 15A (see dashed box), which represent the use of either single guide RNAs, or a combination of guide RNAs, to direct the CRISPr/Cas9-mediated knockdown of expression of the TGFBR2 in combination with the expression of an anti-CD19 CAR. The release of these increased amounts of IFNg are, at least in part, responsible for the enhanced cytotoxicity seen using these doubly-modified immune cells. Similar to IFNg, GM-CSF release was significantly enhanced in these groups. GM-CSF can promote the differentiation of myeloid cells and also as an immunostimulatory adjuvant, thus it's increased release may play a role in the increased cytotoxicity seen with these cells. Similar patterns are seen when assessing the release of Granzyme B (a potent cytotoxic protein released by NK cells) and TNFalpha (another potent cytokine). These data further evidence that increased release of various cytokines are at play in causing the substantial increase in cytotoxicity seen with the gene edited and genetically modified immune cells, as in accordance with several embodiments disclosed herein, as the gene editing aids in resisting immune suppressive effects that would be seen in the tumor microenvironment.

In accordance with additional embodiments, a disruption of, or elimination of, expression of a receptor, pathway or protein on an immune cell can result in the enhanced activity (e.g., cytotoxicity, persistence, etc.) of the immune cell against a target cancer cell. In several embodiments, this results from a disinhibition of the immune cell. Natural killer cells, express a variety of receptors, such particularly those within the Natural Killer Group 2 family of receptors. One such receptor, according to several embodiments disclosed herein, the NKG2D receptor, is used to generate cytotoxic signaling constructs that are expressed by NK cells and lead to enhanced anti-cancer activity of such NK cells. In addition, NK cells express the NKG2A receptor, which is an inhibitory receptor. One mechanism by which tumors develop resistance to immune cells is through the expression of peptide-loaded HLA Class I molecules (HLA-E), which suppresses the activity of NK cells through the ligation of the HLA-E with the NKG2A receptor. Thus, while one approach could be to block the interaction of the HLA-E with the expressed NKG2A receptors on NK cells, according to several embodiments disclosed herein, the expression of NKG2A is disrupted, which short circuits that inhibitory pathway and allows enhanced NK cell cytotoxicity.

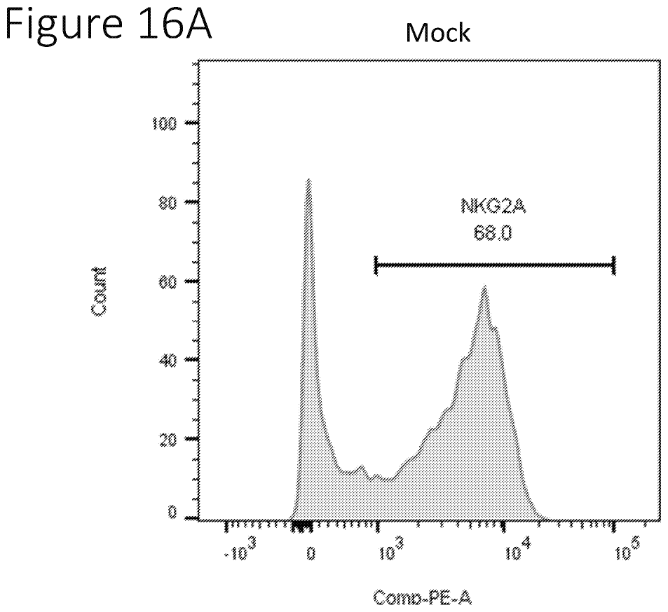

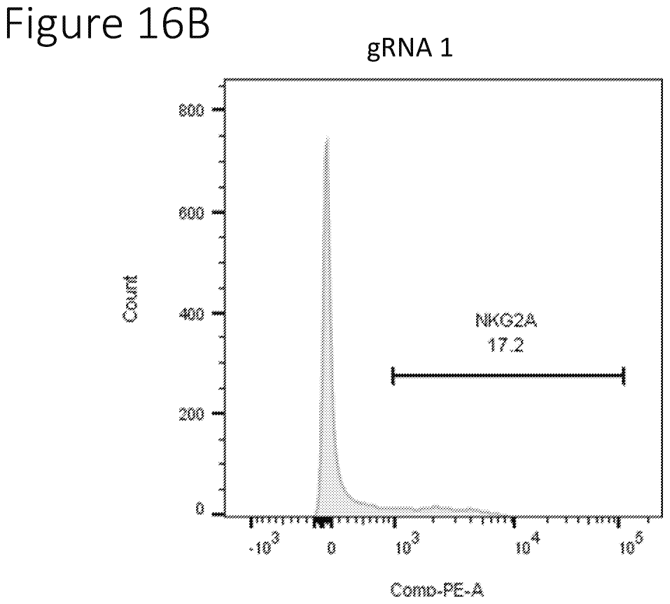

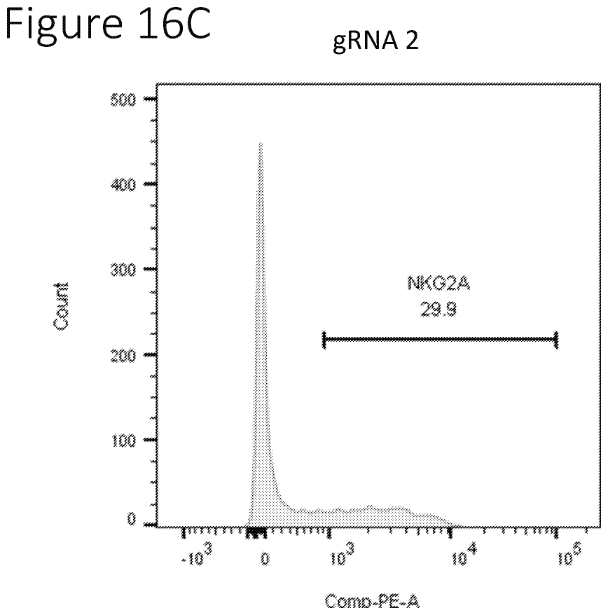

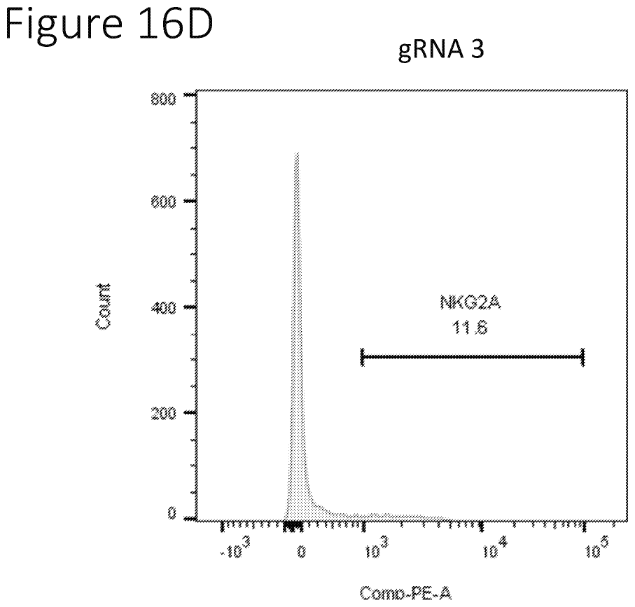

FIGS. 16A-16D show data related to the disruption of expression of NKG2A expression by NK cells. As discussed above with TGFBR2, CRISPr/Cas9 was used to disrupt NKG2A expression using the non-limiting examples of guide RNAs show below in Table 3.

TABLE 3
NKG2A Guide RNAs

| SEQ ID NO: | Name | Sequence | Target |
|---|---|---|---|
| 158 | NKG2A-1 | GGAGCTGATGGTAAATCTGC | Exon 4 |
| 159 | NKG2A-2 | TTGAAGGTTTAATTCCGCAT | Exon 3 |
| 160 | NKG2A-3 | AACAACTATCGTTACCACAG | Exon 4 |

FIG. 16A shows control NKG2A expression by NK cells, with approximately 70% of the NK cells expressing NKG2A. FIG. 16B demonstrates that significant reductions in NKG2A expression can be achieved, with the use of guide RNA 1 reducing NKG2A expression by over 50%. FIG. 16C shows a more modest reduction in NKG2A expression using guide RNA 2, with just under 30% of the NK cells now expressing NKG2A. FIG. 16D shows that use of guide RNA 3 provides the most robust disruption of NKG2A expression by NK cells, with only ˜12% of NK cells expressing NKG2A.

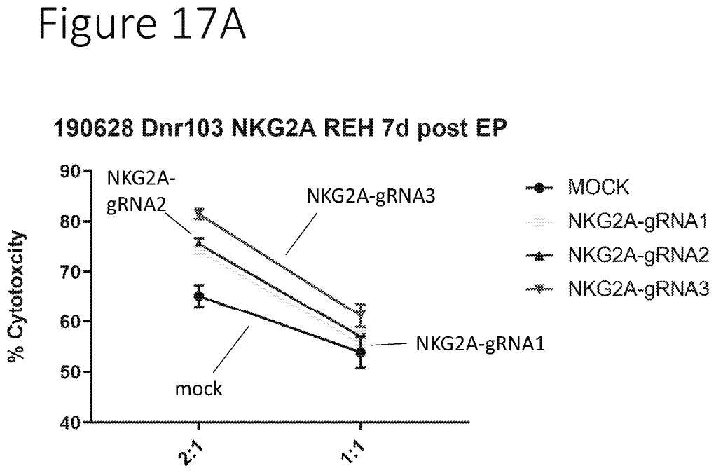

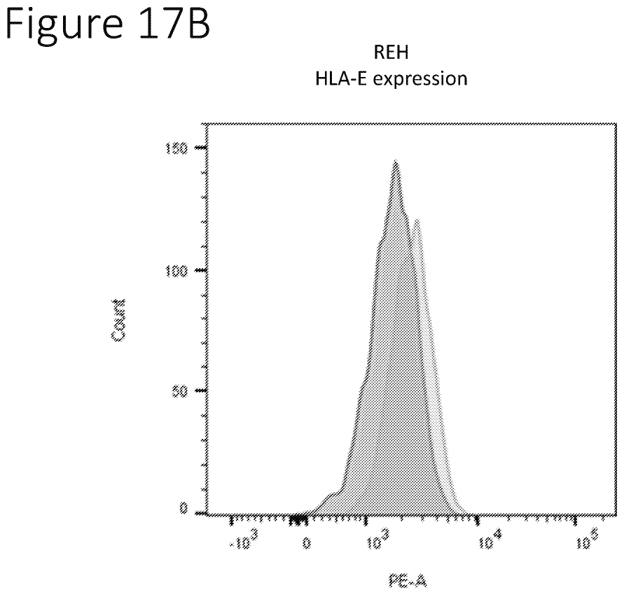

FIG. 17A shows summary cytotoxicity data related to the NK cells with reduced NKG2A expression against Reh tumor cells at 7 days post-electroporation with the gene editing machinery. NK cells were tested at both a 2:1E:T and a 1:1E:T ratio. At 1:1E:T, each of the gene edited NK cell types induced a greater degree of cytotoxicity than the mock NK cells. The improved cytotoxicity detected with guide RNA 1 and guide RNA 2 treated NK cells were slightly enhanced over mock. The guide RNA that induced the greatest disruption of NKG2A expression on NK cells also resulted in the greatest increase of cytotoxicity as compared to mock (see 1:1 NKG2A-gRNA3). At a 2:1 ratio, each of the modified NK cell types significantly outperformed mock NK cells. As with the lower ratio, NK cells edited using guide RNA3 to target the CRISPr/Cas9 showed the most robust increase in cytotoxicity, an inverse relationship with the degree of NKG2A expression disruption. As discussed above, the interaction of HLA-E on tumor cells with the NKG2A on NK cells, absent intervention, can inhibit the NK cell activity. FIG. 17B confirms that Reh tumor cells do in fact express HLA-E molecules, and therefore, in the absence of the gene editing to disrupt NKG2A expression on the NK cells, would have been expected to inhibit NK cell signaling (as seen with the Mock NK cell group in FIG. 17A).

While the disruption of the HLA-E/NKG2A interaction had a clear positive impact on cytotoxicity of NK cells, other pathways were investigated that may impact immune cell signaling. One such example is the CIS/CISH pathway. Cytokine-inducible SH2-containing protein (CIS) is a negative regulator of IL-15 signaling in NK cells, and is encoded by CISH gene in humans. IL-15 signaling can have positive impacts on the NK cell expansion, survival, cytotoxicity and cytokine production. Thus, a disruption of CISH could render NK cells more sensitive to IL-15, thereby increasing their anti-tumor effects.

As discussed above, CRISPr/CAs9 was used to disrupt expression of CISH, though in additional embodiments, other gene editing approaches can be used. Non-limiting examples of CISH-targeting guide RNAs are shown below in Table 4.

TABLE 4
CISH Guide RNAs

| SEQ ID NO: | Name | Sequence | Target |
|---|---|---|---|
| 153 | CISH-1 | CTCACCAGATTCCCGAAGGT | Exon 2 |
| 154 | CISH-2 | CCGCCTTGTCATCAACCGTC | Exon 3 |
| 155 | CISH-3 | TCTGCGTTCAGGGGTAAGCG | Exon 1 |
| 156 | CISH-4 | GCGCTTACCCCTGAACGCAG | Exon 1 |
| 157 | CISH-5 | CGCAGAGGACCATGTCCCCG | Exon 1 |

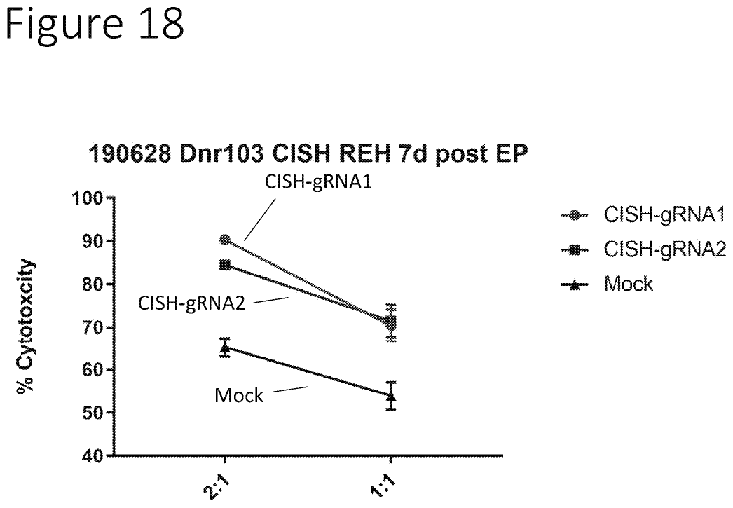

As with NKG2A knockout NK cells, CISH knockout (using guide RNA 1 or Guide RNA 2 (data not shown for CISH-3-5)) gene edited NK cells were challenged with Reh tumor cells at a 1:1 and 2:1E:T ratio 7 days after being electroporated with the gene editing machinery. FIG. 18 shows that while mock NK cells exhibited over 50% cytotoxicity against Reh cells at 1:1, each of the gene edited NK cell groups showed nearly 20% improved cytotoxicity, with an average of ˜70% cytotoxicity against Reh cells. The enhanced cytotoxicity was even more pronounced at a 2:1 ratio. While Mock NK cells killed about 65% of Reh cells, NK cells edited with CISH guide RNA 2 killed approximately 85% of Reh cells and NK cells edited with CISH guide RNA 1 killed over 90% of Reh cells. These data clearly show that CISH knockout has a positive impact on NK cell cytotoxicity, among other positive effects as discussed above.

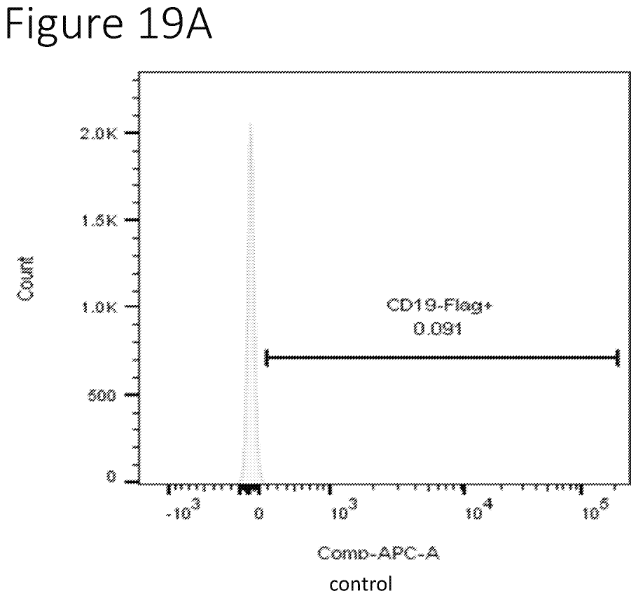

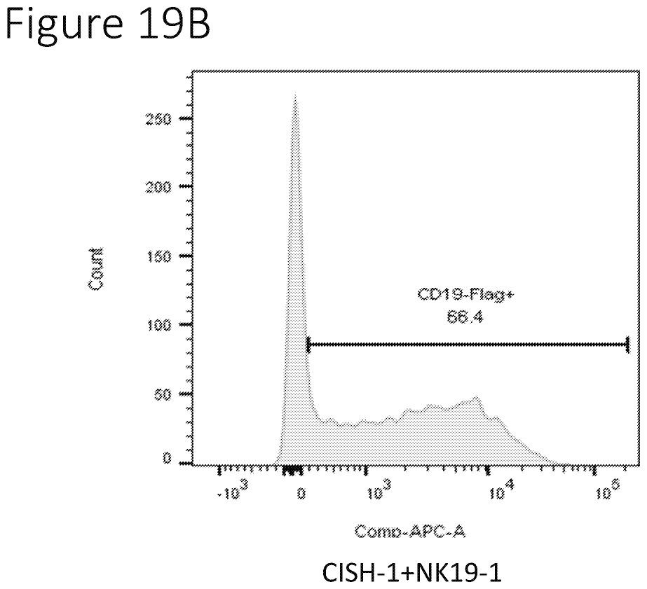

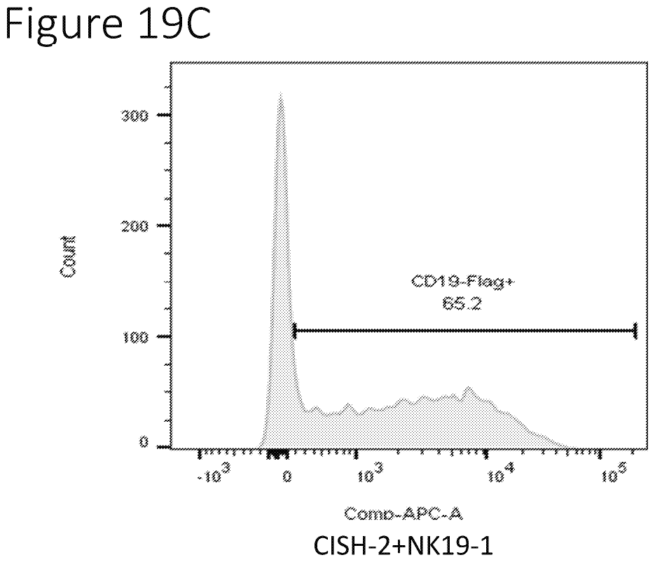

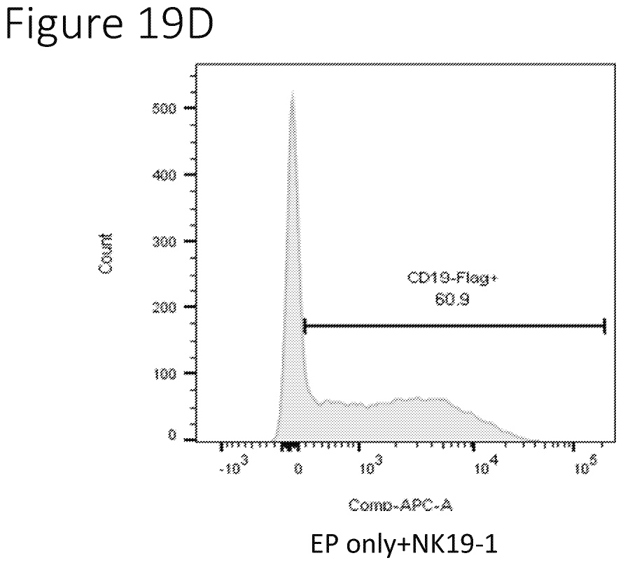

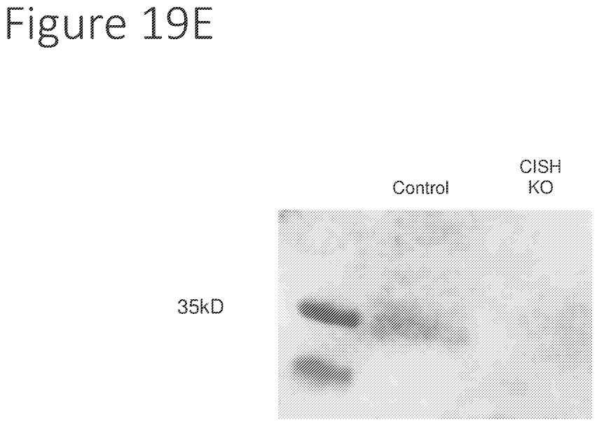

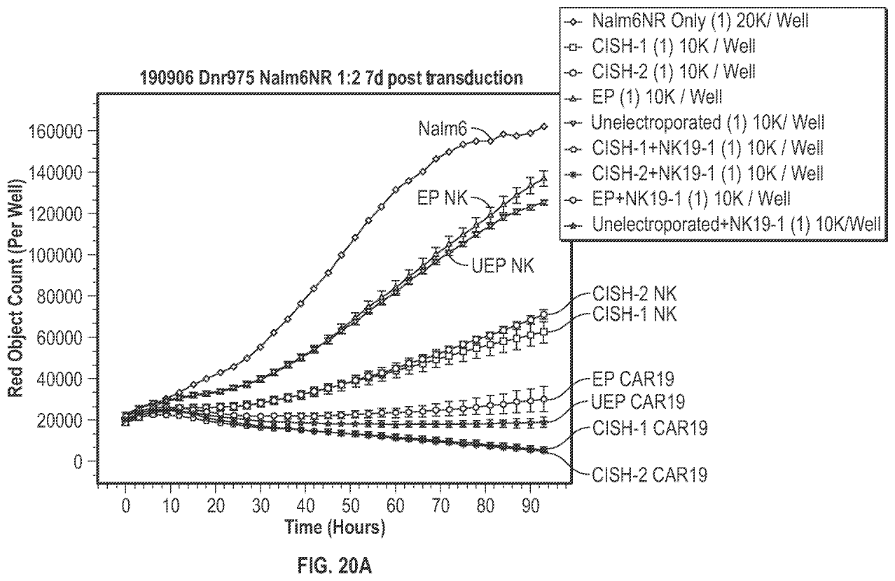

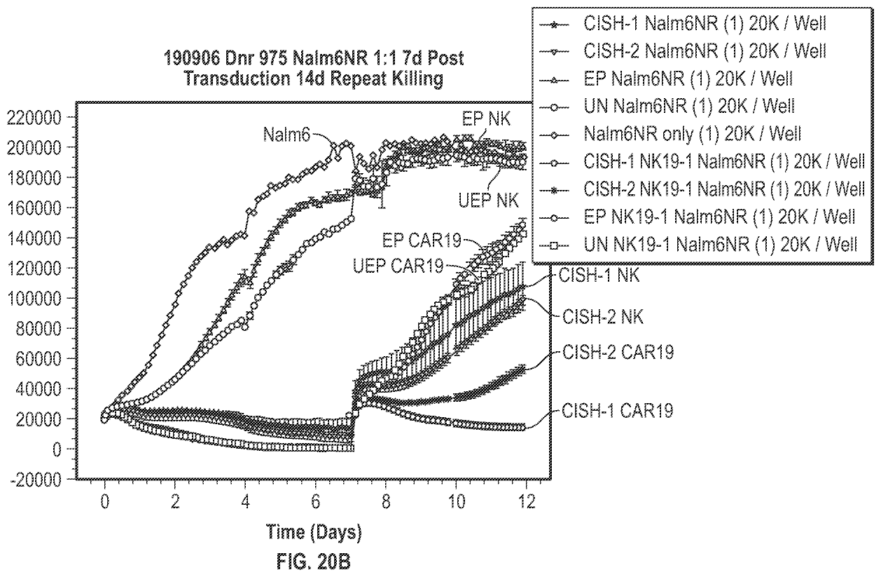

As with experiments described above, it was next evaluated whether the knockdown of CISH expression adversely impacted the ability to further modify the NK cells, for example, by transducing with a non-limiting example of a CAR (here an anti-CD19 CAR, CAR19-1). These data are shown in FIGS. 19A-19D. FIG. 19A shows negative control data for (lack of) expression of a CD19 CAR (based on detection of a Flag tag included in the CAR19-1 construct used, though some embodiments do not employ a Flag, or other, tag). FIG. 19B shows robust expression of the CD19-1 CAR by NK cells previously subjected to gene editing targeted by the CISH guide 1 RNA. FIG. 19C shows similar data for NK cells previously subjected to gene editing targeting by the CISH guide 2 RNA. FIG. 19D shows additional control data, with NK cells exposed to gene editing electroporation protocol, but without actual gene editing, thus demonstrating that the gene editing protocol itself does not adversely affect subsequent transduction of NK cells with CAR-encoding viral constructs. FIG. 20C shows a Western blot confirming the absence of expression of CIS protein (encoded by CISH) after the CISH gene editing was performed. Thus, according to some embodiments, NK cells (or T cells) are both edited, e.g., to knockout CISH expression in order to enhance one or more NK cell (T cell) characteristics through IL15-mediated signaling and are also engineered to express an anti-tumor CAR. The engineering and editing, in several embodiments, yield synergistic enhancements to NK cell function (e.g., expansion, cytotoxicity, and or persistence).

Having established that NK cells could be gene edited to reduce CISH expression and could also be engineered thereafter to express a CAR, the cytotoxicity of such doubly modified NK cells was tested. FIG. 20A shows the results of an Incucyte cytotoxicity assay where the indicated NK cell types were challenged with Nalm6 cells at a 1:2 ratio. Regarding the experimental timeline, at Day 0, NK cells were subjected to electroporation with CRISPr/Cas9, and the various CISH guide RNAs, as discussed above. The NK cells were cultured for 1 day in high IL-2 media, then moved to a low-IL-2 media where they were co-cultured with K562 cells modified to express 4-1 BB and membrane-bound IL15 for expansion. At day 7, the NK cells were transduced with the CAR19-1 viral constructs and cultured for another 7 days, with the IncuCyte cytotoxicity assay performed on Day 14.

As seen in FIG. 20A, both electroporated and un-electroporated NK cells (EP NK, UEP NK, respectively) showed nominal reduction in Nalm6 growth. When gene-edited NK cells were assessed, CISH-1 and CISH-2 NK cells both exhibited significant prevention of Nalm6 growth. Likewise, both electroporated and un-electroporated NK cells expression CAR19-1 further reduced Nalm6 proliferation. Most notably, the doubly modified CISH knockouts that express CAR19-1 exhibited complete control/prevention of Nalm6 cell growth. These results represent the synergistic activities between the two modification approaches undertaken, with gene edited CISH knockout NK cells expressing CAR19-1 showing robust anti-tumor activity, which is in accordance with embodiments disclosed herein.

These tumor-controlling effects were recapitulated in a dual challenge model as well. In this case, the experimental timeline was as described above for FIG. 20A, however, 7 days after the inception of the IncuCyte assay (performed here at 1:1E:T), the wells were washed and re-challenged with an additional dose of Nalm6 tumor cells (20K cells per well). Data are shown in FIG. 20B. As with the single tumor cell challenge, Nalm6 cells exhibited expansion throughout the experiment, with EP and UEP NK cells allowing similar overall Nalm6 growth after the second challenge. Even with the second challenge of Nalm6 tumor cells, NK cells expression CAR19-1 constructs (EPCAR19 and UEPCAR19) curtailed Nalm6 growth more so than NK cells alone. Interestingly, with the second challenge, NK cells that were gene edited to knockout CISH expression exhibited a modestly enhanced ability to prevent Nalm6 growth as compared to those expressing CAR19-1. As discussed above, this may be due to the enhanced signaling through various metabolic pathways that are upregulated due to CISH knockout. Notably, as with the single challenge, the doubly modified NK cells that were gene edited to knockout CISH expression and engineered to express CAR19-1 showed substantial ability to prevent Nalm6 cell growth. CISH guide RNA 1 and CISH guide RNA 2 treated NK cells were on par with one another until the final stages of the experiment, where CISH guide RNA 2 treated NK cells allowed a slight increase in Nalm6 cell number. Regardless, these data show that the doubly modified NK cells possess an enhanced cytotoxic ability against tumor cells. As mentioned above, the editing coupled with engineered approach in several embodiments advantageously results in non-duplicative enhancements to NK cell function, which can synergistically enhance one or more aspects of the NK cells (such as activation, cytotoxicity, persistence etc.).

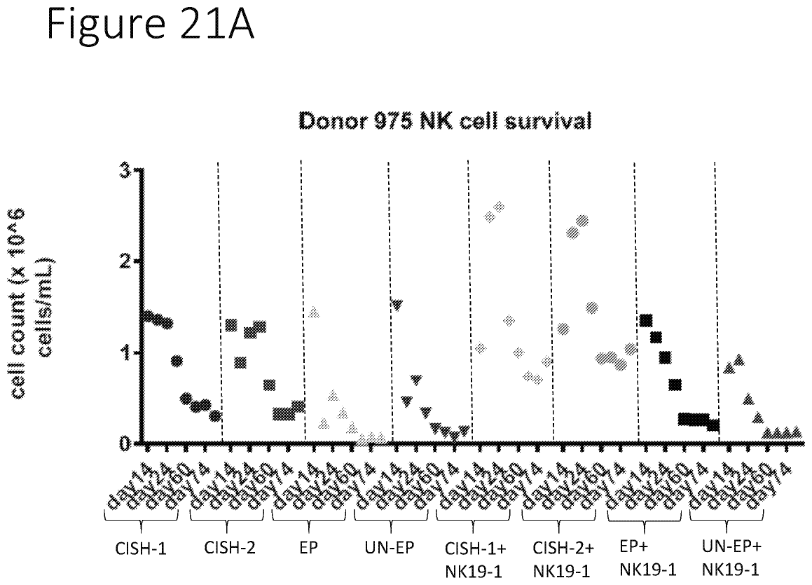

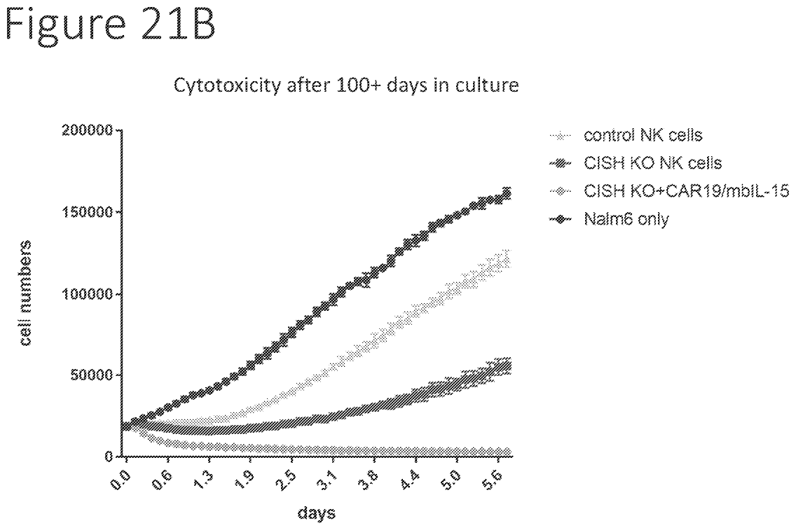

Mechanistically, without being bound by theory, it appears that the double modification of knockdown of CISH and expression of CAR19-1 allow NK cells to survive for a longer period of time, thus imparting them with an enhanced persistence against tumor cells. In several embodiments, this is due, at least in part to the enhanced signaling through various metabolic pathways in the edited cells based on knockout of CISH. Data for this analysis are shown in FIG. 21A, where cell counts were obtained for the indicated groups across 74 days in culture. Six of the eight groups tested showed a steady decline in NK cell count from about 2-3 weeks in culture, through the 74 day time point. However, the two groups of NK cells that were treated both to knockdown CISH expression and to express CAR19-1 exhibited relatively steady population size (but for a transient increase at day 24). These data suggest that the doubly modified NK cells are better able to survive than NK cells modified in only one manner (or unmodified), which may, in part, lead to their enhanced efficacy over a longer-term experiment like the secondary tumor cell challenge shown in FIG. 20B. Additionally, FIG. 21B shows cytotoxicity data for control Nalm6 cells, unmodified NK cells, CISH knockout NK cells and CISH knockout NK cells expressing CD19 CAR. This experiment was performed after each of the cell groups had been cultured for 100 days in culture. Nalm6 cells alone exhibited expansion, as expected. Control knockout NK cells (subject to electroporation only) delayed Nalm6 expansion at the initial stages, but eventually, Nalm6 cells expanded. CISH knockout NK cells showed good anti-tumor effects, with only modest increases in Nalm6 numbers at the later stages of the experiment. The cytotoxicity of NK cells at this late stage of culture is unexpected, given the growth allowed by the control NK cells. As discussed above, in several embodiments the knockout of CISH expression allows greater signaling through various IL15 responsive pathways that lead to one or more of enhanced NK (or T) cell proliferation, cytotoxicity, and/or persistence.

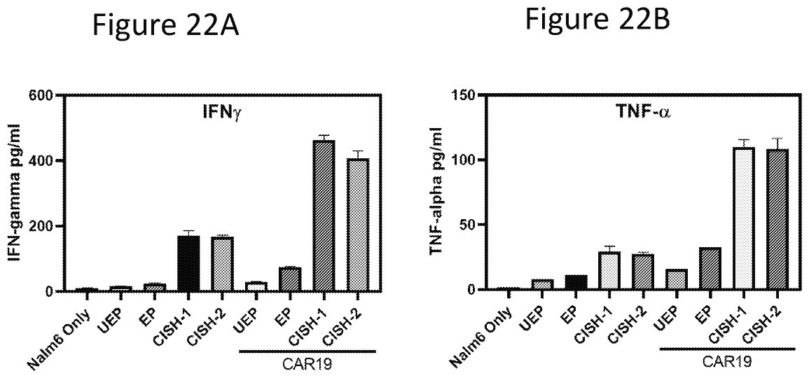

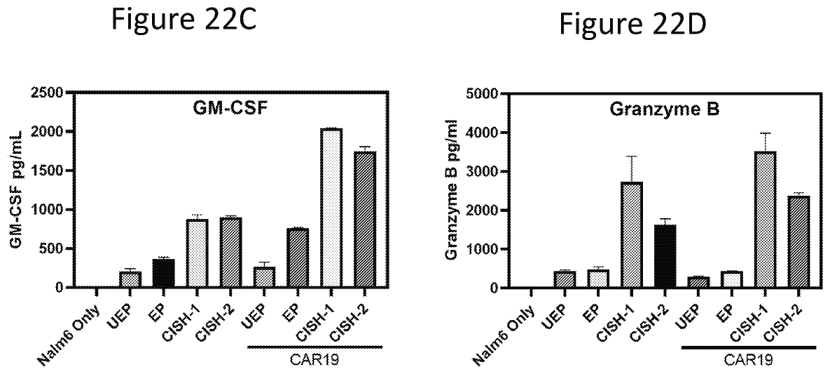

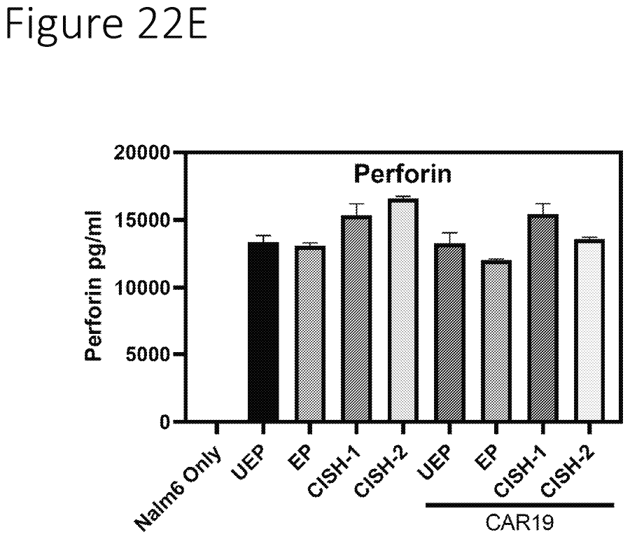

Further investigating the mechanisms by which these doubly modified cells are able to generate significant and persistent cytotoxicity, the cytokine release profiles of each group were assessed. These data are shown in FIGS. 22A-22E, with those groups of NK cells engineered to express CAR19-1 indicated by placement above the “CAR19” line on the right portion of each histogram.

FIG. 22A shows data related to IFNg production, which is notably increased when CISH is knocked out through use of CRISPr/Cas9 and either guide RNA 1 or 2 (as non-limiting embodiments of guide RNA). More interestingly, the combination of CISH knockout and CAR19-1 expression results in nearly 2.5 times more IFNg production than the CISH knockouts and 4-5 times more than any of the other groups. Similar data are shown in FIG. 22B, with respect to TNFalpha production. Likewise, while the CISH knockouts alone and the CISH-normal NKs expressing CAR19-1 release somewhat more GM-CSF, the doubly modified CISH knockout and CAR19-1-expressing NK cells show markedly increased GM-CSF release. Granzyme B release profiles, shown in FIG. 22D, again demonstrates that the doubly modified cells release the most cytokine. Interestingly the levels of Granzyme B expression correlate with the cytotoxicity profiles of the CISH 1 and CISH 2 NK cell groups. Both the CISH 2 NK and CISH 2/CAR19 groups release less Granzyme B than their CISH 1 counterparts, which is reflected in the longer term cytotoxicity data of FIG. 20B, suggesting that reduced CISH expression may be inversely related to Granzyme B release. Finally, FIG. 22E shows release of perforin, which is significantly higher for all NK cell groups, and does not reflect the same patterns seen in FIGS. 22A-22D, suggesting perforin is not a cytotoxicity-limiting cytokine, in these embodiments. However, these data do confirm that immune cells that are subjected to the combination of gene editing (e.g., to reduce expression of an inhibitory factor expressed by the immune cell or to reduce the ability of the immune cell to respond to an inhibitory factor) and the engineering of the cell to express a chimeric cytotoxic signaling complex (such as, for example, a cytotoxicity inducing CAR). In several embodiments, the doubly modified cells exhibit a more robust (e.g., cytotoxicity-inducing) cytokine profile and/or show increased viability/persistence, which allows a greater overall anti-tumor effect, as in accordance with several embodiments disclosed herein. In several embodiments, the double modification of immune cells therefore leads to an overall more efficacious cancer immunotherapy regime, whether using NK cells, T cells, or combinations thereof. Additionally, as discussed above, in several embodiments, the doubly modified cells are also modified in order to reduce their alloreactivity, thereby allowing for a more efficacious allogeneic cell therapy regimen.

CBLB is an E3 ubiquitin ligase that is known to limit T cell activation. In order to determine if disruption of expression of CBLB by NK cells could elicit a more robust anti-tumor response from engineered NK cells, as discussed above, CRISPR/Cas9 was used to disrupt expression of CBLB, though in additional embodiments, other gene editing approaches can be used.

Non-limiting examples of CBLB-targeting guide RNAs are shown below in Table 5.

TABLE 5
CBLB Guide RNAs

| SEQ ID NO: | Name | Sequence | Target |
|---|---|---|---|
| 164 | CBLB-1 | TAATCTGGTGGACCTCATGAAGG | Exon 5 |
| 165 | CBLB-2 | TCGGTTGGCAAACGTCCGAAAGG | Exon 10 |
| 166 | CBLB-3 | AGCAAGCTGCCGCAGATCGCAGG | Exon 2 |

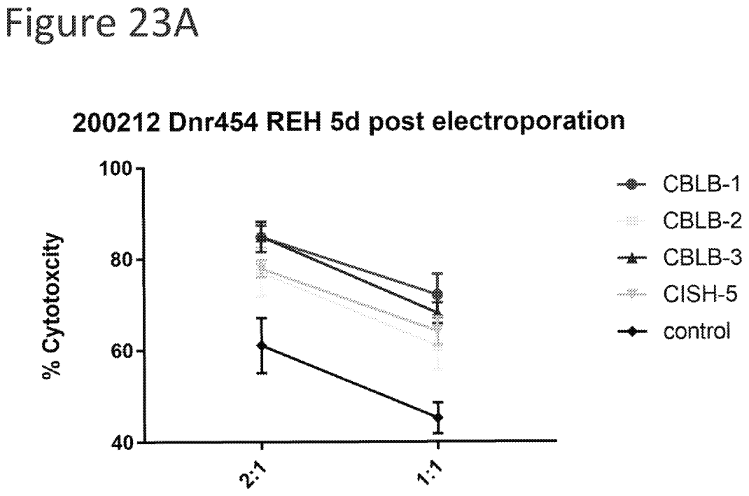

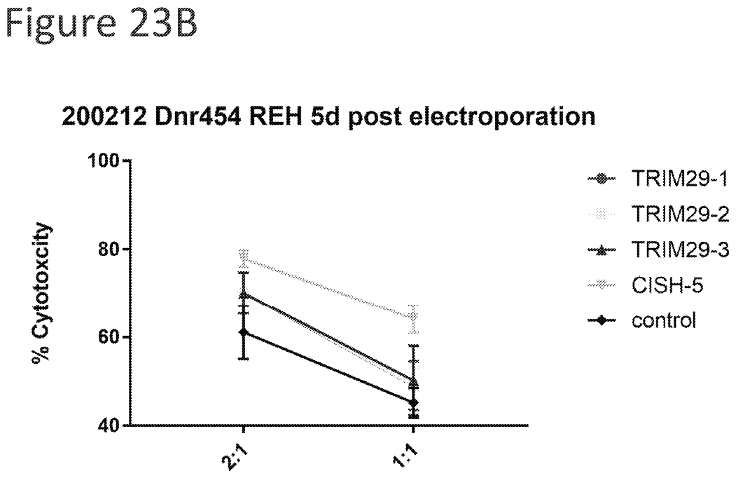

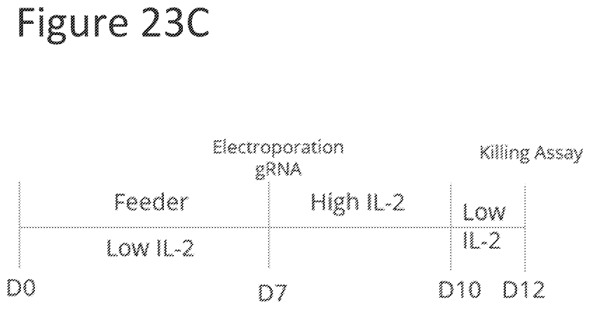

As with the NKG2A and CISH knockout NK cells, Cbl proto-oncogene B (CBLB) knockout (using the guide RNAs shown in Table 5 [SEQ ID NO: 164, 165, 166]) and CISH knockout (using CISH guide RNA 5 [SEQ ID NO: 157]) gene edited NK cells were challenged with Reh tumor cells at a 1:1 and 2:1E:T ratio 5 days after being electroporated with the gene editing machinery. Briefly, parent NK cells were maintained in a low IL-2 media with feeder cells for 7 days, electroporated on day 7, incubated in high IL-2 media on days 7-10, low IL-2 media on days 10-12, then subjected to the Reh tumor challenge assay on day 12 (FIG. 23C). FIG. 23A shows that while mock NK cells exhibited ˜45% cytotoxicity against Reh cells at the 1:1 ratio, each of the CBLB gRNA knockout NK cell groups showed ˜20% greater cytotoxicity, with an average of ˜70% cytotoxicity against Reh cells. For the 2:1 ratio, the corresponding enhanced cytotoxicity is similar to the 1:1 ratio group, with mock NK cells exhibiting ˜60% cytotoxicity, and each of the CBLB knockout NK cell groups showing a ˜20% greater cytotoxicity, with an average of 80% cytotoxicity against Reh cells. The CISH gRNA 5 knockout NK cell group also exhibited similar results, with approximately 65% in the 1:1 ratio and approximately 80% in the 2:1 ratio, consistent with the previous CISH knockout experiment using gRNAs 1 and 2, discussed above. Overall, the increase in cytotoxicity in CBLB knockout NK cells is proportionate with the CISH knockout NK cells. These data shows that CBLB knockout, in accordance with several embodiments disclosed herein, has a positive impact on NK cell cytotoxicity. In several embodiments, combinations of CISH knockout and CBLB knockout are used to further enhance the cytotoxicity of engineered NK cells. In several embodiments, CBLB knockout NK cells exhibit a greater responsiveness to cytokine stimulation, leading, in part to their enhanced cytotoxicity. In several embodiments, the CBLB knockout leads to increased resulting in increased secretion of effector cytokines like IFN-g and TNF-α and upregulation of the activation marker CD69. In several embodiments, knockout of CBLB is employed in conjunction with engineering the NK cells to express a CAR, leading to further enhancement of NK cell cytotoxicity and/or persistence.

Another E3 ubiquitin ligase, TRipartite Motif-containing protein 29 (TRIM29), is a negative regulator of NK cell functions. TRIM29 is generally not expressed by resting NK cells, but is readily upregulated following activation (in particular by IL-12/IL-18 stimulation). As discussed above, CRISPR/Cas9 was also used to disrupt expression of TRIM29, though in additional embodiments, other gene editing approaches can be used. Non-limiting examples of TRIM29-targeting guide RNAs are shown below in Table 6.

TABLE 6
TRIM29 Guide RNAs

| SEQ ID | | | |
|---|---|---|---|
| NO: | Name | Sequence | Target |
| 167 | TRIM29-1 | GAACGGTAGGTCCCCTCTCGTGG | Exon 4 |
| 168 | TRIM29-2 | AGCTGCCTTGGACGACGGGCAGG | Exon 7 |
| 169 | TRIM29-3 | TGAGCCGTAACTTCATTGAGAGG | Exon 4 |

TRIM29 knockout (using the gRNAs shown in Table 6 [SEQ ID NO: 167, 167, 169]) gene edited NK cells were challenged with Reh tumor cells at a 1:1 and 2:1E:T ratio 5 days after being electroporated with the gene editing machinery. The timeline and culture parameters were the same as the CBLB knockout example (FIG. 23C). FIG. 23B shows that TRIM29 knockout has a somewhat less robust impact on enhancing cytotoxicity compared to the CISH or CBLB knockouts. Each of the TRIM29 gRNA NK cell groups had cytotoxicity against Reh cells slightly better than mock cells (˜50% vs ˜45% cytotoxicity at the 1:1 ratio and ˜70% vs ˜60% cytotoxicity at the 2:1 ratio). Comparatively, NK cells transfected with the CISH gRNA 5 had improved cytotoxicity relative to both mock and TRIM29 knockout NK cells in both 1:1 and 2:1 ratio. While these results indicate that TRIM29 only had a minor effect or no effect on NK cell cytotoxicity under these conditions, that may be at least in part due to the target cell type (e.g., the pathways altered in response to changes in TRIM29 expression are not as active as, for example those altered by changes in CBLB expression). In addition, in several embodiments, a combination of engineering the NK cells with a CAR construct, for example a CAR targeting CD19 and knocking out TRIM29 expression results in significantly enhanced NK cell cytotoxicity and/or persistence. In several embodiments, knockout of TRIM29 expression upregulates interferon release by NK cells.

Interleukins, in particular interleukin-15, are important in NK cell function and survival. Suppressor of cytokine signaling (SOCS) proteins are negative regulators of cytokine release by NK cells. The protein tyrosine phosphatase CD45 is an important regulator of NK cell activity through Src-family kinase activity. CD45 expression is involved in ITAM-specific NK-cell functions and processes such as degranulation, cytokine production, and expansion. Thus, knockout of CD45 expression should result in less effective NK cells. As discussed above, CRISPR/Cas9 was used to disrupt expression of CD45 and SOCS2, though in additional embodiments, other gene editing approaches can be used. Non-limiting examples of CD45 and SOCS2-targeting guide RNAs are shown below in Table 7.

TABLE 7
CD45 and SOCS2 Guide RNAs

| SEQ | | | |
|---|---|---|---|
| ID NO: | Name | Sequence | Target |
| 170 | CD45-1 | AGTGCTGGTGTTGGGCGCAC | Exon 25 |
| 171 | SOCS2-1 | GTGAACAGTGCCGTTCCGGGGGG | Exon 3 |
| 172 | SOCS2-2 | GGCACCGGTACATTTGTTAATGG | Exon 3 |
| 173 | SOCS2-3 | TTCGCCAGACGCGCCGCCTGCGG | Exon 2 |

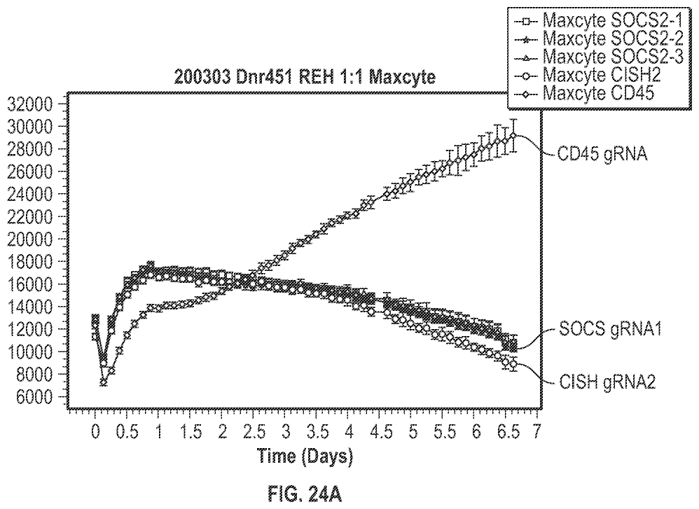

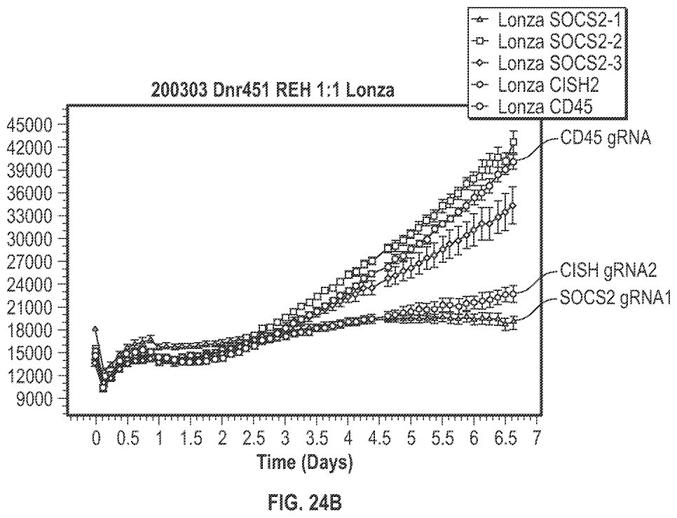

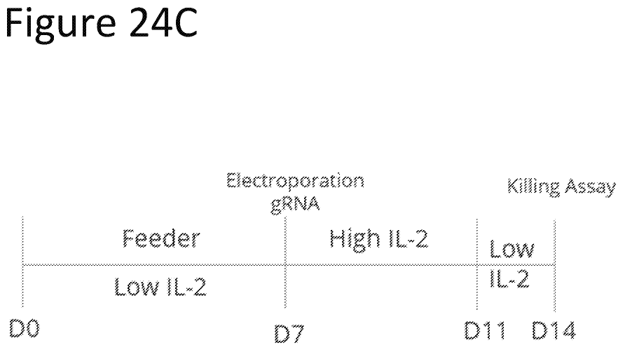

Suppressor of cytokine signaling 2 (SOCS2) knockout (using the gRNAs showed in Table 7 [SEQ ID NO: 171, 172, 173]) gene edited NK cells were assessed in a time course cytotoxicity assay 7 days after being electroporated with the gene editing machinery. Briefly, parent NK cells were maintained in a low IL-2 media with feeder cells for 7 days, electroporated on day 7, incubated in high IL-2 media for days 7-11, low IL-2 media on days 11-14, then subjected to the Incucyte cytotoxicity assay against Reh cells at a 1:1E:T ratio on day 14 (FIG. 24C). FIG. 23A shows the results of the cytotoxicity assay with NK cells electroporated with a first electroporation system. Using this system, NK cells transfected with each of the SOCS2 gRNAs exhibited cytotoxic activity similar to the CISH gRNA 2 NK cell group (described above). The three gRNA curves for SOCS2 are superimposed in FIG. 24A. CD45 knockout NK cells served as the negative control (as discussed above, CD45 is a positive regulator of NK cell activity, so the CD45 knockout should show reduced cytotoxicity). FIG. 23B shows the results of the cytotoxicity assay with NK cells following the same schedule but electroporated with a second electroporation system. In this case, out of the SOCS2 gRNAs examined, SOCS2 gRNA 1 resulted in an improved cytotoxicity against Reh cells. SOCS2 gRNA 2 and 3 yielded less effective NK cells than with the first electroporation system. SOCS2 gRNA 1 knockout NK cells showed a slight enhancement in cytotoxicity compared to CISH gRNA 2 knockout NK cells. These results indicate that, according to several embodiments, knockout of SOCS2 reduces the negative regulation of NK cells and yield NK cells with enhanced cytotoxicity. In several embodiments, specific gRNAs are used to enhance the cytotoxic NK cells, for example SOCS2 gRNA 1. In several embodiments, knockout of SOCS2 is employed in conjunction with engineering the NK cells to express a CAR, leading to further enhancement of NK cell cytotoxicity and/or persistence.

### Example 2—Approaches for Generating Hypoimmune Therapeutic Cells

As discussed herein, in several embodiments, a mixed immune cell population is used in therapy. In several embodiments, a mixture of T cells and NK cells are employed, each optionally engineered to express a CAR targeting a tumor marker and/or edited to reduce or alter expression of an endogenous gene. In several embodiments, the editing is required due to overlap with or similarity between an endogenous marker expressed by one, or both (or more if more than two), of the immune cell populations and the cancer marker being targeted with the CAR. In such circumstances, were the editing not performed, fratricide among the therapeutic cells would result. In some embodiments, editing is required even if only one immune cell type is used, as fratricide can occur within a single cell population as well.

In a mixed cell population, when considered in an autologous context, T cells express MHC-I in complex with a self-peptide, which functions as an inhibitory ligand for NK cell receptors, even though NK cells express both inhibitory and activating receptor. In this context, the MHC-I inhibitory signal overpowers activating signals on NK cells. Thus, using a mixed NK and T cell population from collected from and then delivered to the same donor should be compatible.

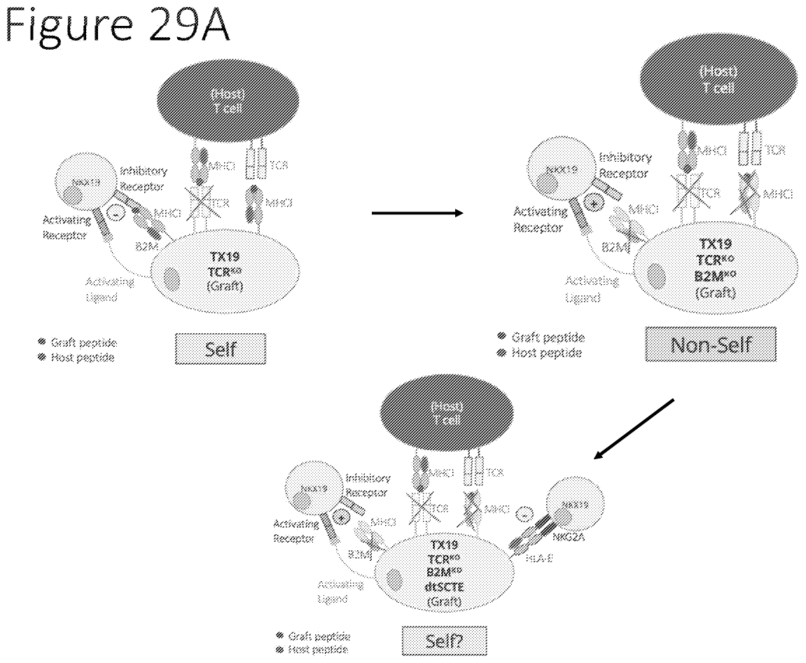

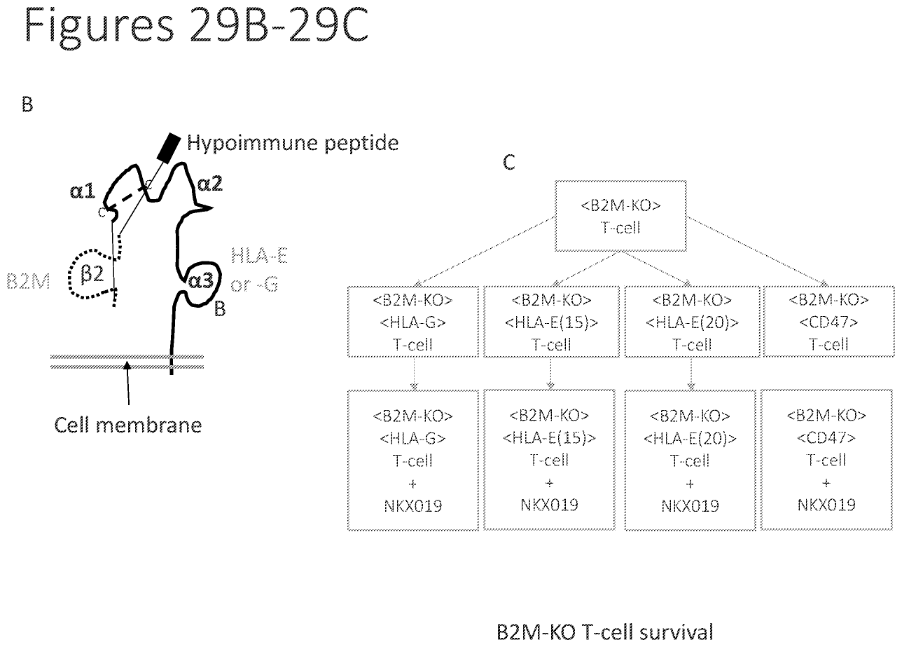

In contrast, in an allogeneic setting, graft versus host rejections can occur between graft and host T cells. In this context, the peptides presented by the host T cells (host “self” peptides) would interact with the graft (engineered) T cell receptor and since the host peptide would be non-self vis-à-vis the graft T cell, the graft T cell would act cytotoxically towards the host T cell. According to some embodiments provided for herein, genetic editing to remove/deplete TCR expression/function in the therapeutic T cell population (graft cells) would prevent their activation by host T cell peptide presentation. Thus, in several embodiments, therapeutic T cells are edited to knockout the TCR (referred to as “TCR^{KO}”) Reversing the perspective, the graft T cells, if un-edited, still present donor peptides on MHCI, which are non-self vis-à-vis the host T cells, which would result in host versus graft effects. However, according to some embodiments disclosed herein, gene editing is performed to remove/deplete B2M expression in therapeutic T cells, which disrupts graft peptide presentation by the therapeutic T cells. Thus, in several embodiments, therapeutic T cells are edited to knockout B2M (referred to as “B2M^{KO}”). A side effect of this B2M knockout, due to the loss of the presentation of self-peptide by the therapeutic T cell, is that the prior inhibitory signal that prevented therapeutic NK cell activity against therapeutic T cells has been removed. Thus, whilst B2M knockout protects therapeutic T cells from host T cells, it also renders them susceptible to therapeutic NK cells. To combat this newly generated susceptibility to therapeutic NK cells, experiments were undertaken to determine if HLA re-expression by therapeutic T cells could confer resistance to the activity of therapeutic NK cells. In several embodiments, HLA-E is re-expressed to protect therapeutic T cells. In several embodiments, HLA-G is re-expressed to protect therapeutic T cells. In several embodiments, HLA-E and G are re-expressed to protect therapeutic T cells. In several embodiments, a disulfilde trap single chain trimer (dtSCT) is used to express HLA-E and/or HLA-G and B2M (see FIG. 29B for a schematic). FIG. 29C shows a schematic of an experimental setup to assess the compatibility of various engineered/edited immune cells in a mixed population. Briefly, T cells are edited according to embodiments disclosed herein to knockout B2M. Those B2M^{KO} cells are split and then further edited to express, via a dtSCT: HLA-G (referred to as “B2M-KO_dtSCTG”), HLA-E (using a 15 amino acid linker to connect the peptide, referred to as “B2M-KO_dtSCTE15”), HLA-E (using a 20 amino acid linker to connect the peptide, referred to as “B2M-KO_dtSCTE20”), or tCD47 (referred to as “B2M-KO_CD47”). Each of those resulting cell groups were then co-cultured with NK cells expressing, as a non-limiting example, an anti-CD19 CAR, in order to assess how the genetic modifications (B2M knockout and expression of various peptides) functioned to establish immune compatibility between the cell types.

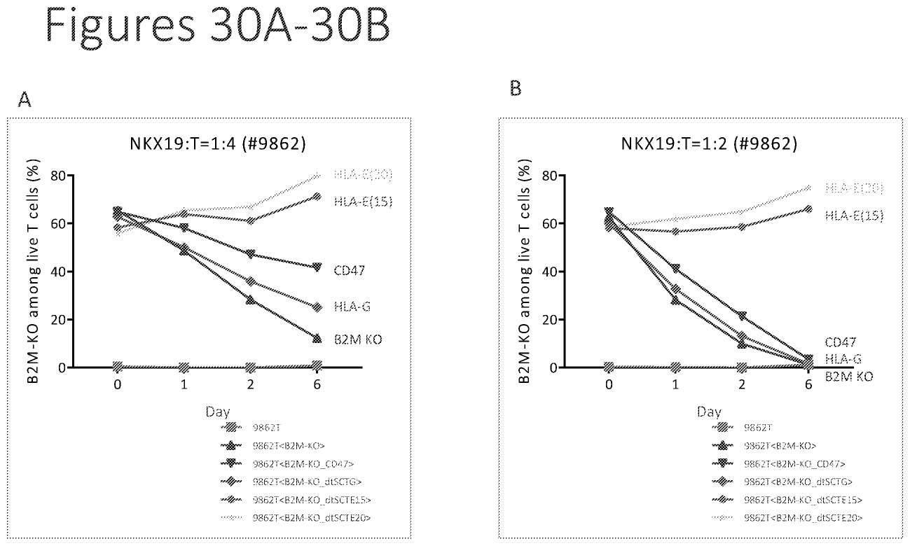

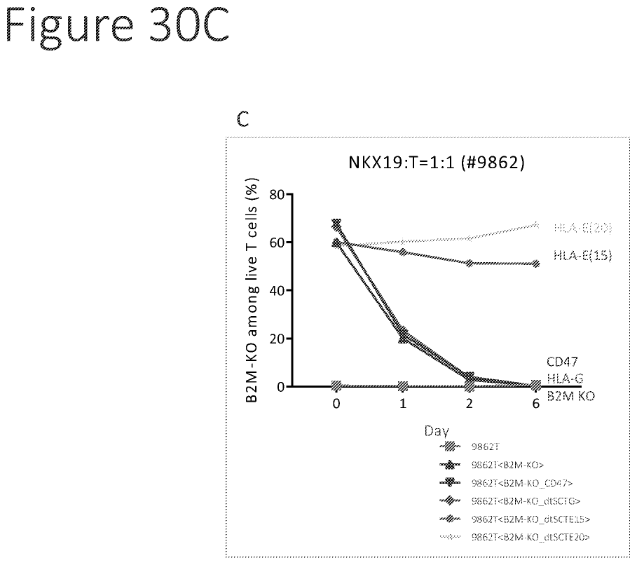

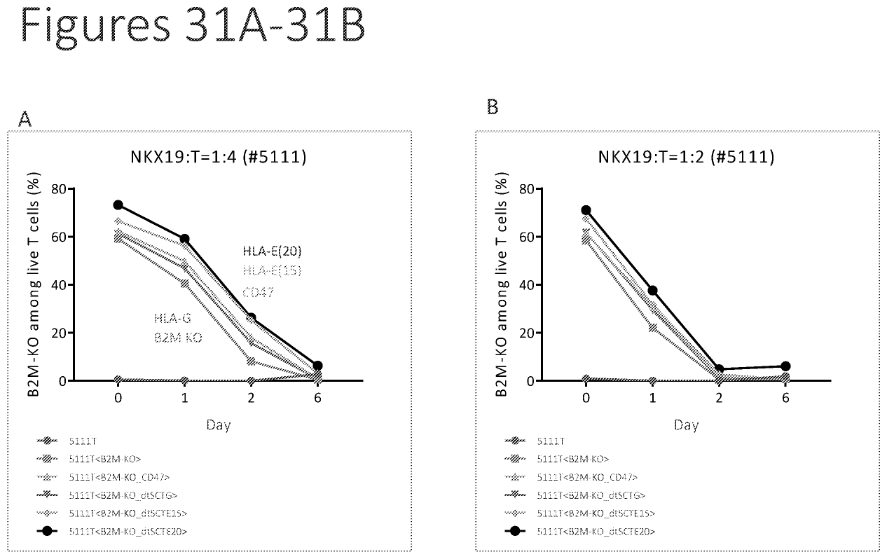

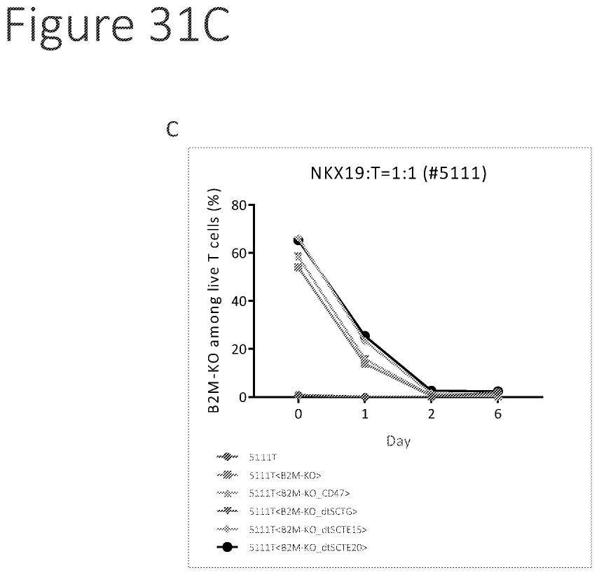

Various NK:T cell ratios were tested, with the resultant data shown in FIGS. 30A-30C. Data are presented as the percentage of B2M^{KO} T cells out of the live T cells present in the co-culture over time. Co-culture was performed for 6 days, with measurements taken at Day 0, 1, 2, and 6. FIG. 30A shows the data for an NK:T cell ratio of 1:4. Unmodified T cells and B2M^{KO} T cells were used as controls. As shown in FIG. 30A, the various genetic modifications and expression of immunosuppressive peptides confers various degrees of compatibility with NK cells, with the expression of HLA-E exhibiting the greatest inhibition of the NK cell-based elimination of B2M^{KO} T cells, and actually allowing a slight expansion of the T cells over time. With a 1:2 NK:T cell ratio (FIG. 30B), there was a greater degree of elimination of B2M^{KO} T cells particularly with HLA-G and tCD47 expression. Notably HLA-E (whether using a 15 or 20 amino acid linker) maintained relatively consistent levels during the co-culture, despite there being present a higher ratio of NK cells in the co-culture. FIG. 30C shows survival data when a 1:1 NK:T cell ratio is used in the co-culture. As with the 1:2 ratio, HLA-E expression conferred the greatest degree of NK and T cell compatibility, with the other constructs showing reduction in T cell numbers over the course of the experiment. FIGS. 31A-31C show corresponding T cell survival data from an additional donor, with these data indicating that there is some degree of donor-specific effect in terms of the ultimate compatibility with the NK cells. For example, these data show that, at a 1:4 NK:T cell ratio, even the cells modified to express HLA-E showed reduced survival over time (FIG. 31A), with the higher ratios (FIGS. 31B and 31C) exhibiting reduced modified T cell numbers at earlier time-points in the co-culture.

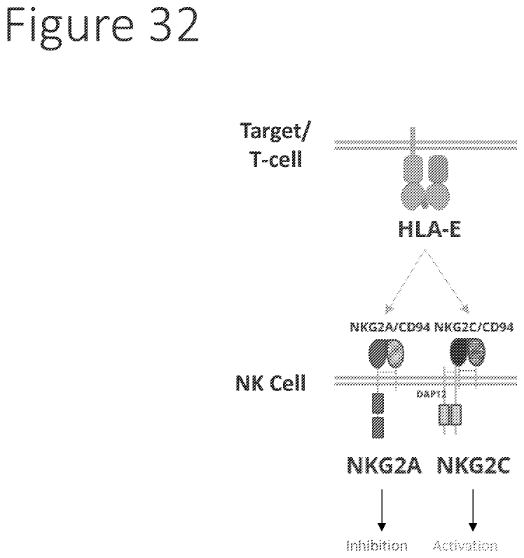

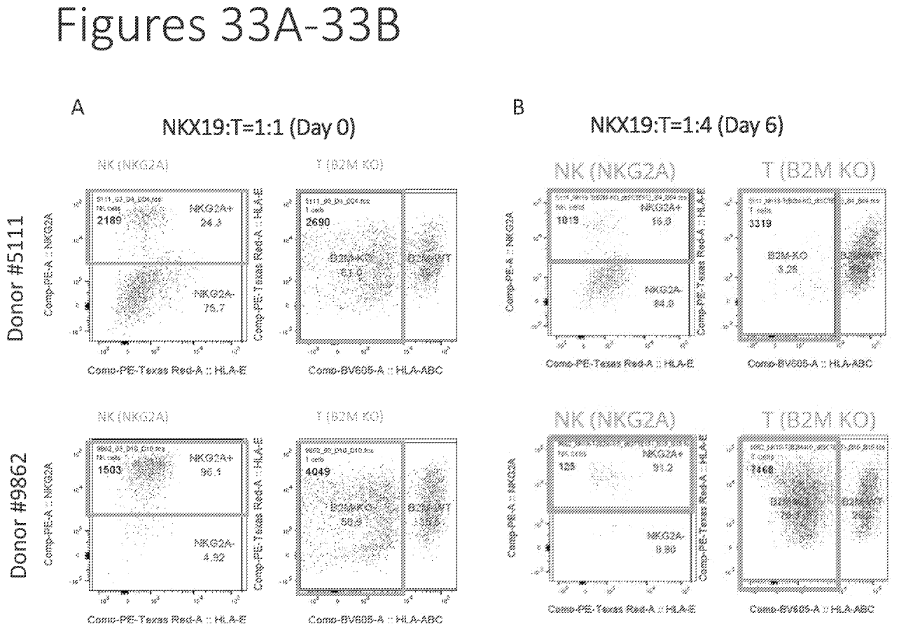

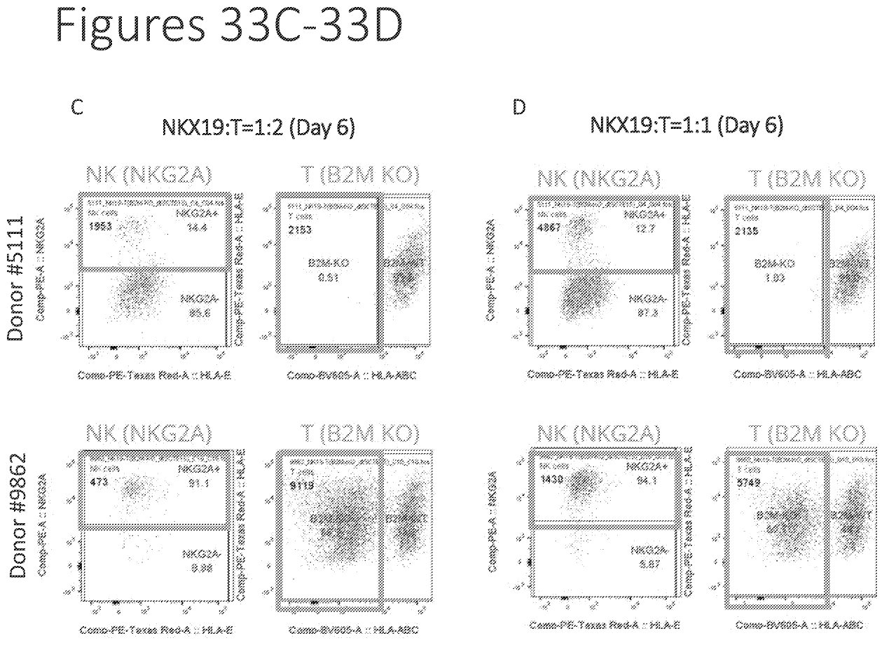

Without wishing to be bound by theory, the mechanism of action of HLA-E conferring “self” identity that is recognized by NK cells (as discussed above) is achieved through a balance of inhibitory and activating receptors on NK cells. NKG2A is the receptor that transduces inhibitory signals, while NKG2C is the receptor that transduces activation signals. Thus, according to several embodiments, the degree of expression of NKG2A and/or NKG2C by NK cells may impact the degree of activity that the NK cells exhibit against T cells (either in the absence of, or even with the re-expression of HLA-E). To evaluate this possibility, cells from two donors (one with HLA-E based protection, FIGS. 30A-30C and one showing reduced HLA-E based protection, FIGS. 31A-31) were evaluated for the degree of NKG2A and B2M expression at various points during the co-culture. FIG. 33A shows data from Day 0 of co-culture at a 1:1 NK:T ratio for the donor showing reduced HLA-E based protection (top row) and the donor showing HLA-E based protection (bottom row). While T cells from both donors exhibited similar levels of B2M expression (˜60% of the cells from each donor were B2M-negative), there were marked differences in NKG2A expression. The donor on the top (non-HLA-E protected) had −24% of the NK cells being NKG2A-positive, with the donor on the bottom (exhibited HLA-based protection) had over 95% of the NK cells expressing NKG2A. FIG. 33A shows data from Day 6 for a 1:4 NK:T ratio with a similar pattern of NKG2A expression exhibited between the two donors. In contrast, however, the degree of B2M^{KO} T cells present at Day 6 was just over 3% in the non-HLA-E protected donor, while the degree of B2M^{KO} T cells present for the HLA-E protected donor was over 70%. FIG. 32C shows corresponding data for a 1:2 ratio and FIG. 32D shows corresponding data for a 1:1 NK:T ratio. Even at the higher ratios (more NK cells for every “target” T cell), the lower degree of NKG2A positivity correlated with a lower degree of B2M^{KO} T cells, and in contrast the greater degree of NKG2A positivity correlated with a greater degree of B2M^{KO} T cells surviving. Given that NKG2A moderates the inhibitory effects of HLA-E, donors expressing greater NKG2A levels may, according to some embodiments, showed reduced fratricide of T cells modified to re-express HLA-E in a mixed NK/T population of therapeutic cells.

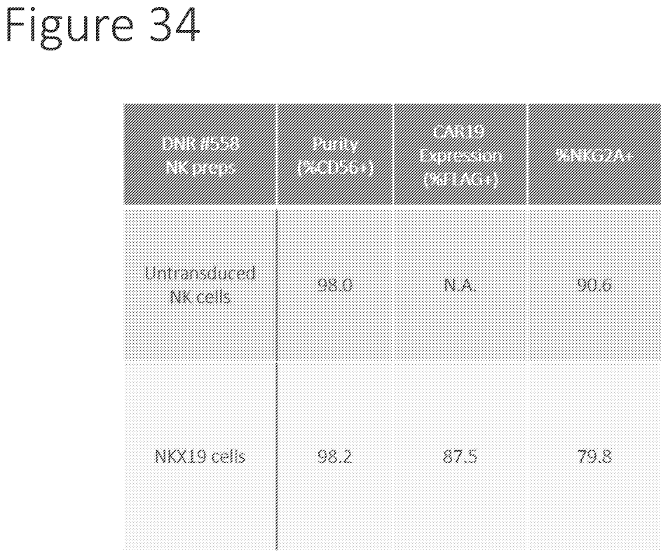

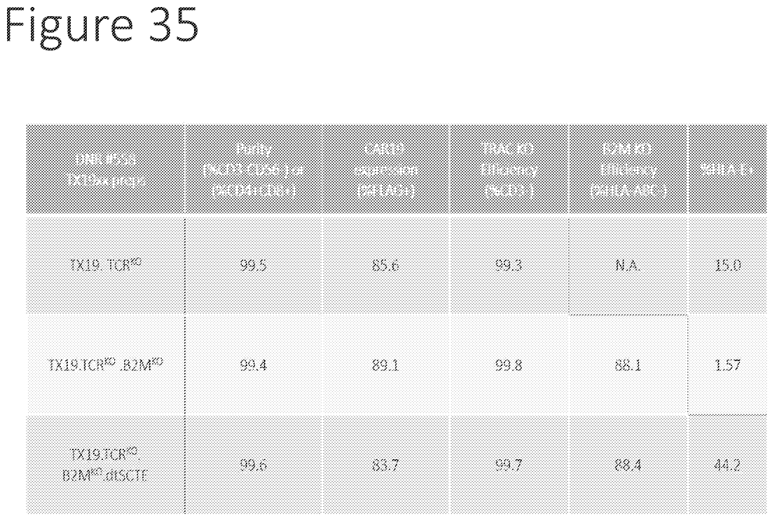

To further investigate the mechanism by which T cells can be protected from cytotoxic effects of NK cells in a mixed therapeutic cell culture, NK cells and T cells from a single donor were collected and phenotypically characterized. FIG. 34 shows the resultant characteristics of NK cells when untransduced (top row) and when engineered to express, as a non-limiting embodiment of CAR, an anti-CD19 CAR (bottom row). These data show that the cell populations are significantly pure NK cells (based on CD56 positivity) and express not only high CAR percentages for the experimental population, but also nearly 80% of the cells are NKG2A positive. FIG. 35 shows corresponding characterization for the T cells. Each of the three groups of T cells were engineered to express an ani-CD19 CAR, and again, were substantially pure populations of T cells (based on CD3/CD56 negativity or CD4/CD8 positivity). Over 80% of the population expressed the CD19 CAR and efficiency of TCR knockout was 99% or more. The first experimental group was not edited to knockout B2M while groups 2 and three showed 88% efficient B2M knockout. The last experimental group was engineered to re-express HLA-E, with about 45% for the cells being HLA-E positive. The experimental groups were designed to aid in determining whether B2M-deficient T cells (including HLA-E re-expressing cells) expressing an anti-tumor CAR are compatible with untransduced NK cells and also whether, in the context of an NK/T mixed therapeutic population, whether T cells (including HLA-E re-expressing cells) expressing an anti-tumor CAR are compatible with NK cells also expressing an anti-tumor CAR (optionally to a different tumor target).

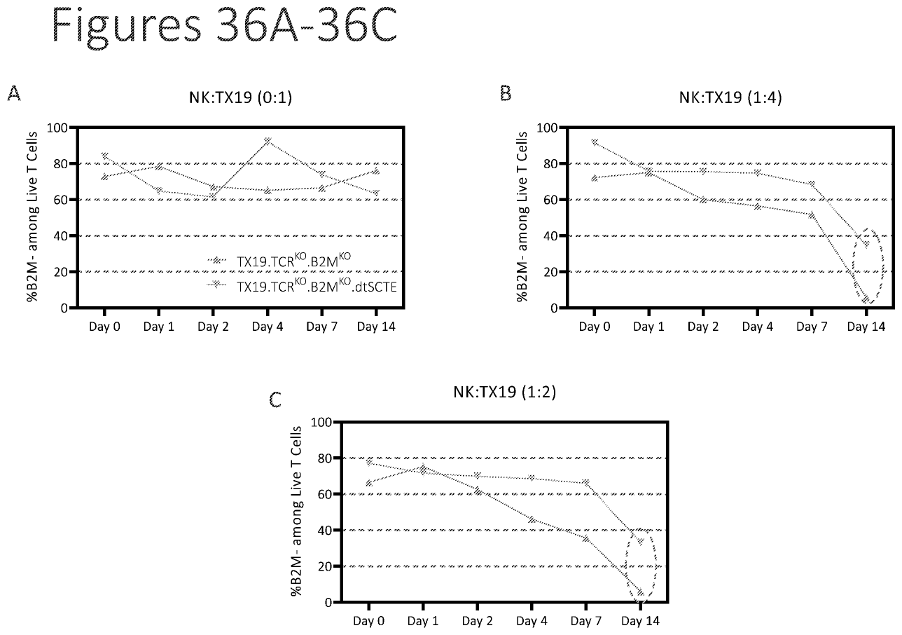

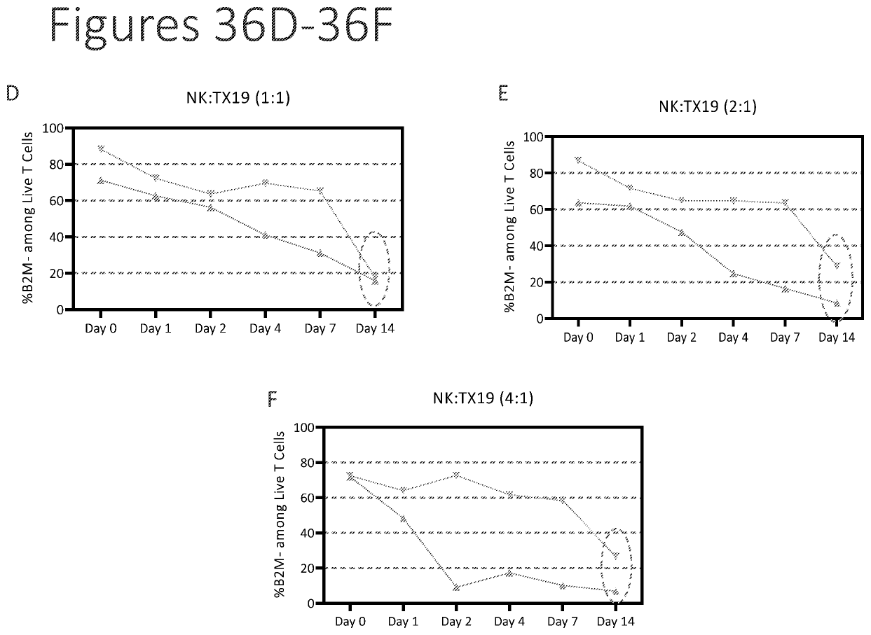

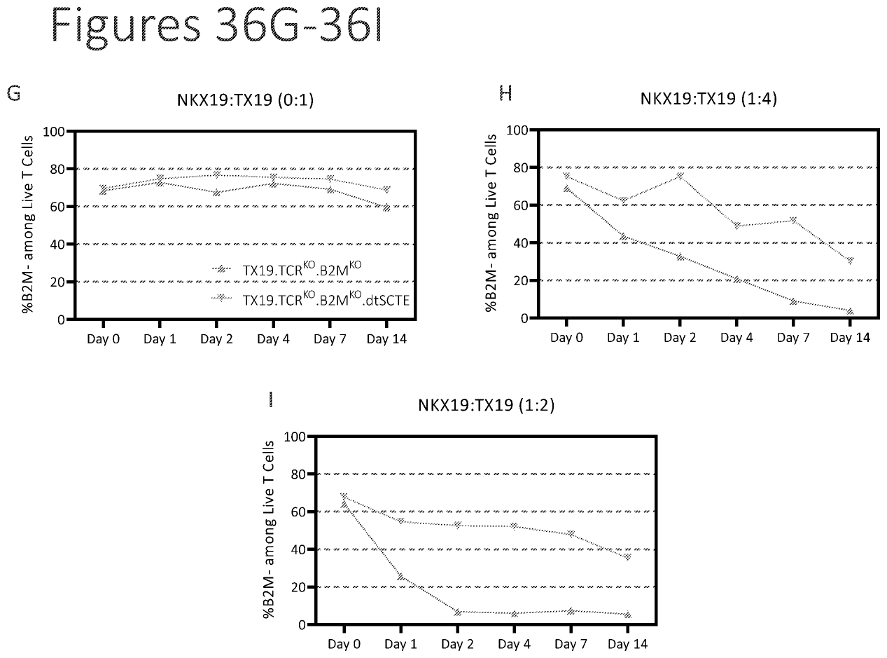

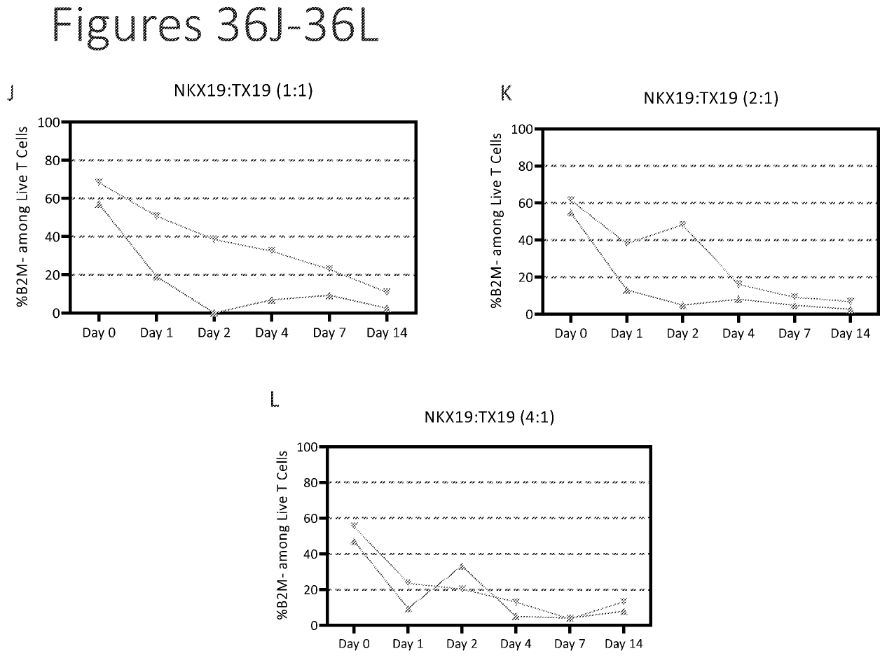

FIGS. 36A-36F show data from coculture of NK cells (unmodified) with T cells expressing an anti-CD19 CAR and that are either TCR/B2M knockout (“TX19.TCR^{K}O.B2M^{K}O”) or TCR/B2M with HLA-E re-expressed (“TX19. TCR^{KO}.B2M^{KO}.dtSCTE”). NK:T cell ratios are (A-F, respectively) 0:1, 1:4, 1:2, 1:1, 2:1, and 4:1. Co-culturing was performed for 14 days and the percentage of B2M^{KO} T cells (out of live T cells) present in the culture was assessed at the indicated timepoints with HLA-E re-expressing data points indicated by the inverted triangles. FIG. 36A shows positive control data, as this represents the culture of the indicated T cells without any NK cells present. FIG. 36B shows the data with the lowest NK:T ratio. As with the prior data for an HLA-E protected donor (expressing relatively high NKG2A), the T cell population was relative stable though 7 days of co-culture. Between 7 and 14 days, the percentage of B2M^{KO} T cells was reduced in both groups, though those cells with re-expressed HLA-E made up nearly 40% of the remaining population of T cells, while those without HLA-E re-expression dropped to nearly zero. A dashed circle surrounds these two data points, which are indicative of HLA-E based protection of the T cells from cytotoxic effects of the NK cells. A similar pattern was seen in the 1:2 NK:T ratio shown in FIG. 36C. As the ratio of NK:T cells increased (FIG. 36C-36F), the basic pattern of T cell survival over the course of the experiment was relatively maintained, though the survival numbers at the final time point for the HLA-E re-expressing group was serially modestly reduced. However, these data indicate that, even at relatively high NK:T ratios (up to at least 4:1), the re-expression of HLA-E on T cells confers a protective effect on those T cells vis-à-vis NK cells, as compared to those T cells without HLA-E re-expression.

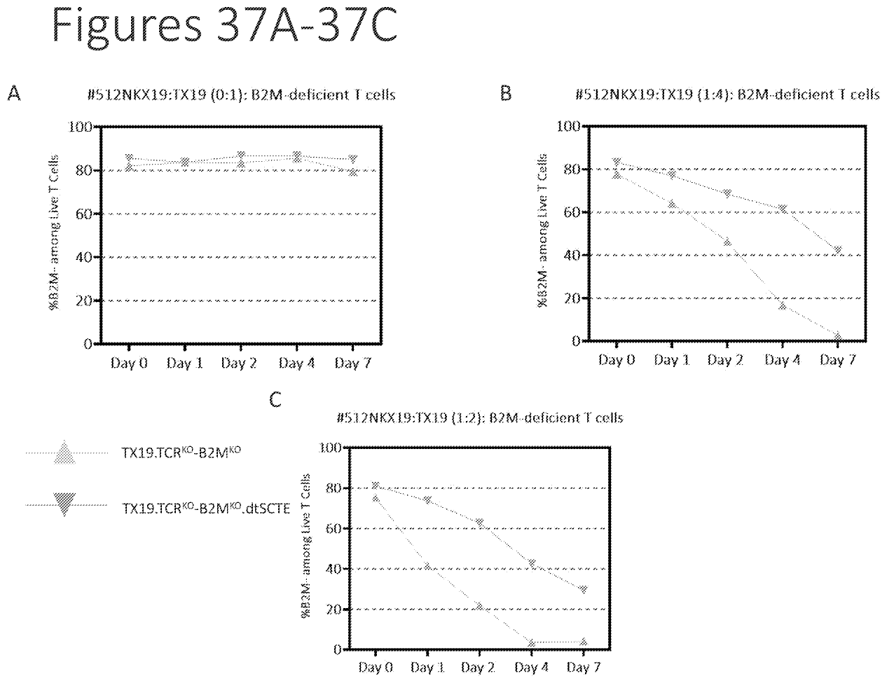

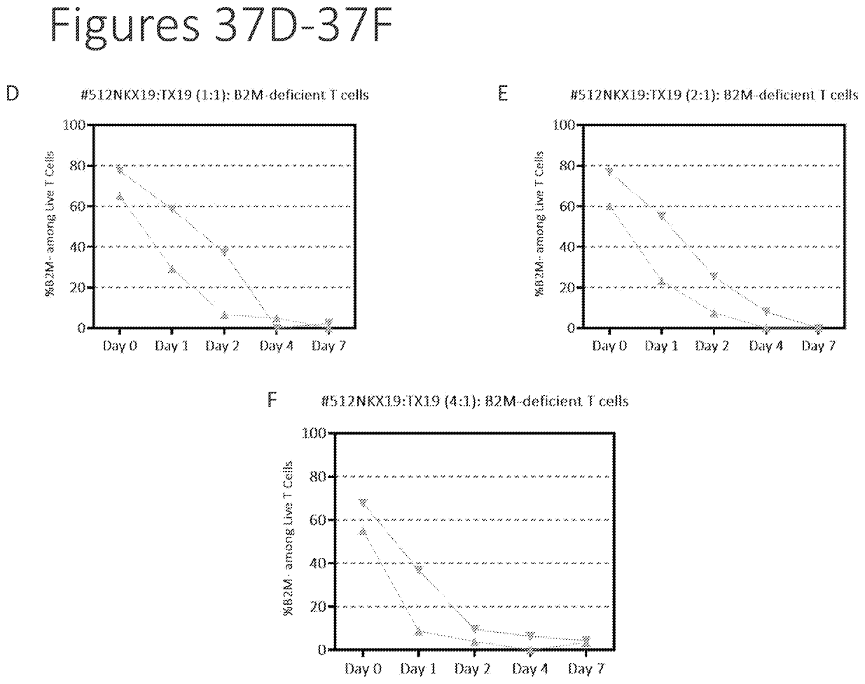

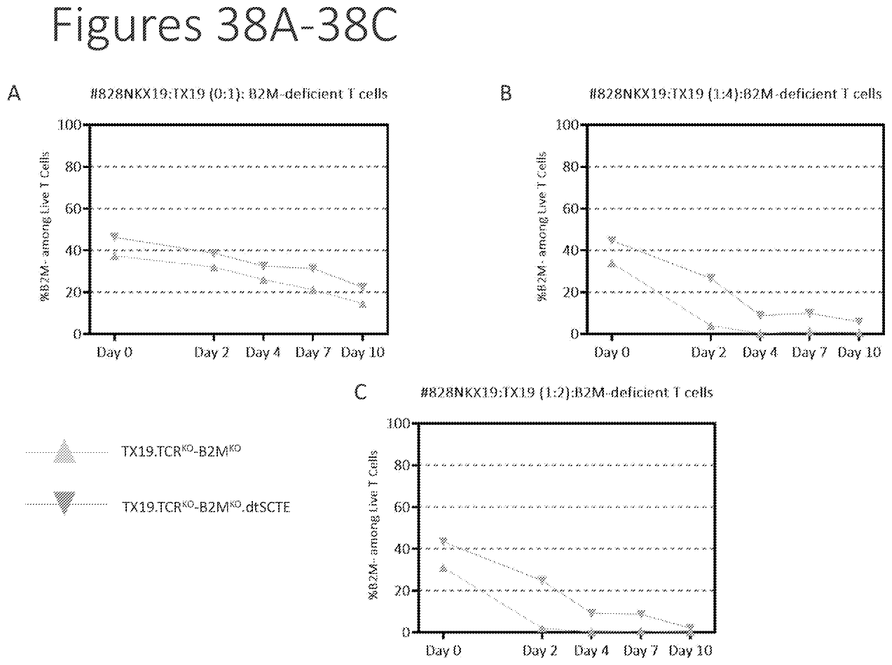

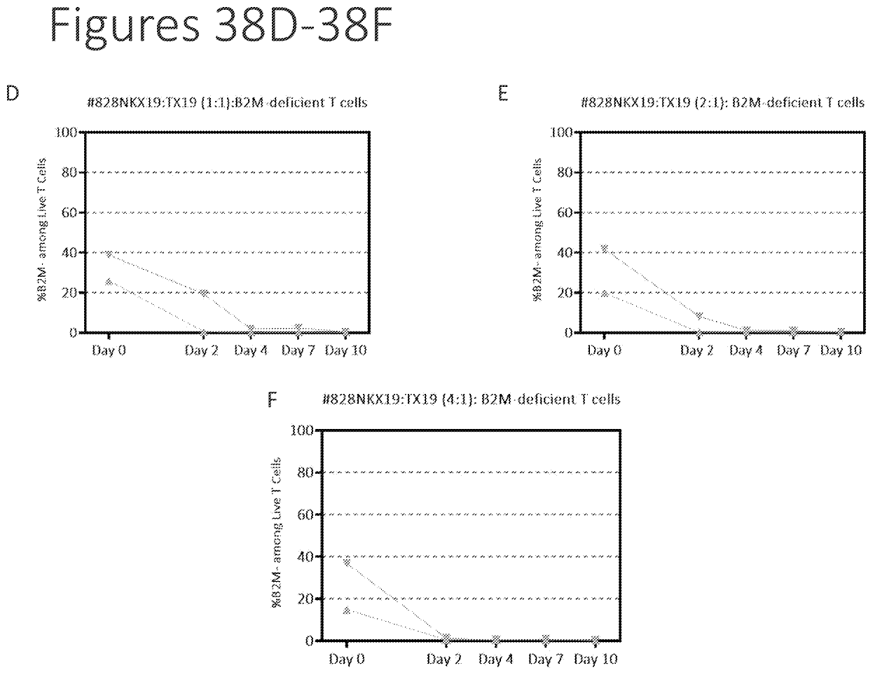

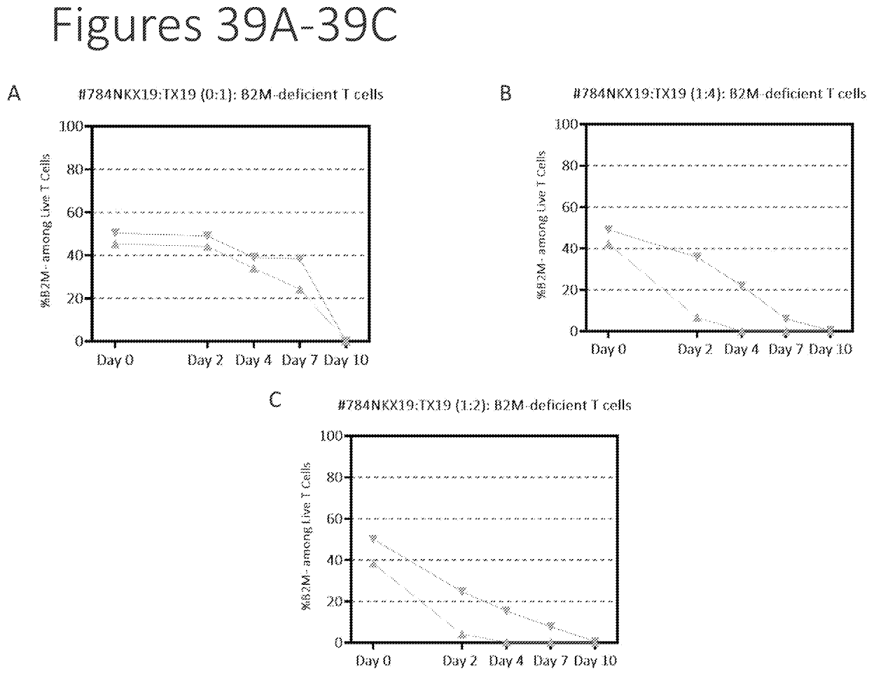

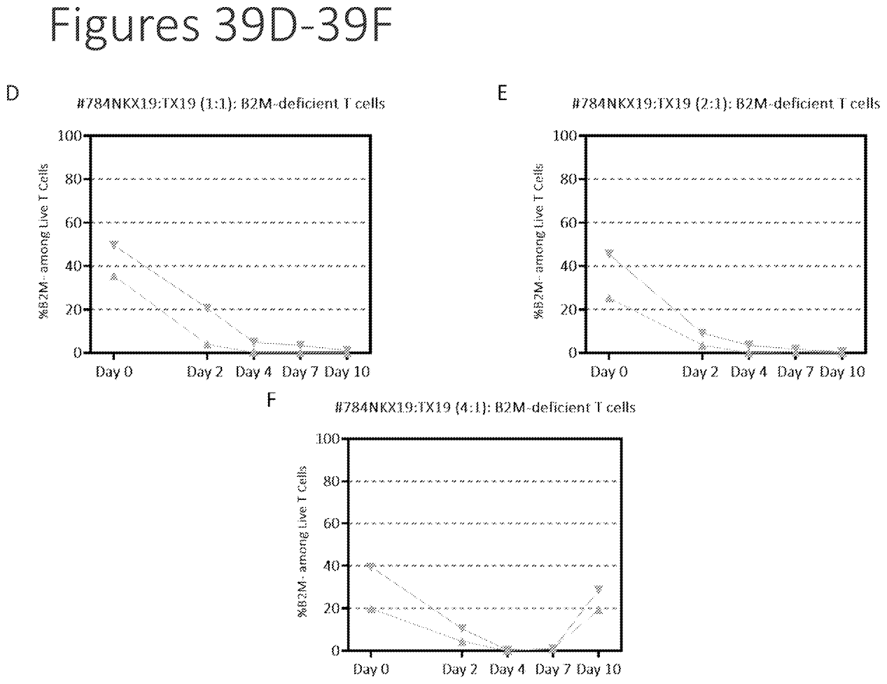

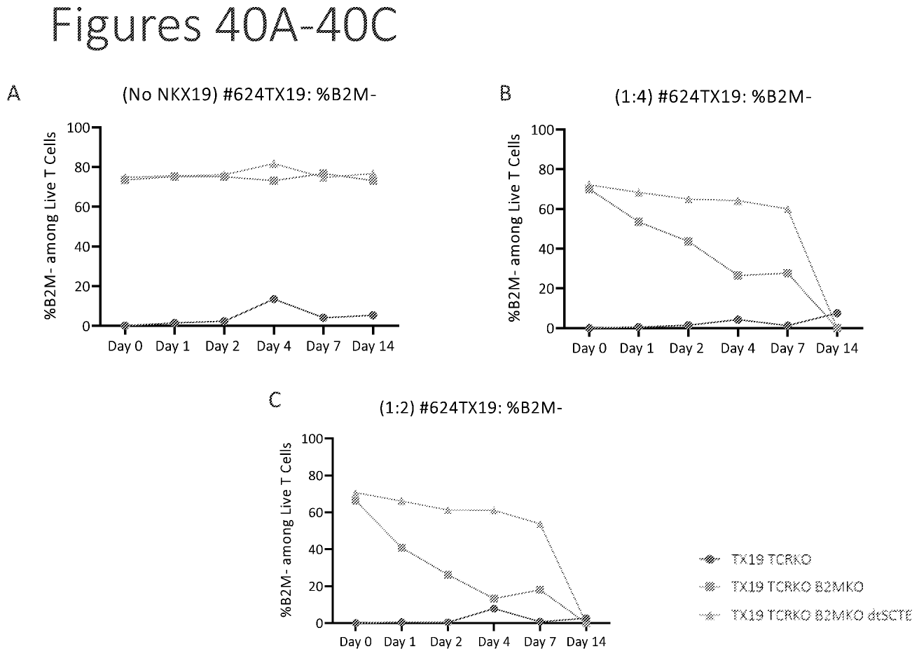

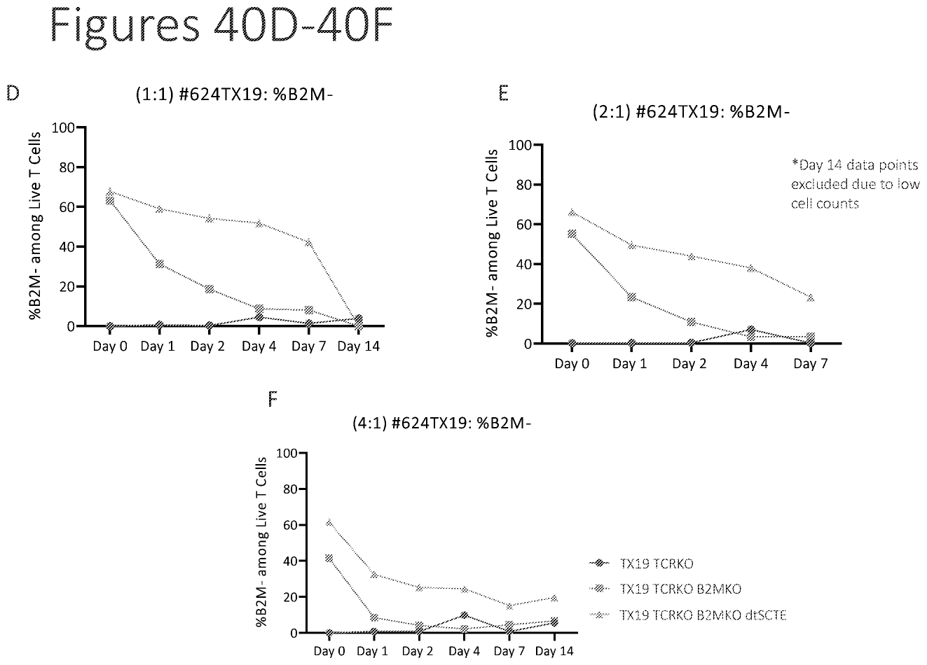

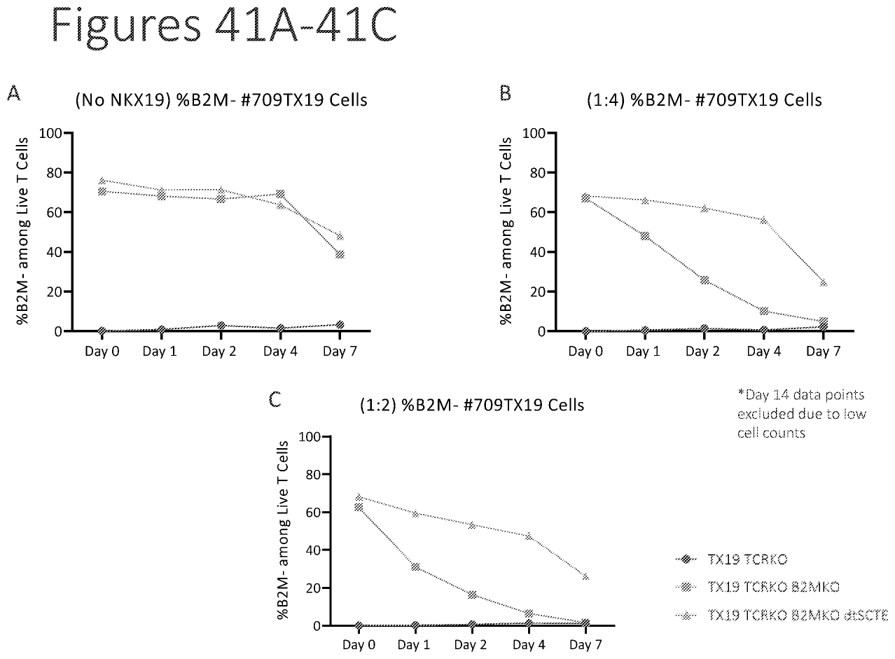

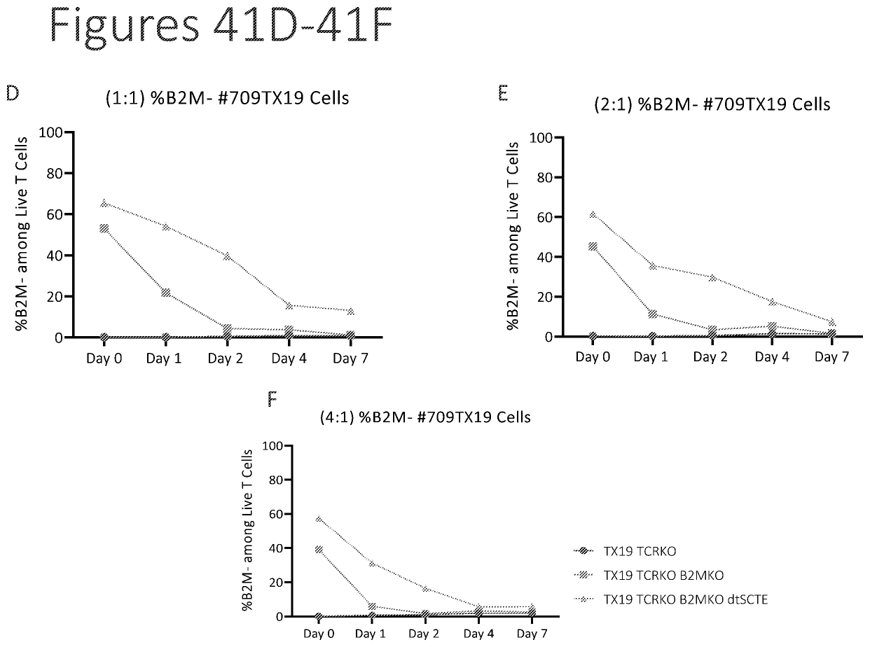

Turning to the question of whether HLA-E can confer protection on T cells vis-à-vis NK cells that are also engineered to express a CAR (e.g., representing the context of a mixed cell therapeutic), FIGS. 36G-36L provide corresponding data for coculture of NK cells expressing an anti-CD19 CAR (NKX19) with T cells expressing an anti-CD19 CAR and that are either TX19.TCR^{KO}.B2M^{KO} or TX19. TCR^{KO}.B2M^{KO}.dtSCTE. NK:T cell ratios are (G-L, respectively) 0:1, 1:4, 1:2, 1:1, 2:1, and 4:1. Co-culturing was performed for 14 days and the percentage of B2M^{KO} T cells (out of live T cells) present in the culture was assessed at the indicated timepoints with HLA-E re-expressing data points indicated by the inverted triangles. FIG. 36G shows positive control data, as this represents the culture of the indicated T cells without any engineered NK cells present. FIGS. 361-36L represent the move from lowest to highest NK:T cell ratio, and while the percentage of B2M^{KO} T cells appeared to be less when cultured with NKX19 NK cells, there appears to be a maintained protection of HLA-E re-expressing T cells at equivalent ratios of NK:T or lower. The data at high NK:T ratio suggest there may still be some protection with the greater degree of NK cells, but the protective effect appears to be somewhat abrogated. FIGS. 37A-37F show corresponding data (NKX19:TX19), at various similar timepoints, for an additional donor. FIGS. 38A-38F show corresponding data (NKX19:TX19), at various similar timepoints, for an additional donor. FIGS. 39A-39F show corresponding data (NKX19:TX19) for an additional donor. FIGS. 40A-40F show corresponding data (NKX19:TX19), at various similar timepoints, for an additional donor, with the inclusion of the TCR^{KO} T cells as a control. FIGS. 41A-41F show corresponding data (NKX19:TX19), at various similar timepoints, for an additional donor, with the inclusion of the TCR^{KO} T cells as a control. While there is some donor variability in terms of the protection conferred on the T cells by HLA-E re-expression, the general trend is that HLA-E re-expression results in a clear protective effect at early time points in co-culture, particularly at low NK:T ratios, the protective effect becomes reduced as the number of NK cells outnumbers the number of T cells. Nevertheless, these data demonstrate better survival of the HLA-E re-expressing T cells at ratios of ˜1:1 or lower. According to some embodiment, a 1:1 NK:T cell ratio (or lower) is used in a dosing preparation for a mixed cell therapeutic product. According to additional embodiments, a 1:2, 1:3, 1:4, 1:5, or lower (including ratios between those listed) is used in a dosing preparation for a mixed cell therapeutic product. Additionally, as disclosed herein, additional edits to, or engineering of, T cells (or NK cells) are provided for to reduce the fratricide between different cell types in a mixed therapeutic cell context.

### Example 3—Additional Immune Evasion Constructs and Uses Thereof

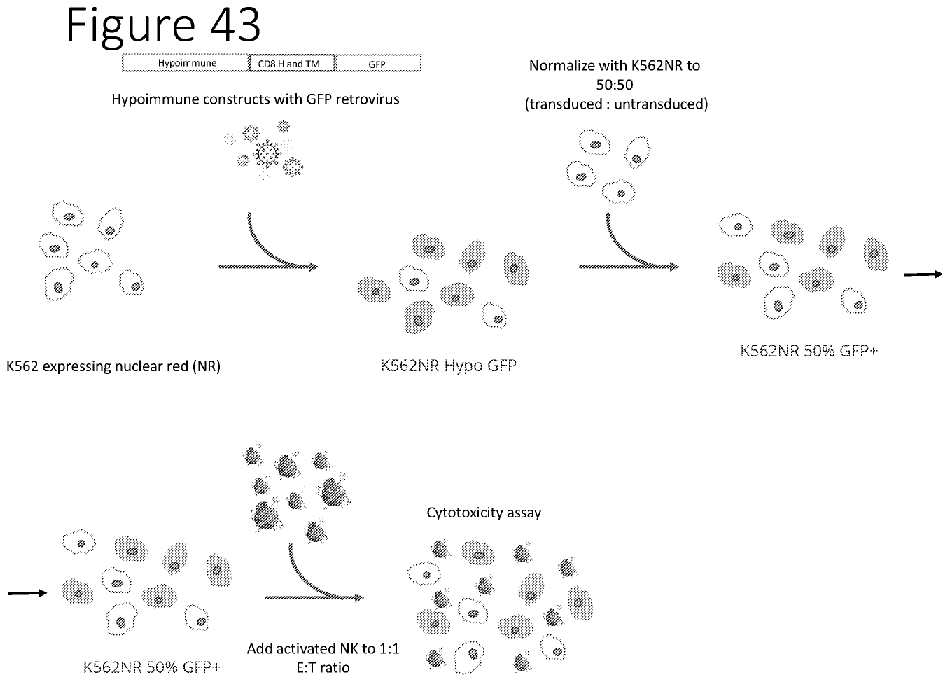

As discussed herein, various immunosuppressive constructs are provided for in order to confer on, for example an NK or a T cell in a mixed NK and T cell therapeutic population, reduced immunogenicity vis-à-vis the other therapeutic cells, as we as vis-à-vis host T cells. In several embodiments, the immunosuppressive constructs and resulting engineered (and/or edited) cells are less fratricide from another member of the therapeutic cell population, and also reduce risk of graft versus host and host versus graft side effects. FIGS. 42A and 42B (and FIG. 29C) and FIG. 43 show non-limiting schematics of how an immunosuppressive construct according to embodiments provided for herein might be constructed. Table 8 shows the corresponding SEQ ID NO: for certain non-limiting immunosuppressive constructs provided for herein.

TABLE 8
Selected Immunosuppressive Constructs Sequence Identifiers

| SEQ | |
|---|---|
| ID | |
| NO | Construct Identifier |
| 683 | UL18 trimer_SS VMAPRTLFL_IRES_GFP |
| 684 | UL18 trimer_SS VMAPRTLFL_IRES_GFP |
| 685 | UL18 trimer_SS VMAPRTLFL - AA [no Flag or GFP] |
| 686 | UL18 trimer_SS VMAPRTLFL - NT [no Flag or GFP] |
| 687 | HLA-E trimer_SS VMAPRTLFL_IRES_GFP |
| 688 | HLA-E trimer_SS VMAPRTLFL |
| 689 | HLA-E trimer_SS VMAPRTLFL - AA - [no flag or gfp] |
| 690 | HLA-E trimer_SS VMAPRTLFL [no flag or gfp] |
| 691 | CKS17-8aHTM_aa |
| 692 | CKS17-8aHTM |
| 693 | CKS17-8aHTM_aa [no flag or gfp] |
| 694 | CKS17-8aHTM [no flag or gfp] |
| 695 | p15E-L-8aHTM_IRES_GFP |
| 696 | p15E-L-8aHTM_IRES_GFP |
| 697 | p15E-L-8aHTM [no flag/gfp] |
| 698 | p15E-L-8aHTM_[no flag/gfp |
| 699 | HTLV-8aHTM_IRES_GFP |
| 700 | HTLV-8aHTM_IRES_GFP |
| 701 | HTLV-8aHTM_[no flag/gfp] |
| 702 | HTLV-8aHTM_[no flag/gfp] |
| 703 | HIV_L-8aHTM_IRES_GFP |
| 704 | HIV_L-8aHTM_IRES_GFP |
| 705 | HIV_L-8aHTM_[no flag/gfp] |
| 706 | HIV_L-8aHTM_[no flag/gfp] |
| 707 | HIV_S-8aHTM_IRES_GFP |
| 708 | HIV_S-8aHTM_IRES_GFP |
| 709 | HIV_S-8aHTM_no flag/gfp |
| 710 | HIV_S-8aHTM_no flga/gfp |
| 711 | TCR Synthetic 3x-8aHTM_IRES_GFP |
| 712 | TCR Synthetic 3x-8aHTM_IRES_GFP |
| 713 | TCR Synthetic 3x-8aHTM_no flag/gfp |
| 714 | TCR Synthetic 3x-8aHTM_no flag/gfp |
| 715 | tCD47-8aHTM_IRES_GFP |
| 716 | tCD47-8aHTM_IRES_GFP |
| 717 | tCD47-8aHTM_no flag/gfp |
| 718 | tCD47-8aHTM_no falg/gfp |
| 719 | p15Ex3-8aHTM_IRES_GFP |
| 720 | p15Ex3-8aHTM_IRES_GFP |
| 721 | p15Ex3-8aHTM_no flag/gfp |
| 722 | p15Ex3-8aHTM_no flag/gfp |
| 723 | p15E_tCD47-8aHTM_IRES_GFP |
| 724 | p15E_tCD47-8aHTM_IRES_GFP |
| 725 | p15E_tCD47-8aHTM_no flag/gfp |
| 726 | p15E_tCD47-8aHTM_no flag/gfp |
| 727 | tCD47_p15E-8aHTM_IRES_GFP |
| 728 | tCD47_p15E-8aHTM_IRES_GFP |
| 729 | tCD47_p15E-8aHTM_no flag gfp |
| 730 | tCD47_p15E-8aHTM_no flag/gfp |
| 731 | HIV_L_tCD47-8aHTM_IRES_GFP |
| 732 | HIV_L_tCD47-8aHTM_IRES_GFP |
| 733 | HIV_L_tCD47-8aHTM_no flag/gfp |
| 734 | HIV_L_tCD47-8aHTM_no flag/gfp |
| 735 | HIV_S_tCD47-8aHTM_IRES_GFP |
| 736 | HIV_S_tCD47-8aHTM_IRES_GFP |
| 737 | HIV_S_tCD47-8aHTM_no flag/gfp |
| 738 | HIV_S_tCD47-8aHTM_no flag/gfp |
| 739 | HTLV_tCD47-8aHTM_IRES_GFP |
| 740 | HTLV_tCD47-8aHTM_IRES_GFP |
| 741 | HTLV_tCD47-8aHTM_no flag/gfp |
| 742 | HTLV_tCD47-8aHTM_no flag/gfp |
| 743 | tCD47_p15Ex2-8aHTM_IRES_GFP |
| 744 | tCD47_p15Ex2-8aHTM_IRES_GFP |
| 745 | tCD47_p15Ex2-8aHTM_noflag/gfp |
| 746 | tCD47_p15Ex2-8aHTM_no flag/gfp |
| 747 | CKS17-8aHTM_eGFP_aa |
| 748 | CKS17-8aHTM_eGFP |
| 749 | CKS17-8aHTM_aa_noGFP |
| 750 | CKS17-8aHTM_NT_no GFP |
| 751 | CD47-eGFP_aa |
| 752 | CD47-eGFP |
| 753 | CD47-_ aa_no_GGFP |
| 754 | CD47-NT_no_GFP |
| 755 | fGP41_HIVtm_sfGFP_aa |
| 756 | fGP41_HIVtm_sfGFP |
| 757 | fGP41_HIVtm_aa_no_GFP |
| 758 | fGP41_HIVtm_NT_no_GFP |
| 759 | p15Etm_sfGFP_aa |
| 760 | p15Etm_sfGFP |
| 761 | p15Etm_aa_no_GFP |
| 762 | p15Etm_NT_noGFP |
| 763 | CD43_TM_sfGFP_aa |
| 764 | CD43_TM_sfGFP |
| 765 | CD43_TM_aa_No_GFP |
| 766 | CD43_TM_NT_no_GFP |
| 767 | LMP1_TM-sfGFP_aa |
| 768 | LMP1_TM-sfGFP |
| 769 | LMP1_TM_aa_no_GFP |
| 770 | LMP1_TM-NT_No-GFP |
| 771 | fgD_TM_sfGFP_aa |
| 772 | fgD_TM_sfGFP |
| 773 | fgD_TM_AA-NO_GFP |
| 774 | fgD_TM_NT_NO_GFP |
| 775 | fLLT1_TM-sfGFP_aa |
| 776 | fLLT1_TM-sfGFP |
| 777 | fLLT1_TM-_aa_No_GFP |
| 778 | fLLT1_TM-NT_No_GFP |
| 779 | CD47tm162_sfGFP_aa |
| 780 | CD47tm162_sfGFP |
| 781 | CD47tm162_aa_No_GFP |
| 782 | CD47tm162_NT_NoGFP |
| 783 | LMP_L-CD8HTM_sfGFP_aa |
| 784 | LMP_L-CD8HTM_sfGFP |
| 785 | LMP_L-CD8HTM_aa_No_GFP |
| 786 | LMP_L-CD8HTM_NT_No_GFP |
| 787 | LALLFWLx5-CD8HTM_sfGFP_aa |
| 788 | LALLFWLx5-CD8HTM_sfGFP |
| 789 | LALLFWLx5-CD8HTM_aa_no_GFP |
| 790 | LALLFWLx5-CD8HTM_NT_No_GFP |
| 791 | GP41fptm_sfGFP_aa |
| 792 | GP41fptm_sfGFP |
| 793 | GP41fptm_aa_No-GFP |
| 794 | GP41fptm_NT_No_GFP |
| 795 | CKS17-8aTM_sfGFP_aa |
| 796 | CKS17-8aTM_sfGFP |
| 797 | CKS17-8aTM_aa_No_GFP |
| 798 | CKS17-8aTM_NT_No_GFP |
| 799 | CKS_HTLV-8aTM_sfGFP_aa |
| 800 | CKS_HTLV-8aTM_NT_No_GFP |
| 801 | CKS_HTLV-8aTM_aa_No_GFP |
| 802 | CKS_HTLV-8aTM_sfGFP |
| 803 | CKS_LMP-8aTM_sfGFP_aa |
| 804 | CKS_LMP-8aTM_sfGFP |
| 805 | CKS_LMP-8aTM_aa_no_GFP |
| 806 | CKS_LMP-8aTM_NT_No_GFP |
| 807 | CKS_GP41fptm_sfGFP_aa |
| 808 | CKS_GP41fptm_sfGFP |
| 809 | CKS_GP41fptm_aa_No_GFP |
| 810 | CKS_GP41fptm_NT_No_GFP |
| 811 | LMP_S-CD8HTM_sfGFP_aa |
| 812 | LMP_S-CD8HTM_sfGFP |
| 813 | LMP_S-CD8HTM_aa_No_GFP |
| 814 | LMP_S-CD8HTM_NT_No_GFP |
| 815 | LMP_CD47tm162_sfGFP_aa |
| 816 | LMP_CD47tm162_sfGFP |
| 817 | LMP_CD47tm162_aa_No_GFP |
| 818 | LMP_CD47tm162_NT_No_GFP |
| 819 | p15E_CD47tm162_sfGFP_aa |
| 820 | p15E_CD47tm162_sfGFP |
| 821 | p15E_CD47tm162_aa_No_GFP |
| 822 | p15E_CD47tm162_NT_No_GFP |
| 823 | CD47_GP41fptm_sfGFP_aa |
| 824 | CD47_GP41fptm_sfGFP |
| 825 | CD47_GP41fptm_aa_No_GFP |
| 826 | CD47_GP41fptm_NT_No_GFP |
| 827 | HLA-E_STE20_sfGFP_aa |
| 828 | HLA-E_STE20_sfGFP |
| 829 | HLA-E_STE20_aa_NO_GFP |
| 830 | HLA-E_STE20_NT_NO_GFP |
| 831 | anti-SIRPa agonist_vHL_sfGFP_aa |
| 832 | anti-SIRPa agonist_vHL_sfGFP |
| 833 | anti-SIRPa agonist_vHL_aa_NO_GFP |
| 834 | anti-SIRPa agonist_vHL_NT_NO_GFP |
| 835 | HTLV1_fGP62_sfGFP_aa |
| 836 | HTLV1_fGP62_sfGFP |
| 837 | HTLV1_fGP62_aa_NO_GFP |
| 838 | HTLV1_fGP62_NO_GFP |
| 839 | HTLV1_GP21_sfGFP_aa |
| 840 | HTLV1_GP21_sfGFP |
| 841 | HTLV1_GP21_aa_NO_GFP |
| 842 | HTLV1_GP21_NO_GFP |
| 843 | LASV_fGP2_sfGFP_aa |
| 844 | LASV_fGP2_sfGFP |
| 845 | LASV_fGP2_aa_NO_GFP - |
| 846 | LASV_fGP2_NT_NO_GFP |
| 847 | SEBOV_fGP_sfGFP_aa |
| 848 | SEBOV_fGP_sfGFP |
| 849 | SEBOV_fGP_aa_NO_GFP |
| 850 | SEBOV_fGP_NT_NO_GFP |
| 851 | SEBOV_GP2_sfGFP_aa |
| 852 | SEBOV_GP2_sfGFP |
| 853 | SEBOV_GP2_aa_NO_GFP |
| 854 | SEBOV_GP2_NT_NO_GFP |
| 855 | SCoV_S2_sfGFP_aa |
| 856 | SCoV_S2_sfGFP |
| 857 | SCoV_S2_aa_NO_GFP |
| 858 | SCoV_S2_NT_NO_GFP |
| 859 | LGALS3BP_sfGFP_aa |
| 860 | LGALS3BP_sfGFP |
| 861 | LGALS3BP_aa_NO_GFP |
| 862 | LGALS3BP_NT_No_GFP |
| 863 | CD24_sfGFP_aa |
| 864 | CD24_sfGFP |
| 865 | CD24_aa_NO_GFP |
| 866 | CD24_NT_NO_GFP |
| 867 | HCV_E2_sfGFP_aa |
| 868 | HCV_E2_sfGFP |
| 869 | HCV_E2_aa_NO_GFP |
| 870 | HCV_E2_NT_NO_GFP |
| 871 | anti-SIRPa agonist_vLH_sfGFP_aa |
| 872 | anti-SIRPa agonist_vLH_sfGFP |
| 873 | anti-SIRPa agonist_vLH_aa_NO_GFP |
| 874 | anti-SIRPa agonist_vLH_NT_NO_GFP |
| 875 | CEACAM1_sfGFP_aa |
| 876 | CEACAM1_sfGFP |
| 877 | CEACAM1_aa_NO_GFP |
| 878 | CEACAM1_NT_NO_GFP |
| 879 | CD155tm_3M_sfGFP_aa |
| 880 | CD155tm_3M_sfGFP |
| 881 | CD155tm_3M_aa_NO_GFP |
| 882 | CD155tm_3M_NT_NO_GFP |
| 883 | CD31tm_sfGFP_aa |
| 884 | CD31tm_sfGFP |
| 885 | CD31tm_aa_NO_GFP |
| 886 | CD31tm_NT_NO_GFP |
| 887 | CD111tm_sfGFP_aa |
| 888 | CD111tm_sfGFP |
| 889 | CD111tm_aa_NO_GFP |
| 890 | CD111tm_NT_NO_GFP |
| 891 | CD200tm_sfGFP_aa |
| 892 | CD200tm_sfGFP |
| 893 | CD200tm_aa_NO_GFP |
| 894 | CD200tm_NO_GFP |
| 895 | CD8a signal peptide AA |
| 896 | CD8a signal peptide NT |

As discussed above, re-expression of HLA-E or HLA-G on a therapeutic cell should impart to the cell the ability to immunosuppress cytotoxicity from co-administered therapeutic cells (as well as host T cells). FIG. 43 shows a schematic process flow for testing the immunosuppressive effects of HLA re-expression. In brief, K562 cells expressing nuclear red dye (K562NR) are prepared as are various hypoimmune constructs as provided for herein. While these test constructs are engineered to include GFP (or a FLAG tag), it shall be appreciated that any of the immunosuppressive constructs provided herein can also be made without a tag. The immunosuppressive constructs are packaged in a retroviral vector and used to transduce the K562NR cells such that at least a portion of the K562 cells express the immunosuppressive construct and GFP (yielding a K562NR Hypo GFP population). Additional K562NR cells are added to the cells to achieve a 1:1 ratio of K562 cells that express (or do not express) a hypoimmune construct (K562 50% GFP). These are now considered the target cells in an assay. Thereafter, activated NK cells are co-cultured with the K562 50% GFP target cells at a 1:1 ratio, and the cytotoxic effects of the NK cells against the K562 50% GFP target cells is assessed.

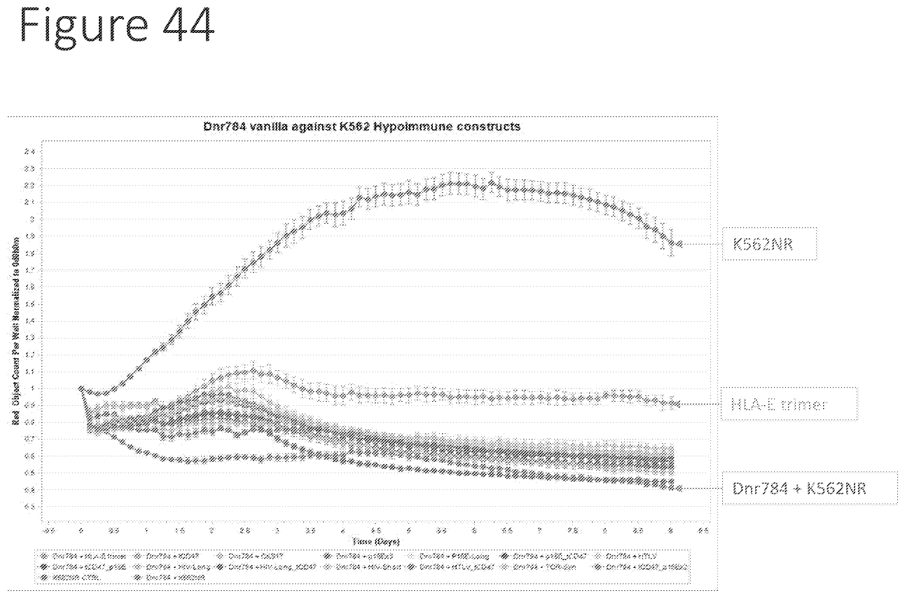

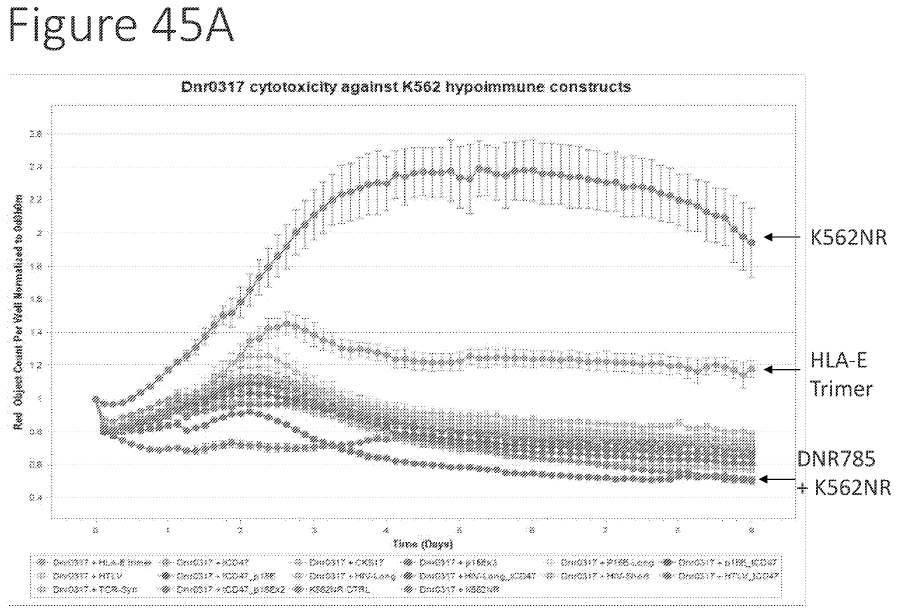

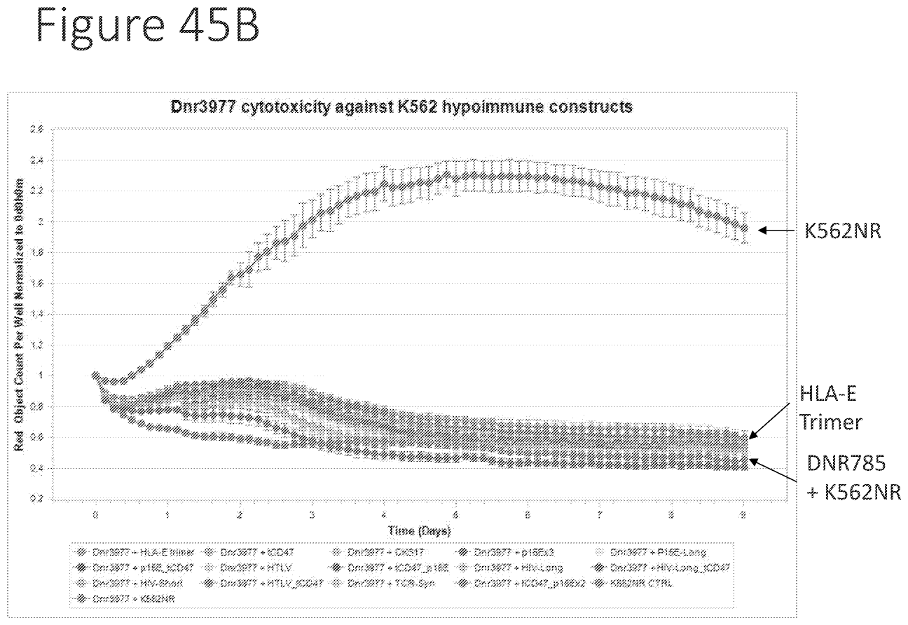

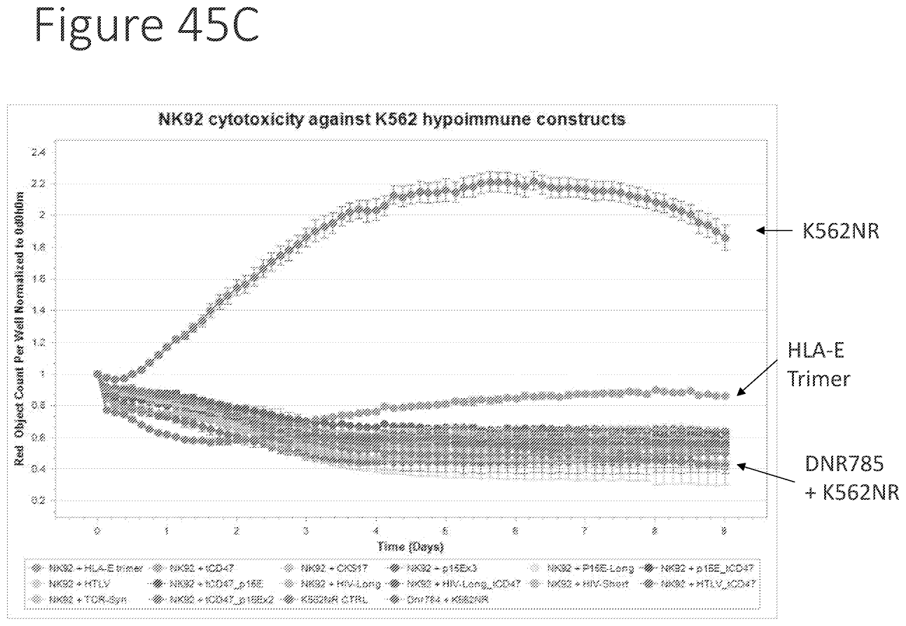

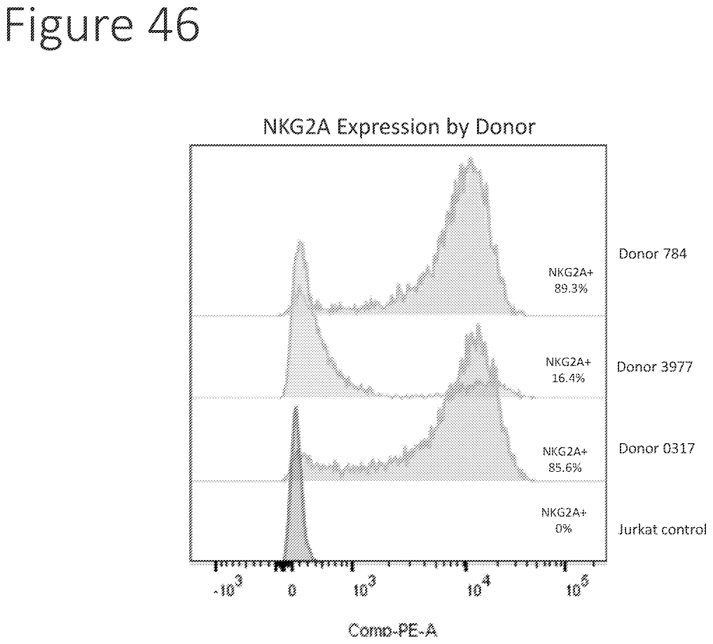

FIG. 44 shows the results of one experiment testing multiple immunosuppressive constructs and their ability to protect against cytotoxicity from NK cells. K562NR cells alone showed an increase in red object count (representing number of cells in culture) as a control. As can be seen, many of the immunosuppressive constructs tested did not confer protection against the NK cells. A notable exception is the immunosuppressive HLA-E construct that comprises a trimer of HLA-E peptides. FIG. 45A shows an additional experimental replicate evaluating the protective effects of the immunosuppressive constructs against NK cells from other donors. In FIG. 45A, there was some modest immunoprotective effect for several constructs, but again, the HLA-E trimer construct offered the most protective effects. In FIG. 45B, none of the constructs provided any protection against activated NK cells from a different donor. FIG. 45C shows the protective effect of the HLA-E trimer construct, even against immortalized NK92 cells. As discussed above, the mechanism of action of HLA-E inhibition of NK cell activation is through the balance of the inhibitory and activating receptors on an NK cell, with NKG2A producing NK cell inhibitory signals upon binding to HLA. FIG. 46 shows that level of HLA expression by donor. As can be seen, donor #784 and donor #0317 both have NK cells that express high levels of NKG2A, over 85% of the NK cells are NKG2A positive for each donor. These donors correspond to those experimental results of FIG. 44 and FIG. 45A, where the re-expression of HLA-E through the use of the HLA-E trimer immunosuppressive construct conferred protection of the K562 “target” cells from the donor cells. These data suggest that, according to some embodiments, donor NK cells that express elevated levels of NKG2A (e.g., above about 60%, about 70%, about 80% about 85% about 90% or more) are unexpectedly useful in a mixed therapeutic cell population wherein at least a portion of the therapeutic cells are engineered to express an immunosuppressive construct, as the high-NKG2A cells are inhibited from fratricide against other therapeutic cells.

### Example 4—Cytotoxic Evaluation of Selected Hypoimmune Constructs

As discussed herein, various immunosuppressive constructs are provided for in order to confer on, for example an NK or a T cell in a mixed NK and T cell therapeutic population, reduced immunogenicity vis-à-vis the other therapeutic cells, as we as vis-à-vis host T cells. Several experiments were run to assess the ability of selected hypoimmune constructs provided for herein to confer characteristics of immune evasion on cells to express the constructs.

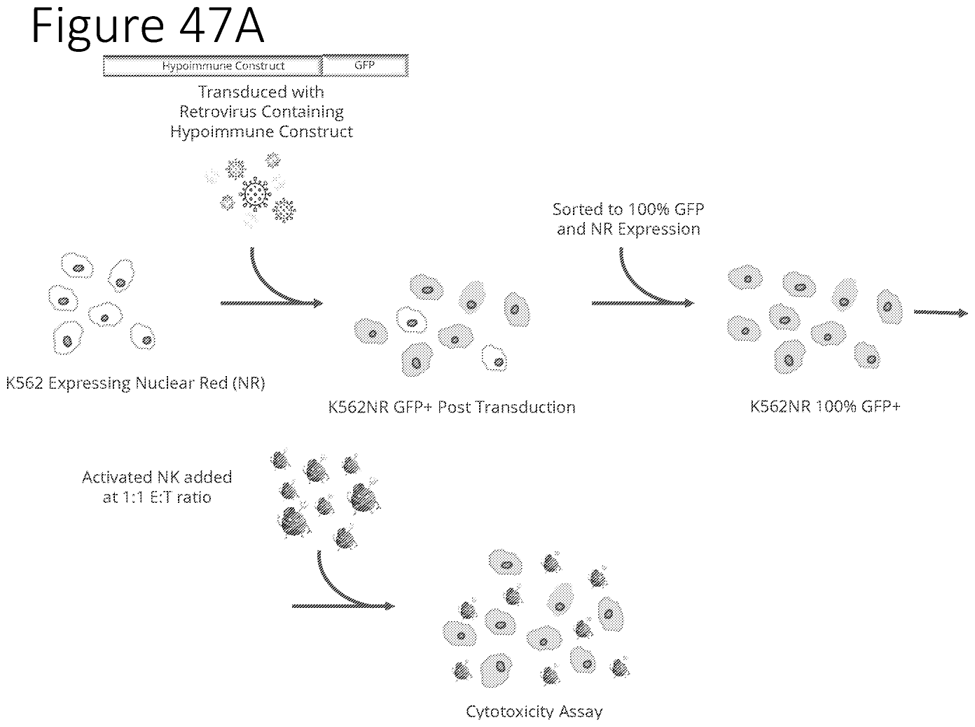

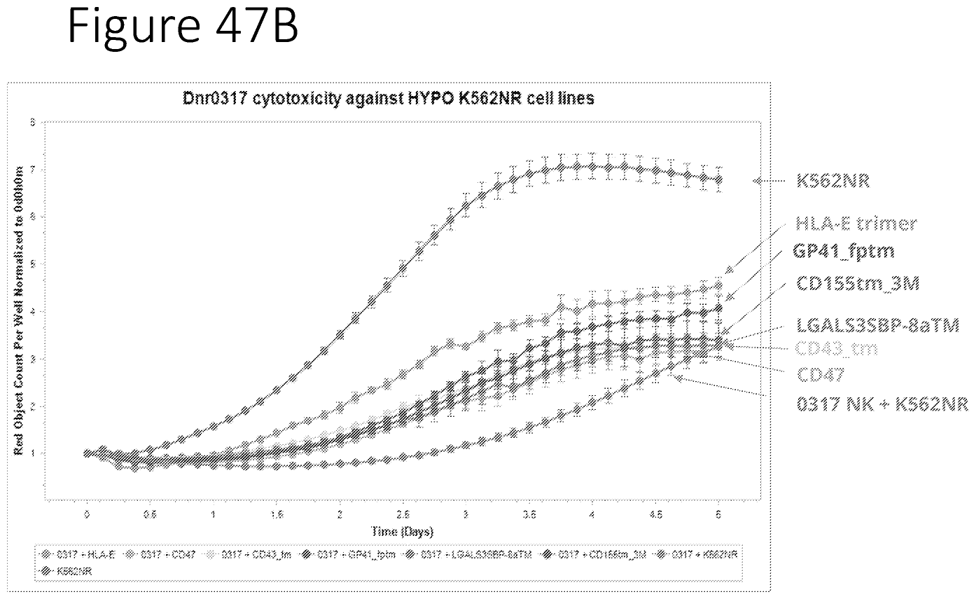

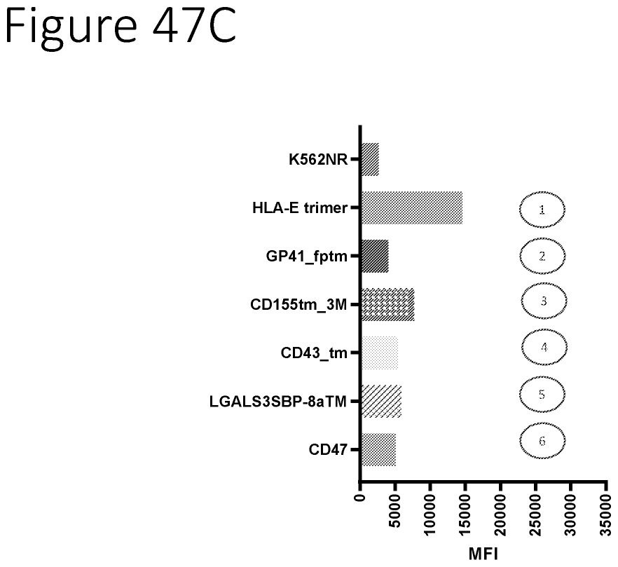

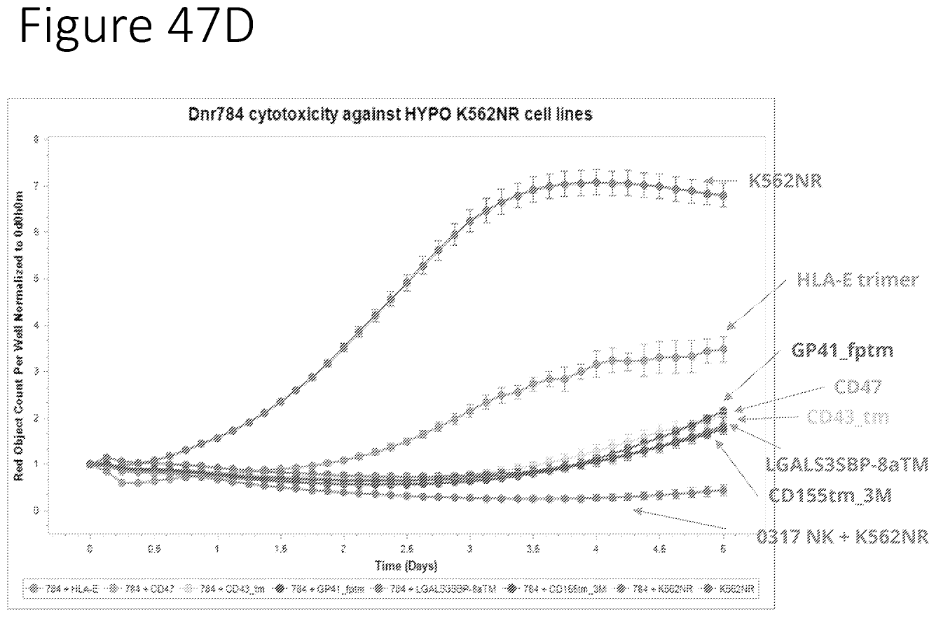

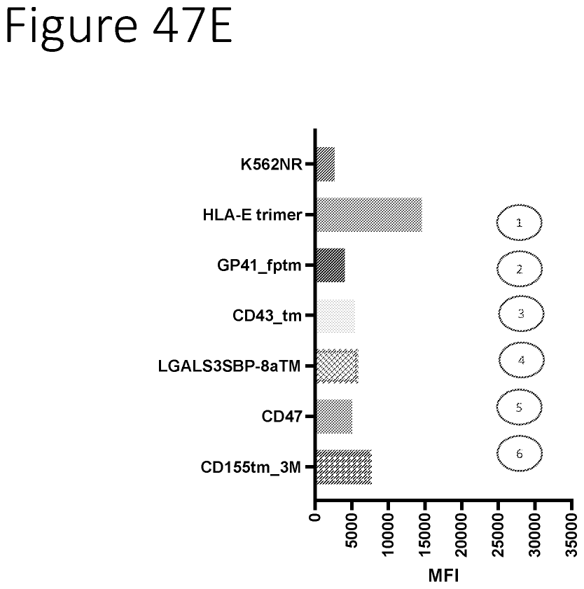

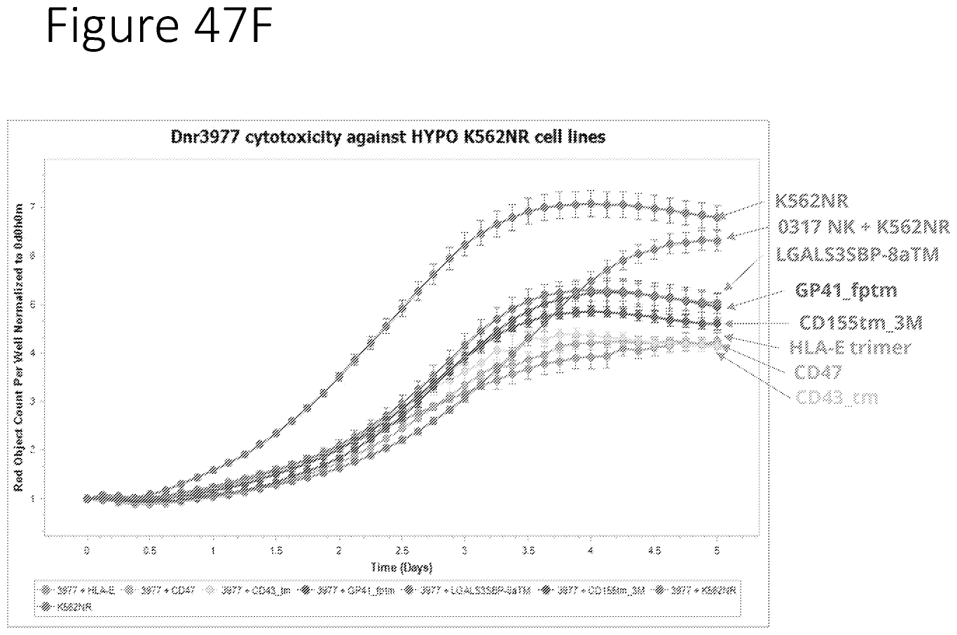

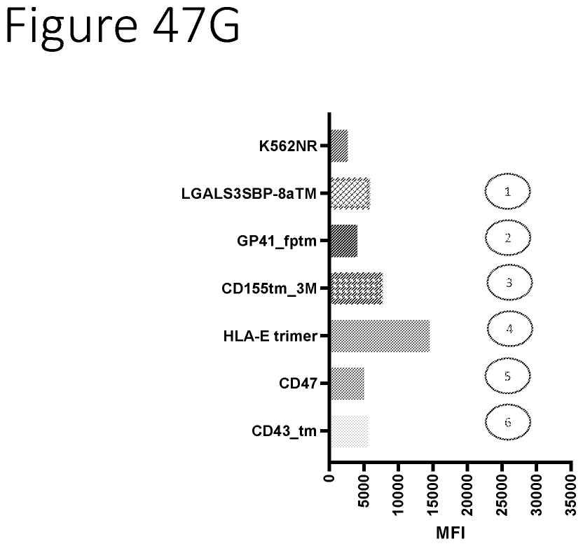

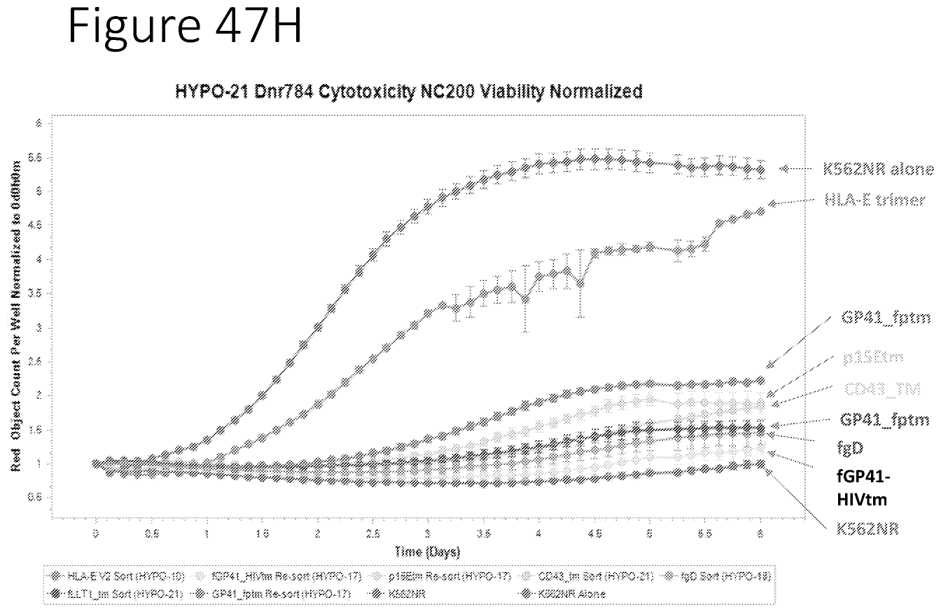

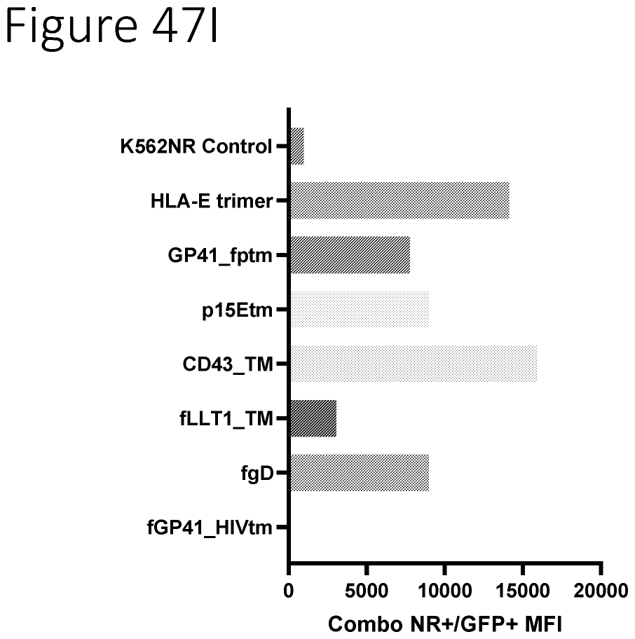

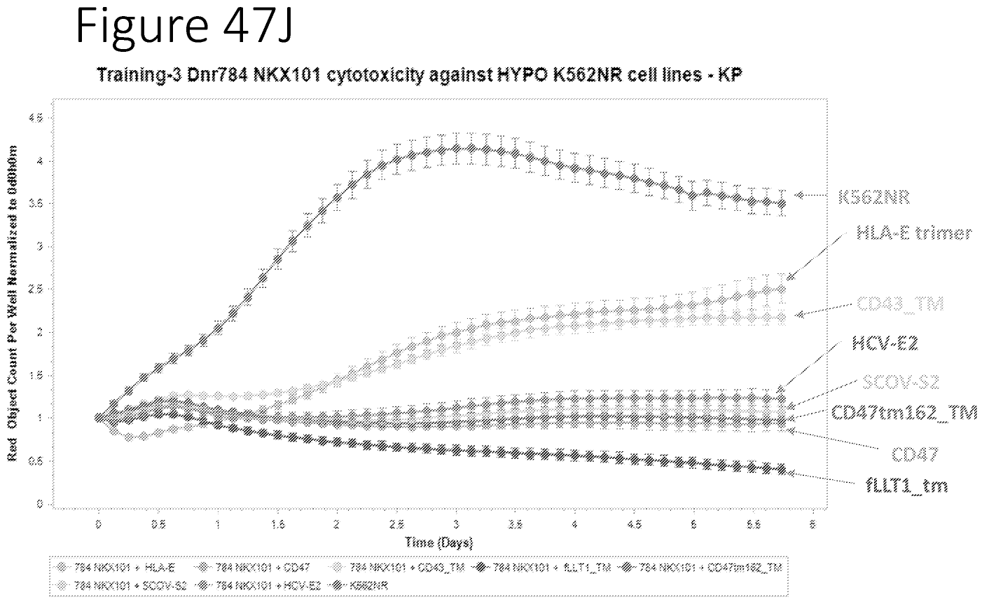

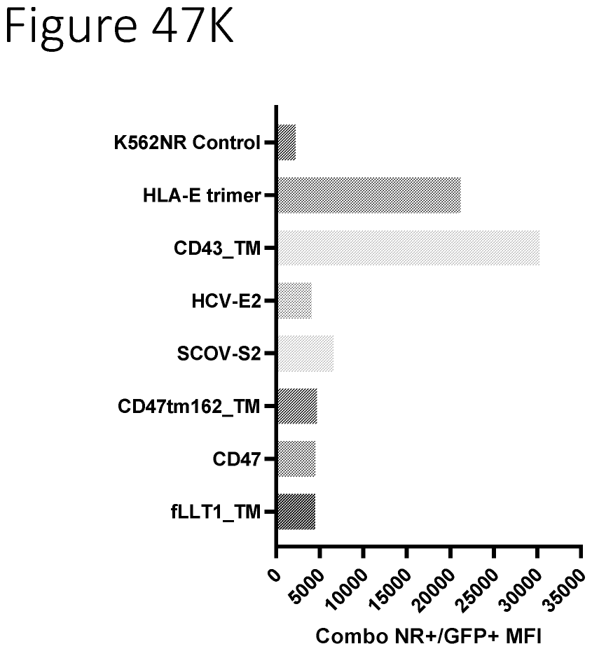

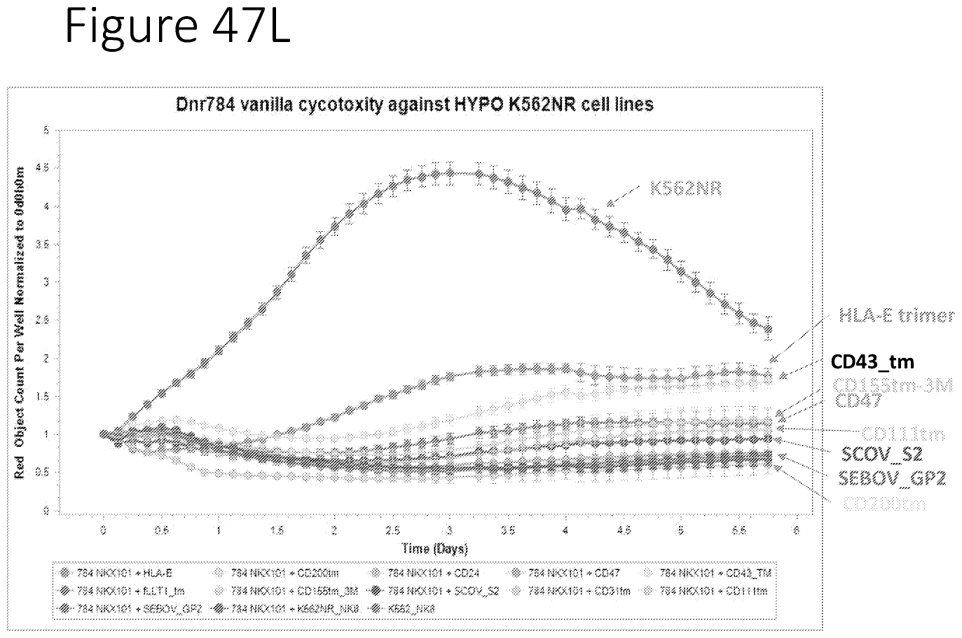

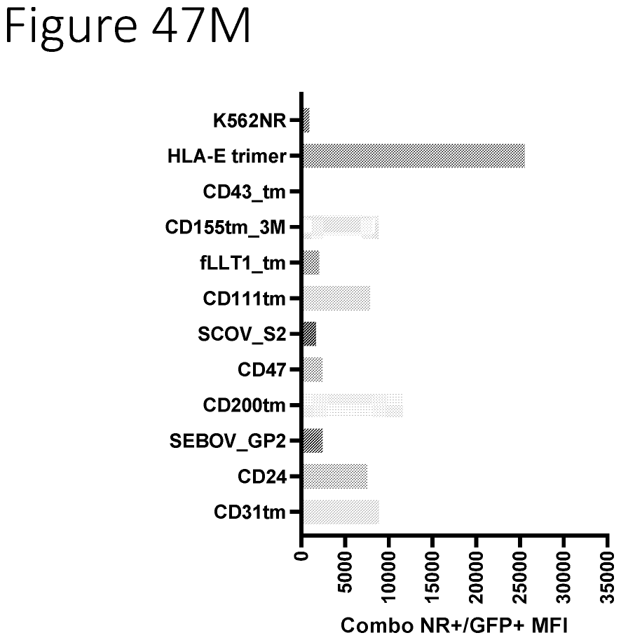

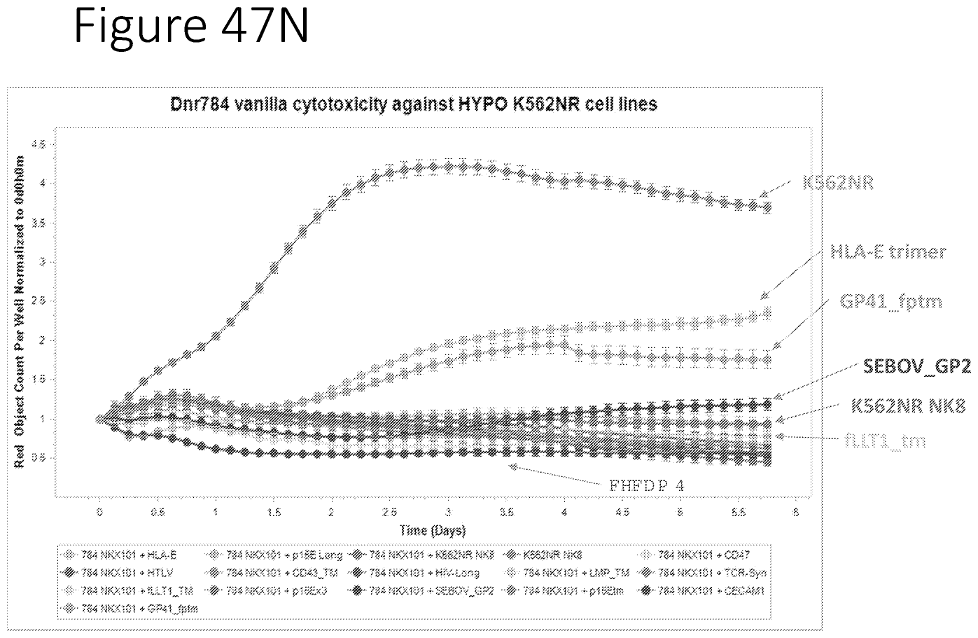

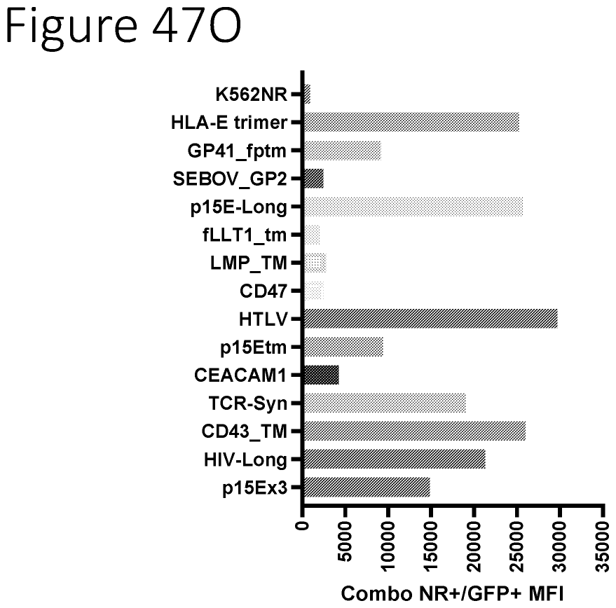

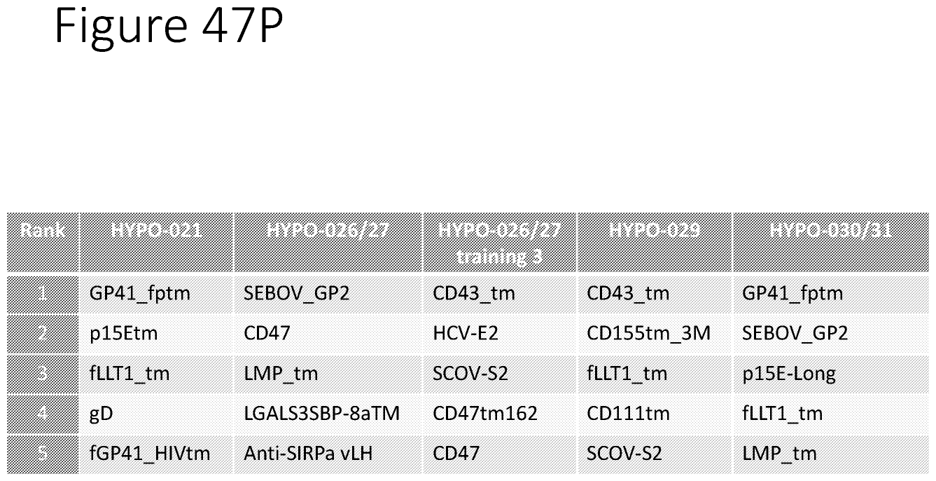

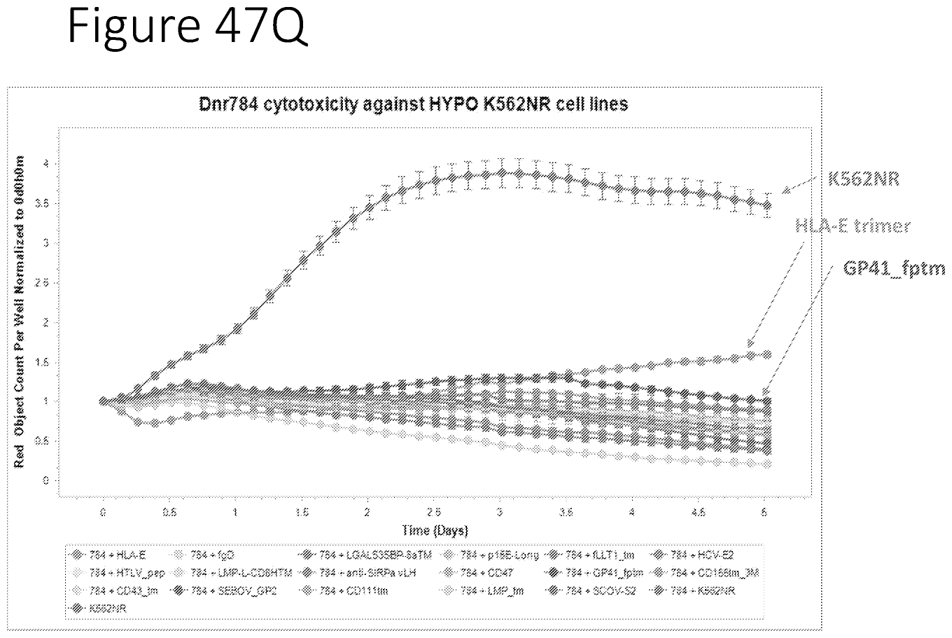

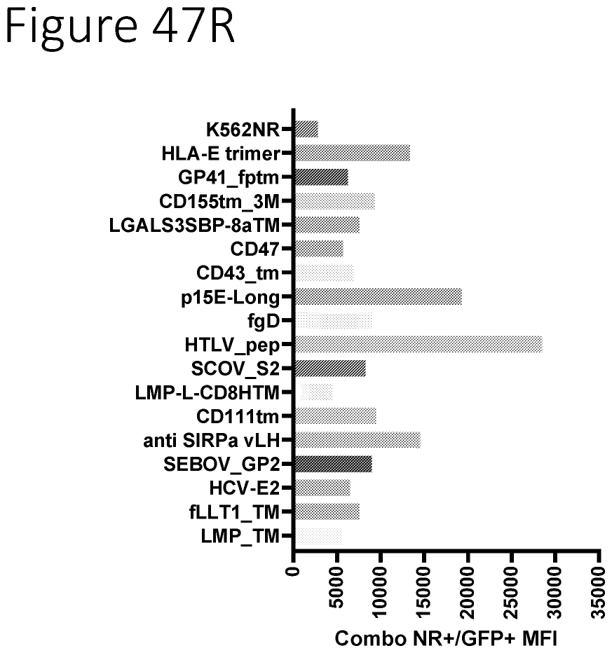

FIG. 47A shows a schematic process flow for testing the immunosuppressive effects of HLA re-expression that is a modification of that shown in FIG. 43. In brief, K562 cells expressing nuclear red dye (K562NR) are prepared as are various hypoimmune constructs as provided for herein. While these test constructs are engineered to include GFP (or a FLAG tag), it shall be appreciated that any of the immunosuppressive constructs provided herein can also be made without a tag. The immunosuppressive constructs are packaged in a retroviral vector and used to transduce the K562NR cells such that at least a portion of the K562 cells express the immunosuppressive construct and GFP (yielding a K562NR Hypo GFP population). These cells are then sorted such that 100% of the cells are NR/GFP positive, meaning that all cells express a hypoimmune construct. As discussed above, these are now considered the target cells in an assay. Thereafter, activated NK cells are co-cultured with the K562NR target cells at a 1:1 ratio, and the cytotoxic effects of the NK cells against the K562 NR target cells is assessed.

FIG. 47B shows cytotoxicity traces resulting from co-culture of the “target” K562 cells that have been modified to express the indicated immune evasion constructs with activated NK cells from a first donor. The top trace (K562NR, K562 modified to express nuclear red, but not expressing any GFP-tagged immune evasion construct) were cultured alone (no activated donor NK cells) and thus represent a positive control (maximal “target” cell growth), As a result, the closer a given experimental cytotoxicity trace is to that positive control, the greater degree of immune evasion was imparted to the target K562 cells. As shown in FIG. 47B, the HLA-E trimer conferred the greater degree of immune evasion to the K562 cells. This was followed by the GP41-fptm constructs. The D155tm-3M, LKAGLS3SBP-8aTM, CD43_tm and CD47 constructs all performed relatively similarly. The data in FIG. 47C relate to the level of expression of the indicated immune evasion constructs by the K562 cells (histogram bars represent the MFI of NR+/GFP+ cells) as well as their relative cytotoxicity ranking (e.g., 1-6). K562NR cells do not express an immune evasion construct, therefore express no GFP, and have relatively low MFl. In this experiment, the HLA-E trimer was expressed most robustly by the K562 cells and was also the most immune protective for the K562 cells against the cytotoxic effects of the activated donor NK cells. Interestingly, the next best performer in terms of immune protection, the GP41-fptm construct, was expressed at the lowest level by the K562 cells.

Similar cytotoxicity and expression data when activated NK cells from a second donor are provided in FIGS. 47D-47E. As with the data in FIG. 47B, the HLA-E trimer conferred the greatest degree of immune protection to the target K562 cells, with the other immune evasion constructs performing relatively similarly. FIG. 47E reveals again that the HLA-E construct was most robustly expressed and also conferred the most immune protection. Again, the GP41_fptm construct was not highly expressed, but was the second best at conferring immune protection the target K562 cells. FIG. 47F shows additional data when immune evasion construct-modified K562 cells were challenged with activated NK cells from a third donor. Interestingly, the constructs conferred varied amounts of immune protection as compared to challenge with cells from the other two donors. There was not a clear leader in terms of avoiding cytotoxicity, despite (see FIG. 47G) expression of HLA-E trimer remaining the most robust. Notably, the GP41-fptm construct retained its position at the second best at conferring immune evasion.

FIGS. 47H-47I show similar data for additional immune evasion constructs. FIG. 47H shows the degree of immune protection conferred by the various constructs and FIG. 47I shows the corresponding expression data. FIGS. 47J-47K show data for additional immune evasion constructs. FIG. 47J shows the degree of immune protection conferred by the various constructs and FIG. 47K shows the corresponding expression data. FIGS. 47L-47M show similar data for additional immune evasion constructs. FIG. 47L shows the degree of immune protection conferred by the various constructs and FIG. 47M shows the corresponding expression data. FIGS. 47N-47O show similar data for additional immune evasion constructs. FIG. 47N shows the degree of immune protection conferred by the various constructs and FIG. 47O shows the corresponding expression data. FIG. 47P shows a summary of data ranking various immune evasion constructs. The data from FIGS. 47H-47O are the screening data that led to the testing of the lead ranking constructs (data in 47A-47G). FIGS. 47Q and 47R show a large-scale comparison of the immune protection conferred on K562 cells expressing the indicated constructs (47Q) and the expression levels of the constructs (47R).

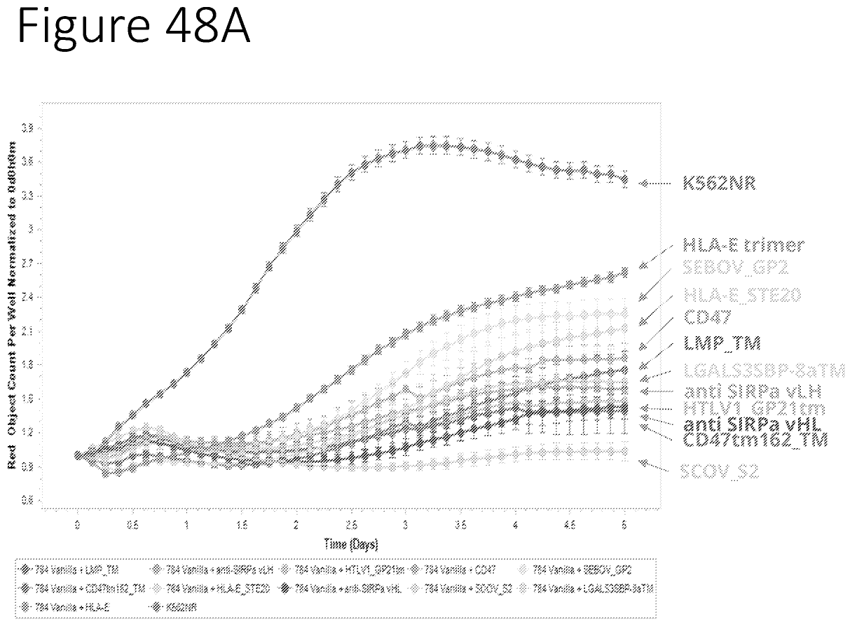

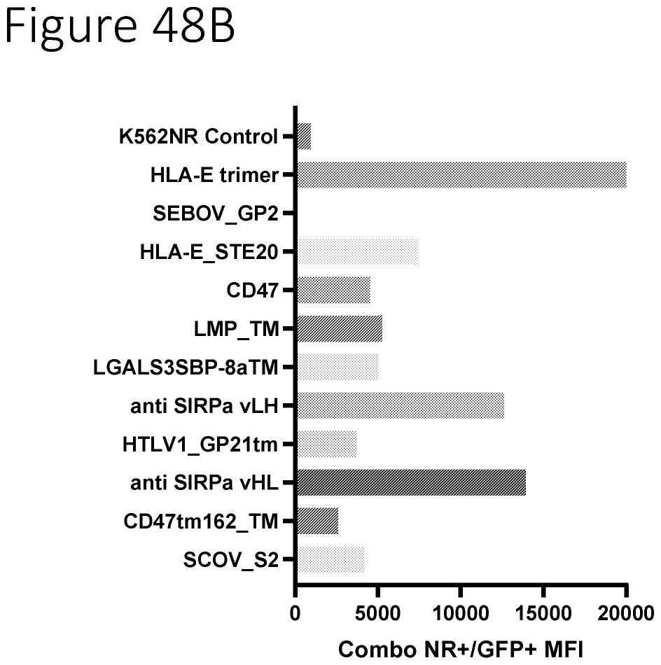

FIG. 48A shows additional data related to the indicated immune constructs and their ability to confer protection on K562 cells. This experiment used activated NK cells from the same donor as used to generate the data in FIG. 47D. As shown in FIG. 48A, the HLA-E trimer conferred the most protection on the target K562NR cells followed by the SEBOV-GP2 construct, HLA-E_STE20 and CD47. The other constructs did confer some degree of protection on the K562 target cells, but in lower proportions. FIG. 48B shows the rank order of the constructs (most to least effective top to bottom) as well as their degree of expression by the K562 cells. As seen with data discussed above, the HLA-E trimer was robustly expressed and, while it conferred the most protection to the K562 cells, other constructs provided significant protection, despite being expressed at lower levels.

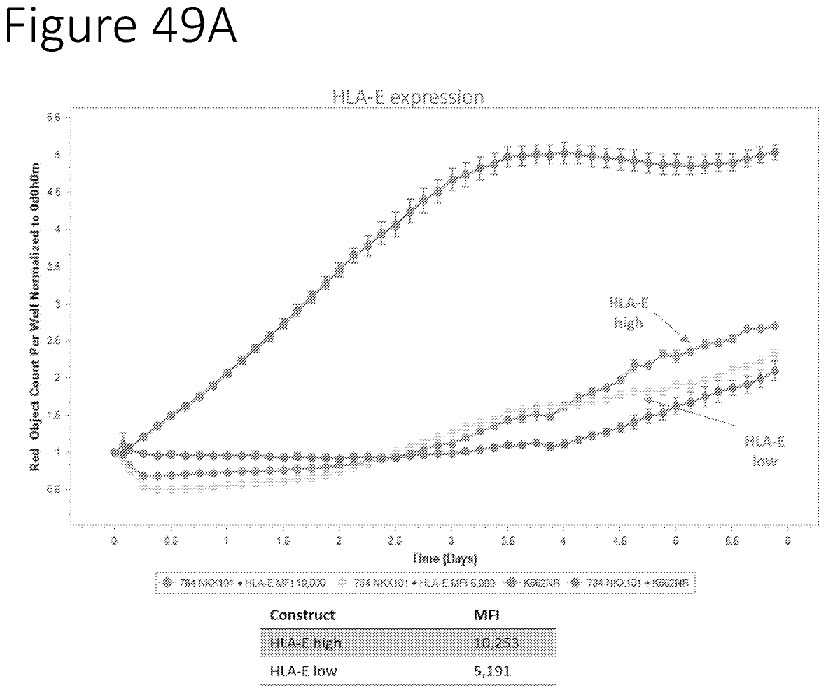

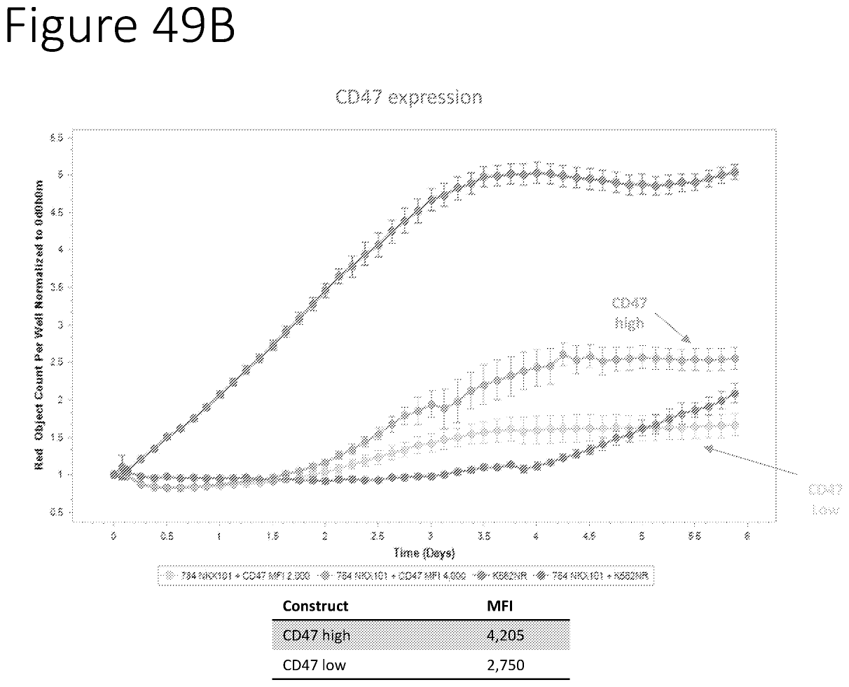

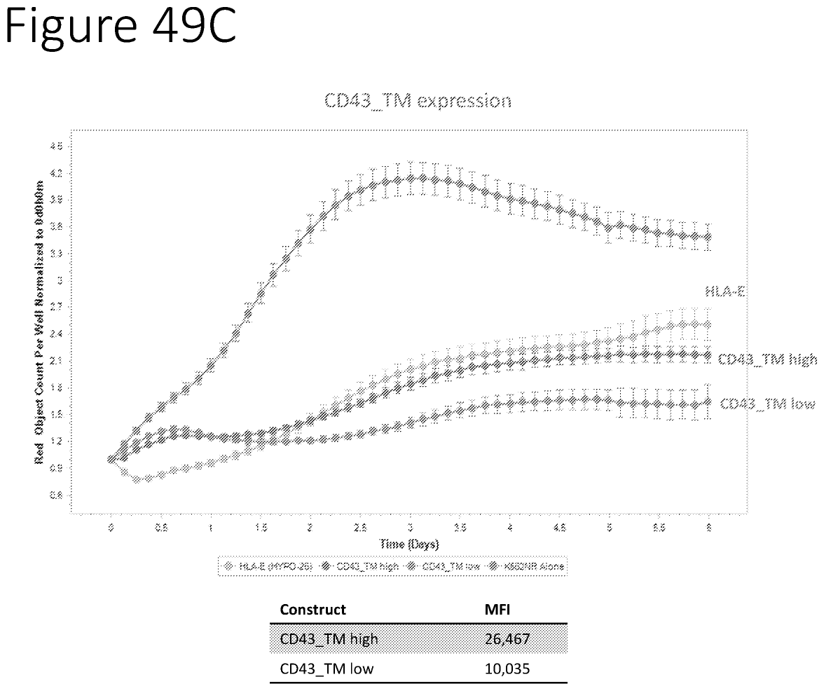

FIGS. 49A-49C show data related to the relative expression of certain constructs and the related immune protection that is conferred on cells expression the constructs. As with the approach above, K562NR cells were engineered to express a selected construct at either a relatively high abundance or a relatively low abundance. FIG. 49A shows data for HLA-E. As indicated in the summary table, the high-HLA-E expressing K562NR cells had approximately twice the expression of the HLA-E construct. As reflected in the curves, the high expressing K562 cells enjoyed a greater degree of immune protection from activated donor NK cells. A similar pattern is shown in FIG. 49B for CD47 expression and also in FIG. 49C for the CD43_TM construct. Thus, while the data from FIGS. 47 and 48 demonstrate that across constructs, expression levels may not correlate with the immune protection, FIG. 49 confirms that within a construct, an increase in the expression of a given construct does appear to correlate with immune protection. Thus, according to some embodiments, therapeutic cells are subjected to one or more steps to enhance expression of one or more immune evasion constructs (e.g., infection with a larger vector dose, re-exposure to vector to increase the immune construct payload, enriching a cell population for those with enhanced expression prior to treatment).

Taken together, these data taken together suggest that certain of the immune evasion constructs may act through distinct mechanisms of action that are more, or less, effective depending on the context. These include, but are not limited to, variations in expression, activity, blocking binding of NK cells, and the like. These data also suggest that certain constructs exhibit robust expression and, generally, robust immune evasion, while others express relatively poorly but exhibit efficient immune evasion. Thus, in several embodiments, modifications of the immune evasion constructs to improve expression by human cells (e.g., codon optimization) coordinately improves the immune protection conferred. Likewise, these data suggest that there may be donor to donor variability that cause engineered NK cells (e.g., those engineered to express a CAR) to respond differently to activating/inhibitory receptor signals on target cells that alter the cytotoxicity of the engineered cells. Thus, in several embodiments, a particular donor cell profile (e.g., donor NK or T cell phenotype) may be preferred for use in an allogeneic cell therapy context. More information on an advantageous donor cell profile can be found in U.S. Provisional Patent Application Nos. 63/203,703 and 63/262,544, filed Jul. 28, 2021 and Oct. 14, 2021 (respectively), the entire contents of each of which is incorporated by reference herein. As provided for herein, an overall immune evasion strategy can employ one or more approaches to confer protection on immune cells, such as HLA-E expression modifications, expression of one or more viral immunosuppressive peptide, disruption of TCR activation, and/or use of inhibitory ligands. For example, in some embodiments, the one or more approaches to confer protection on immune cells may involve expression and/or modulation of HLA-E, HLA-E trimer, HLA-E STE20, SEBOV-GP2, CD47, LMP, LGAL3S, SIRPa (vH or vL), HTLV1, or SCOV. In several embodiments, the overall approach allows for a more effective allogeneic cell therapeutic regimen.

### Example 5—Further Gene Editing of Immune Cells to Alter Cell Function

As provided for herein, gene editing (e.g., using a Crispr/Cas system) is used to enhance the functionality (e.g., persistence, cytotoxicity, and/or other characteristics) of immune cells, such as NK cells and/or T cells. Edits may be made at a single gene to reduce, substantially reduce, or eliminate expression and/or function of the protein encoded by the gene. Additionally, edits may be made at multiple gene targets (e.g., two, three, four, or more targets) to reduce, substantially reduce, or eliminate expression and/or function of the protein encoded by each of the genes edited, with the lack of expression or function of the multiple proteins imparting to the edited cells an unexpectedly effective phenotype, either in terms of persistence, degree of cytotoxic signaling (either amount and/or duration), resistance to tumor microenvironment suppressive effects, or other beneficial characteristics.

For example, as discussed herein, in several embodiments, gene edits are made at a target site in a CISH gene and a target site in a CBLB gene. In some embodiments, a double edit, e.g., CISH/CBLB is made in NK cells and/or T cells for use in therapy. In several embodiments, a combination CISH/CBLB gene edit is made in an NK cell that does not include an additional edit. In several embodiments, a combination CISH/CBLB gene edit is made in an NK cell that does not express any one or combination of any of an anti-CD70 CAR, an anti-CD19 CAR, or an anti-NKG2D chimeric receptor, but are optionally engineered to express a CAR targeting another tumor target.

For example, as discussed herein, in several embodiments, gene edits are made at a target site in a CISH gene and a target site in a TGFBR2 gene. In some embodiments, a double edit, e.g., CISH/TGFBR2 is made in NK cells and/or T cells for use in therapy. In several embodiments, a combination CISH/TGFBR2 gene edit is made in an NK cell that does not include an additional edit. In several embodiments, a combination CISH/TGFBR2 gene edit is made in an NK cell that does not express any one or combination of any of an anti-CD70 CAR, an anti-CD19 CAR, or an anti-NKG2D chimeric receptor, but are optionally engineered to express a CAR targeting another tumor target.

For example, as discussed herein, in several embodiments, gene edits are made at a target site in a CISH gene and a target site in a TIGIT gene. In some embodiments, a double edit, e.g., CISH/TIGIT made in NK cells and/or T cells for use in therapy. In several embodiments, a combination CISH/TIGIT gene edit is made in an NK cell that does not include an additional edit. In several embodiments, a combination CISH/TIGIT gene edit is made in an NK cell that does not express any one or combination of any of an anti-CD70 CAR, an anti-CD19 CAR, or an anti-NKG2D chimeric receptor, but are optionally engineered to express a CAR targeting another tumor target.

As discussed above, CRISPR/Cas9 was used to specifically target and reduce the expression of selected gene and gene combinations in NK cells and an evaluation of the resultant cytotoxicity of the edited cells was performed.

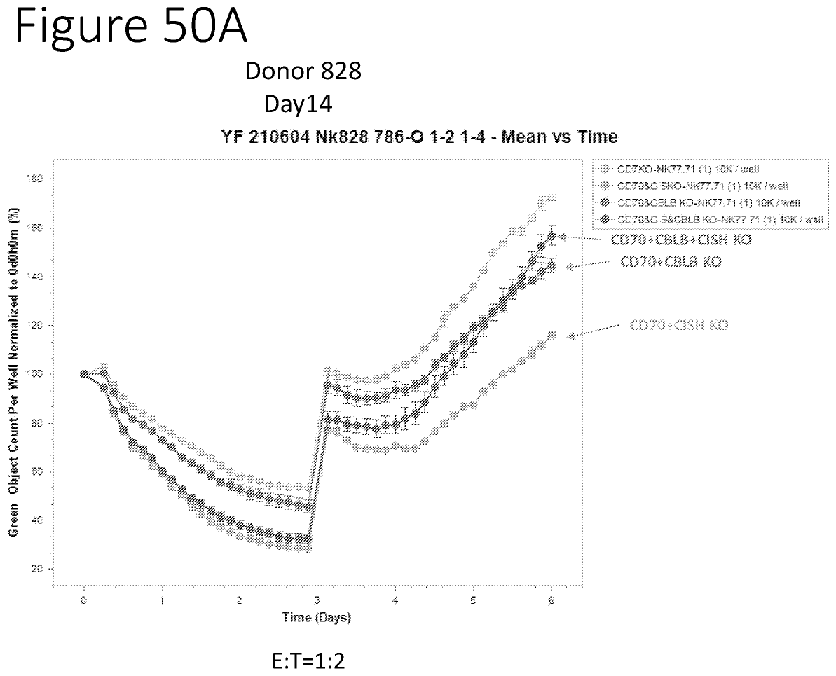

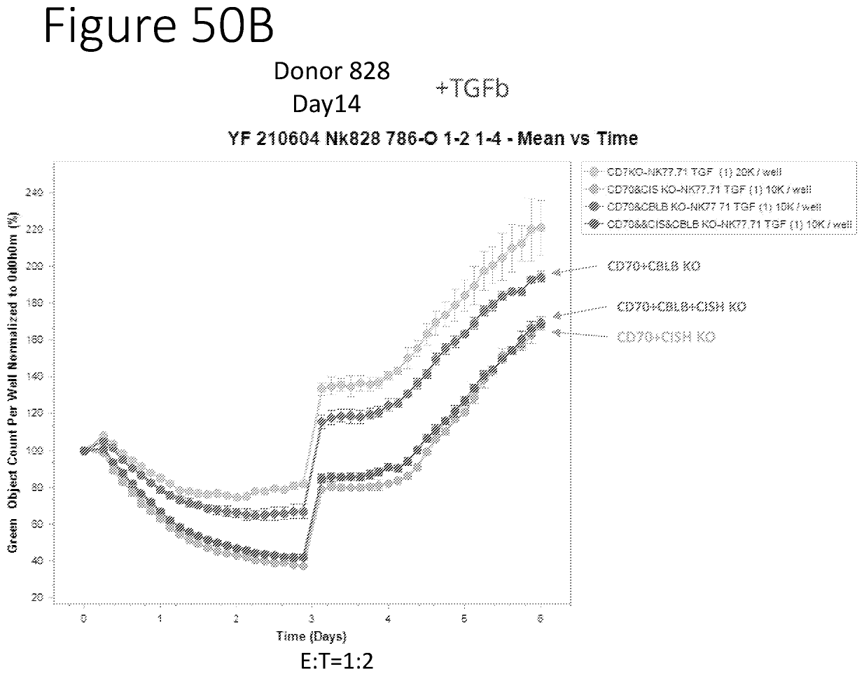

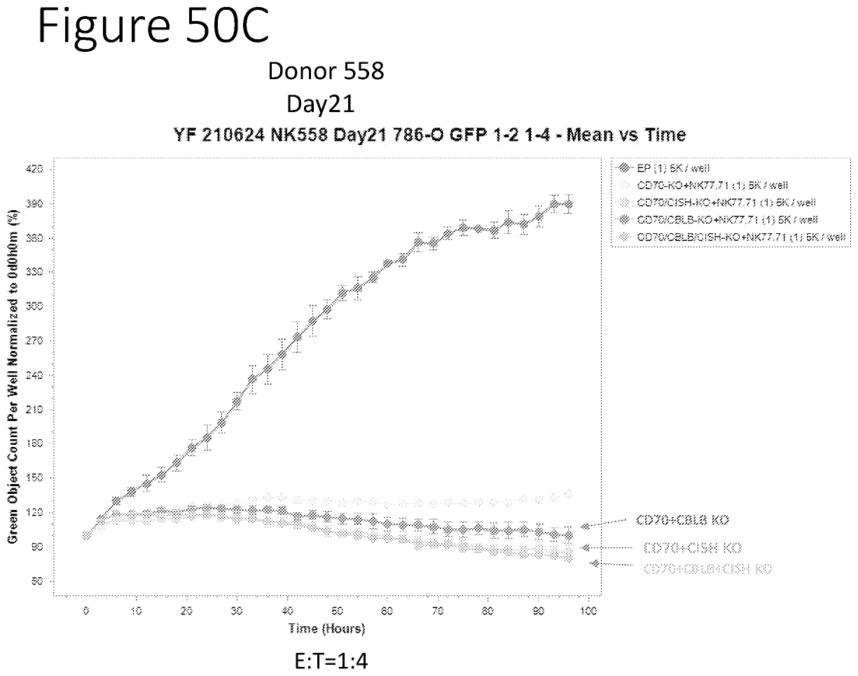

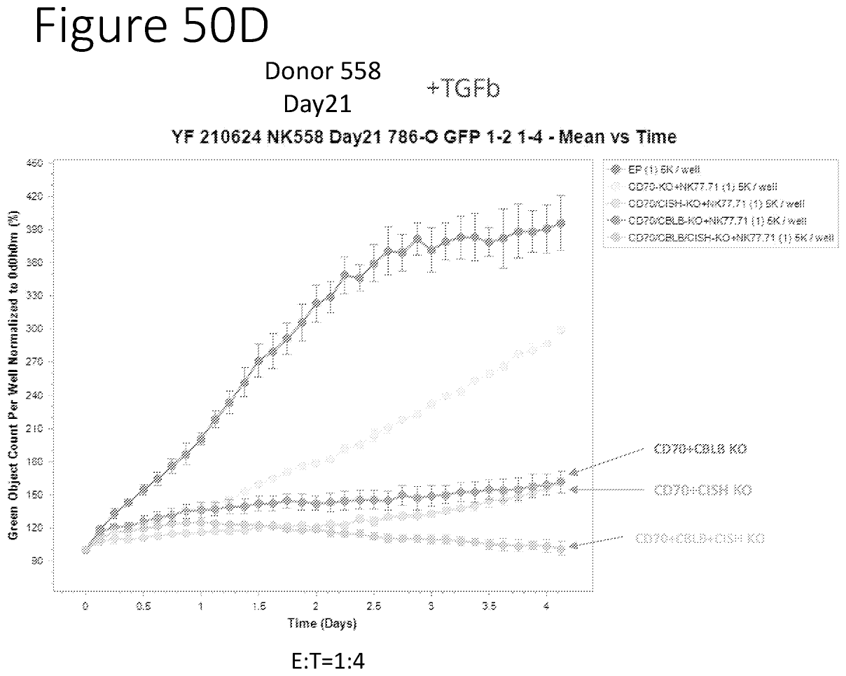

FIG. 50A shows cytotoxicity data for NK cells edited at selected gene target or targets and co-cultured with 786-O renal cell carcinoma cells. It shall be appreciated that, while 786-O cells express a prevalent amount of CD70 and therefore certain experiments discussed herein relate to edited cells expressing a non-limiting example of an anti-tumor CAR (here an anti-CD70 CAR), the use of the CD70 CAR (and any associated edits to CD70 expression which are made to avoid/reduce fratricide) are specific to these experiments, and the edited cells provided for herein may also be engineered to target tumor markers other than CD70. The data in FIG. 50A were collected 14 days after the indicated gene edits were made and using an effector:target (E:T ratio of 1:2). As shown in FIG. 50A, edits to were made to the CD70 gene for all groups. Edits to CBLB alone, or CBLB and CISH together did not appear to enhance the cytotoxicity of the NK cells in this particular experiment. However, as shown in FIG. 50B, in which the co-culture with target 786-O cells was performed in the presence of TGFb (a known cytokine released into the TME to suppress immune cell activity against tumors) the combination of a CISH/CBLB edit made a marked difference in the cytotoxicity of those cells, improving their cytotoxicity as compared to CBLB edits alone, and rendering them equally as effective as CISH-edited cells. This data shows that the dual edit of CISH/CBLB confers the dual-edited cells with resistance to certain suppressive effectors in the TME, which enhances their cytotoxicity profile. FIG. 50C shows similar data for cells for another donor edited at the indicated gene or genes and evaluated against 786-O cells at 21 days post-editing. As shown, each of a CISH edit, a CBLB edit and a combination CISH/CBLB edit resulted in significant cytotoxicity against the tumor cells, even at a 1:4E:T. FIG. 50D shows additional striking data in the presence of TGFb, in which the single edits to either CISH or CBLB resulted in reduce control of tumor cell growth, but the dual CISH/CBLB edit allowed the donor NK cells to maintain robust cytotoxic effects against the target cells. This data shows that, in several embodiments, a dual edit to CISH/CBLB imparts significant improvements in immune cell function against a target tumor. In several embodiments, the edits are in NK cells, while such dual edits (or triple edits, or more) are possible in T cells as well.

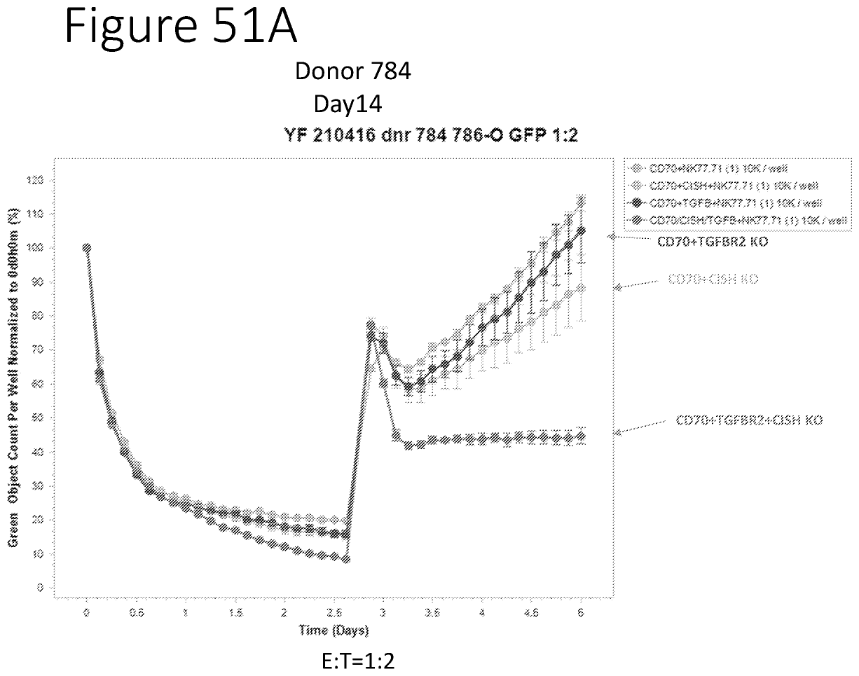

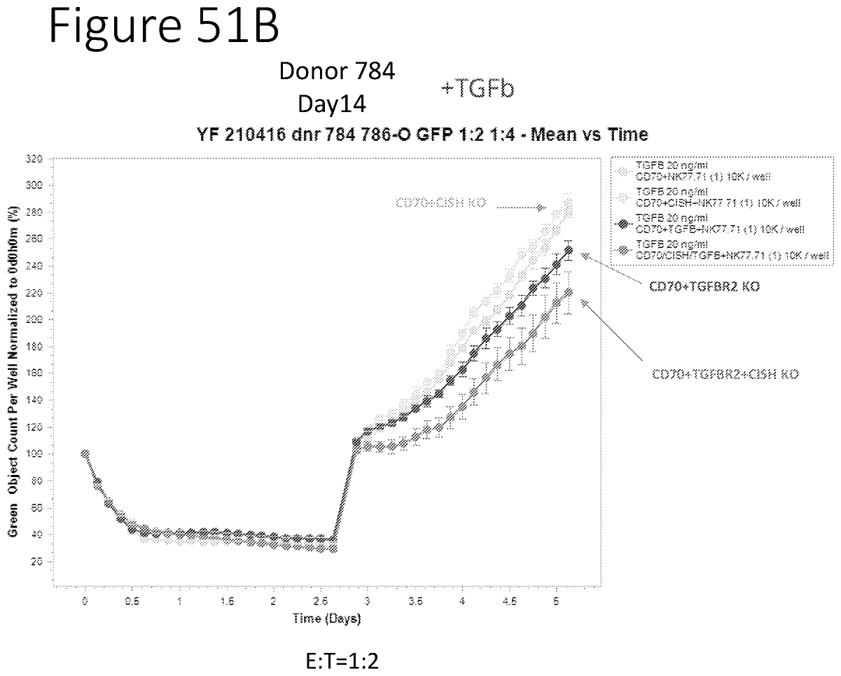

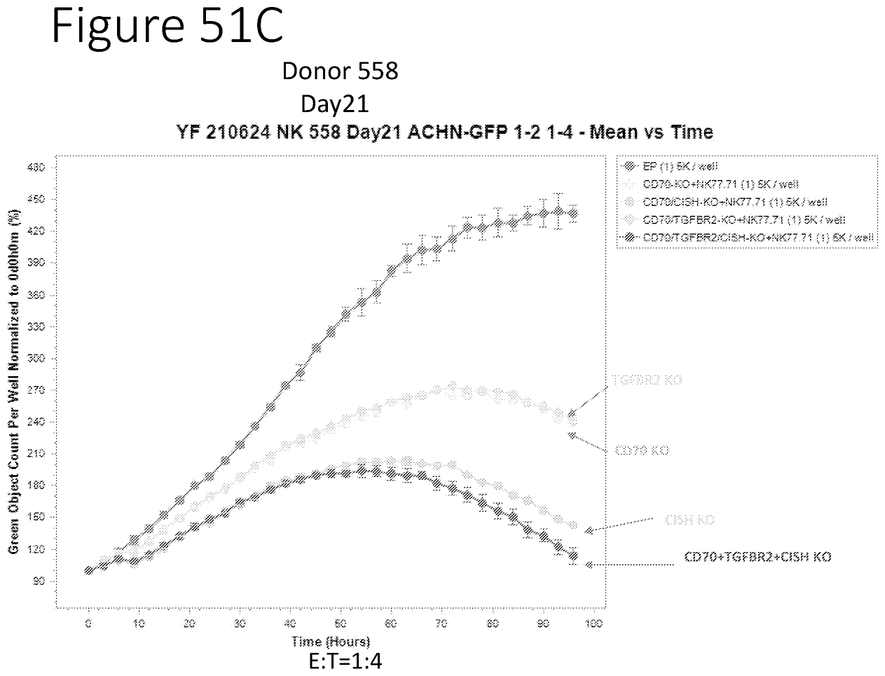

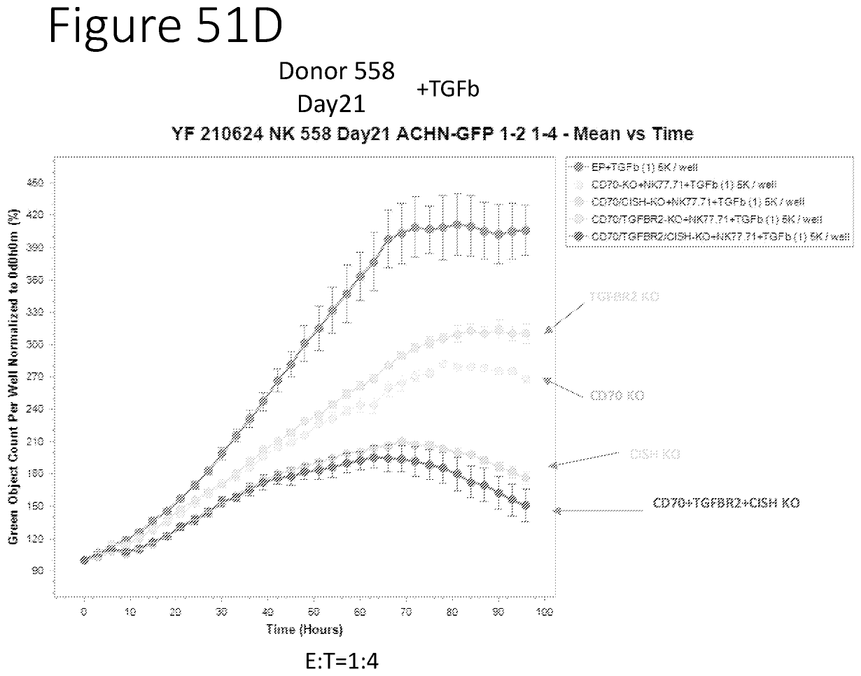

FIG. 51A shows data with respect to donor NK cells edits at CISH, TGFBR2 or at CISH/TGFBR2 in combination and evaluated for cytotoxicity against target 786-O cells (again with the use of the CD70 CAR and CD70 edit being specific to this experimental setup) 14-days post-editing. The data show that the combination CISH/TGFBR2 edit yields NK cells that have a much more robust cytotoxicity profile than cells edited as CISH or TGFBR2. FIG. 51B shows similar data, but in the presence of TGFb. In this experiment, the TGFb appears to successfully suppress the effects of each of the edited cell types, reflecting its suppressive power in the TME. FIG. 51C shows related data, collected at 21 days post-edit and with a co-culture of the edited NK cells with ACHN target cells at a 1:4E:T ratio. It shall be appreciated that, while ACHN cells also express a prevalent amount of CD70 and therefore certain aspects of these experiments relate to edited cells expressing a non-limiting example of an anti-tumor CAR (here an anti-CD70 CAR), the use of the CD70 CAR (and any associated edits to CD70 expression which are made to avoid/reduce fratricide) are specific to these experiments, and the edited cells provided for herein may also be engineered to target tumor markers other than CD70. FIG. 51C shows that the combination of CISH/TGFBR2 edit confers the greatest degree of cytotoxicity on the edited cells, as compared to editing TGFBR2 or CISH. FIG. 51D shows additional data where co-culturing was done in the presence of TGFb. In this experiment the ability of the TGFb to negate the cytotoxic effects of the CISH/TGFBR2 edited cells was significantly reduced, showing that in some embodiments an immune cell edited at CISH and TGFBR2 and expressing an anti-tumor CAR has enhanced cytotoxicity (and/or persistence, in some embodiments).

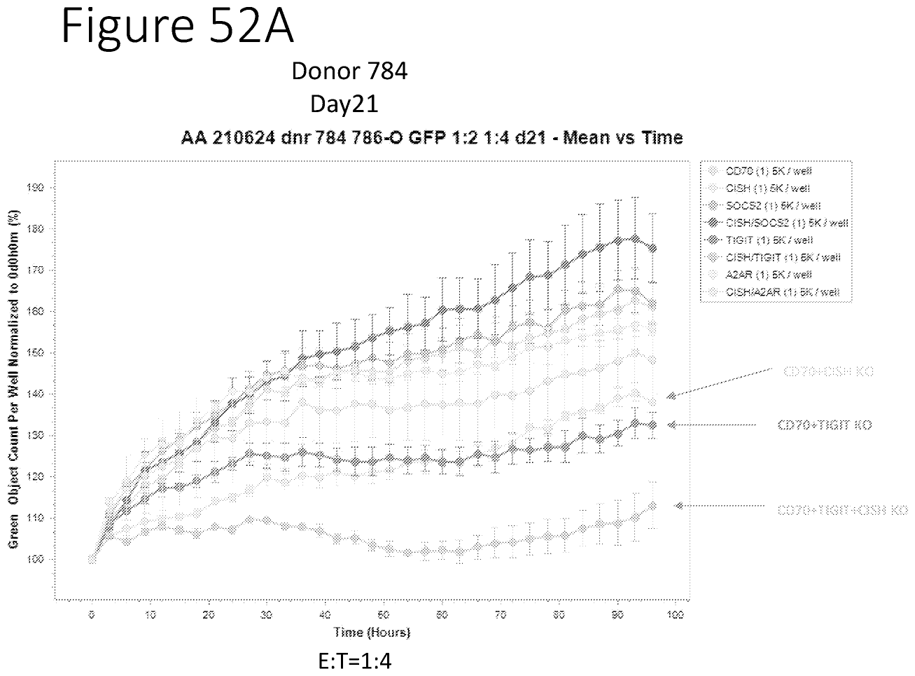

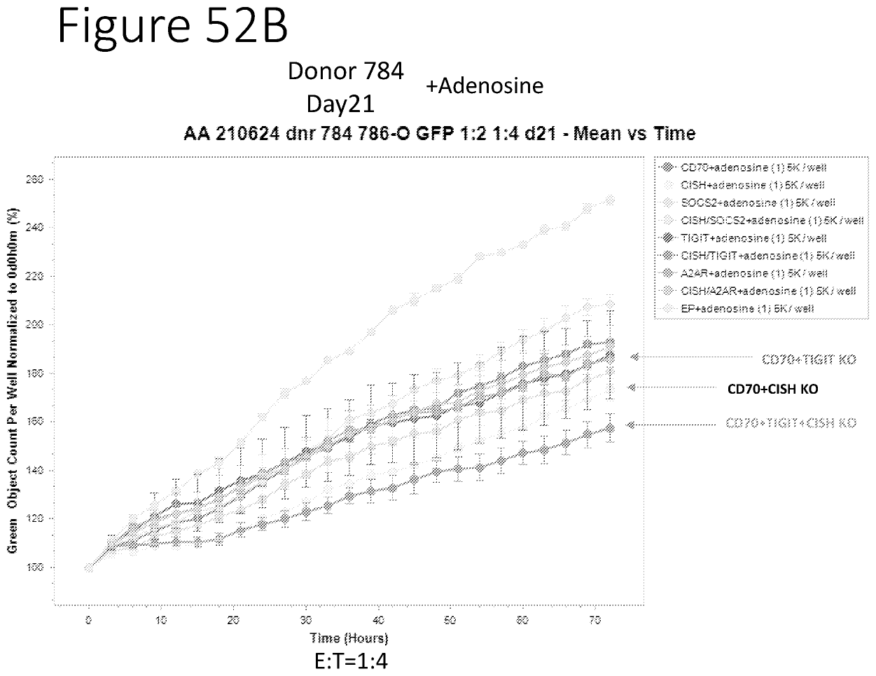

FIGS. 52A and 52B relate to edits made to TIGIT. Here, a 1:4E:T ratio was used to co-culture NK cells edited at CISH, TIGIT, or a combination of CISH/TIGIT with 786-O target cells at day 21 post-edit. As with the CISH/CBLB and CISH/TGFBR2 edits, the combination edit of CISH and TIGIT led to superior cytotoxicity against the target cells. FIG. 52B shows experimental data when co-culture was performed in the presence of adenosine, a well-established suppressor of immune cell function. While the adenosine did reduce the cytotoxicity of each of the tested edited cells, the CISH/TIGIT edited cells remained the most cytotoxic, reinforcing the that edits to CISH in combination with another gene, like TIGIT (or others disclosed herein) enhances the cytotoxic activity (and/or persistence) of the edited NK cells.

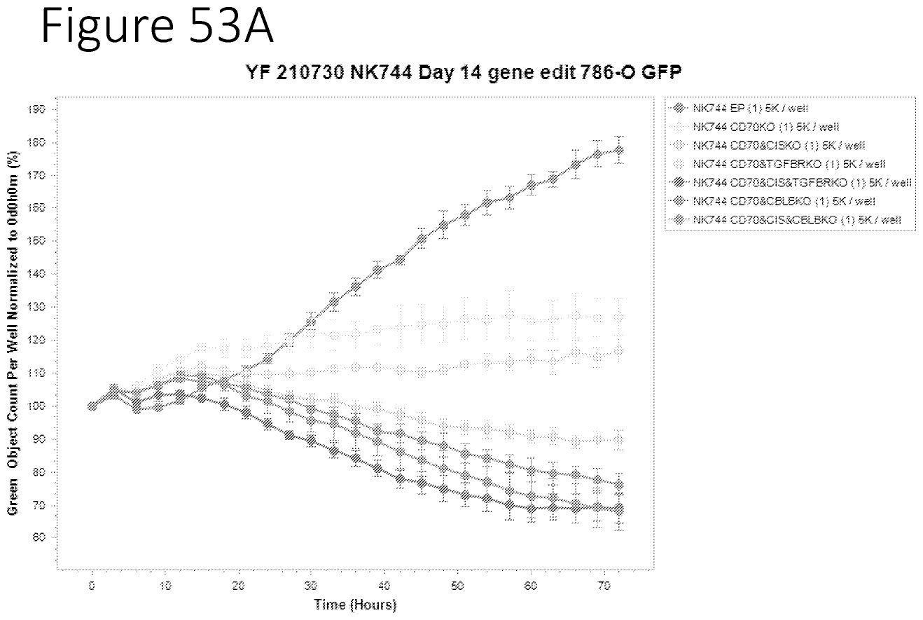

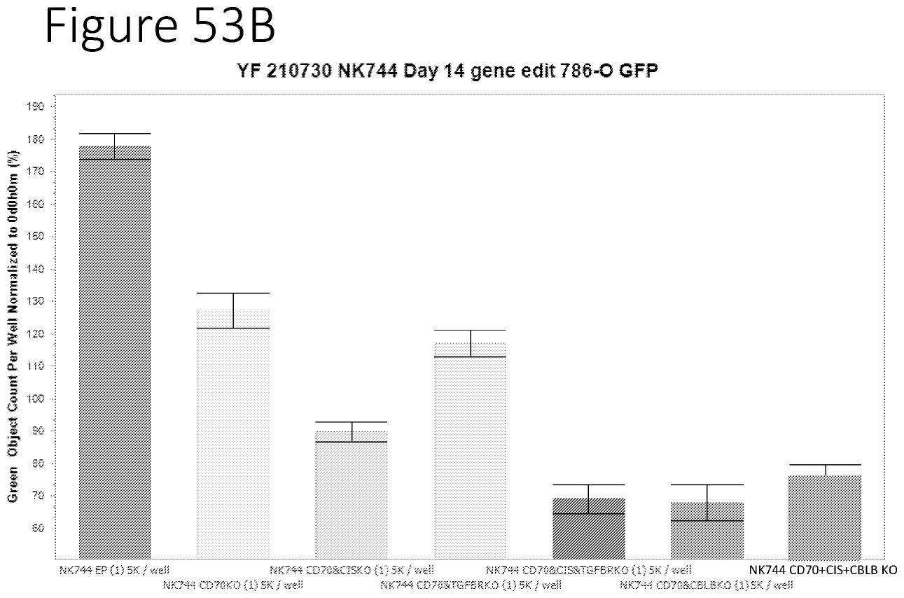

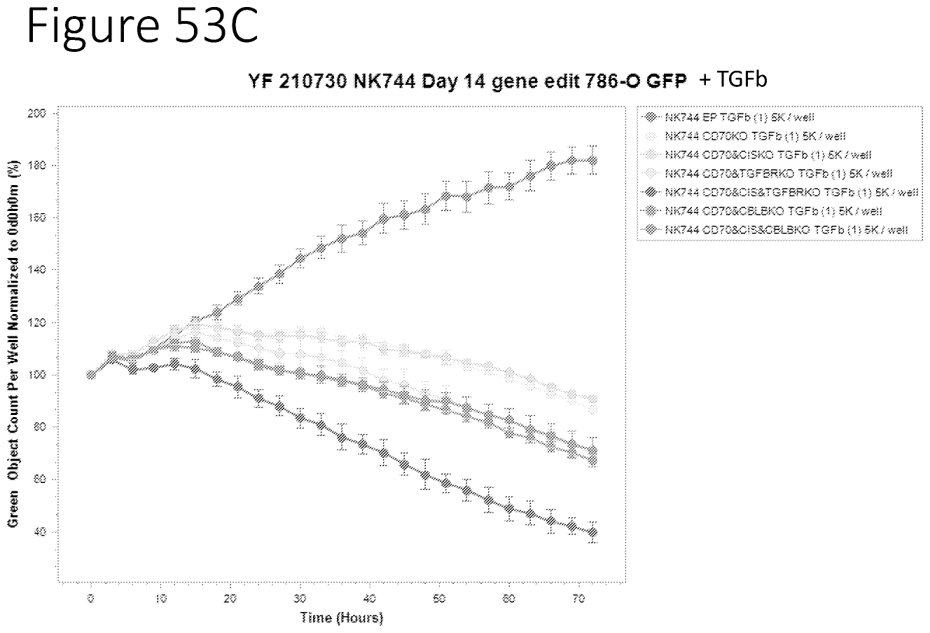

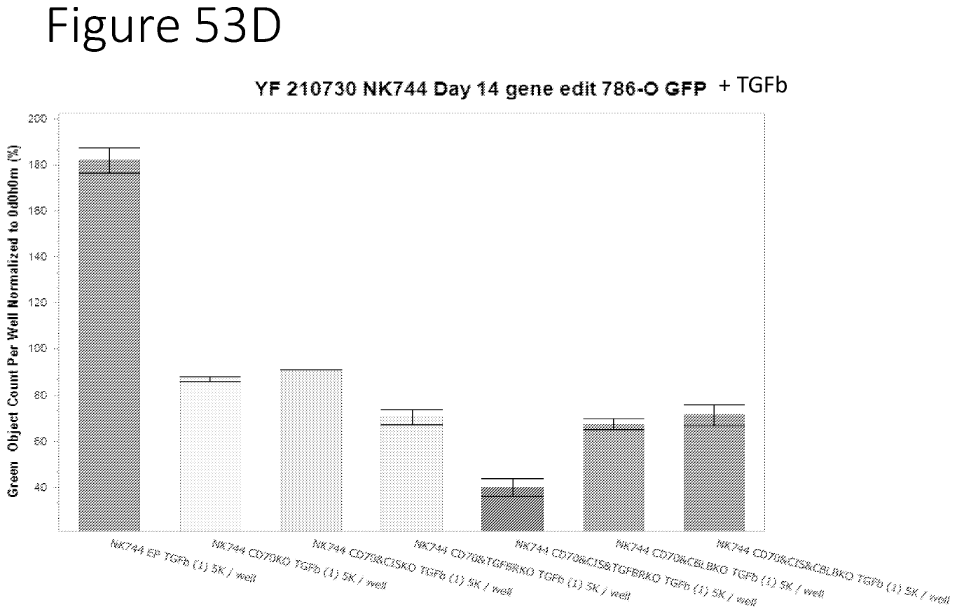

FIGS. 53A-53D show additional data related to the effects of certain edits provided for herein. FIG. 53A shows a cytotoxicity curve for the various indicated edits against 786-O target cells at 14 days post-edit. FIG. 53B shows a histogram that reflects a summary of the data in FIG. 53A. The edits in this experiment were to CD70 (which as discussed above is specific to the model and avoiding fratricide based on the non-limiting example of a CD70 CAR), CISH, TGFBR2, CBLB, CISH/TGFBR2, or CISH/CBLB. As best seen in FIG. 53B, the edits that include CISH/CBLB (far right) and CISH/TGFBR2 (3^{rd} from right) yield some of the most cytotoxic NK cells tested. The edit to CBLB also imparts significant cytotoxicity to the edited NK cells. FIG. 53C shows corresponding data, wherein the co-culture was performed in the presence of TGFb. The summary data in FIG. 53D again reflect the immunosuppressive power of TGFb and the effectiveness of edits at reducing this effect. While the cytotoxicity of the edited NK cells was blunted in the presence of TGFb, the combination edits of CISH/TGFBR2 and CISH/CBLB still exhibited relatively robust cytotoxic effects on the target cells.

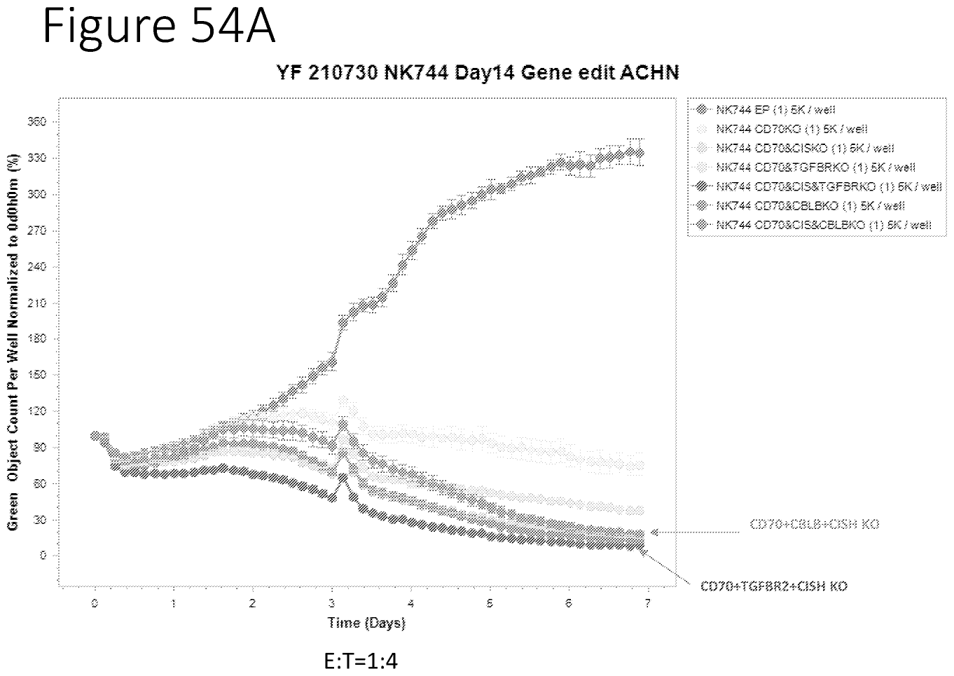

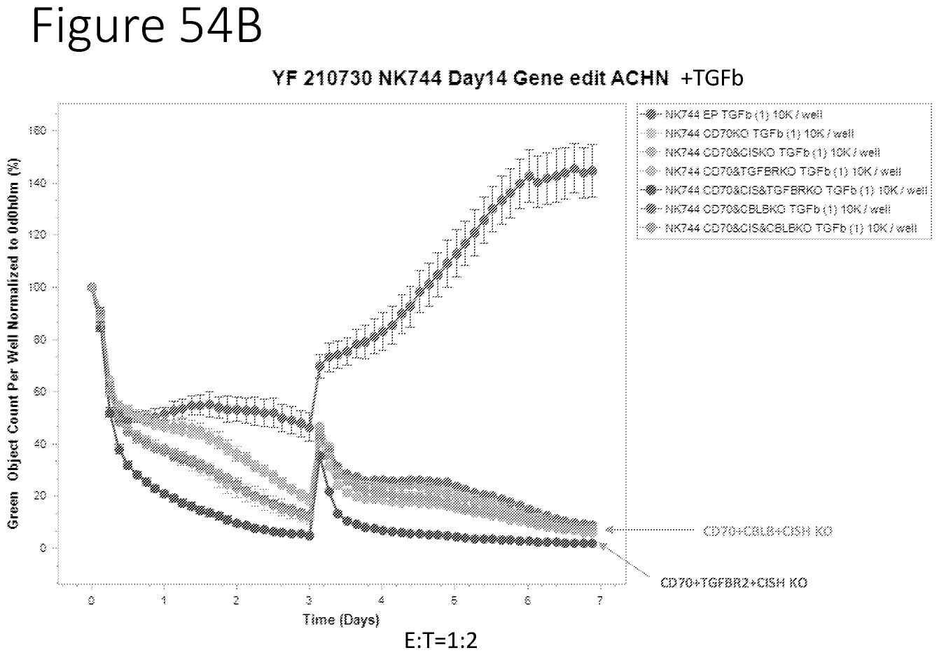

Similar experiments were performed using an alternative target cell, the ACHN RCC cells. FIGS. 54A-154B show the collected data. As depicted in FIG. 54A, each of the edited cells tested showed measurable control of tumor growth at a 1:4E:T. The CISH/TGFBR2 and CISH/CBLB (arrows) performed as well, if not better, than the cells not edited at those combinations. FIG. 54B shows a related experiment, using an E:T of 1:2 and in the presence of TGFb. Again, the CISH/TGFBR2 and CISH/CBLB (arrows) performed as well, if not better, than the cells not edited at those combinations.

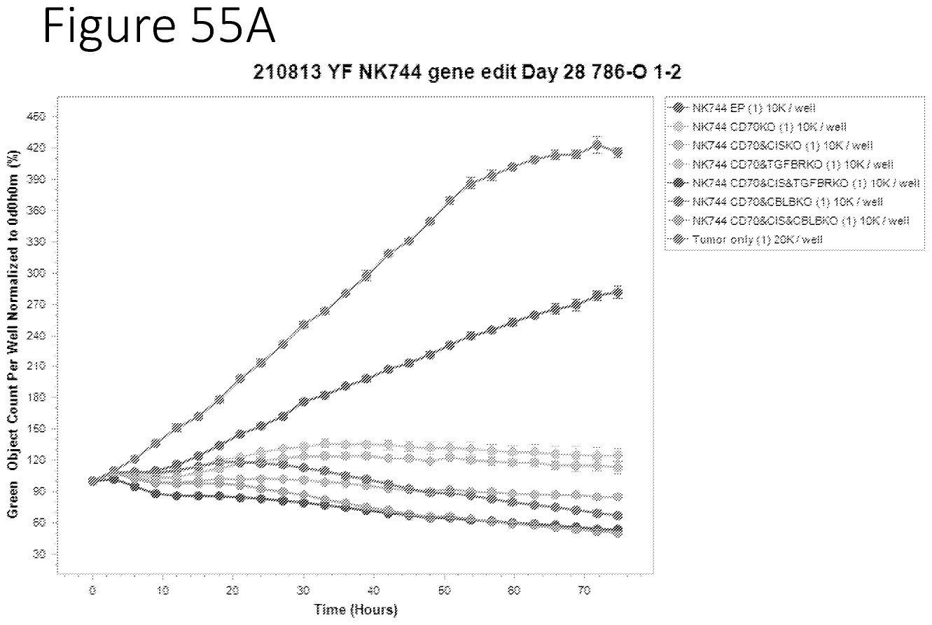

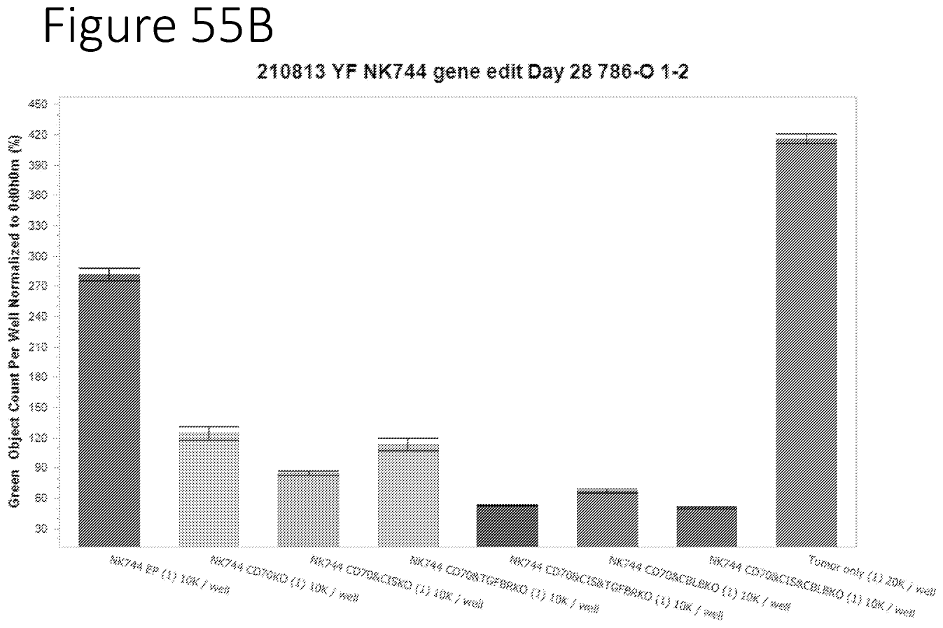

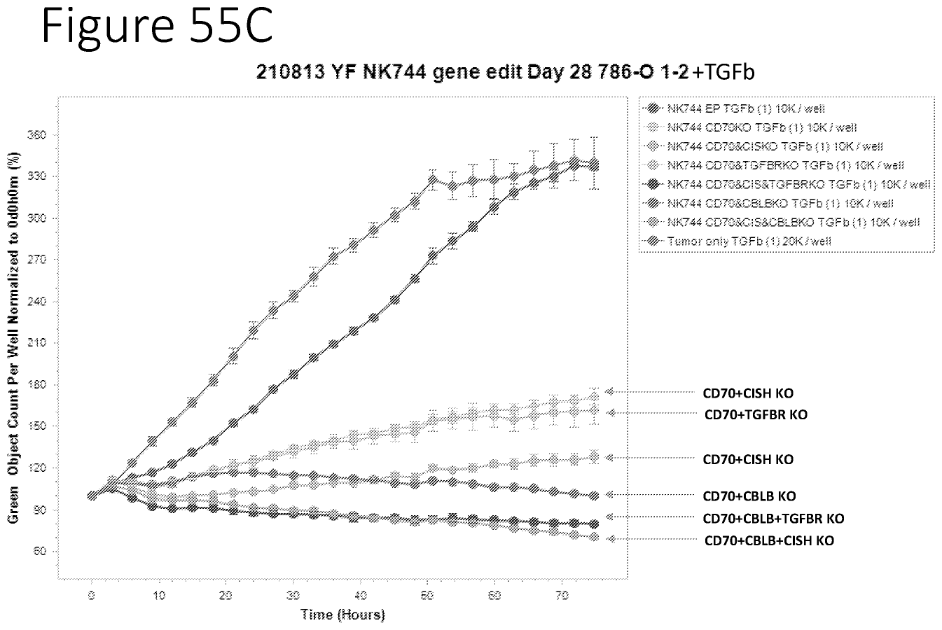

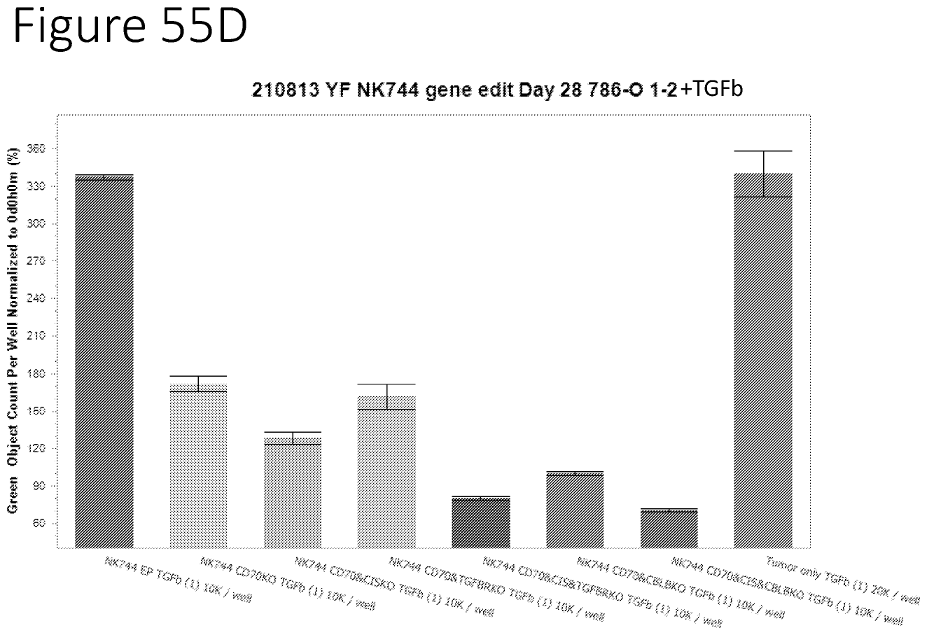

FIGS. 55A-55D show additional data related to the effects of certain edits provided for herein. FIG. 55A shows a cytotoxicity curve for the various indicated edits against 786-O target cells at 28 days post-edit. FIG. 55B shows a histogram that reflects a summary of the data in FIG. 55A. As depicted in FIG. 55B, the combined edit of CISH/CBLB or CISH/TGFBR2 resulted in the most robust cytotoxicity against the target cells of any of the edits tested. FIGS. 55C/55D show corresponding data when TGFb was present in the co-culture. Notably, again, the combined edit of CISH/CBLB or CISH/TGFBR2 resulted in the most robust cytotoxicity against the target cells of any of the edits tested, despite the suppressive effects of the TGFb.

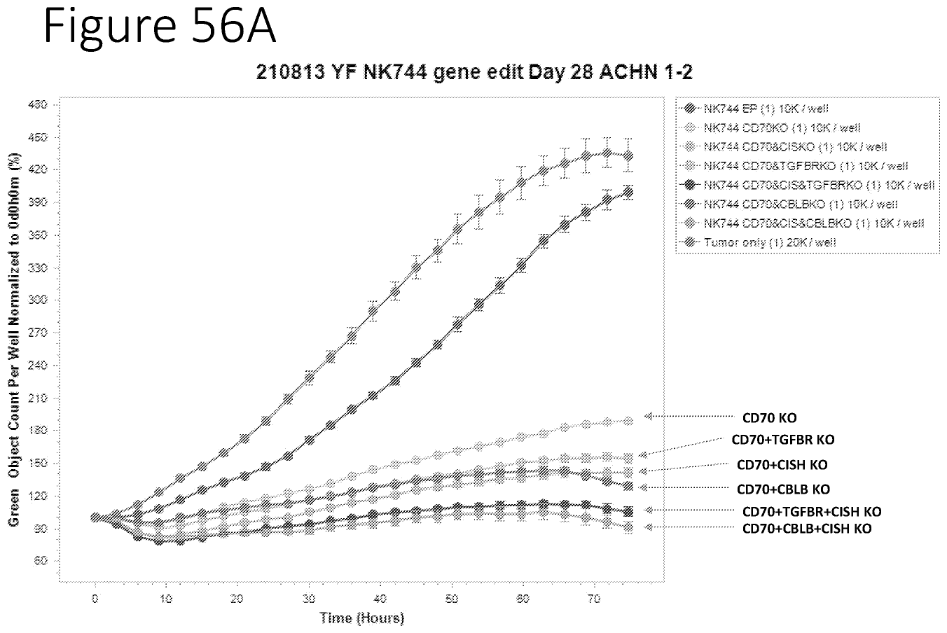

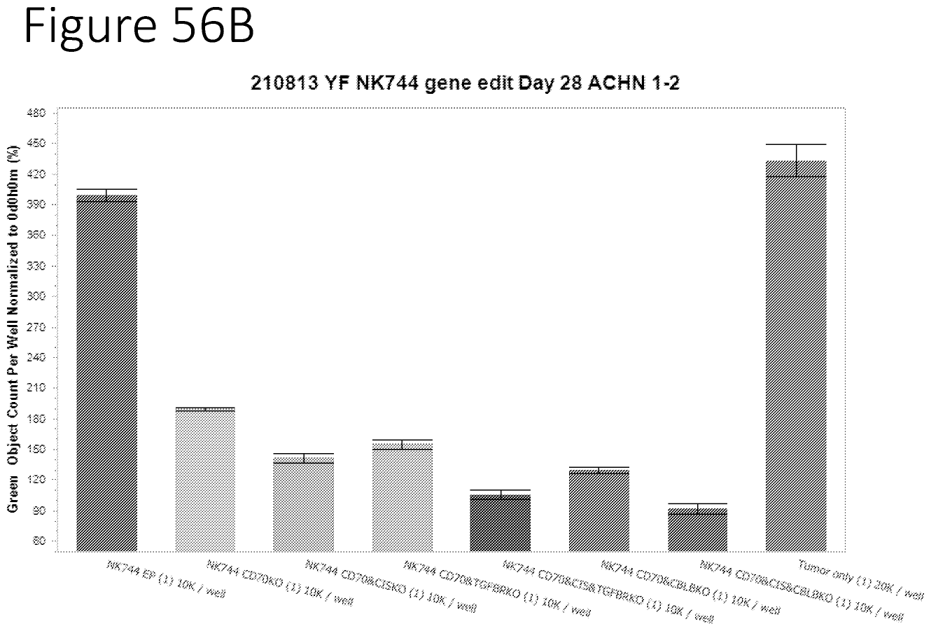

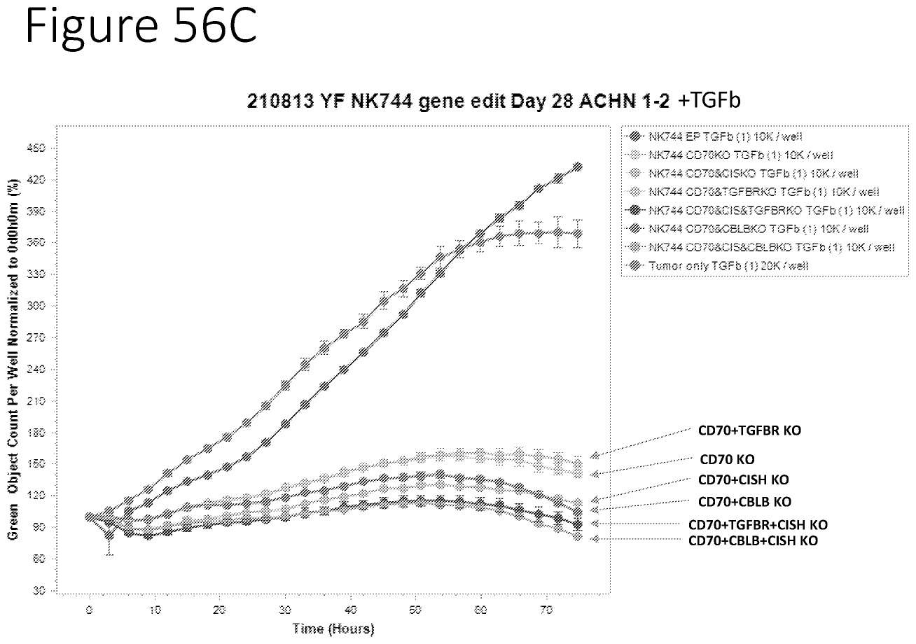

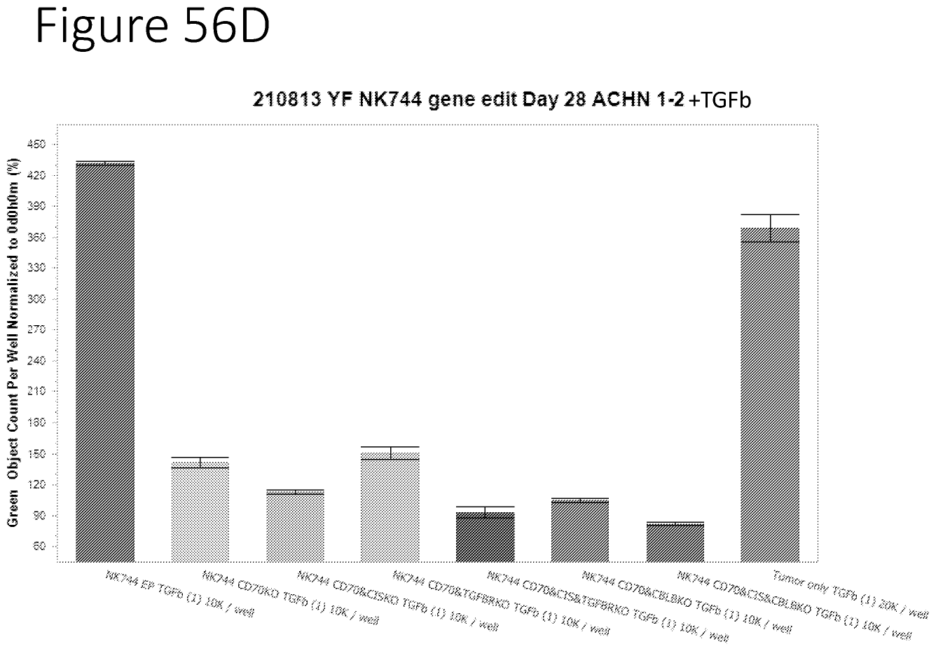

FIGS. 56A-56D show additional data related to the effects of certain edits provided for herein using ACHN cells as the target cells. FIG. 56A shows a cytotoxicity curve for the various indicated edits against ACHN target cells at 28 days post-edit. FIG. 56B shows a histogram that reflects a summary of the data in FIG. 56A. As depicted in FIG. 56B, the combined edit of CISH/CBLB or CISH/TGFBR2 resulted in the most robust cytotoxicity against the target cells of any of the edits tested. FIGS. 56C/56D show corresponding data when TGFb was present in the co-culture. Notably, again, the combined edit of CISH/CBLB or CISH/TGFBR2 resulted in the most robust cytotoxicity against the target cells of any of the edits tested, despite the suppressive effects of the TGFb.

Taken together, these data demonstrate that there is an unexpectedly powerful enhancement to the cytotoxicity of edited immune cells, such as NK cells (or T cells) when edited at multiple gene targets. For example, the combination of editing to reduce, substantially eliminate, or eliminate CIS protein expression or function, in combination with another target yields enhanced cytotoxicity and/or persistence of the edited cells. In several embodiments, edits to CISH and edits to reduce, substantially eliminate, or eliminate CBLB expression are provided for and result in enhanced anti-tumor effects, regardless of the tumor type (e.g., if used, for example with any CAR that targets a tumor). In several embodiments, a CISH/CBLB combination edit is used without an edit to a CD70 gene, and/or without expression of an anti-CD70 CAR. In several embodiments, a CISH/CBLB combination edit is used without expression of an anti-CD19 CAR. In several embodiments, a CISH/CBLB combination edit is used without an additional edit and without expression of an anti-CD19 CAR.

In additional embodiments, the combination of editing to reduce, substantially eliminate, or eliminate CIS protein expression or function, in combination with another target yields enhanced cytotoxicity and/or persistence of the edited cells. In several embodiments, edits to CISH and edits to reduce, substantially eliminate, or eliminate TGFBR2 expression are provided for and result in enhanced anti-tumor effects, regardless of the tumor type (e.g., if used, for example with any CAR that targets a tumor). In several embodiments, a CISH/TGFBR2 combination edit is used without an edit to a CD70 gene, and/or without expression of an anti-CD70 CAR. In several embodiments, a CISH/TGFBR2 combination edit is used without expression of an anti-CD19 CAR. In several embodiments, a CISH/TGFBR2 combination edit is used without an additional edit and without expression of an anti-CD19 CAR.

In additional embodiments, the combination of editing to reduce, substantially eliminate, or eliminate CIS protein expression or function, in combination with another target yields enhanced cytotoxicity and/or persistence of the edited cells. In several embodiments, edits to CISH and edits to reduce, substantially eliminate, or eliminate TIGIT expression are provided for and result in enhanced anti-tumor effects, regardless of the tumor type (e.g., if used, for example with any CAR that targets a tumor). In several embodiments, a CISH/TIGIT combination edit is used without an edit to a CD70 gene, and/or without expression of an anti-CD70 CAR. In several embodiments, a CISH/TIGIT combination edit is used without expression of an anti-CD19 CAR. In several embodiments, a CISH/TIGIT combination edit is used without an additional edit and without expression of an anti-CD19 CAR.

### Additional Embodiments

In several embodiments, there is provided a population of genetically engineered immune cells, comprising one or more of genetically engineered NK cells and genetically engineered T cells; wherein the plurality of genetically engineered immune cells are engineered to express a cytotoxic receptor comprising an extracellular ligand binding domain, a transmembrane domain, and a cytotoxic signaling complex, wherein the genetically engineered immune cells are genetically engineered to express at least one immunosuppressive effector, wherein the at least one immunosuppressive effector exerts suppressive effects on undesired cytotoxic activity of suppressive cells, wherein, optionally, the genetically engineered immune cells are further engineered to express one or more of the following: IL-15, mblL-15, at least one viral immunosuppressive peptide integrated within an extracellular region, or extracellular regions, of the cytotoxic receptor, at least one viral immunosuppressive peptide expressed on a cell membrane of the genetically engineered immune cells, at least one portion of a human immunosuppressive protein or protein complex integrated within an extracellular region, or extracellular regions, of the cytotoxic receptor, at least one portion of a human immunosuppressive protein or protein complex expressed on a cell membrane of the genetically engineered immune cells, and/or at least one chimeric viral-human immunosuppressive construct expressed on a cell membrane of the genetically engineered immune cells and/or integrated at one or more extracellular regions of the cytotoxic receptor.

In several embodiments, there is also provided a population of genetically engineered immune cells, comprising: a plurality of genetically engineered NK cells and a plurality of genetically engineered T cells; wherein each of the plurality of genetically engineered NK cells and the plurality of genetically engineered T cells are engineered to express a cytotoxic receptor comprising an extracellular ligand binding domain, a transmembrane domain, and a cytotoxic signaling complex, wherein the genetically engineered NK cells are also engineered to express membrane-bound IL15, wherein the genetically engineered immune cells are genetically engineered to express at least one immunosuppressive effector, wherein the at least one immunosuppressive effector exerts suppressive effects on undesired cytotoxic activity of suppressive cells, wherein the at least one immunosuppressive effector expressed by the plurality of genetically engineered T cells comprises HLA-E or a viral immunosuppressive peptide, wherein the HLA-E or the viral immunosuppressive peptide is configured to temporarily suppress NK cell activity against the genetically engineered T cells, wherein the NK cell activity arises either from host NK cells after administration to a subject or from the plurality of genetically engineered NK cells, and wherein the temporary suppression of activity of the genetically engineered NK cells reduces genetically engineered NK cell exhaustion and prolongs the persistence of the population of genetically engineered cells after administration.

In several embodiments, the is additionally provided a population of genetically engineered immune cells for cancer immunotherapy, comprising: a plurality of genetically engineered NK cells; wherein the plurality of genetically engineered immune cells are engineered to express a cytotoxic receptor, wherein the cytotoxic receptor comprises an extracellular ligand binding domain, a transmembrane domain, and a cytotoxic signaling complex, wherein the genetically engineered NK cells are engineered to express membrane bound IL-15, wherein the genetically engineered immune cells are genetically engineered to express at least one immunosuppressive effector, wherein the at least one immunosuppressive effector exerts suppressive effects on undesired cytotoxic activity of suppressive cells, wherein the genetically engineered immune cells exhibit one or more of enhanced expansion capability, enhanced cytotoxicity against target cells, and enhanced persistence, as compared to cells that do not comprise said immunosuppressive effector.

In several embodiments, the population, further comprises a second type of immune cells. In several embodiments, the second type of immune cells comprises genetically engineered T cells or other immune cells.

In several embodiments, there is also provided a population of genetically engineered immune cells for cancer immunotherapy, comprising genetically engineered immune cells that express a cytotoxic receptor comprising an extracellular ligand binding domain, a transmembrane domain, and a cytotoxic signaling complex, wherein the immune cells are genetically edited at one or more target sites in the genome of the immune cell to yield reduced levels of expression of a protein which is encoded by a gene which comprises an edited target site as compared to a non-edited immune cell, wherein the edits are made using a Crispr/Cas system, and wherein the genetically engineered immune cells are further engineered to express at least one immunosuppressive effector, wherein the at least one immunosuppressive effector exerts suppressive effects on the cytotoxic activity of natural killer cells and/or T cells, and wherein the genetically engineered and edited immune cells exhibit one or more of enhanced expansion capability, enhanced cytotoxicity against target cells, and enhanced persistence, as compared to cells that do not comprise said immunosuppressive effector and said edited gene.

In several embodiments, there is also provided a population of genetically engineered immune cells for cancer immunotherapy, comprising one or more of genetically engineered NK cells and genetically engineered T cells; wherein the genetically engineered immune cells are engineered to express a cytotoxic receptor, wherein the cytotoxic receptor comprises an extracellular ligand binding domain, a transmembrane domain, and a cytotoxic signaling complex, wherein the immune cells are genetically edited at one or more target sites in the genome of the immune cell to yield reduced levels of expression of a protein which is encoded by a gene which comprises an edited target site as compared to a non-edited immune cell, wherein the edits are made using an RNA-guided endonuclease, wherein the genetically engineered immune cells are further genetically engineered to express at least one immunosuppressive effector, wherein the at least one immunosuppressive effector exerts suppressive effects on the cytotoxic activity of undesired cells, wherein the undesired cells comprise one or more of non-engineered natural killer cells, non-engineered T cells, or undesired engineered cells, and wherein the genetically engineered and edited immune cells exhibit one or more of enhanced expansion capability, enhanced cytotoxicity against target cells, and enhanced persistence, as compared to cells that do not comprise said immunosuppressive effector and said edited gene.

In several embodiments, there is provided a population of genetically engineered immune cells for cancer immunotherapy, comprising one or more of genetically engineered NK cells and genetically engineered T cells; wherein the plurality of genetically engineered immune cells are engineered to express a cytotoxic receptor, wherein the cytotoxic receptor comprises an extracellular ligand binding domain, a transmembrane domain, and a cytotoxic signaling complex, wherein the immune cells are genetically edited at one or more target sites in the genome of the immune cell to yield reduced levels of expression of a protein which is encoded by a gene which comprises an edited target site as compared to a non-edited immune cell, immune cells are further genetically engineered to express at least one immunosuppressive effector, wherein the at least one immunosuppressive effector exerts suppressive effects on undesired cytotoxic activity of suppressive cells, wherein the genetically engineered immune cells exhibit one or more of enhanced expansion capability, enhanced cytotoxicity against target cells, and enhanced persistence, as compared to cells that do not comprise said immunosuppressive effector.

In several embodiments, the suppressive cells comprise host cells, non-engineered natural killer cells, non-engineered T cells, and/or suppressive engineered cells. In several embodiments, the suppressive engineered cells comprise the genetically engineered immune cells.

In several embodiments, the cells that do not comprise said immunosuppressive effector are either non-engineered or engineered cells.

In several embodiments, the genetically engineered immune cells are also genetically edited to reduce expression of beta-2 microglobulin (B2M), wherein the reduced expression of B2M enables the immune cells to be used in allogeneic cancer immunotherapy with reduced host versus graft rejection as compared to immune cells expressing endogenous levels of B2M.

In several embodiments, the cytotoxic receptor targets one or more of NKG2D, CD19, BCMA, CD70, and CD38 expressed by target tumor cells, and wherein the cytotoxic signaling complex comprises an OX40 subdomain or a 4-1 BB domain, and a CD3zeta subdomain.

In several embodiments, the at least one immunosuppressive effector comprises a virally-derived peptide, a peptide derived from a retrovirus, a peptide derived from an envelope protein of a retrovirus, at least a portion of a human protein and/or at least a portion of a human protein complex.

In several embodiments, the at least one immunosuppressive effector comprises a chimeric construct comprises at least one virally-derived peptide and at least a portion of a human protein and/or at least a portion of a human protein complex.

In several embodiments, the immunosuppressive effector is encoded by a nucleic acid or comprises a peptide having at least 85% sequence identity to one or more of the nucleotide or amino acid sequences of SEQ ID NOs: 683-894.

In several embodiments, the immunosuppressive effector comprises a peptide having at least 95% sequence identity to SEQ ID NO: 689.

In several embodiments, the immunosuppressive effector is encoded by a nucleic acid having at least 95% sequence identity to SEQ ID NO: 690.

In several embodiments, the immune cells comprise genetically engineered Natural Killer (NK) cells, genetically engineered T cells, or combinations thereof.

In several embodiments, the genetically engineered immune cells are suitable for use in allogeneic cancer cell therapy with reduced risk of graft versus host disease.

In several embodiments, the genetically engineered immune cells are suitable for use in allogeneic cancer cell therapy with reduced risk of cytotoxic activity between the genetically engineered immune cells.

In several embodiments, at least a portion of the genetically engineered immune cells are engineered to express membrane bound IL-15.

In several embodiments, at least about 60% of the NK cells are positive for NKG2A.

In several embodiments, at least about 80% of the NK cells are positive for NKG2A.

In several embodiments, the at least one immunosuppressive effector is integrated into the cytotoxic receptor: (i) between the transmembrane domain and the extracellular ligand-binding domain, (ii) within the extracellular ligand-binding domain, (iii) wherein the extracellular ligand-binding domain comprises an scFv and the at least one immunosuppressive effector is integrated into a linker region of the scFv, (iv) within an N-terminal region of the cytotoxic receptor distally positioned from the extracellular ligand-binding domain, and/or (v) at a plurality of locations within an extracellular region of the cytotoxic receptor.

In several embodiments, the at least one immunosuppressive effector is bound to an extracellular membrane of the immune cells.

In several embodiments, the at least one immunosuppressive effector comprises a transmembrane protein, wherein the transmembrane protein is selected from CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor.

In several embodiments, the immunosuppressive effector is expressed on the immune cells by a disulfide trap single chain trimer (dtSCT).

In several embodiments, there are provided methods for the treatment of cancer in a subject comprising administering to the subject genetically engineered immune cells according to any of embodiments disclosed herein. In several embodiments, there is provided a use of genetically engineered immune cells according to embodiments disclosed herein for the treatment of cancer or for the preparation of a medicament for the treatment of cancer.

In several embodiments, there is provided a method of manufacturing a population of genetically engineered immune cells for cancer immunotherapy, comprising contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell; contacting the population of immune cells with a polynucleotide encoding a cytotoxic receptor comprising an extracellular ligand binding domain, a transmembrane domain, and a cytotoxic signaling complex; contacting the population of immune cells with an additional polynucleotide encoding at least one immunosuppressive effector, wherein the immunosuppressive effector is encoded by a nucleic acid or comprises a peptide having at least 85% sequence identity to one or more of the nucleotide or amino acid sequences of SEQ ID NOs: 683-894; wherein the at least one immunosuppressive effector exerts suppressive effects on the cytotoxic activity of suppressive cells, and wherein the genetically engineered immune cells exhibit one or more of enhanced expansion capability, enhanced cytotoxicity against target cells, and enhanced persistence, as compared to cells that do not comprise said at least one immunosuppressive effector.

In several embodiments, the target of the genetic editing is one or more of a CISH gene, a B2M gene, a CD70 gene, an adenosine receptor gene, an NKG2A gene, a CIITA gene, a TGFBR gene, or any combination thereof.

Also, there is provided a method of engineering a population of genetically engineered immune cells for enhanced allogeneic cancer immunotherapy, comprising genetically editing a mixed population of immune cells comprising NK cells and T cells to disrupt Beta-2 microglobulin (B2M) expression and coordinately reduce expression Human Leukocyte Antigen (HLA) on the surface of the immune cells, wherein reduced expression of HLA on the surface of the immune cells reduces T cell-mediated cytotoxicity against the edited population of immune cells, wherein reduced expression of HLA on the surface of the cells renders the edited population of immune cells susceptible to NK-mediated cytotoxicity against the edited immune cells; wherein at least about 60% of the NK cells in the mixed population are positive for NKG2A expression; and genetically engineering the edited cells to express one or more immunosuppressive effectors that reduce NK-mediated cytotoxicity against the mixed population of immune cells, wherein the one or more immunosuppressive effectors comprises one or more of a viral immunosuppressive peptide, a viral protein that is an HLA homolog, HLA-E, HLA-G, a human protein or fragment thereof that reduces phagocytosis of cells, a chimeric construct comprising a viral immunosuppressive peptide and a human protein or fragment thereof that reduces phagocytosis of cells, or combinations thereof, wherein the reduced T cell-mediated cytotoxicity reduces engineered T cell-mediated fratricidal cytotoxicity against engineered NK cells and, upon administration, host T-cell mediated cytotoxicity against engineered NK cells, and wherein the wherein the reduced NK cell-mediated cytotoxicity reduces engineered NK cell-mediated fratricidal cytotoxicity against engineered T cells and, upon administration, host NK cell-mediated cytotoxicity against engineered NK and engineered T cells, thereby allowing for enhanced persistence of the engineering mixed population of immune cells upon administration to an allogeneic subject and allowing for enhanced allogeneic cancer immunotherapy.

In several embodiments, the method further comprises contacting the mixed population of immune cells with a polynucleotide encoding a cytotoxic receptor comprising an extracellular ligand binding domain, a transmembrane domain, and a cytotoxic signaling complex.

In several embodiments, the polynucleotide encodes a cytotoxic receptor targeting one or more of NKG2D, CD19, CD70, BCMA, and CD38 expressed by target tumor cells.

In several embodiments, the method further comprises genetically editing the DNA of the genetically engineered immune cells to alter the expression of one or more of a CISH gene, a CD70 gene, an adenosine receptor gene, an NKG2A gene, a CIITA gene, a TGFBR gene, or any combination thereof.

In several embodiments, wherein the gene editing to reduce expression or the gene editing to induce expression is made using a CRISPR-Cas system, wherein the Cas is Cas9, Cas12, Cas13, CasX or CasY.

Additionally provided is a population of genetically engineered immune cells, comprising one or more of genetically engineered NK cells and genetically engineered T cells; wherein at least about 70% of the genetically engineered NK cells express NKG2A, wherein the plurality of genetically engineered immune cells are engineered to express a cytotoxic receptor comprising an extracellular ligand binding domain, a transmembrane domain, and a cytotoxic signaling complex, wherein the genetically engineered immune cells are genetically engineered to express at least one immunosuppressive effector derived from a Human Leukocyte Antigen (HLA), wherein the at least one immunosuppressive effector exerts suppressive effects on undesired cytotoxic activity of suppressive cells, wherein, optionally, the genetically engineered immune cells are further engineered to express one or more of the following: IL-15, mblL-15, at least one viral immunosuppressive peptide integrated within an extracellular region, or extracellular regions, of the cytotoxic receptor, at least one viral immunosuppressive peptide expressed on a cell membrane of the genetically engineered immune cells, at least one portion of a human immunosuppressive protein or protein complex integrated within an extracellular region, or extracellular regions, of the cytotoxic receptor, at least one portion of a human immunosuppressive protein or protein complex expressed on a cell membrane of the genetically engineered immune cells, and/or at least one chimeric viral-human immunosuppressive construct expressed on a cell membrane of the genetically engineered immune cells and/or integrated at one or more extracellular regions of the cytotoxic receptor.

In several embodiments, the immunosuppressive effector derived from an HLA comprises an HLA-E peptide.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% sequence identity or homology includes 96%, 97%, 98%, 99%, and 100% sequence identity or homology to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence. Any titles or subheadings used herein are for organization purposes and should not be used to limit the scope of embodiments disclosed herein.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### Sequences

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, or other types of modifications. Additionally, it shall be appreciated that certain nucleic acid sequences, even if not expressly included within the sequence, comprise one of the three standard stop codons (TAA, TAG, or TGA) known in the standard genetic code.

In accordance with some embodiments described herein, any of the sequences may be used, or a truncated or mutated form of any of the sequences disclosed herein (and/or included in the accompanying sequence listing) may be used and in any combination.

1-100. (canceled)

101. A population of genetically engineered and gene edited immune cells, comprising:
genetically engineered immune cells that express a cytotoxic receptor comprising an extracellular ligand binding domain, a transmembrane domain, and a cytotoxic signaling complex,
wherein the extracellular ligand binding domain targets a tumor marker expressed by a target tumor cell,
wherein the immune cells are genetically edited at one or more target locations in a CISH gene that encodes a CIS protein, wherein the edits yield reduced expression and/or function of CIS as compared to an immune cell not edited at the location or locations in the CISH gene;
wherein the immune cells are genetically edited at one or more target locations in a Cbl proto-oncogene B protein (CBLB) gene that encodes CBLB protein, wherein the edits yield reduced expression and/or function of CBLB protein as compared to an immune cell not edited at the location or locations in the CBLB gene; and
wherein the genetically engineered and edited immune cells exhibit one or more of enhanced expansion capability, enhanced cytotoxicity against target cells, and enhanced persistence, as compared to immune cells that do not comprise said genetic edits at the CISH and CBLB genes.

102. The population of genetically engineered and gene edited immune cells of claim 101, wherein the immune cells are edited at one or more additional target sites in the genome of the immune cell to yield reduced levels of expression of the protein which is encoded by a gene which comprises an edited target site as compared to a non-edited immune cell.

103. The population of genetically engineered and gene edited immune cells of claim 101, wherein the genetically engineered and gene edited cells are edited at an additional target site in a gene encoding: transforming growth factor receptor beta 2 (TGFBR2), TIGIT, NKG2A, SOCS2, B2M, PD-1, TIM-3, or TCR alpha.

104. The population of genetically engineered and gene edited immune cells of claim 101, wherein:
(i) a guide sequence of any of SEQ ID NO: 153-157 or 562-565 is used to target the CISH gene; and/or
(ii) a guide sequence of any of SEQ ID NO: 164 to 166 or 552-555 is used to target the CBLB gene;

105. The population of genetically engineered and gene edited immune cells of claim 101, wherein the genetically engineered and gene edited immune cells express membrane-bound interleukin-15 (mbIL15).

106. The population of genetically engineered and gene edited immune cells of claim 101, wherein the cytotoxic signaling complex comprises an OX40 subdomain and CD3zeta subdomain.

107. The population of genetically engineered and gene edited immune cells of claim 101, wherein the genetically engineered and gene edited immune cells comprise Natural Killer (NK) cells, T cells, or combinations thereof.

108. The population of genetically engineered and gene edited immune cells of claim 101, wherein the cytotoxic receptor targets ligands of NKG2D, CD19, CD70, BCMA, CD38, GPRCSD, DLL3, EGFR, PSMA, or FLT3.

109. A method for the treatment of cancer in a subject comprising administering to the subject the population of genetically engineered and gene edited immune cells according to claim 101.

110. The method of claim 109, wherein the genetically engineered and gene edited immune cells express membrane-bound interleukin-15 (mbIL15).

111. The method of claim 109, wherein the genetically engineered and gene edited immune cells comprise Natural Killer (NK) cells.

112. The method of claim 111, wherein the genetically engineered and gene edited immune cells are allogeneic to the subject.

113. A method of manufacturing a population of genetically edited immune cells, comprising:
(a) contacting a population of immune cells with a first gene editing complex,
wherein the first gene editing complex edits at one or more target sites in a CISH gene of the immune cell to yield reduced levels of expression of CIS protein encoded by the CISH gene as compared to an immune cell not edited at the CISH gene; and
(b) contacting the population of immune cells with a second gene editing complex,
wherein the second gene editing complex edits at one or more target sites in:
(i) a CBLB gene of the immune cell to yield reduced levels of expression of CBLB protein encoded by the CBLB gene as compared to an immune cell not edited at the CBLB gene;
(ii) a TGFBR2 gene of the immune cell to yield reduced levels of expression of TGFBR2 protein encoded by the TGFBR2 gene as compared to an immune cell not edited at the TGFBR2 gene;
(iii) a TIGIT gene of the immune cell to yield reduced levels of expression of TIGIT protein encoded by the TIGIT gene as compared to an immune cell not edited at the TIGIT gene; or
(iv) an additional gene of the immune cell to yield reduced levels of expression of a protein encoded by the additional gene as compared to an immune cell not edited at the additional gene; and
wherein the genetically edited immune cells exhibit one or more of enhanced expansion capability, enhanced cytotoxicity against target cells, and enhanced persistence, as compared to immune cells that do not comprise said genetically edited target site or sites.

114. The method of claim 113, further comprising contacting the population of immune cells with a vector comprising a polynucleotide encoding a cytotoxic receptor comprising an extracellular ligand binding domain, a transmembrane domain, and a cytotoxic signaling complex.

115. The method of claim 113, wherein the immune cells comprise Natural Killer (NK) cells, T cells, or combinations thereof.

116. A population of genetically engineered and gene edited immune cells, comprising:
genetically engineered immune cells that express a cytotoxic receptor comprising an extracellular ligand binding domain, a transmembrane domain, and a cytotoxic signaling complex,
wherein the extracellular ligand binding domain targets a tumor marker selected from the group consisting of NKG2D ligands, CD19, CD70, BCMA, CD38, GPRC5D, CD138 DLL3, EGFR, PSMA, FLT3, and KREMEN2,
wherein at least a portion of the genetically engineered immune cells are engineered to express membrane bound IL-15,
wherein the immune cells are genetically edited at one or more target sites in the genome of the immune cell to yield reduced levels of expression of the protein which is encoded by a gene which comprises an edited target site as compared to a non-edited immune cell,
wherein the protein is selected from the group consisting of CISH, adenosine A2 receptor, SMAD3, MAPKAPK3, CEACAM1, DDIT4, TGFBR2R, NKG2A, SOCS2, CBLB, B2M, TIGIT, PD-1, TIM-3, CD38, TCR alpha, and any combination thereof, and
wherein the genetically engineered and edited immune cells exhibit one or more of enhanced expansion capability, enhanced cytotoxicity against target cells, and enhanced persistence, as compared to immune cells that do not comprise said genetically edited target site or sites.

117. A method for the treatment of cancer in a subject comprising administering to the subject at least a portion of the population of immune cells according to claim 116.

118. A population of genetically engineered immune cells, comprising:
genetically engineered immune cells that express a cytotoxic receptor comprising an extracellular ligand binding domain, a transmembrane domain, and a cytotoxic signaling complex,
wherein at least a portion of the genetically engineered immune cells are engineered to express membrane bound IL-15,
wherein the genetically engineered immune cells are engineered to express at least one immunosuppressive effector comprising: (i) two or more of a truncated human CD47, a p15E peptide, an HIV peptide, and an HTLV peptide, (ii) a truncated human CD47 domain and at least one p15E peptide, an HIV peptide, and an HTLV peptide, (iii) a truncated human CD47 domain and at least one HIV peptide, (iv) a truncated human CD47 domain and at least HTLV peptide, (v) a sequence having at least 95% sequence identity to one or more of SEQ ID Nos: 256, 259, 262, 265, 268, 271, or (vi) a viral UL18 protein,
wherein the at least one immunosuppressive effector exerts suppressive effects on undesired cytotoxic activity of suppressive cells, and
wherein the genetically engineered immune cells exhibit one or more of enhanced expansion capability, enhanced cytotoxicity against target cells, and enhanced persistence, as compared to cells that do not comprise said immunosuppressive effector.

119. A method of engineering a population of genetically engineered immune cells, comprising:
(a) genetically editing a mixed population of immune cells comprising NK cells and T cells to reduce expression Human Leukocyte Antigen (HLA) on the surface of the immune cells,
wherein reduced expression of HLA on the surface of the immune cells reduces T cell-mediated cytotoxicity against the edited population of immune cells,
wherein reduced expression of HLA on the surface of the cells renders the edited population of immune cells susceptible to NK-mediated cytotoxicity against the edited immune cells; and
(b) genetically engineering the edited cells to express one or more immunosuppressive effectors that reduce NK-mediated cytotoxicity against the mixed population of immune cells,
wherein the one or more immunosuppressive effectors comprises one or more of a viral immunosuppressive peptide, a viral protein that is an HLA homolog, HLA-E, HLA-G, a human protein or fragment thereof that reduces phagocytosis of cells, a chimeric construct comprising a viral immunosuppressive peptide and a human protein or fragment thereof that reduces phagocytosis of cells, or combinations thereof,
wherein the reduced T cell-mediated cytotoxicity reduces engineered T cell-mediated fratricidal cytotoxicity against engineered NK cells and, upon administration to a subject, host T-cell mediated cytotoxicity against engineered NK cells, and
wherein the reduced NK cell-mediated cytotoxicity reduces engineered NK cell-mediated fratricidal cytotoxicity against engineered T cells and, upon administration to a subject, host NK cell-mediated cytotoxicity against engineered NK and engineered T cells.

120. The method of claim 119, wherein the immune cells comprise Natural Killer (NK) cells.
